SEQUENCE LISTING (715 sequences)

SEQ ID NO: 1 - protein, 274 aa, Artificial Sequence
  Description of Artificial Sequence: Synthetic polypeptide
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNSDSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 2 - protein, 310 aa, Artificial Sequence
  Description of Artificial Sequence: Synthetic polypeptide
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSLGGESEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 3 - protein, 274 aa, Artificial Sequence
  Description of Artificial Sequence: Synthetic polypeptide
MMKSLRVLLVILWLQLSWVWSQQKEVEQNSGPLSVPEGAIASLNCTYSDRGSQAFFWYRQYSGKSPELIMSIYSNGDKEDGRFTAQLNKASQYVSLLIRDSQPSDSATYLCAVNSQSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 4 - protein, 274 aa, Artificial Sequence
  Description of Artificial Sequence: Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 5 - protein, 310 aa, Artificial Sequence
  Description of Artificial Sequence: Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSR

SEQ ID NO: 6 - protein, 205 aa, Artificial Sequence
  Description of Artificial Sequence: Synthetic polypeptide
QKEVEQNSGPLSVPEGAIASLNCTYSDRGSQAFFWYRQYSGKSPELIMSIYSNGDKEDGRFTAQLNKASQYVSLLIRDSQPSDSATYLCAVNSDSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 7 - protein, 205 aa, Artificial Sequence
  Description of Artificial Sequence: Synthetic polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSDSSYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 8 - protein, 205 aa, Artificial Sequence
  Description of Artificial Sequence: Synthetic polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSDSGVALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 9 - protein, 205 aa, Artificial Sequence
  Description of Artificial Sequence: Synthetic polypeptide
QKEVEQNSGPLSVPEGAIASLNCTYSDRGSQAFFWYRQYSGKSPELIMSIYSNGDKEDGRFTAQLNKASQYVSLLIRDSQPSDSATYLCAVNSDSGVALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 10 - protein, 205 aa, Artificial Sequence
  Description of Artificial Sequence: Synthetic polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSQSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 11 - protein, 205 aa, Artificial Sequence
  Description of Artificial Sequence: Synthetic polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSQSGYSLNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 12 - protein, 205 aa, Artificial Sequence
  Description of Artificial Sequence: Synthetic polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSQSSYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 13 - protein, 205 aa, Artificial Sequence
  Description of Artificial Sequence: Synthetic polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSQSGVALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 14 - protein, 205 aa, Artificial Sequence
  Description of Artificial Sequence: Synthetic polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSQNGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 15 - protein, 205 aa, Artificial Sequence
  Description of Artificial Sequence: Synthetic polypeptide
QKEVEQNSGPLSVPEGAIASLNCTYSDRGSFSFFWYRQYSGKSPELIMSIYSNGDKEDGRFTAQLNKASQYVSLLIRDSQPSDSATYLCAVNSDSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 16 - protein, 205 aa, Artificial Sequence
  Description of Artificial Sequence: Synthetic polypeptide
QKEVEQNSGPLSVPEGAIASLNCTYSDRGSYSFFWYRQYSGKSPELIMSIYSNGDKEDGRFTAQLNKASQYVSLLIRDSQPSDSATYLCAVNSDSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 17 - protein, 205 aa, Artificial Sequence
  Description of Artificial Sequence: Synthetic polypeptide
QKEVEQNSGPLSVPEGAIASLNCTYSDRGSYSFFWYRQYSGKSPELIMSIYSNGDKEDGRFTAQLNKASQYVSLLIRDSQPSDSATYLCAVNSDSSYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 18 - protein, 204 aa, Artificial Sequence
  Description of Artificial Sequence: Synthetic polypeptide
KEVEQNSGPLSVPEGAIASLNCTYSDRGSYSFFWYRQYSGKSPELIMSIYSNGDKEDGRFTAQLNKASQYVSLLIRDSQPSDSATYLCAVNSDSGVALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 19 - protein, 205 aa, Artificial Sequence
  Description of Artificial Sequence: Synthetic polypeptide
QKEVEQNSGPLSVPEGAIASLNCTYSDRGSYSFFWYRQYSGKSPELIMSIYSNGDKEDGRFTAQLNKASQYVSLLIRDSQPSDSATYLCAVNSQSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 20 - protein, 113 aa, Artificial Sequence
  Description of Artificial Sequence: Synthetic polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVWGKSGGYNKLIFGAGTRLAVHP

SEQ ID NO: 21 - protein, 117 aa, Artificial Sequence
  Description of Artificial Sequence: Synthetic polypeptide
NAGVTQTPKFGILKTGQSMTLQCAQDMNHEYMSWYRQDPGMGLRLIHYSVGAGITDKGEVPNGYNVSRSTTEDFPLRLELAAPSQTSVYFCASSYSSPLRQDNSPLHFGNGTRLTVT

SEQ ID NO: 22 - protein, 111 aa, Artificial Sequence
  Description of Artificial Sequence: Synthetic polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSPGNARLMFGDGTQLVVKP

SEQ ID NO: 23 - protein, 113 aa, Artificial Sequence
  Description of Artificial Sequence: Synthetic polypeptide
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYWRGGLEQYFGPGTRLTVT

SEQ ID NO: 24 - protein, 112 aa, Artificial Sequence
  Description of Artificial Sequence: Synthetic polypeptide
AQSVTQLDSHVSVSEGTPVLLRCNYSSSVPPYLFWYVQYPNQGLQLLLKYTTGATLVKGINGFEAEFKKSETSFHLTKPSAHMSDAAEYFCAVSEDGNKLVFGAGTILRVKS

SEQ ID NO: 25 - protein, 117 aa, Artificial Sequence
  Description of Artificial Sequence: Synthetic polypeptide
AAGVIQSPRHLIKEKRETATLKCYPIPRHDTVYWYQQGPGQDPQFLISFYEKMQSDKGSIPDRFSAQQFSDYHSELNMSSLELGDSALYFCASRLTGVSGEGDYGYTFGSGTRLTVV

SEQ ID NO: 26 - protein, 9 aa, Artificial Sequence
  Description of Artificial Sequence: Synthetic peptide
PLFQVPEPV

SEQ ID NO: 27 - protein, 9 aa, Artificial Sequence
  Description of Artificial Sequence: Synthetic peptide
FMNKFIYEI

SEQ ID NO: 28 - protein, 10 aa, Artificial Sequence
  Description of Artificial Sequence: Synthetic peptide
GLSPNLNRFL

SEQ ID NO: 29 - protein, 9 aa, Artificial Sequence
  Description of Artificial Sequence: Synthetic peptide
GVALQTMKQ

SEQ ID NO: 30 - protein, 9 aa, Artificial Sequence
  Description of Artificial Sequence: Synthetic peptide
AMNKFIYEI

SEQ ID NO: 31 - protein, 9 aa, Artificial Sequence
  Description of Artificial Sequence: Synthetic peptide
FMAKFIYEI

SEQ ID NO: 32 - protein, 9 aa, Artificial Sequence
  Description of Artificial Sequence: Synthetic peptide
FMNAFIYEI

SEQ ID NO: 33 - protein, 9 aa, Artificial Sequence
  Description of Artificial Sequence: Synthetic peptide
FMNKAIYEI

SEQ ID NO: 34 - protein, 9 aa, Artificial Sequence
  Description of Artificial Sequence: Synthetic peptide
FMNKFAYEI

SEQ ID NO: 35 - protein, 9 aa, Artificial Sequence
  Description of Artificial Sequence: Synthetic peptide
FMNKFIAEI

SEQ ID NO: 36 - protein, 9 aa, Artificial Sequence
  Description of Artificial Sequence: Synthetic peptide
FMNKFIYAI

SEQ ID NO: 37 - protein, 9 aa, Artificial Sequence
  Description of Artificial Sequence: Synthetic peptide
SLLMWITQC

SEQ ID NO: 38 - protein, 10 aa, Artificial Sequence
  Description of Artificial Sequence: Synthetic peptide
FLTPKKLQCV

SEQ ID NO: 39 - protein, 10 aa, Artificial Sequence
  Description of Artificial Sequence: Synthetic peptide
VISNDVCAQV

SEQ ID NO: 40 - protein, 12 aa, Artificial Sequence
  Description of Artificial Sequence: Synthetic peptide
KVMDLPTQEPAL

SEQ ID NO: 41 - protein, 124 aa, Artificial Sequence
  Description of Artificial Sequence: Synthetic polypeptide
EVQLLESGGGLVQPGGSLRLSCAASGFTFSSYAMTWVRQAPGKGLEWVSAISGSGYSTYYADSEKGRFTISRDNSKNTLYLQMNSLRAEDTAVYYCAKGFQYGSGSYYTHFDYWGQGTLVTVSS

SEQ ID NO: 42 - protein, 107 aa, Artificial Sequence
  Description of Artificial Sequence: Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 43 - protein, 291 aa, Artificial Sequence
  Description of Artificial Sequence: Synthetic polypeptide
MKYLLPTAAAGLLLLAAQPAMAQVQLVQSGGGLVQPGRSLRLSCAASGFTFDDYAMHWVRQAPGKGLEWVSGISWNSGSIGYADSVKGRFTISRDNAKNSLYLQMNSLRPEDTAVYYCARDLGAKQWLEGFDYWGQGTLVTVSSGGGGSGGGGSGGGGSNFMLTQDPAVSVALGQTVRITCQGDSLRSYYASWYQQKPGQAPVLVIYGKNNRPSGIPDRFSGSTSGNSASLTITGAQAEDEADYYCNSRDSSGNHWVFGGGTKVTVLGAAAEQKLISEEDLNGAAHHHHHH

SEQ ID NO: 44 - protein, 472 aa, Artificial Sequence [sequence omitted]

SEQ ID NO: 45 - protein, 5 aa, Artificial Sequence
  Description of Artificial Sequence: Synthetic peptide
DYYMN

SEQ ID NO: 46 - protein, 19 aa, Artificial Sequence
  Description of Artificial Sequence: Synthetic peptide
LIRNKANGYTTEYNASVKG

SEQ ID NO: 47 - protein, 12 aa, Artificial Sequence
  Description of Artificial Sequence: Synthetic peptide
DSDGYYEYYFDY

SEQ ID NO: 48 - protein, 236 aa, Artificial Sequence
  Description of Artificial Sequence: Synthetic polypeptide
MDMRVPAHVFGFLLLWFPGTRCDIQMTQSPSSLSASLGERVSLTCRASQEISDYLSWLQQKPDGTIKRLIFAASTLDSGVPKRFSGSRSGSDFSLSISSLESEDFADYYCLQYASYPYTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 11 aa, Artificial Sequence
  Description of Artificial Sequence: Synthetic peptide
RASQEISDYLS

SEQ ID NO: 50 - protein, 7 aa, Artificial Sequence
  Description of Artificial Sequence: Synthetic peptide
AASTLDS

SEQ ID NO: 51 - protein, 9 aa, Artificial Sequence
  Description of Artificial Sequence: Synthetic peptide
LQYASYPYT

SEQ ID NO: 52 - protein, 480 aa, Artificial Sequence
  Description of Artificial Sequence: Synthetic polypeptide
MKHLWFFLLLVAAPRWVLSQVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARDGPLGYCSSTSCPVTGEYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 53 - protein, 7 aa, Artificial Sequence
  Description of Artificial Sequence: Synthetic peptide
SGGYYWS

SEQ ID NO: 54 - protein, 16 aa, Artificial Sequence
  Description of Artificial Sequence: Synthetic peptide
YIYYSGSTYYNPSLKS

SEQ ID NO: 55 - protein, 25 aa, Artificial Sequence
  Description of Artificial Sequence: Synthetic peptide
LGPLGYCSSTSCPVTGEYYYYGMDV

SEQ ID NO: 56 - protein, 236 aa, Artificial Sequence
  Description of Artificial Sequence: Synthetic polypeptide
METPAQLLFLLLLWLPDTTGEIVLTQSPGTLSLSPGERATLSCRASQSVSNNYLAWYQQKPGQAPRLLIFGASSRATGIPDRFSGSGSGTDFTLTISRLEPEDFAVYYCQQYDISPMYSFGQGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 12 aa, Artificial Sequence
  Description of Artificial Sequence: Synthetic peptide
RASQSVSNNYLA

SEQ ID NO: 58 - protein, 7 aa, Artificial Sequence
  Description of Artificial Sequence: Synthetic peptide
GASSRAT

SEQ ID NO: 59 - protein, 10 aa, Artificial Sequence
  Description of Artificial Sequence: Synthetic peptide
QQYDISPMYS

SEQ ID NO: 60 - protein, 120 aa, Artificial Sequence
  Description of Artificial Sequence: Synthetic polypeptide
EVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSS

SEQ ID NO: 61 - protein, 113 aa, Artificial Sequence
  Description of Artificial Sequence: Synthetic polypeptide
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIK

SEQ ID NO: 62 - protein, 137 aa, Artificial Sequence
  Description of Artificial Sequence: Synthetic polypeptide
MAWVWTLLFLMAAAQSAGAQIQLVQSGPEVKKPGETVKISCKASGYTFTNYGMNWVKQAPGKGLKWMGWINTYTGEPTYADAFKGRFAFSLETSASTAYLQINNLKNEDTATYFCARDYGDYGMDYWGQGTSVTVSS

SEQ ID NO: 63 - protein, 10 aa, Artificial Sequence
  Description of Artificial Sequence: Synthetic peptide
GYTFTNYGMN

SEQ ID NO: 64 - protein, 16 aa, Artificial Sequence
  Description of Artificial Sequence: Synthetic peptide
WINTYTGEPTYADAFK

SEQ ID NO: 65 - protein, 9 aa, Artificial Sequence
  Description of Artificial Sequence: Synthetic peptide
DYGDYGMDY

SEQ ID NO: 66 - protein, 132 aa, Artificial Sequence
  Description of Artificial Sequence: Synthetic polypeptide
METDTLLLWVLLLWVPGSTGDIVLTQSPASLAVSLGQRATISCRASKSVSTSGYSFMHWYQQKPGQPPKLLIYLASNLESGVPARFSGSGSGTDFTLNIHPVEEEDAATYYCQHSREVPWTFGGGTKLEIKR

SEQ ID NO: 67 - protein, 15 aa, Artificial Sequence
  Description of Artificial Sequence: Synthetic peptide
RASKSVSTSGYSFMH

SEQ ID NO: 68 - protein, 7 aa, Artificial Sequence
  Description of Artificial Sequence: Synthetic peptide
LASNLES

SEQ ID NO: 69 - protein, 9 aa, Artificial Sequence
  Description of Artificial Sequence: Synthetic peptide
QHSREVPWT

SEQ ID NO: 70 - protein, 119 aa, Artificial Sequence
  Description of Artificial Sequence: Synthetic polypeptide
QVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTTVTVSS

SEQ ID NO: 71 - protein, 7 aa, Artificial Sequence
  Description of Artificial Sequence: Synthetic peptide
NNNYYWT

SEQ ID NO: 72 - protein, 16 aa, Artificial Sequence
  Description of Artificial Sequence: Synthetic peptide
YIYYSGSTFYNPSLKS

SEQ ID NO: 73 - protein, 9 aa, Artificial Sequence
  Description of Artificial Sequence: Synthetic peptide
EDTMTGLDV

SEQ ID NO: 74 - protein, 106 aa, Artificial Sequence
  Description of Artificial Sequence: Synthetic polypeptide
DIQMTQSPSSLSASVGDRVTITCRASQSINNYLNWYQQKPGKAPTLLIYAASSLQSGVPSRFSGSRSGTDFTLTISSLQPEDFAAYFCQQTYSNPTFGQGTKVEVK

SEQ ID NO: 75 - protein, 11 aa, Artificial Sequence
  Description of Artificial Sequence: Synthetic peptide
RASQSINNYLN

SEQ ID NO: 76 - protein, 7 aa, Artificial Sequence
  Description of Artificial Sequence: Synthetic peptide
AASSLQS

SEQ ID NO: 77 - protein, 8 aa, Artificial Sequence
  Description of Artificial Sequence: Synthetic peptide
QQTYSNPT

SEQ ID NO: 78 - protein, 119 aa, Artificial Sequence
  Description of Artificial Sequence: Synthetic polypeptide
EVQLVESGGGLVQPGGSLRLSCAVSGFSLTNYGVHWVRQATGKGLEWLGVIWSGGNTDYNTPFTSRLTISKENAKNSVYLQMNSLRAGDTAVYYCARALTYYDYEFAYWGQGTMVTVSS

SEQ ID NO: 79 - protein, 5 aa, Artificial Sequence
  Description of Artificial Sequence: Synthetic peptide
NYGVH

SEQ ID NO: 80 - protein, 16 aa, Artificial Sequence
  Description of Artificial Sequence: Synthetic peptide
GVIWSGGNTDYNTPFT

SEQ ID NO: 81 - protein, 13 aa, Artificial Sequence
  Description of Artificial Sequence: Synthetic peptide
RALTYYDYEFAYW

SEQ ID NO: 82 - protein, 107 aa, Artificial Sequence
  Description of Artificial Sequence: Synthetic polypeptide
EIVLTQSPATLSLSPGERATLSCRASQSIGTNIHWYQQRPGQAPRLLIYYASESISGIPARFSGSGSGTDFTLTISSLEPEDFAVYYCQQNNNWPTTFGGGTKVEIK

SEQ ID NO: 83 - protein, 11 aa, Artificial Sequence
  Description of Artificial Sequence: Synthetic peptide
RASQSIGTNIH

SEQ ID NO: 84 - protein, 7 aa, Artificial Sequence
  Description of Artificial Sequence: Synthetic peptide
YASESIS

SEQ ID NO: 85 - protein, 7 aa, Artificial Sequence
  Description of Artificial Sequence: Synthetic peptide
QQNNNWP

SEQ ID NO: 86 - protein, 242 aa, Artificial Sequence
  Description of Artificial Sequence: Synthetic polypeptide
QVKLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEIK

SEQ ID NO: 87 - protein, 9 aa, Artificial Sequence
  Description of Artificial Sequence: Synthetic peptide
GYSSTSYAM

SEQ ID NO: 88 - protein, 9 aa, Artificial Sequence
  Description of Artificial Sequence: Synthetic peptide
LQSFNVPLT

SEQ ID NO: 89 - protein, 15 aa, Artificial Sequence
  Description of Artificial Sequence: Synthetic peptide
KTITATGVLFVRLGP

SEQ ID NO: 90 - protein, 118 aa, Artificial Sequence
  Description of Artificial Sequence: Synthetic polypeptide
EVQLVQSGAEVKKPGESLKISCQGSGYRFSKYWIGWVRQMPGKGLEWMGIIYPGDSDTRYSPSFQGQVTISADKSISTAYLQWSSLKASDTAMYYCARSFSSFIYDYWGQGTLVTVSS

SEQ ID NO: 91 - protein, 8 aa, Artificial Sequence
  Description of Artificial Sequence: Synthetic peptide
GYRFSKYW

SEQ ID NO: 92 - protein, 8 aa, Artificial Sequence
  Description of Artificial Sequence: Synthetic peptide
IYPGDSDT

SEQ ID NO: 93 - protein, 11 aa, Artificial Sequence
  Description of Artificial Sequence: Synthetic peptide
ARSFSSFIYDY

SEQ ID NO: 94 - protein, 116 aa, Artificial Sequence
  Description of Artificial Sequence: Synthetic polypeptide
QVQLVESGAEVKKPGESLKISCKASGYSFSNYWIGWVRQMPGKGLEWMGIIYPDDSDTRYSPSFQGQVTISADKSISTAYLQWYSLKVADTAKYYCVRPRGAFDIWGQGTTVTVSS

SEQ ID NO: 95 - protein, 8 aa, Artificial Sequence
  Description of Artificial Sequence: Synthetic peptide
GYSFSNYW

SEQ ID NO: 96 - protein, 8 aa, Artificial Sequence
  Description of Artificial Sequence: Synthetic peptide
IYPDDSDT

SEQ ID NO: 97 - protein, 9 aa, Artificial Sequence
  Description of Artificial Sequence: Synthetic peptide
VRPRGAFDI

SEQ ID NO: 98 - protein, 121 aa, Artificial Sequence
  Description of Artificial Sequence: Synthetic polypeptide
EVQLVESGPGLVKPSQTLSLTCTVSGGSISSGGYYWSWIRQHPGKGLEWIGYIYYSGSTYYNPSLKSRVTISVDTSKNQFSLKLSSVTAADTAMYYCARGGLYWTYSQDVWGQGTLVTVSS

SEQ ID NO: 99 - protein, 10 aa, Artificial Sequence
  Description of Artificial Sequence: Synthetic peptide
GGSISSGGYY

SEQ ID NO: 100 - protein, 7 aa, Artificial Sequence
  Description of Artificial Sequence: Synthetic peptide
IYYSGST

SEQ ID NO: 101 - protein, 13 aa, Artificial Sequence
  Description of Artificial Sequence: Synthetic peptide
ARGGLYWTYSQDV

SEQ ID NO: 102 - protein, 121 aa, Artificial Sequence
  Description of Artificial Sequence: Synthetic polypeptide
QITLKESGPELVKPTQTLTLTCTFSGFSLSTSGMSVSWIRQPPGKALEWLARIDWDDDKYYSTSLKTRLTISKDTSKNQVVLTMTNTDPVDTATYYCARGFYLAYGSYDSWGQGTLVTVSS

SEQ ID NO: 103 - protein, 10 aa, Artificial Sequence
  Description of Artificial Sequence: Synthetic peptide
GFSLSTSGMS

SEQ ID NO: 104 - protein, 7 aa, Artificial Sequence
  Description of Artificial Sequence: Synthetic peptide
IDWDDDK

SEQ ID NO: 105 - protein, 13 aa, Artificial Sequence
  Description of Artificial Sequence: Synthetic peptide
ARGFYLAYGSYDS

SEQ ID NO: 106 - protein, 120 aa, Artificial Sequence
  Description of Artificial Sequence: Synthetic polypeptide
EVQLVQSGAEVKKPGASVKVSCKASGYTFTNYYIHWVRQAPGQGLEWMGIINPTSGRTRYAQRFQGRVTMTRDTSTNTVYMDLSSLRSEDTAMYYCARSGYYWGVNGDQWGQGTLVTVSS

SEQ ID NO: 107 - protein, 8 aa, Artificial Sequence
  Description of Artificial Sequence: Synthetic peptide
GYTFTNYY

SEQ ID NO: 108 - protein, 8 aa, Artificial Sequence
  Description of Artificial Sequence: Synthetic peptide
INPTSGRT

SEQ ID NO: 109 - protein, 13 aa, Artificial Sequence
  Description of Artificial Sequence: Synthetic peptide
ARSGYYWGVNGDQ

SEQ ID NO: 110 - protein, 108 aa, Artificial Sequence
  Description of Artificial Sequence: Synthetic polypeptide
ETTLTQSPGTLSVSPGERATLSCRASQSVSSNLAWYQQKRGQAPRLLIYGASTRATGIPVRFSGSGSGTEFTLTISRLEPEDFAVYYCQQYGRSPLTFGGGTKVDIKR

SEQ ID NO: 111 - protein, 6 aa, Artificial Sequence
  Description of Artificial Sequence: Synthetic peptide
QSVSSN

SEQ ID NO: 112 - protein, 3 aa, Artificial Sequence
  Description of Artificial Sequence: Synthetic peptide
GAS

SEQ ID NO: 113 - protein, 9 aa, Artificial Sequence
  Description of Artificial Sequence: Synthetic peptide
QQYGRSPLT

SEQ ID NO: 114 - protein, 112 aa, Artificial Sequence
  Description of Artificial Sequence: Synthetic polypeptide
QSVLTQPPSVSGAPGQRVTISCTGSSSNIGAGHAVHWYQQLPGTAPKLLIYDNANRPSGVPDRFSGSQSGTSASLAITGLQTGDEADYYCGTWDDSPSAYVFGTGTKVTVLG

SEQ ID NO: 115 - protein, 9 aa, Artificial Sequence
  Description of Artificial Sequence: Synthetic peptide
SSNIGAGHA

SEQ ID NO: 116 - protein, 3 aa, Artificial Sequence
  Description of Artificial Sequence: Synthetic peptide
DNA

SEQ ID NO: 117 - protein, 11 aa, Artificial Sequence
  Description of Artificial Sequence: Synthetic peptide
GTWDDSPSAYV

SEQ ID NO: 118 - protein, 111 aa, Artificial Sequence
  Description of Artificial Sequence: Synthetic polypeptide
QPVLTQPPSASGTPGQRVTISCSGSSSNIGSDYVSWYQQLPGTAPKLLIYRNDQRPSGVPDRFSGSKSGTSASLAISGLRSEDEADYYCVAWDDSLSGYVFGSGTKVTVLG

SEQ ID NO: 119 - protein, 8 aa, Artificial Sequence
  Description of Artificial Sequence: Synthetic peptide
SSNIGSDY

SEQ ID NO: 120 - protein, 3 aa, Artificial Sequence
  Description of Artificial Sequence: Synthetic peptide
RND

SEQ ID NO: 121 - protein, 11 aa, Artificial Sequence
  Description of Artificial Sequence: Synthetic peptide
VAWDDSLSGYV

SEQ ID NO: 122 - protein, 110 aa, Artificial Sequence
  Description of Artificial Sequence: Synthetic polypeptide
QSALTQPASVSGSPGQSITISCTGTSGDVGGYNYVSWYQHHPGKAPKLIIYDVNKRPSGFSDRFSGSKSGNTASLTISGLQAEDEADYYCSSYTSTSTVFGGGTKLTVLG

SEQ ID NO: 123 - protein, 9 aa, Artificial Sequence
  Description of Artificial Sequence: Synthetic peptide
SGDVGGYNY

SEQ ID NO: 124 - protein, 3 aa, Artificial Sequence
  Description of Artificial Sequence: Synthetic peptide
DVN

SEQ ID NO: 125 - protein, 9 aa, Artificial Sequence
  Description of Artificial Sequence: Synthetic peptide
SSYTSTSTV

SEQ ID NO: 126 - protein, 107 aa, Artificial Sequence
  Description of Artificial Sequence: Synthetic polypeptide
SYELTQPLSVSVALGQTARITCGGNNIGSKNVHWYQQKPGQAPVLVIYRDSNRPSGIPERFSGSNSGNTATLTISRAQAGDEADYYCQVWDSSIVVFGGGTKLTVLG

SEQ ID NO: 127 - protein, 6 aa, Artificial Sequence
  Description of Artificial Sequence: Synthetic peptide
NIGSKN

SEQ ID NO: 128 - protein, 3 aa, Artificial Sequence
  Description of Artificial Sequence: Synthetic peptide
RDS

SEQ ID NO: 129 - protein, 9 aa, Artificial Sequence
  Description of Artificial Sequence: Synthetic peptide
QVWDSSIVV

SEQ ID NO: 130 - protein, 115 aa, Artificial Sequence
  Description of Artificial Sequence: Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARQSYITDSWGQGTLVTVSS

SEQ ID NO: 131 - protein, 8 aa, Artificial Sequence
  Description of Artificial Sequence: Synthetic peptide
GGSFSGYY

SEQ ID NO: 132 - protein, 7 aa, Artificial Sequence
  Description of Artificial Sequence: Synthetic peptide
INHSGST

SEQ ID NO: 133 - protein, 9 aa, Artificial Sequence
  Description of Artificial Sequence: Synthetic peptide
ARQSYITDS

SEQ ID NO: 134 - protein, 120 aa, Artificial Sequence
  Description of Artificial Sequence: Synthetic polypeptide
QVQLQQWGAGLLKPSETLSLTCAVYGGSFSGYYWSWIRQPPGKGLEWIGEINHSGSTNYNPSLKSRIIMSVDTSKRQFSLKLRSATAADTAVYYCARWSPFSYKQMYDYWGQGTLVTVSS

SEQ ID NO: 135 - protein, 8 aa, Artificial Sequence
  Description of Artificial Sequence: Synthetic peptide
GGSFSGYY

SEQ ID NO: 136 - protein, 7 aa, Artificial Sequence
  Description of Artificial Sequence: Synthetic peptide
INHSGST

SEQ ID NO: 137 - protein, 14 aa, Artificial Sequence
  Description of Artificial Sequence: Synthetic peptide
ARWSPFSYKQMYDY

SEQ ID NO: 138 - protein, 108 aa, Artificial Sequence
  Description of Artificial Sequence: Synthetic polypeptide
DIQLTQSPSAVSASVGDRVTITCRASQDVSKWLAWYQQKPGKAPRLLISAASGLQSWVPSRFSGSGSGTEFTLSISSLQPEDFATYYCQQANSFPWTFGQGTKVEIKR

SEQ ID NO: 139 - protein, 6 aa, Artificial Sequence
  Description of Artificial Sequence: Synthetic peptide
QDVSKW

SEQ ID NO: 140 - protein, 3 aa, Artificial Sequence
  Description of Artificial Sequence: Synthetic peptide
AAS

SEQ ID NO: 141 - protein, 9 aa, Artificial Sequence
  Description of Artificial Sequence: Synthetic peptide
QQANSFPWT

SEQ ID NO: 142 - protein, 112 aa, Artificial Sequence
  Description of Artificial Sequence: Synthetic polypeptide
NFMLTQPHSVSESPGKTVTISCTRSRGSIASAYVQWYQQRPGSAPITVIYEDYERPSEIPDRFSGSIDSSSNSASLTISGLKTEDEADYYCQSYDDNDHVIFGGGTKVTVLG

SEQ ID NO: 143 - protein, 8 aa, Artificial Sequence
  Description of Artificial Sequence: Synthetic peptide
RGSIASAY

SEQ ID NO: 144 - protein, 3 aa, Artificial Sequence
  Description of Artificial Sequence: Synthetic peptide
EDY

SEQ ID NO: 145 - protein, 10 aa, Artificial Sequence
  Description of Artificial Sequence: Synthetic peptide
QSYDDNDHVI

SEQ ID NO: 146 - protein, 123 aa, Artificial Sequence
  Description of Artificial Sequence: Synthetic polypeptide
EVKLQESGGGFVKPGGSLRVSCAASGFTFSSYAMSWVRLAPEMRLEWVATISSAGGYIFYSDSVQGRFTISRDNAKNSLHLQMGSLRSGDTAMYYCARQGFGNYGDYYAMDYWGQGTTVTVSS

SEQ ID NO: 147 - protein, 123 aa, Artificial Sequence
  Description of Artificial Sequence: Synthetic polypeptide
EVQLVESGGGLVKPGGSLRVSCAASGFTFSSYAMSWVRLAPGKGLEWVATISSAGGYIFYSDSVQGRFTISRDNAKNSLYLQMNSLRAEDTAMYYCARQGFGNYGDYYAMDYWGQGTLVTVSS

SEQ ID NO: 148 - protein, 123 aa, Artificial Sequence
  Description of Artificial Sequence: Synthetic polypeptide
QVTLKESGPGILQPTQTLTLTCTFSGFSLSTVGMGVGWSRQPSGKGLEWLAHIWWDDEDKYYNPALKSRLTITKDTSKNQVFLKITNVDTADTATYYCTRIGTAQATDALDYWGQGTLVTVSS

SEQ ID NO: 149 - protein, 123 aa, Artificial Sequence
  Description of Artificial Sequence: Synthetic polypeptide
QVTLKESGPTLVKPTQTLTLTCTFSGFSLSTVGMGVGWSRQPSGKGLEWLAHIWWDDEDKYYNPALKSRLTITKDTSKNQVVLTITNVDPVDTATYYCTRIGTAQATDALDYWGQGTLVTVSS

SEQ ID NO: 150 - protein, 113 aa, Artificial Sequence
  Description of Artificial Sequence: Synthetic polypeptide
DIELTQSPSSLAVSAGERVTMNCKSSQSLLNSRTRKNQLAWYQQKPGQSPELLIYWASTRQSGVPDRFSGSGSGTDFTLTISSVQAEDVAVYYCQQSYNLLTFGPGTKLEIKR

SEQ ID NO: 151 - protein, 113 aa, Artificial Sequence
  Description of Artificial Sequence: Synthetic polypeptide
DIVLTQSPDSLAVSLGERVTMNCKSSQSLLNSRTRKNQLAWYQQKPGQSPELLIYWASTRQSGVPDRFSGSGSGTDFTLTISSVQAEDVAVYYCQQSYNLLTFGQGTKLEIKR

SEQ ID NO: 152 - protein, 113 aa, Artificial Sequence
  Description of Artificial Sequence: Synthetic polypeptide
DIVMTQSAPSVPVTPGESVSISCRSSKSLLHSNGNTYLYWFLQKPGQSPQRLIYYMSNLASGVPDRFSGRGSGTDFTLKISRVEAEDVGVYYCMQSLEYPLTFGGGTKLEIKR

SEQ ID NO: 153 - protein, 113 aa, Artificial Sequence
  Description of Artificial Sequence: Synthetic polypeptide
DIVMTQSALSLPVTPGEPVSISCRSSKSLLHSNGNTYLYWFLQKPGQSPQRLIYYMSNLASGVPDRFSGSGSGTDFTLKISRVEAEDVGVYYCMQSLEYPLTFGGGTKLEIKR

SEQ ID NO: 154 - protein, 116 aa, Artificial Sequence
  Description of Artificial Sequence: Synthetic polypeptide
EVQLVQSGVEVKKPGESLKISCKGSGYDFSDYWITWVRQMPGRGLEWMARIDPSDSNIDYSPSFQGHVTISADKSISTAYLQWSSLKASDTAMYYCARSHGGGDYWGQGTLVTVSS

SEQ ID NO: 155 - protein, 8 aa, Artificial Sequence
  Description of Artificial Sequence: Synthetic peptide
GYDFSDYW

SEQ ID NO: 156 - protein, 8 aa, Artificial Sequence
  Description of Artificial Sequence: Synthetic peptide
IDPSDSNI

SEQ ID NO: 157 - protein, 9 aa, Artificial Sequence
  Description of Artificial Sequence: Synthetic peptide
ARSHGGGDY

SEQ ID NO: 158 - protein, 121 aa, Artificial Sequence
  Description of Artificial Sequence: Synthetic polypeptide
EVQLVESGGGVVQPGRSLRLSCVASGFSFSTYAMHWVRQAPGKGLEWVASILDDGSKKDYADSVEGRFVISRDNSKNTLFLEVNSLSPEDTAVYYCARYSSWSSFGYPDYWGQGTLVTVSS

SEQ ID NO: 159 - protein, 8 aa, Artificial Sequence
  Description of Artificial Sequence: Synthetic peptide
GFSFSTYA

SEQ ID NO: 160 - protein, 8 aa, Artificial Sequence
  Description of Artificial Sequence: Synthetic peptide
ILDDGSKK

SEQ ID NO: 161 - protein, 14 aa, Artificial Sequence
  Description of Artificial Sequence: Synthetic peptide
ARYSSWSSFGYPDY

SEQ ID NO: 162 - protein, 118 aa, Artificial Sequence
  Description of Artificial Sequence: Synthetic polypeptide
EVQLVQSGAEVKKPGSSVKVSCKASGGTFSSYAISWVRQAPGQGLEWMGWMNPNSGNTGYAQKFQGRVTMTRNTSISTAYMELSSLRSEDTAVYYCARRSRTTGYDHWGQGTLVTVSS

SEQ ID NO: 163 - protein, 8 aa, Artificial Sequence
  Description of Artificial Sequence: Synthetic peptide
GGTFSSYA

SEQ ID NO: 164 - protein, 8 aa, Artificial Sequence
  Description of Artificial Sequence: Synthetic peptide
MNPNSGNT

SEQ ID NO: 165 - protein, 11 aa, Artificial Sequence
  Description of Artificial Sequence: Synthetic peptide
ARRSRTTGYDH

SEQ ID NO: 166 - protein, 107 aa, Artificial Sequence
  Description of Artificial Sequence: Synthetic polypeptide
SYVLTQPPSVSVSPGQTASITCSGDNLGDKYVSWYQQKPGQSPVLVIYQDTKRPSRIPERFSGSNSGNTATLTISGTQAMDDADYYCLTWDSSTGVFGAGTKVTVLG

SEQ ID NO: 167 - protein, 6 aa, Artificial Sequence
  Description of Artificial Sequence: Synthetic peptide
NLGDKY

SEQ ID NO: 168 - protein, 3 aa, Artificial Sequence
  Description of Artificial Sequence: Synthetic peptide
QDT

SEQ ID NO: 169 - protein, 9 aa, Artificial Sequence
  Description of Artificial Sequence: Synthetic peptide
LTWDSSTGV

SEQ ID NO: 170 - protein, 112 aa, Artificial Sequence
  Description of Artificial Sequence: Synthetic polypeptide
NFMLTQPHSVSESLGKTVTISCTRSSGSIGTGYVQWYQLRPGSAPIMVIYEDNRRPSGVPGRFSGSIDSSSNSASLTISGLMSEDEADYYCQSFDYFNQGVFGGGTKVTVLG

SEQ ID NO: 171 - protein, 8 aa, Artificial Sequence
  Description of Artificial Sequence: Synthetic peptide
SGSIGTGY

SEQ ID NO: 172 - protein, 3 aa, Artificial Sequence
  Description of Artificial Sequence: Synthetic peptide
EDN

SEQ ID NO: 173 - protein, 10 aa, Artificial Sequence
  Description of Artificial Sequence: Synthetic peptide
QSFDYFNQGV

SEQ ID NO: 174 - protein, 108 aa, Artificial Sequence
  Description of Artificial Sequence: Synthetic polypeptide
DIQMTQSPSTLSASVGDRVTITCQANQDISNDLNWYQQKPGKAPKLLIYDTSTLEIGVPSRFSGSGSGTNFTFTISSLQPEDIATYYCQQFYNLPITFGQGTRLEIKR

SEQ ID NO: 175 - protein, 6 aa, Artificial Sequence
  Description of Artificial Sequence: Synthetic peptide
QDISND

SEQ ID NO: 176 - protein, 3 aa, Artificial Sequence
  Description of Artificial Sequence: Synthetic peptide
DTS

SEQ ID NO: 177 - protein, 9 aa, Artificial Sequence
  Description of Artificial Sequence: Synthetic peptide
QQFYNLPIT

SEQ ID NO: 178 - protein, 274 aa, Artificial Sequence; identical to SEQ ID NO: 1
  Description of Artificial Sequence: Synthetic polypeptide

SEQ ID NO: 179 - protein, 310 aa, Artificial Sequence; identical to SEQ ID NO: 2
  Description of Artificial Sequence: Synthetic polypeptide

SEQ ID NO: 180 - protein, 274 aa, Artificial Sequence; identical to SEQ ID NO: 3
  Description of Artificial Sequence: Synthetic polypeptide

SEQ ID NO: 181 - protein, 555 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 182 - protein, 390 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 183 - protein, 379 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 184 - protein, 395 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 185 - protein, 404 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSP

SEQ ID NO: 186 - protein, 408 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCRSKRSRLLHSDYMNMTPRRPGPTRKHYQPYAPPRDFAAYRS

SEQ ID NO: 187 - protein, 409 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCKRGRKKLLYIFKQPFMRPVQTTQEEDGCSCRFPEEEEGGCEL

SEQ ID NO: 188 - protein, 409 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCALYLLRRDQRLPPDAHKPPGGGSFRTPIQEEQADAHSTLAKI

SEQ ID NO: 189 - protein, 415 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCQRRKYRSNKGESPVEPAEPCRYSCPREEEGSTIPIQEDYRKPEPACSP

SEQ ID NO: 190 - protein, 539 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGIEVMYPPPYLDNEKSNGTIIHVKGKHLCPSPLFPGPSKPFWVLVVVGGVLACYSLLVTVAFIIFWVHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 191 - protein, 393 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCEL

SEQ ID NO: 192 - protein, 393 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGIEVMYPPPYLDNEKSNGTIIHVKGKHLCPSPLFPGPSKPFWVLVVVGGVLACYSLLVTVAFIIFWVALYLLRRDQRLPPDAHKPPGGGSFRTPIQEEQADAHSTLAKI

SEQ ID NO: 193 - protein, 399 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGIEVMYPPPYLDNEKSNGTIIHVKGKHLCPSPLFPGPSKPFWVLVVVGGVLACYSLLVTVAFIIFWVQRRKYRSNKGESPVEPAEPCRYSCPREEEGSTIPIQEDYRKPEPACSP

SEQ ID NO: 194 - protein, 374 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGIEVMYPPPYLDNEKSNGTIIHVKGKHLCPSPLFPGPSKPFWVLVVVGGVLACYSLLVTVAFIIFWVCARPRRSPAQEDGKVYINMPGRG

SEQ ID NO: 195 - protein, 525 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGPADLSPGASSVTPPAPAREPGHSPQIISFFLALTSTALLFLLFFLTLRFSVVHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 196 - protein, 378 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGPADLSPGASSVTPPAPAREPGHSPQIISFFLALTSTALLFLLFFLTLRFSVVRSKRSRLLHSDYMNMTPRRPGPTRKHYQPYAPPRDFAAYRS

SEQ ID NO: 197 - protein, 379 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGPADLSPGASSVTPPAPAREPGHSPQIISFFLALTSTALLFLLFFLTLRFSVVALYLLRRDQRLPPDAHKPPGGGSFRTPIQEEQADAHSTLAKI

SEQ ID NO: 198 - protein, 385 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGPADLSPGASSVTPPAPAREPGHSPQIISFFLALTSTALLFLLFFLTLRFSVVQRRKYRSNKGESPVEPAEPCRYSCPREEEGSTIPIQEDYRKPEPACSP

SEQ ID NO: 199 - protein, 541 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGDRDPPATQPQETQGPPARPITVQPTEAWPRTSQGPSTRPVEVPGGRAVAAILGLGLVLGLLGPLAILLHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 200 - protein, 394 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGDRDPPATQPQETQGPPARPITVQPTEAWPRTSQGPSTRPVEVPGGRAVAAILGLGLVLGLLGPLAILLRSKRSRLLHSDYMNMTPRRPGPTRKHYQPYAPPRDFAAYRS

SEQ ID NO: 201 - protein, 395 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGDRDPPATQPQETQGPPARPITVQPTEAWPRTSQGPSTRPVEVPGGRAVAAILGLGLVLGLLGPLAILLKRGRKKLLYIFKQPFMRPVQTTQEEDGCSCRFPEEEEGGCEL

SEQ ID NO: 202 - protein, 401 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGDRDPPATQPQETQGPPARPITVQPTEAWPRTSQGPSTRPVEVPGGRAVAAILGLGLVLGLLGPLAILLQRRKYRSNKGESPVEPAEPCRYSCPREEEGSTIPIQEDYRKPEPACSP

SEQ ID NO: 203 - protein, 544 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGPTHLPYVSEMLEARTAGHMQTLADFRQLPARTLSTHWPPQRSLCSSDFIRILVIFSGMFLVFTLAGALFLHHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 204 - protein, 397 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGPTHLPYVSEMLEARTAGHMQTLADFRQLPARTLSTHWPPQRSLCSSDFIRILVIFSGMFLVFTLAGALFLHRSKRSRLLHSDYMNMTPRRPGPTRKHYQPYAPPRDFAAYRS

SEQ ID NO: 205 - protein, 398 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGPTHLPYVSEMLEARTAGHMQTLADFRQLPARTLSTHWPPQRSLCSSDFIRILVIFSGMFLVFTLAGALFLHKRGRKKLLYIFKQPFMRPVQTTQEEDGCSCRFPEEEEGGCEL

SEQ ID NO: 206 - protein, 398 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGPTHLPYVSEMLEARTAGHMQTLADFRQLPARTLSTHWPPQRSLCSSDFIRILVIFSGMFLVFTLAGALFLHALYLLRRDQRLPPDAHKPPGGGSFRTPIQEEQADAHSTLAKI

SEQ ID NO: 207 - protein, 542 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGTTTPAPRPPTPAPTIASQPLSLRPEACRPAAGGAVHTRGLDFACDIYIWAPLAGTCGVLLLSLVITLYCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 208 - protein, 395 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRS

SEQ ID NO: 209 - protein, 396 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCEL

SEQ ID NO: 210 - protein, 396 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGTTTPAPRPPTPAPTIASQPLSLRPEACRPAAGGAVHTRGLDFACDIYIWAPLAGTCGVLLLSLVITLYCALYLLRRDQRLPPDAHKPPGGGSFRTPIQEEQADAHSTLAKI

SEQ ID NO: 211 - protein, 402 aa, Artificial Sequence
  Description of Artificial Sequence: Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSEQKLISEEDLAAATGTTTPAPRPPTPAPTIASQPLSLRPEACRPAAGGAVHTRGLDFACDIYIWAPLAGTCGVLLLSLVITLYCQRRKYRSNKGESPVEPAEPCRYSCPREEEGSTIPIQEDYRKPEPACSP

SEQ ID NO: 212 - protein, 247 aa, Artificial Sequence
  Description of Artificial Sequence: Synthetic polypeptide
QPVLTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVLGSRGGGGSGGGGSGGGGSLEMAQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARWHGGPYDYWGQGTLVTVSS

SEQ ID NO: 213 - protein, 249 aa, Artificial Sequence
  Description of Artificial Sequence: Synthetic polypeptide
QSVLTQPPSVSVAPGKTARITCGGNNIGSKSVHWYQQKPGQAPVLVVYDDSDRPSGIPERFSGSNSGNTATLTISRVEAGDEADYYCQVWDSSSDHVFGTGTKVTVLGSRGGGGSGGGGSGGGGSLEMAQVQLVQSGADVRKPGASVKVSCKASGYTFASHGISWVRQAPGQGLEWLGWISPYTGNTNYAQKFQGRVTMATDTSTSTAYMELRSLRSDDTAIYYCARGKRTLASCFDYWGQGTLVTVSS

SEQ ID NO: 214 - protein, 254 aa, Artificial Sequence
  Description of Artificial Sequence: Synthetic polypeptide
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSMGSSLYASSDVWGQGTLVTVSS

SEQ ID NO: 215 - protein, 249 aa, Artificial Sequence
  Description of Artificial Sequence: Synthetic polypeptide
QAVLTQPPSASGTPGQRVTISCSGSSSNIGSNTVNWYQQLPGTAPKLLMYSNNQRPSGVPDRFSGSKSGTSASLAISGLQSEDEADYYCAAWDDSLNGYVFGTGTKVTVLGSRGGGGSGGGGSGGGGSLEMAEVQLVQSGAEMKKPGESLKISCKGSGYNFASYWVGWVRQMPGKGLEWMGTIYPDDSDTRYGPAFQGQVTISADKSISTAYLQWSSLKASDTAMYYCARDSYYGIDVWGQGTLVTVSS

SEQ ID NO: 216 - protein, 248 aa, Artificial Sequence
  Description of Artificial Sequence: Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRSRGGGGSGGGGSGGGGSLEMAQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSS

SEQ ID NO: 217 - protein, 270 aa, Artificial Sequence
  Description of Artificial Sequence: Synthetic polypeptide
APPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 218 - protein, 107 aa, Artificial Sequence
  Description of Artificial Sequence: Synthetic polypeptide
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 219 - protein, 94 aa, Artificial Sequence
  Description of Artificial Sequence: Synthetic polypeptide
PADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 220 - protein, 110 aa, Artificial Sequence
  Description of Artificial Sequence: Synthetic polypeptide
DRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 221 - protein, 119 aa, Artificial Sequence
  Description of Artificial Sequence: Synthetic polypeptide
PTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSP

SEQ ID NO: 222 - protein, 45 aa, Artificial Sequence
  Description of Artificial Sequence: Synthetic polypeptide
LLAGLVAADAVASLLIVGAVFLCARPRRSPAQEDGKVYINMPGRG

SEQ ID NO: 223 - protein, 254 aa, Artificial Sequence
  Description of Artificial Sequence: Synthetic polypeptide
IEVMYPPPYLDNEKSNGTIIHVKGKHLCPSPLFPGPSKPFWVLVVVGGVLACYSLLVTVAFIIFWVHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 224 - protein, 240 aa, Artificial Sequence
  Description of Artificial Sequence: Synthetic polypeptide
PADLSPGASSVTPPAPAREPGHSPQIISFFLALTSTALLFLLFFLTLRFSVVHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 225 - protein, 256 aa, Artificial Sequence
  Description of Artificial Sequence: Synthetic polypeptide
DRDPPATQPQETQGPPARPITVQPTEAWPRTSQGPSTRPVEVPGGRAVAAILGLGLVLGLLGPLAILLHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 226 - protein, 259 aa, Artificial Sequence
  Description of Artificial Sequence: Synthetic polypeptide
PTHLPYVSEMLEARTAGHMQTLADFRQLPARTLSTHWPPQRSLCSSDFIRILVIFSGMFLVFTLAGALFLHHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 227 - protein, 257 aa, Artificial Sequence
  Description of Artificial Sequence: Synthetic polypeptide
TTTPAPRPPTPAPTIASQPLSLRPEACRPAAGGAVHTRGLDFACDIYIWAPLAGTCGVLLLSLVITLYCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 228 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 229 - protein, 21 aa, Artificial Sequence
  Description of Artificial Sequence: Synthetic peptide
IYIWAPLAGTCGVLLLSLVIT

SEQ ID NO: 230 - protein, 27 aa, Artificial Sequence
  Description of Artificial Sequence: Synthetic peptide
IISFFLALTSTALLFLLFFLTLRFSVV

SEQ ID NO: 231 - protein, 21 aa, Artificial Sequence
  Description of Artificial Sequence: Synthetic peptide
ILVIFSGMFLVFTLAGALFLH

SEQ ID NO: 232 - protein, 27 aa, Artificial Sequence
  Description of Artificial Sequence: Synthetic peptide
FWVLVVVGGVLACYSLLVTVAFIIFWV

SEQ ID NO: 233 - protein, 28 aa, Artificial Sequence
  Description of Artificial Sequence: Synthetic peptide
PVLDAGPVLFWVILVLVVVVGSSAFLLC

SEQ ID NO: 234 - protein, 21 aa, Artificial Sequence
  Description of Artificial Sequence: Synthetic peptide
VAAILGLGLVLGLLGPLAILL

SEQ ID NO: 235 - protein, 42 aa, Artificial Sequence
  Description of Artificial Sequence: Synthetic polypeptide
KRGRKKLLYIFKQPFMRPVQTTQEEDGCSCRFPEEEEGGCEL

SEQ ID NO: 236 - protein, 48 aa, Artificial Sequence
  Description of Artificial Sequence: Synthetic polypeptide
QRRKYRSNKGESPVEPAEPCRYSCPREEEGSTIPIQEDYRKPEPACSP

SEQ ID NO: 237 - protein, 41 aa, Artificial Sequence
  Description of Artificial Sequence: Synthetic polypeptide
RSKRSRLLHSDYMNMTPRRPGPTRKHYQPYAPPRDFAAYRS

SEQ ID NO: 238 - protein, 188 aa, Artificial Sequence
  Description of Artificial Sequence: Synthetic polypeptide
HRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 239 - protein, 42 aa, Artificial Sequence
  Description of Artificial Sequence: Synthetic polypeptide
ALYLLRRDQRLPPDAHKPPGGGSFRTPIQEEQADAHSTLAKI

SEQ ID NO: 240 - protein, 23 aa, Artificial Sequence
  Description of Artificial Sequence: Synthetic peptide
CARPRRSPAQEDGKVYINMPGRG

SEQ ID NO: 241 - protein, 112 aa, Artificial Sequence
  Description of Artificial Sequence: Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 242 - protein, 21 aa, Artificial Sequence
  Description of Artificial Sequence: Synthetic peptide
SRGGGGSGGGGSGGGGSLEMA

SEQ ID NO: 243 - protein, 5 aa, Artificial Sequence
  Description of Artificial Sequence: Synthetic peptide
GGGGS

SEQ ID NO: 244 - protein, 4 aa, Artificial Sequence
  Description of Artificial Sequence: Synthetic peptide
GGSG

SEQ ID NO: 245 - protein, 4 aa, Artificial Sequence
  Description of Artificial Sequence: Synthetic peptide
SGGG

SEQ ID NO: 246 - protein, 4 aa, Artificial Sequence
  Description of Artificial Sequence: Synthetic peptide
GSGS

SEQ ID NO: 247 - protein, 6 aa, Artificial Sequence
  Description of Artificial Sequence: Synthetic peptide
GSGSGS

SEQ ID NO: 248 - protein, 8 aa, Artificial Sequence
  Description of Artificial Sequence: Synthetic peptide
GSGSGSGS

SEQ ID NO: 249 - protein, 10 aa, Artificial Sequence
  Description of Artificial Sequence: Synthetic peptide
GSGSGSGSGS

SEQ ID NO: 250 - protein, 6 aa, Artificial Sequence
  Description of Artificial Sequence: Synthetic peptide
GGSGGS

SEQ ID NO: 251 - protein, 9 aa, Artificial Sequence
  Description of Artificial Sequence: Synthetic peptide
GGSGGSGGS

SEQ ID NO: 252 - protein, 12 aa, Artificial Sequence
  Description of Artificial Sequence: Synthetic peptide
GGSGGSGGSGGS

SEQ ID NO: 253 - protein, 4 aa, Artificial Sequence
  Description of Artificial Sequence: Synthetic peptide
GGSG

SEQ ID NO: 254 - protein, 8 aa, Artificial Sequence
  Description of Artificial Sequence: Synthetic peptide
GGSGGGSG

SEQ ID NO: 255 - protein, 12 aa, Artificial Sequence
  Description of Artificial Sequence: Synthetic peptide
GGSGGGSGGGSG

SEQ ID NO: 256 - protein, 21 aa, Artificial Sequence
  Description of Artificial Sequence: Synthetic peptide
SRGGGGSGGGGSGGGGSLEMA

SEQ ID NO: 257 - protein, 6 aa, Artificial Sequence
  Description of Artificial Sequence: Synthetic 6xHis tag
HHHHHH

SEQ ID NO: 258 - protein, 9 aa, Artificial Sequence
  Description of Artificial Sequence: Synthetic peptide
YPYDVPDYA

SEQ ID NO: 259 - protein, 10 aa, Artificial Sequence
  Description of Artificial Sequence: Synthetic peptide
YPYDVPDYAS

SEQ ID NO: 260 - protein, 8 aa, Artificial Sequence
  Description of Artificial Sequence: Synthetic peptide
DYKDDDDK

SEQ ID NO: 261 - protein, 10 aa, Artificial Sequence
  Description of Artificial Sequence: Synthetic peptide
EQKLISEEDL

SEQ ID NO: 262 - protein, 117 aa, Artificial Sequence
  Description of Artificial Sequence: Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARWHGGPYDYWGQGTLVTVSS

SEQ ID NO: 263 - protein, 109 aa, Artificial Sequence
  Description of Artificial Sequence: Synthetic polypeptide
QPVLTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVLG

SEQ ID NO: 264 - protein, 120 aa, Artificial Sequence
  Description of Artificial Sequence: Synthetic polypeptide
QVQLVQSGADVRKPGASVKVSCKASGYTFASHGISWVRQAPGQGLEWLGWISPYTGNTNYAQKFQGRVTMATDTSTSTAYMELRSLRSDDTAIYYCARGKRTLASCFDYWGQGTLVTVSS

SEQ ID NO: 265 - protein, 108 aa, Artificial Sequence
  Description of Artificial Sequence: Synthetic polypeptide
QSVLTQPPSVSVAPGKTARITCGGNNIGSKSVHWYQQKPGQAPVLVVYDDSDRPSGIPERFSGSNSGNTATLTISRVEAGDEADYYCQVWDSSSDHVFGTGTKVTVLG

SEQ ID NO: 266 - DNA, 180 bp, Artificial Sequence
  Description of Artificial Sequence: Synthetic polynucleotide
ggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgt

SEQ ID NO: 267 - DNA, 149 bp, Artificial Sequence
  Description of Artificial Sequence: Synthetic polynucleotide
gccgccccgactgcatctgcgtgttccaattcgccaatgacaagacgctgggcggggtttgtgtcatcatagaactaaagacatgcaaatatatttcttccggggacaccgccagcaaacgcgagcaacgggccacggggatgaagcag

SEQ ID NO: 268 - DNA, 335 bp, Artificial Sequence
  Description of Artificial Sequence: Synthetic polynucleotide
ggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtctcgaggccgccccgactgcatctgcgtgttccaattcgccaatgacaagacgctgggcggggtttgtgtcatcatagaactaaagacatgcaaatatatttcttccggggacaccgccagcaaacgcgagcaacgggccacggggatgaagcag

SEQ ID NO: 269 - protein, 248 aa, Artificial Sequence
  Description of Artificial Sequence: Synthetic polypeptide
QPVLTQPPSASGTPGQRVTISCSGSSSNIGSNNVIWYQQLPGAAPKLLIYSNHRRPSGVPDRFSGSRSGTSASLAISGLQSEDEADYYCAAWDDSLDGYLFGTGTKVTVLGSRGGGGSGGGGSGGGGSLEMAQVQLQQWGAGLLKPSETLSLTCAVYGGSFSGYYWSWIRQPPGKGLEWIGEINHSGSTNYNPSLKSRVTISVDTSKNQFSLELSSVTAADTAVYYCARGYGGRFDYWGQGTLVTVSS

SEQ ID NO: 270 - protein, 250 aa, Artificial Sequence
  Description of Artificial Sequence: Synthetic polypeptide
QSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSS

SEQ ID NO: 271 - protein, 248 aa, Artificial Sequence
  Description of Artificial Sequence: Synthetic polypeptide
QSVLTQPPSVSGTPGQRVIISCPGSTSNIGTNTVNWYQQFPGTAPKLLIYSNNQRPSGVPDRFSGSKSGTSASLAISGLQSEDEADYYCAAWDDSLNGVVFGGGTKLTVLGSRGGGGSGGGGSGGGGSLEMAQMQLVQSGGGLVKPGGSLRLSCAASGFTFSDYYMSWIRQAPGKGLEWVSYISSSGSTIYYADSVKGRFTISRDNAKNSLYLQMNSLRAEDTAVYYCARASDLYGDWGQGTLVTVSS

SEQ ID NO: 272 - protein, 252 aa, Artificial Sequence
  Description of Artificial Sequence: Synthetic polypeptide
QAVLTQPPSVSTPGQRVTISCSGSSSNFGSNTVHWYQQVPGTAPKLLIFSNTQRPSEIPDRFSGSKSGTSASLAISGLQSEDEADYYCAAWDDSLTGVVFGGGTKLTVLGSRGGGGSGGGGSGGGGSLEMAQVQLVQSGAEVKKPGASVTVSCKASGYRFSNYGVSWVRQAPGQGLEWMGWISGSNGNTNYAQKFLGRVTMTTDTSTTTAYMELSSLRSDDTAVYYCARGNRRYYSPIIDPWGQGTLVTVSS

SEQ ID NO: 273 - protein, 247 aa, Artificial Sequence
  Description of Artificial Sequence: Synthetic polypeptide
DVVMTQSPLSLPVTPGEPASVSCRSSQSLLHSNGYNYLDWYLQKPGQSPQLLIYLGSNRASGVPDRFSGSGSGTDFTLKISRVEAEDVGVYYCMQALQTPWTFGQGTKVEIKRSRGGGGSGGGGSGGGGSLEMAEVQLVQSGAEVRKPGSSVKVSCQASGGTFGSYAISWVRQAPGQGLEWMGRIIPVLGRTKYAQKFQGRVTVTADTSTSTVYMELTSLTSEDTAVYYCARTNDSWGQGTLVTVSS

SEQ ID NO: 274 - protein, 116 aa, Artificial Sequence
  Description of Artificial Sequence: Synthetic polypeptide
QVQLQQWGAGLLKPSETLSLTCAVYGGSFSGYYWSWIRQPPGKGLEWIGEINHSGSTNYNPSLKSRVTISVDTSKNQFSLELSSVTAADTAVYYCARGYGGRFDYWGQGTLVTVSS

SEQ ID NO: 275 - protein, 111 aa, Artificial Sequence
  Description of Artificial Sequence: Synthetic polypeptide
QPVLTQPPSASGTPGQRVTISCSGSSSNIGSNNVIWYQQLPGAAPKLLIYSNHRRPSGVPDRFSGSRSGTSASLAISGLQSEDEADYYCAAWDDSLDGYLFGTGTKVTVLG

SEQ ID NO: 276 - protein, 118 aa, Artificial Sequence
  Description of Artificial Sequence: Synthetic polypeptide
QVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSS

SEQ ID NO: 277 - protein, 111 aa, Artificial Sequence
  Description of Artificial Sequence: Synthetic polypeptide
QSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLG

SEQ ID NO: 278 - protein, 116 aa, Artificial Sequence
  Description of Artificial Sequence: Synthetic polypeptide
QMQLVQSGGGLVKPGGSLRLSCAASGFTFSDYYMSWIRQAPGKGLEWVSYISSSGSTIYYADSVKGRFTISRDNAKNSLYLQMNSLRAEDTAVYYCARASDLYGDWGQGTLVTVSS

SEQ ID NO: 279 - protein, 111 aa, Artificial Sequence
  Description of Artificial Sequence: Synthetic polypeptide
QSVLTQPPSVSGTPGQRVIISCPGSTSNIGTNTVNWYQQFPGTAPKLLIYSNNQRPSGVPDRFSGSKSGTSASLAISGLQSEDEADYYCAAWDDSLNGVVFGGGTKLTVLG

SEQ ID NO: 280 - protein, 121 aa, Artificial Sequence
  Description of Artificial Sequence: Synthetic polypeptide
QVQLVQSGAEVKKPGASVTVSCKASGYRFSNYGVSWVRQAPGQGLEWMGWISGSNGNTNYAQKFLGRVTMTTDTSTTTAYMELSSLRSDDTAVYYCARGNRRYYSPIIDPWGQGTLVTVSS

SEQ ID NO: 281 - protein, 111 aa, Artificial Sequence
  Description of Artificial Sequence: Synthetic polypeptide
QAVLTQPPSVSGTPGQRVTISCSGSSSNFGSNTVHWYQQVPGTAPKLLIFSNTQRPSEIPDRFSGSKSGTSASLAISGLQSEDEADYYCAAWDDSLTGVVFGGGTKLTVLG

SEQ ID NO: 282 - protein, 117 aa, Artificial Sequence; identical to SEQ ID NO: 262
  Description of Artificial Sequence: Synthetic polypeptide

SEQ ID NO: 283 - protein, 109 aa, Artificial Sequence; identical to SEQ ID NO: 263
  Description of Artificial Sequence: Synthetic polypeptide

SEQ ID NO: 284 - protein, 120 aa, Artificial Sequence; identical to SEQ ID NO: 264
  Description of Artificial Sequence: Synthetic polypeptide

SEQ ID NO: 285 - protein, 108 aa, Artificial Sequence; identical to SEQ ID NO: 265
  Description of Artificial Sequence: Synthetic polypeptide

SEQ ID NO: 286 - protein, 113 aa, Artificial Sequence
  Description of Artificial Sequence: Synthetic polypeptide
EVQLVQSGAEVRKPGSSVKVSCQASGGTFGSYAISWVRQAPGQGLEWMGRIIPVLGRTKYAQKFQGRVTVTADTSTSTVYMELTSLTSEDTAVYYCARTNDSWGQGTLVTVSS

SEQ ID NO: 287 - protein, 113 aa, Artificial Sequence
  Description of Artificial Sequence: Synthetic polypeptide
DVVMTQSPLSLPVTPGEPASVSCRSSQSLLHSNGYNYLDWYLQKPGQSPQLLIYLGSNRASGVPDRFSGSGSGTDFTLKISRVEAEDVGVYYCMQALQTPWTFGQGTKVEIKR

SEQ ID NO: 288 - protein, 553 aa, Artificial Sequence
  Description of Artificial Sequence: Synthetic polypeptide
METDTLLLWVLLLWVPGSTGQPVLTQPPSASGTPGQRVTISCSGSSSNIGSNNVIWYQQLPGAAPKLLIYSNHRRPSGVPDRFSGSRSGTSASLAISGLQSEDEADYYCAAWDDSLDGYLFGTGTKVTVLGSRGGGGSGGGGSGGGGSLEMAQVQLQQWGAGLLKPSETLSLTCAVYGGSFSGYYWSWIRQPPGKGLEWIGEINHSGSTNYNPSLKSRVTISVDTSKNQFSLELSSVTAADTAVYYCARGYGGRFDYWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 289 - protein, 553 aa, Artificial Sequence
  Description of Artificial Sequence: Synthetic polypeptide
METDTLLLWVLLLWVPGSTGQSVLTQPPSVSGTPGQRVIISCPGSTSNIGTNTVNWYQQFPGTAPKLLIYSNNQRPSGVPDRFSGSKSGTSASLAISGLQSEDEADYYCAAWDDSLNGVVFGGGTKLTVLGSRGGGGSGGGGSGGGGSLEMAQMQLVQSGGGLVKPGGSLRLSCAASGFTFSDYYMSWIRQAPGKGLEWVSYISSSGSTIYYADSVKGRFTISRDNAKNSLYLQMNSLRAEDTAVYYCARASDLYGDWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 290 - protein, 557 aa, Artificial Sequence
  Description of Artificial Sequence: Synthetic polypeptide
METDTLLLWVLLLWVPGSTGQAVLTQPPSVSTPGQRVTISCSGSSSNFGSNTVHWYQQVPGTAPKLLIFSNTQRPSEIPDRFSGSKSGTSASLAISGLQSEDEADYYCAAWDDSLTGVVFGGGTKLTVLGSRGGGGSGGGGSGGGGSLEMAQVQLVQSGAEVKKPGASVTVSCKASGYRFSNYGVSWVRQAPGQGLEWMGWISGSNGNTNYAQKFLGRVTMTTDTSTTTAYMELSSLRSDDTAVYYCARGNRRYYSPIIDPWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 291 - protein, 552 aa, Artificial Sequence
  Description of Artificial Sequence: Synthetic polypeptide
METDTLLLWVLLLWVPGSTGQPVLTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVLGSRGGGGSGGGGSGGGGSLEMAQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARWHGGPYDYWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 292 - protein, 554 aa, Artificial Sequence
  Description of Artificial Sequence: Synthetic polypeptide
METDTLLLWVLLLWVPGSTGQSVLTQPPSVSVAPGKTARITCGGNNIGSKSVHWYQQKPGQAPVLVVYDDSDRPSGIPERFSGSNSGNTATLTISRVEAGDEADYYCQVWDSSSDHVFGTGTKVTVLGSRGGGGSGGGGSGGGGSLEMAQVQLVQSGADVRKPGASVKVSCKASGYTFASHGISWVRQAPGQGLEWLGWISPYTGNTNYAQKFQGRVTMATDTSTSTAYMELRSLRSDDTAIYYCARGKRTLASCFDYWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 293 - protein, 552 aa, Artificial Sequence
  Description of Artificial Sequence: Synthetic polypeptide
METDTLLLWVLLLWVPGSTGDVVMTQSPLSLPVTPGEPASVSCRSSQSLLHSNGYNYLDWYLQKPGQSPQLLIYLGSNRASGVPDRFSGSGSGTDFTLKISRVEAEDVGVYYCMQALQTPWTFGQGTKVEIKRSRGGGGSGGGGSGGGGSLEMAEVQLVQSGAEVRKPGSSVKVSCQASGGTFGSYAISWVRQAPGQGLEWMGRIIPVLGRTKYAQKFQGRVTVTADTSTSTVYMELTSLTSEDTAVYYCARTNDSWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 294 - protein, 20 aa, Artificial Sequence
  Description of Artificial Sequence: Synthetic peptide
METDTLLLWVLLLWVPGSTG

SEQ ID NO: 295 - protein, 120 aa, Artificial Sequence
  Description of Artificial Sequence: Synthetic polypeptide
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDSYYYYYGMDVWGQGTTVTVSS

SEQ ID NO: 296 - protein, 110 aa, Artificial Sequence
  Description of Artificial Sequence: Synthetic polypeptide
QAVVTQEPSLTVSPGGTVTLTCGSSTGAVTSGHYPYWFQQKPGQAPRTLIYDASDKHSWTPARFSGSLLGGKAALTLSGAQPEDEAEYYCLLSYSDALVFGGGTKLTVLG

SEQ ID NO: 297 - protein, 117 aa, Artificial Sequence
  Description of Artificial Sequence: Synthetic polypeptide
EVQLVQSGAEVKKPGESLTISCKASGYSFPNYWITWVRQMSGGGLEWMGRIDPGDSYTTYNPSFQGHVTISIDKSTNTAYLHWNSLKASDTAMYYCARYYVSLVDIWGQGTLVTVSS

SEQ ID NO: 298 - protein, 111 aa, Artificial Sequence
  Description of Artificial Sequence: Synthetic polypeptide
QSVLTQPASVSGSPGQSITISCTGTSSDVGGYNYVSWYQQHPGKAPKLMIYDVNNRPSEVSNRFSGSKSGNTASLTISGLQAEDEADYYCSSYTTGSRAVFGGGTKLTVLG

SEQ ID NO: 299 - protein, 124 aa, Artificial Sequence
  Description of Artificial Sequence: Synthetic polypeptide
EVQLVESGGGLVKPGGSLRLSCAASGFTFSNAWMSWVRQAPGKGLEWVGFIRSKAYGGTTEYAASVKGRFTISRDDSKSIAYLQMNNLKTEDTAVYYCARDGLYSSSWYDSDYWGQGTLVTVSS

SEQ ID NO: 300 - protein, 111 aa, Artificial Sequence
  Description of Artificial Sequence: Synthetic polypeptide
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDGSLYTMLFGGGTKLTVLG

SEQ ID NO: 301 - protein, 126 aa, Artificial Sequence
  Description of Artificial Sequence: Synthetic polypeptide
QMQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKDIHSGSYYGLLYYAMDVWGQGTTVTVSS

SEQ ID NO: 302 - protein, 113 aa, Artificial Sequence
  Description of Artificial Sequence: Synthetic polypeptide
QSVLTQPPSVSGAPGQRVTISCTGSSSNIGAGYDVHWYQQLPGTAPKLLIFGNSNRPSGVPDRFSGFKSGTSASLAITGLQAEDEADYFCQSYDSSLSGSGVFGTGTKVTVLG

SEQ ID NO: 303 - protein, 121 aa, Artificial Sequence
  Description of Artificial Sequence: Synthetic polypeptide
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARFQDWWYLGQFDQWGQGTLVTVSS

SEQ ID NO: 304 - protein, 113 aa, Artificial Sequence
  Description of Artificial Sequence: Synthetic polypeptide
QSALTQPASVSGSPGQSITISCTATGSDVGVYYYVSWYQQHPGKAPKVMIYDVDNRPPGVSNRFSGSKSGNTASLTISGLQAEDEADYYCASYTNRNSLGYVFGTGTKVTVLG

SEQ ID NO: 305 - protein, 6 aa, Artificial Sequence
  Description of Artificial Sequence: Synthetic peptide
DRGSQS

SEQ ID NO: 306 - protein, 6 aa, Artificial Sequence
  Description of Artificial Sequence: Synthetic peptide
IYSNGD

SEQ ID NO: 307 - protein, 12 aa, Artificial Sequence
  Description of Artificial Sequence: Synthetic peptide
AVNSDSGYALNF

SEQ ID NO: 308 - protein, 5 aa, Artificial Sequence
  Description of Artificial Sequence: Synthetic peptide
SGDLS

SEQ ID NO: 309 - protein, 6 aa, Artificial Sequence
  Description of Artificial Sequence: Synthetic peptide
YYNGEE

SEQ ID NO: 310 - protein, 13 aa, Artificial Sequence
  Description of Artificial Sequence: Synthetic peptide
CASSLGGESEQYF

SEQ ID NO: 311 - protein, 5 aa, Artificial Sequence
  Description of Artificial Sequence: Synthetic peptide
DRGSQ

SEQ ID NO: 312 - protein, 6 aa, Artificial Sequence
  Description of Artificial Sequence: Synthetic peptide
IYSNGD

SEQ ID NO: 313 - protein, 12 aa, Artificial Sequence
  Description of Artificial Sequence: Synthetic peptide
AVNSQSGYALNF

SEQ ID NO: 314 - protein, 110 aa, Artificial Sequence
  Description of Artificial Sequence: Synthetic polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSDSGYALNFGKGTSLLVT

SEQ ID NO: 315 - protein, 109 aa, Artificial Sequence
  Description of Artificial Sequence: Synthetic polypeptide
VTQTPKHLITATGQRVTLRCSPRSGDLSVYWYQQSLDQGLQFLIQYYNGEERAKGNILERFSAQQFPDLHSELNLSSLELGDSALYFCASSLGGESEQYFGPGTRLTVT

SEQ ID NO: 316 - protein, 110 aa, Artificial Sequence
  Description of Artificial Sequence: Synthetic polypeptide
QKEVEQNSGPLSVPEGAIASLNCTYSDRGSQAFFWYRQYSGKSPELIMSIYSNGDKEDGRFTAQLNKASQYVSLLIRDSQPSDSATYLCAVNSQSGYALNFGKGTSLLVT

SEQ ID NO: 317 - protein, 8 aa, Artificial Sequence
  Description of Artificial Sequence: Synthetic peptide
GGSFSGYY

SEQ ID NO: 318 - protein, 7 aa, Artificial Sequence
  Description of Artificial Sequence: Synthetic peptide
INHSGST

SEQ ID NO: 319 - protein, 10 aa, Artificial Sequence
  Description of Artificial Sequence: Synthetic peptide
ARGYGGRFDY

SEQ ID NO: 320 - protein, 8 aa, Artificial Sequence
  Description of Artificial Sequence: Synthetic peptide
SSNIGSNN

SEQ ID NO: 321 - protein, 3 aa, Artificial Sequence
  Description of Artificial Sequence: Synthetic peptide
SNH

SEQ ID NO: 322 - protein, 11 aa, Artificial Sequence
  Description of Artificial Sequence: Synthetic peptide
AAWDDSLDGYL

SEQ ID NO: 323 - protein, 8 aa, Artificial Sequence
  Description of Artificial Sequence: Synthetic peptide
GFTFSSYA

SEQ ID NO: 324 - protein, 8 aa, Artificial Sequence
  Description of Artificial Sequence: Synthetic peptide
IYSGGSST

SEQ ID NO: 325 - protein, 11 aa, Artificial Sequence
  Description of Artificial Sequence: Synthetic peptide
ARTSYLNHGDY

SEQ ID NO: 326 - protein, 8 aa, Artificial Sequence
  Description of Artificial Sequence: Synthetic peptide
RSNIGSDY

SEQ ID NO: 327 - protein, 3 aa, Artificial Sequence
  Description of Artificial Sequence: Synthetic peptide
GDN

SEQ ID NO: 328 - protein, 11 aa, Artificial Sequence
  Description of Artificial Sequence: Synthetic peptide
GTWDYTLNGVV

SEQ ID NO: 329 - protein, 8 aa, Artificial Sequence
  Description of Artificial Sequence: Synthetic peptide
GFTFSDYY

SEQ ID NO: 330 - protein, 8 aa, Artificial Sequence
  Description of Artificial Sequence: Synthetic peptide
ISSSGSTI

SEQ ID NO: 331 - protein, 9 aa, Artificial Sequence
  Description of Artificial Sequence: Synthetic peptide
ARASDLYGD

SEQ ID NO: 332 - protein, 8 aa, Artificial Sequence
  Description of Artificial Sequence: Synthetic peptide
TSNIGTNT

SEQ ID NO: 333 - protein, 3 aa, Artificial Sequence
  Description of Artificial Sequence: Synthetic peptide
SNN

SEQ ID NO: 334 - protein, 11 aa, Artificial Sequence
  Description of Artificial Sequence: Synthetic peptide
AAWDDSLNGVV

SEQ ID NO: 335 - protein, 8 aa, Artificial Sequence
  Description of Artificial Sequence: Synthetic peptide
GYRFSNYG

SEQ ID NO: 336 - protein, 8 aa, Artificial Sequence
  Description of Artificial Sequence: Synthetic peptide
ISGSNGNT

SEQ ID NO: 337 - protein, 14 aa, Artificial Sequence
  Description of Artificial Sequence: Synthetic peptide
ARGNRRYYSPIIDP

SEQ ID NO: 338 - protein, 8 aa, Artificial Sequence
  Description of Artificial Sequence: Synthetic peptide
SSNFGSNT

SEQ ID NO: 339 - protein, 3 aa, Artificial Sequence
  Description of Artificial Sequence: Synthetic peptide
SNT

SEQ ID NO: 340 - protein, 11 aa, Artificial Sequence
  Description of Artificial Sequence: Synthetic peptide
AAWDDSLTGVV

SEQ ID NO: 341 - protein, 8 aa, Artificial Sequence
  Description of Artificial Sequence: Synthetic peptide
GYTFTSYY

SEQ ID NO: 342 - protein, 8 aa, Artificial Sequence
  Description of Artificial Sequence: Synthetic peptide
INPSGGST

SEQ ID NO: 343 - protein, 10 aa, Artificial Sequence
  Description of Artificial Sequence: Synthetic peptide
ARWHGGPYDY

SEQ ID NO: 344 - protein, 6 aa, Artificial Sequence
  Description of Artificial Sequence: Synthetic peptide
NIGSKS

SEQ ID NO: 345 - protein, 3 aa, Artificial Sequence
  Description of Artificial Sequence: Synthetic peptide
YDS

SEQ ID NO: 346 - protein, 11 aa, Artificial Sequence
  Description of Artificial Sequence: Synthetic peptide
QVWDSSSDHYV

SEQ ID NO: 347 - protein, 8 aa, Artificial Sequence
  Description of Artificial Sequence: Synthetic peptide
GYTFASHG

SEQ ID NO: 348 - protein, 8 aa, Artificial Sequence
  Description of Artificial Sequence: Synthetic peptide
ISPYTGNT

SEQ ID NO: 349 - protein, 13 aa, Artificial Sequence
  Description of Artificial Sequence: Synthetic peptide
ARGKRTLASCFDY

SEQ ID NO: 350 - protein, 6 aa, Artificial Sequence
  Description of Artificial Sequence: Synthetic peptide
NIGSKS

SEQ ID NO: 351 - protein, 3 aa, Artificial Sequence
  Description of Artificial Sequence: Synthetic peptide
DDS

SEQ ID NO: 352 - protein, 10 aa, Artificial Sequence
  Description of Artificial Sequence: Synthetic peptide
QVWDSSSDHV

SEQ ID NO: 353 - protein, 8 aa, Artificial Sequence
  Description of Artificial Sequence: Synthetic peptide
GGTFGSYA

SEQ ID NO: 354 - protein, 8 aa, Artificial Sequence
  Description of Artificial Sequence: Synthetic peptide
IIPVLGRT

SEQ ID NO: 355 - protein, 6 aa, Artificial Sequence
  Description of Artificial Sequence: Synthetic peptide
ARTNDS

SEQ ID NO: 356 - protein, 11 aa, Artificial Sequence
  Description of Artificial Sequence: Synthetic peptide
QSLLHSNGYNY

SEQ ID NO: 357 - protein, 3 aa, Artificial Sequence
  Description of Artificial Sequence: Synthetic peptide
LGS

SEQ ID NO: 358 - protein, 9 aa, Artificial Sequence
  Description of Artificial Sequence: Synthetic peptide
MQALQTPWT

SEQ ID NO: 359 - protein, 6 aa, Artificial Sequence
  Description of Artificial Sequence: Synthetic peptide
DSAIYN

SEQ ID NO: 360 - protein, 7 aa, Artificial Sequence
  Description of Artificial Sequence: Synthetic peptide
IQSSQRE

SEQ ID NO: 361 - protein, 13 aa, Artificial Sequence
  Description of Artificial Sequence: Synthetic peptide
AVRPTSGGSYIPT

SEQ ID NO: 362 - protein, 100 aa, Artificial Sequence
  Description of Artificial Sequence: Synthetic polypeptide
QEVTQIPAALSVPEGENLVLNCSFTLQWFRQDPGKGLTSLLLQTSGRLNASLDKSSGRSTLYIAASQPGDSATYLCAVRPTSGGSYIPTFGRGTSLIVHP

SEQ ID NO: 363 - protein, 7 aa, Artificial Sequence
  Description of Artificial Sequence: Synthetic peptide
MNHEYMS

SEQ ID NO: 364 - protein, 6 aa, Artificial Sequence
  Description of Artificial Sequence: Synthetic peptide
SVGAGI

SEQ ID NO: 365 - protein, 12 aa, Artificial Sequence
  Description of Artificial Sequence: Synthetic peptide
ASSYVGNTGELF

SEQ ID NO: 366 - protein, 109 aa, Artificial Sequence
  Description of Artificial Sequence: Synthetic polypeptide
VTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTV

SEQ ID NO: 367 - protein, 119 aa, Artificial Sequence
  Description of Artificial Sequence: Synthetic polypeptide
QVKLQQSGTEVVKPGASVKLSCKASGYIFTSYDIDWVRQTPEQGLEWIGWIFPGEGSTEYNEKFKGRATLSVDKSSSTAYMELTRLTSEDSAVYFCARGDYYRRYFDLWGQGTTVTVSS

SEQ ID NO: 368 - protein, 110 aa, Artificial Sequence
  Description of Artificial Sequence: Synthetic polypeptide
DIELTQSPAIMSASPGERVTMTCSASSSIRYIYWYQQKPGSSPRLLIYDTSNVAPGVPFRFSGSGSGTSYSLTINRMEAEDAATYYCQEWSGYPYTFGGGTKLELKRAAA

SEQ ID NO: 369 - protein, 119 aa, Artificial Sequence
  Description of Artificial Sequence: Synthetic polypeptide
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 370 - protein, 449 aa, Artificial Sequence
  Description of Artificial Sequence: Synthetic polypeptide
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 110 aa, Artificial Sequence
  Description of Artificial Sequence: Synthetic polypeptide
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 372 - protein, 217 aa, Artificial Sequence
  Description of Artificial Sequence: Synthetic polypeptide
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 373 - protein, 7 aa, Artificial Sequence
  Description of Artificial Sequence: Synthetic peptide
NIGAGYD

SEQ ID NO: 374 - protein, 3 aa, Artificial Sequence
  Description of Artificial Sequence: Synthetic peptide
GNS

SEQ ID NO: 375 - protein, 11 aa, Artificial Sequence
  Description of Artificial Sequence: Synthetic peptide
QSYDSSLSGYV

SEQ ID NO: 376 - protein, 114 aa, Artificial Sequence
  Description of Artificial Sequence: Synthetic polypeptide
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGYVFGTGTKVTVLGSR

SEQ ID NO: 377 - protein, 8 aa, Artificial Sequence
  Description of Artificial Sequence: Synthetic peptide
GYSFTSYW

SEQ ID NO: 378 - protein, 8 aa, Artificial Sequence
  Description of Artificial Sequence: Synthetic peptide
IYPGDSDT

SEQ ID NO: 379 - protein, 14 aa, Artificial Sequence
  Description of Artificial Sequence: Synthetic peptide
ARSMGSSLYASSDV

SEQ ID NO: 380 - protein, 125 aa, Artificial Sequence
  Description of Artificial Sequence: Synthetic polypeptide
LEMAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSMGSSLYASSDVWGQGTLVTVSS

SEQ ID NO: 381 - protein, 8 aa, Artificial Sequence
  Description of Artificial Sequence: Synthetic peptide
SSNIGSNT

SEQ ID NO: 382 - protein, 3 aa, Artificial Sequence
  Description of Artificial Sequence: Synthetic peptide
SNN

SEQ ID NO: 383 - protein, 11 aa, Artificial Sequence
  Description of Artificial Sequence: Synthetic peptide
AAWDDSLNGYV

SEQ ID NO: 384 - protein, 113 aa, Artificial Sequence
  Description of Artificial Sequence: Synthetic polypeptide
QAVLTQPPSASGTPGQRVTISCSGSSSNIGSNTVNWYQQLPGTAPKLLMYSNNQRPSGVPDRFSGSKSGTSASLAISGLQSEDEADYYCAAWDDSLNGYVFGTGTKVTVLGSR

SEQ ID NO: 385 - protein, 8 aa, Artificial Sequence
  Description of Artificial Sequence: Synthetic peptide
GYNFASYW

SEQ ID NO: 386 - protein, 8 aa, Artificial Sequence
  Description of Artificial Sequence: Synthetic peptide
IYPDDSDT

SEQ ID NO: 387 - protein, 10 aa, Artificial Sequence
  Description of Artificial Sequence: Synthetic peptide
ARDSYYGIDV

SEQ ID NO: 388 - protein, 121 aa, Artificial Sequence
  Description of Artificial Sequence: Synthetic polypeptide
LEMAEVQLVQSGAEMKKPGESLKISCKGSGYNFASYWVGWVRQMPGKGLEWMGTIYPDDSDTRYGPAFQGQVTISADKSISTAYLQWSSLKASDTAMYYCARDSYYGIDVWGQGTLVTVSS

SEQ ID NO: 389 - protein, 8 aa, Artificial Sequence
  Description of Artificial Sequence: Synthetic peptide
GFTFSSYA

SEQ ID NO: 390 - protein, 8 aa, Artificial Sequence
  Description of Artificial Sequence: Synthetic peptide
ISGSGYST

SEQ ID NO: 391 - protein, 17 aa, Artificial Sequence
  Description of Artificial Sequence: Synthetic peptide
AKGFQYGSGSYYTHFDY

SEQ ID NO: 392 - protein, 6 aa, Artificial Sequence
  Description of Artificial Sequence: Synthetic peptide
QGISSW

SEQ ID NO: 393 - protein, 3 aa, Artificial Sequence
  Description of Artificial Sequence: Synthetic peptide
AAS

SEQ ID NO: 394 - protein, 9 aa, Artificial Sequence
  Description of Artificial Sequence: Synthetic peptide
QQYNSYPYT

SEQ ID NO: 395 - protein, 8 aa, Artificial Sequence
  Description of Artificial Sequence: Synthetic peptide
GFTFDDYA

SEQ ID NO: 396 - protein, 8 aa, Artificial Sequence
  Description of Artificial Sequence: Synthetic peptide
ISWNSGSI

SEQ ID NO: 397 - protein, 15 aa, Artificial Sequence
  Description of Artificial Sequence: Synthetic peptide
ARDLGAKQWLEGFDY

SEQ ID NO: 398 - protein, 142 aa, Artificial Sequence
  Description of Artificial Sequence: Synthetic polypeptide
MKYLLPTAAAGLLLLAAQPAMAQVQLVQSGGGLVQPGRSLRLSCAASGFTFDDYAMHWVRQAPGKGLEWVSGISWNSGSIGYADSVKGRFTISRDNAKNSLYLQMNSLRPEDTAVYYCARDLGAKQWLEGFDYWGQGTLVTV

SEQ ID NO: 399 - protein, 6 aa, Artificial Sequence
  Description of Artificial Sequence: Synthetic peptide
SLRSYY

SEQ ID NO: 400 - protein, 3 aa, Artificial Sequence
  Description of Artificial Sequence: Synthetic peptide
GKN

SEQ ID NO: 401 - protein, 11 aa, Artificial Sequence
  Description of Artificial Sequence: Synthetic peptide
NSRDSSGNHWV

SEQ ID NO: 402 - protein, 126 aa, Artificial Sequence
  Description of Artificial Sequence: Synthetic polypeptide
NFMLTQDPAVSVALGQTVRITCQGDSLRSYYASWYQQKPGQAPVLVIYGKNNRPSGIPDRFSGSTSGNSASLTITGAQAEDEADYYCNSRDSSGNHWVFGGGTKVTVLGAAAEQKLISEEDLNGAA

SEQ ID NO: 403 - protein, 8 aa, Artificial Sequence
  Description of Artificial Sequence: Synthetic peptide
GYAFTNYW

SEQ ID NO: 404 - protein, 8 aa, Artificial Sequence
  Description of Artificial Sequence: Synthetic peptide
IFPGSGNI

SEQ ID NO: 405 - protein, 12 aa, Artificial Sequence
  Description of Artificial Sequence: Synthetic peptide
ARLRNWDEPMDY

SEQ ID NO: 406 - protein, 12 aa, Artificial Sequence
  Description of Artificial Sequence: Synthetic peptide
QSLLNSGNQKNY

SEQ ID NO: 407 - protein, 3 aa, Artificial Sequence
  Description of Artificial Sequence: Synthetic peptide
WAS

SEQ ID NO: 408 - protein, 9 aa, Artificial Sequence
  Description of Artificial Sequence: Synthetic peptide
QNDYSYPLT

SEQ ID NO: 409 - protein, 8 aa, Artificial Sequence
  Description of Artificial Sequence: Synthetic peptide
GFTFRKFG

SEQ ID NO: 410 - protein, 8 aa, Artificial Sequence
  Description of Artificial Sequence: Synthetic peptide
ISTGGYNT

SEQ ID NO: 411 - protein, 13 aa, Artificial Sequence
  Description of Artificial Sequence: Synthetic peptide
TRGYSSTSYAMDY

SEQ ID NO: 412 - protein, 118 aa, Artificial Sequence
  Description of Artificial Sequence: Synthetic polypeptide
QVKLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTV

SEQ ID NO: 413 - protein, 6 aa, Artificial Sequence
  Description of Artificial Sequence: Synthetic peptide
TDIDDD

SEQ ID NO: 414 - protein, 3 aa, Artificial Sequence
  Description of Artificial Sequence: Synthetic peptide
EGN

SEQ ID NO: 415 - protein, 9 aa, Artificial Sequence
  Description of Artificial Sequence: Synthetic peptide
LQSFNVPLT

SEQ ID NO: 416 - protein, 107 aa, Artificial Sequence
  Description of Artificial Sequence: Synthetic polypeptide
DIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEIK

SEQ ID NO: 417 - protein, 8 aa, Artificial Sequence
  Description of Artificial Sequence: Synthetic peptide
GYIFTSYD

SEQ ID NO: 418 - protein, 8 aa, Artificial Sequence
  Description of Artificial Sequence: Synthetic peptide
IFPGEGST

SEQ ID NO: 419 - protein, 12 aa, Artificial Sequence
  Description of Artificial Sequence: Synthetic peptide
ARGDYYRRYFDL

SEQ ID NO: 420 - protein, 7 aa, Artificial Sequence
  Description of Artificial Sequence: Synthetic peptide
ASSSIRY

SEQ ID NO: 421 - protein, 3 aa, Artificial Sequence
  Description of Artificial Sequence: Synthetic peptide
DTS

SEQ ID NO: 422 - protein, 9 aa, Artificial Sequence
  Description of Artificial Sequence: Synthetic peptide
QEWSGYPYT

SEQ ID NO: 423 - protein, 5 aa, Artificial Sequence
  Description of Artificial Sequence: Synthetic peptide
GYGLS

SEQ ID NO: 424 - protein, 13 aa, Artificial Sequence
  Description of Artificial Sequence: Synthetic peptide
MISSGGSYTYYAD

SEQ ID NO: 425 - protein, 10 aa, Artificial Sequence
  Description of Artificial Sequence: Synthetic peptide
HGDDPAWFAY

SEQ ID NO: 426 - protein, 12 aa, Artificial Sequence
  Description of Artificial Sequence: Synthetic peptide
SVSSSISSNNLH

SEQ ID NO: 427 - protein, 7 aa, Artificial Sequence
  Description of Artificial Sequence: Synthetic peptide
GTSNLAS

SEQ ID NO: 428 - protein, 11 aa, Artificial Sequence
  Description of Artificial Sequence: Synthetic peptide
QQWSSYPYMYT

SEQ ID NO: 429 - protein, 8 aa, Artificial Sequence
  Description of Artificial Sequence: Synthetic peptide
GFTFSSYA

SEQ ID NO: 430 - protein, 8 aa, Artificial Sequence
  Description of Artificial Sequence: Synthetic peptide
ISSAGGYI

SEQ ID NO: 431 - protein, 16 aa, Artificial Sequence
  Description of Artificial Sequence: Synthetic peptide
ARQGFGNYGDYYAMDY

SEQ ID NO: 432 - protein, 12 aa, Artificial Sequence
  Description of Artificial Sequence: Synthetic peptide
QSLLNSRTRKNQ

SEQ ID NO: 433 - protein, 3 aa, Artificial Sequence
  Description of Artificial Sequence: Synthetic peptide
WAS

SEQ ID NO: 434 - protein, 8 aa, Artificial Sequence
  Description of Artificial Sequence: Synthetic peptide
QQSYNLLT

SEQ ID NO: 435 - protein, 10 aa, Artificial Sequence
  Description of Artificial Sequence: Synthetic peptide
GFSLSTVGMG

SEQ ID NO: 436 - protein, 8 aa, Artificial Sequence
  Description of Artificial Sequence: Synthetic peptide
IWWDDEDK

SEQ ID NO: 437 - protein, 14 aa, Artificial Sequence
  Description of Artificial Sequence: Synthetic peptide
TRIGTAQATDALDY

SEQ ID NO: 438 - protein, 11 aa, Artificial Sequence
  Description of Artificial Sequence: Synthetic peptide
KSLLHSNGNTY

SEQ ID NO: 439 - protein, 3 aa, Artificial Sequence
  Description of Artificial Sequence: Synthetic peptide
YMS

SEQ ID NO: 440 - protein, 9 aa, Artificial Sequence
  Description of Artificial Sequence: Synthetic peptide
MQSLEYPLT

SEQ ID NO: 441 - protein, 245 aa, Artificial Sequence
  Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSFGPGTKVDIKRSRGGGGSGGGGSGGGGSLEMAQVQLVQSGTEVKKPGSSVKVSCQASGGSLSSHGVSWLRQAPGQGLEWVGRIIPMFGVTDYAQKFQDRVTITADKSTSTVYMELISLGSDDTAVYFCARESRGATFEYWGQGTLVTVSS

SEQ ID NO: 442 - protein, 251 aa, Artificial Sequence
  Description of Artificial Sequence: Synthetic polypeptide
QSVLTQPASVSGSPGQSITISCTGTSSDFGDYDYVSWYQQHPGKAPKLMIYDVSDRPSGVSNRFSGSKSGNTASLTISGLQAEDEADYFCSSLTTSSTLVFGGGTKLTVLGSRGGGGSGGGGSGGGGSLEMAQLQLQESGPGLVKPSETLSLTCTVSGGSISSSSYYWGWIRQPPGKGLEWIGSIYYSGSTYYNPSLKSRVTISVDTSKNQFSLKLGSVTAADTAVYYCARHDGTDAFDIWGQGTTVTVSS

SEQ ID NO: 443 - protein, 23 aa, Artificial Sequence
  Description of Artificial Sequence: Synthetic peptide
KNDAPVVQEPRRLSFRSTIYGSR

SEQ ID NO: 444 - protein, 15 aa, Artificial Sequence
  Description of Artificial Sequence: Synthetic peptide
AANCIRIGIPMADPI

SEQ ID NO: 445 - protein, 19 aa, Artificial Sequence
  Description of Artificial Sequence: Synthetic peptide
SSTGVLFVKFGPPPTASPG

SEQ ID NO: 446 - protein, 15 aa, Artificial Sequence
  Description of Artificial Sequence: Synthetic peptide
SNPMILMRLKLPNCE

SEQ ID NO: 447 - protein, 10 aa, Artificial Sequence
  Description of Artificial Sequence: Synthetic peptide
GGSLSSHGVS

SEQ ID NO: 448 - protein, 17 aa, Artificial Sequence
  Description of Artificial Sequence: Synthetic peptide
RIIPMFGVTDYAQKFQD

SEQ ID NO: 449 - protein, 9 aa, Artificial Sequence
  Description of Artificial Sequence: Synthetic peptide
ESRGATFEY

SEQ ID NO: 450 - protein, 118 aa, Artificial Sequence
  Description of Artificial Sequence: Synthetic polypeptide
QVQLVQSGTEVKKPGSSVKVSCQASGGSLSSHGVSWLRQAPGQGLEWVGRIIPMFGVTDYAQKFQDRVTITADKSTSTVYMELISLGSDDTAVYFCARESRGATFEYWGQGTLVTVSS

SEQ ID NO: 451 - protein, 12 aa, Artificial Sequence
  Description of Artificial Sequence: Synthetic peptide
RASQSVSSSYLA

SEQ ID NO: 452 - protein, 7 aa, Artificial Sequence
  Description of Artificial Sequence: Synthetic peptide
GASSRAT

SEQ ID NO: 453 - protein, 6 aa, Artificial Sequence
  Description of Artificial Sequence: Synthetic peptide
QQYGSS

SEQ ID NO: 454 - protein, 106 aa, Artificial Sequence
  Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSFGPGTKVDIKR

SEQ ID NO: 455 - protein, 12 aa, Artificial Sequence
  Description of Artificial Sequence: Synthetic peptide
GGSISSSSYYWG

SEQ ID NO: 456 - protein, 16 aa, Artificial Sequence
  Description of Artificial Sequence: Synthetic peptide
SIYYSGSTYYNPSLKS

SEQ ID NO: 457 - protein, 9 aa, Artificial Sequence
  Description of Artificial Sequence: Synthetic peptide
HDGTDAFDI

SEQ ID NO: 458 - protein, 119 aa, Artificial Sequence
  Description of Artificial Sequence: Synthetic polypeptide
QLQLQESGPGLVKPSETLSLTCTVSGGSISSSSYYWGWIRQPPGKGLEWIGSIYYSGSTYYNPSLKSRVTISVDTSKNQFSLKLGSVTAADTAVYYCARHDGTDAFDIWGQGTTVTVSS

SEQ ID NO: 459 - protein, 14 aa, Artificial Sequence
  Description of Artificial Sequence: Synthetic peptide
TGTSSDFGDYDYVS

SEQ ID NO: 460 - protein, 7 aa, Artificial Sequence
  Description of Artificial Sequence: Synthetic peptide
DVSDRPS

SEQ ID NO: 461 - protein, 10 aa, Artificial Sequence
  Description of Artificial Sequence: Synthetic peptide
SSLTTSSTLV

SEQ ID NO: 462 - protein, 111 aa, Artificial Sequence
  Description of Artificial Sequence: Synthetic polypeptide
QSVLTQPASVSGSPGQSITISCTGTSSDFGDYDYVSWYQQHPGKAPKLMIYDVSDRPSGVSNRFSGSKSGNTASLTISGLQAEDEADYFCSSLTTSSTLVFGGGTKLTVLG

SEQ ID NO: 463 - protein, 9 aa, Artificial Sequence
  Description of Artificial Sequence: Synthetic peptide
LVVVGAGGV

SEQ ID NO: 464 - protein, 10 aa, Artificial Sequence
  Description of Artificial Sequence: Synthetic peptide
KLVVVGAGGV

SEQ ID NO: 465 - protein, 9 aa, Artificial Sequence
  Description of Artificial Sequence: Synthetic peptide
LVVVGAVGV

SEQ ID NO: 466 - protein, 9 aa, Artificial Sequence
  Description of Artificial Sequence: Synthetic peptide
LVVVGACGV

SEQ ID NO: 467 - protein, 9 aa, Artificial Sequence
  Description of Artificial Sequence: Synthetic peptide
LVVVGADGV

SEQ ID NO: 468 - protein, 10 aa, Artificial Sequence
  Description of Artificial Sequence: Synthetic peptide
KLVVVGAVGV

SEQ ID NO: 469 - protein, 10 aa, Artificial Sequence
  Description of Artificial Sequence: Synthetic peptide
KLVVVGACGV

SEQ ID NO: 470 - protein, 10 aa, Artificial Sequence
  Description of Artificial Sequence: Synthetic peptide
KLVVVGADGV

SEQ ID NO: 471 - protein, 10 aa, Artificial Sequence
  Description of Artificial Sequence: Synthetic peptide
KLVVVGASGV

SEQ ID NO: 472 - protein, 9 aa, Artificial Sequence
  Description of Artificial Sequence: Synthetic peptide
VLPLTVAEV

SEQ ID NO: 473 - protein, 9 aa, Artificial Sequence
  Description of Artificial Sequence: Synthetic peptide
SLLFLLFSL

SEQ ID NO: 474 - protein, 10 aa, Artificial Sequence
  Description of Artificial Sequence: Synthetic peptide
ALYVDSLFFL

SEQ ID NO: 475 - protein, 9 aa, Artificial Sequence
  Description of Artificial Sequence: Synthetic peptide
NLTHVLYPV

SEQ ID NO: 476 - protein, 9 aa, Artificial Sequence
  Description of Artificial Sequence: Synthetic peptide
VLDGLDVLL

SEQ ID NO: 477 - protein, 10 aa, Artificial Sequence
  Description of Artificial Sequence: Synthetic peptide
SLYSFPEPEA

SEQ ID NO: 478 - protein, 9 aa, Artificial Sequence
  Description of Artificial Sequence: Synthetic peptide
SLLQHLIGL

SEQ ID NO: 479 - protein, 9 aa, Artificial Sequence
  Description of Artificial Sequence: Synthetic peptide
RMFPNAPYL

SEQ ID NO: 480 - protein, 10 aa, Artificial Sequence
  Description of Artificial Sequence: Synthetic peptide
RKSAPSTGGV

SEQ ID NO: 481 - protein, 10 aa, Artificial Sequence
  Description of Artificial Sequence: Synthetic peptide
RMSAPSTGGV

SEQ ID NO: 482 - protein, 10 aa, Artificial Sequence
  Description of Artificial Sequence: Synthetic peptide
GVYDGREHTV

SEQ ID NO: 483 - protein, 9 aa, Artificial Sequence
  Description of Artificial Sequence: Synthetic peptide
KVLEHVVRV

SEQ ID NO: 484 - protein, 9 aa, Artificial Sequence
  Description of Artificial Sequence: Synthetic peptide
NYKRCFPVI

SEQ ID NO: 485 - protein, 9 aa, Artificial Sequence
  Description of Artificial Sequence: Synthetic peptide
RLARLALVL

SEQ ID NO: 486 - protein, 13 aa, Artificial Sequence
  Description of Artificial Sequence: Synthetic peptide
CAVPDDAGNMLTF

SEQ ID NO: 487 - protein, 16 aa, Artificial Sequence
  Description of Artificial Sequence: Synthetic peptide
CASSELPAGGTNEQFF

SEQ ID NO: 488 - protein, 15 aa, Artificial Sequence
  Description of Artificial Sequence: Synthetic peptide
CASMYSGGGADGLTF

SEQ ID NO: 489 - protein, 14 aa, Artificial Sequence
  Description of Artificial Sequence: Synthetic peptide
CASSFFSNTGELFF

SEQ ID NO: 490 - protein, 12 aa, Artificial Sequence
  Description of Artificial Sequence: Synthetic peptide
CASGGGADGLTF

SEQ ID NO: 491 - protein, 12 aa, Artificial Sequence
  Description of Artificial Sequence: Synthetic peptide
CASSFLTDTQYF

SEQ ID NO: 492 - protein, 12 aa, Artificial Sequence
  Description of Artificial Sequence: Synthetic peptide
CAGGGGADGLTF

SEQ ID NO: 493 - protein, 12 aa, Artificial Sequence
  Description of Artificial Sequence: Synthetic peptide
CASSYMGPEAFF

SEQ ID NO: 494 - protein, 12 aa, Artificial Sequence
  Description of Artificial Sequence: Synthetic peptide
CSSGGGADGLTF

SEQ ID NO: 495 - protein, 12 aa, Artificial Sequence
  Description of Artificial Sequence: Synthetic peptide
CASMDLAFKQYF

SEQ ID NO: 496 - protein, 17 aa, Artificial Sequence
  Description of Artificial Sequence: Synthetic peptide
CAYRSGSDGGSQGNLIF

SEQ ID NO: 497 - protein, 13 aa, Artificial Sequence
  Description of Artificial Sequence: Synthetic peptide
CASSQVSGYEQYF

SEQ ID NO: 498 - protein, 13 aa, Artificial Sequence
  Description of Artificial Sequence: Synthetic peptide
CAVRDDYGQNFVF

SEQ ID NO: 499 - protein, 13 aa, Artificial Sequence
  Description of Artificial Sequence: Synthetic peptide
CASSPQGDNEQFF

SEQ ID NO: 500 - protein, 10 aa, Artificial Sequence
  Description of Artificial Sequence: Synthetic peptide
MVNTVAGAMK

SEQ ID NO: 501 - protein, 14 aa, Artificial Sequence
  Description of Artificial Sequence: Synthetic peptide
CALSSQTGANNLFF

SEQ ID NO: 502 - protein, 19 aa, Artificial Sequence
  Description of Artificial Sequence: Synthetic peptide
CASSLVSPSSGSYTGELFF

SEQ ID NO: 503 - protein, 12 aa, Artificial Sequence
  Description of Artificial Sequence: Synthetic peptide
CAASSNNNDMRF

SEQ ID NO: 504 - protein, 14 aa, Artificial Sequence
  Description of Artificial Sequence: Synthetic peptide
CASSLVRGSTEAFF

SEQ ID NO: 505 - protein, 10 aa, Artificial Sequence
  Description of Artificial Sequence: Synthetic peptide
KLYGLDWAEL

SEQ ID NO: 506 - protein, 11 aa, Artificial Sequence
  Description of Artificial Sequence: Synthetic peptide
CAYFGAQKLVF

SEQ ID NO: 507 - protein, 15 aa, Artificial Sequence
  Description of Artificial Sequence: Synthetic peptide
CASRPSRGTNYGYTF

SEQ ID NO: 508 - protein, 11 aa, Artificial Sequence
  Description of Artificial Sequence: Synthetic peptide
CARYYGQNFVF

SEQ ID NO: 509 - protein, 13 aa, Artificial Sequence
  Description of Artificial Sequence: Synthetic peptide
CASSERGMVEAFF

SEQ ID NO: 510 - protein, 15 aa, Artificial Sequence
  Description of Artificial Sequence: Synthetic peptide
CALDDRGSTLGRLYF

SEQ ID NO: 511 - protein, 18 aa, Artificial Sequence
  Description of Artificial Sequence: Synthetic peptide
CASSPRNLGPSGSYEQYF

SEQ ID NO: 512 - protein, 9 aa, Artificial Sequence
  Description of Artificial Sequence: Synthetic peptide
TYDTVHRHL

SEQ ID NO: 513 - protein, 13 aa, Artificial Sequence
  Description of Artificial Sequence: Synthetic peptide
CILRDVGNYQLIF

SEQ ID NO: 514 - protein, 13 aa, Artificial Sequence
  Description of Artificial Sequence: Synthetic peptide
CASSLDREDEQYF

SEQ ID NO: 515 - protein, 9 aa, Artificial Sequence
  Description of Artificial Sequence: Synthetic peptide
ELLVRINRL

SEQ ID NO: 516 - protein, 15 aa, Artificial Sequence
  Description of Artificial Sequence: Synthetic peptide
CAVRDRGTGGFKTIF

SEQ ID NO: 517 - protein, 14 aa, Artificial Sequence
  Description of Artificial Sequence: Synthetic peptide
CASITKDRAYEQYF

SEQ ID NO: 518 - protein, 10 aa, Artificial Sequence
  Description of Artificial Sequence: Synthetic peptide
ALDPHSGHFV

SEQ ID NO: 519 - protein, 14 aa, Artificial Sequence
  Description of Artificial Sequence: Synthetic peptide
CVVSDLYNTDKLIF

SEQ ID NO: 520 - protein, 11 aa, Artificial Sequence
  Description of Artificial Sequence: Synthetic peptide
CASSQNYEQYF

SEQ ID NO: 521 - protein, 15 aa, Artificial Sequence
  Description of Artificial Sequence: Synthetic peptide
CAMSSLSGNTGKLIF

SEQ ID NO: 522 - skipped

SEQ ID NO: 523 - protein, 15 aa, Artificial Sequence
  Description of Artificial Sequence: Synthetic peptide
CASSYSWGAGYEQYF

SEQ ID NO: 524 - protein, 11 aa, Artificial Sequence
  Description of Artificial Sequence: Synthetic peptide
CALPYGNKLVF

SEQ ID NO: 525 - protein, 13 aa, Artificial Sequence
  Description of Artificial Sequence: Synthetic peptide
CASTPTGAYEQYF

SEQ ID NO: 526 - protein, 14 aa, Artificial Sequence
  Description of Artificial Sequence: Synthetic peptide
CAVILRSNDYKLSF

SEQ ID NO: 527 - protein, 10 aa, Artificial Sequence
  Description of Artificial Sequence: Synthetic peptide
CSAGTGELFF

SEQ ID NO: 528 - protein, 11 aa, Artificial Sequence
  Description of Artificial Sequence: Synthetic peptide
CALPYGNKLVF

SEQ ID NO: 529 - protein, 13 aa, Artificial Sequence
  Description of Artificial Sequence: Synthetic peptide
CASTPTGAYEQYF

SEQ ID NO: 530 - protein, 10 aa, Artificial Sequence
  Description of Artificial Sequence: Synthetic peptide
VLLGVKLFGV

SEQ ID NO: 531 - protein, 14 aa, Artificial Sequence
  Description of Artificial Sequence: Synthetic peptide
CAVEGDTGFQKLVF

SEQ ID NO: 532 - protein, 17 aa, Artificial Sequence
  Description of Artificial Sequence: Synthetic peptide
CASSSPRSSRDTDTQYF

SEQ ID NO: 533 - protein, 12 aa, Artificial Sequence
  Description of Artificial Sequence: Synthetic peptide
CADVPYGQNFVF

SEQ ID NO: 534 - protein, 18 aa, Artificial Sequence
  Description of Artificial Sequence: Synthetic peptide
CASSYSKAGGPGEDTQYF

SEQ ID NO: 535 - protein, 15 aa, Artificial Sequence
  Description of Artificial Sequence: Synthetic peptide
GYNSYSVSNSEKHIM

SEQ ID NO: 536 - protein, 13 aa, Artificial Sequence
  Description of Artificial Sequence: Synthetic peptide
CVVFTGGGNKLTF

SEQ ID NO: 537 - protein, 13 aa, Artificial Sequence
  Description of Artificial Sequence: Synthetic peptide
CASTPQVNYGYTF

SEQ ID NO: 538 - protein, 13 aa, Artificial Sequence
  Description of Artificial Sequence: Synthetic peptide
CVVFGGNNARLMF

SEQ ID NO: 539 - protein, 13 aa, Artificial Sequence
  Description of Artificial Sequence: Synthetic peptide
CASTLQANYGYTF

SEQ ID NO: 540 - protein, 9 aa, Artificial Sequence
  Description of Artificial Sequence: Synthetic peptide
LYPEFIASI

SEQ ID NO: 541 - protein, 13 aa, Artificial Sequence
  Description of Artificial Sequence: Synthetic peptide
CAVRGTSGYALNF

SEQ ID NO: 542 - protein, 16 aa, Artificial Sequence
  Description of Artificial Sequence: Synthetic peptide
CATSFAPGQGEHGYTF

SEQ ID NO: 543 - protein, 13 aa, Artificial Sequence
  Description of Artificial Sequence: Synthetic peptide
CAASGISGSRLTF

SEQ ID NO: 544 - protein, 14 aa, Artificial Sequence
  Description of Artificial Sequence: Synthetic peptide
CASRDHRVIYGYTF

SEQ ID NO: 545 - protein, 25 aa, Artificial Sequence
  Description of Artificial Sequence: Synthetic peptide
DMKTALALYEFPSMGLLSALDHGII

SEQ ID NO: 546 - protein, 13 aa, Artificial Sequence
  Description of Artificial Sequence: Synthetic peptide
CAVRDNYGQNFVF

SEQ ID NO: 547 - protein, 16 aa, Artificial Sequence
  Description of Artificial Sequence: Synthetic peptide
CASSLSWGSSYNEQFF

SEQ ID NO: 548 - protein, 15 aa, Artificial Sequence
  Description of Artificial Sequence: Synthetic peptide
NHVVDISKSGLITIA

SEQ ID NO: 549 - protein, 15 aa, Artificial Sequence
  Description of Artificial Sequence: Synthetic peptide
CAVTPNSGGYQKVTF

SEQ ID NO: 550 - protein, 17 aa, Artificial Sequence
  Description of Artificial Sequence: Synthetic peptide
CASTAGLLGSSYNEQFF

SEQ ID NO: 551 - protein, 8 aa, Artificial Sequence
  Description of Artificial Sequence: Synthetic peptide
YRPGTVTL

SEQ ID NO: 552 - protein, 13 aa, Artificial Sequence
  Description of Artificial Sequence: Synthetic peptide
CAMREDNNARLMF

SEQ ID NO: 553 - protein, 17 aa, Artificial Sequence
  Description of Artificial Sequence: Synthetic peptide
CASSQEFVGAVLDTQYF

SEQ ID NO: 554 - protein, 12 aa, Artificial Sequence
  Description of Artificial Sequence: Synthetic peptide
YVMAYVMAGVGS

SEQ ID NO: 555 - protein, 12 aa, Artificial Sequence
  Description of Artificial Sequence: Synthetic peptide
CAVSVNTDKLIF

SEQ ID NO: 556 - protein, 14 aa, Artificial Sequence
  Description of Artificial Sequence: Synthetic peptide
CSAPPLAGDETQYF

SEQ ID NO: 557 - protein, 10 aa, Artificial Sequence
  Description of Artificial Sequence: Synthetic peptide
MPYGYVLNEF

SEQ ID NO: 558 - protein, 12 aa, Artificial Sequence
  Description of Artificial Sequence: Synthetic peptide
CAVGTSYDKVIF

SEQ ID NO: 559 - protein, 15 aa, Artificial Sequence
  Description of Artificial Sequence: Synthetic peptide
CASRPWTGANEKLFF

SEQ ID NO: 560 - protein, 11 aa, Artificial Sequence
  Description of Artificial Sequence: Synthetic peptide
CAVHRDYKLSF

SEQ ID NO: 561 - protein, 13 aa, Artificial Sequence
  Description of Artificial Sequence: Synthetic peptide
CASSPGPNQPQHF

SEQ ID NO: 562 - protein, 11 aa, Artificial Sequence
  Description of Artificial Sequence: Synthetic peptide
CAVSADYKLSF

SEQ ID NO: 563 - protein, 13 aa, Artificial Sequence
  Description of Artificial Sequence: Synthetic peptide
CASSPGPNQPQHF

SEQ ID NO: 564 - protein, 11 aa, Artificial Sequence
  Description of Artificial Sequence: Synthetic peptide
CAVSADYKLSF

SEQ ID NO: 565 - protein, 13 aa, Artificial Sequence
  Description of Artificial Sequence: Synthetic peptide
CASTPTGNTEAFF

SEQ ID NO: 566 - protein, 25 aa, Artificial Sequence
  Description of Artificial Sequence: Synthetic peptide
KKPRHDLPPYRVYLTPYTVDSPICD

SEQ ID NO: 567 - protein, 11 aa, Artificial Sequence
  Description of Artificial Sequence: Synthetic peptide
CAVADGQKLLF

SEQ ID NO: 568 - protein, 15 aa, Artificial Sequence
  Description of Artificial Sequence: Synthetic peptide
CASSSTGADSHEQFF

SEQ ID NO: 569 - protein, 25 aa, Artificial Sequence
  Description of Artificial Sequence: Synthetic peptide
REKQQREALERAPARLERRHSALQR

SEQ ID NO: 570 - protein, 15 aa, Artificial Sequence
  Description of Artificial Sequence: Synthetic peptide
CGADFLMNRDDKIIF

SEQ ID NO: 571 - protein, 14 aa, Artificial Sequence
  Description of Artificial Sequence: Synthetic peptide
CASRAVINTDTQYF

SEQ ID NO: 572 - protein, 15 aa, Artificial Sequence
  Description of Artificial Sequence: Synthetic peptide
CAMRERGTSYDKVIF

SEQ ID NO: 573 - protein, 14 aa, Artificial Sequence
  Description of Artificial Sequence: Synthetic peptide
CASRDGRVHQPQHF

SEQ ID NO: 574 - protein, 15 aa, Artificial Sequence
  Description of Artificial Sequence: Synthetic peptide
CATDPSYSSASKIIF

SEQ ID NO: 575 - protein, 15 aa, Artificial Sequence
  Description of Artificial Sequence: Synthetic peptide
CASSLLKAANYGYTF

SEQ ID NO: 576 - protein, 25 aa, Artificial Sequence
  Description of Artificial Sequence: Synthetic peptide
MTEYKLVVVGAVGVGKSALTIQLIQ

SEQ ID NO: 577 - protein, 15 aa, Artificial Sequence
  Description of Artificial Sequence: Synthetic peptide
CLVGDMDQAGTALIF

SEQ ID NO: 578 - protein, 14 aa, Artificial Sequence
  Description of Artificial Sequence: Synthetic peptide
CASSLGQTNYGYTF

SEQ ID NO: 579 - protein, 16 aa, Artificial Sequence
  Description of Artificial Sequence: Synthetic peptide
CAVGRSNSGGYQKVTF

SEQ ID NO: 580 - protein, 15 aa, Artificial Sequence
  Description of Artificial Sequence: Synthetic peptide
CAWSALAGARDTQYF

SEQ ID NO: 581 - protein, 16 aa, Artificial Sequence
  Description of Artificial Sequence: Synthetic peptide
CAVTVVNAGNNRKLIW

SEQ ID NO: 582 - protein, 15 aa, Artificial Sequence
  Description of Artificial Sequence: Synthetic peptide
CASSLGLPGTDTQYF

SEQ ID NO: 583 - protein, 15 aa, Artificial Sequence
  Description of Artificial Sequence: Synthetic peptide
CLVGDMDQAGTALIF

SEQ ID NO: 584 - protein, 15 aa, Artificial Sequence
  Description of Artificial Sequence: Synthetic peptide
CASSLGEGRVDGYTF

SEQ ID NO: 585 - protein, 13 aa, Artificial Sequence
  Description of Artificial Sequence: Synthetic peptide
CAAAMDSSYKLIF

SEQ ID NO: 586 - protein, 12 aa, Artificial Sequence
  Description of Artificial Sequence: Synthetic peptide
CASSDPGTEAFF

SEQ ID NO: 587 - protein, 15 aa, Artificial Sequence
  Description of Artificial Sequence: Synthetic peptide
CLVGDRDQAGTALIF

SEQ ID NO: 588 - protein, 15 aa, Artificial Sequence
  Description of Artificial Sequence: Synthetic peptide
CASSFGQSSTYGYTF

SEQ ID NO: 589 - protein, 15 aa, Artificial Sequence
  Description of Artificial Sequence: Synthetic peptide
CLVGDMDQAGTALIF

SEQ ID NO: 590 - protein, 15 aa, Artificial Sequence
  Description of Artificial Sequence: Synthetic peptide
CASSLGRASNQPQHF

SEQ ID NO: 591 - protein, 10 aa, Artificial Sequence
  Description of Artificial Sequence: Synthetic peptide
GVYDGREHTV

SEQ ID NO: 592 - protein, 208 aa, Artificial Sequence
  Description of Artificial Sequence: Synthetic polypeptide
MKNQVEQSPQSLIILEGKNVTLQCNYTVSPFSNLRWYKQDTGRGPVSLTIMTFSENTKSNGRYTATLDADTKQSSLHITASQLSDSASYICVVNHSGGSYIPTFGRGTSLIVHPYIQKPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 593 - protein, 247 aa, Artificial Sequence
  Description of Artificial Sequence: Synthetic polypeptide
MDVKVTQSSRYLVKRTGEKVFLECVQDMDHENMFWYRQDPGLGLRLIYFSYDVKMKEKGDIPEGYSVSREKKERFSLILESASTNQTSMYLCASSFLMTSGDPYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 594 - protein, 11 aa, Artificial Sequence
  Description of Artificial Sequence: Synthetic peptide
LMKVDPIGHVY

SEQ ID NO: 595 - protein, 11 aa, Artificial Sequence
  Description of Artificial Sequence: Synthetic peptide
CAVDNFNKFYF

SEQ ID NO: 596 - protein, 13 aa, Artificial Sequence
  Description of Artificial Sequence: Synthetic peptide
CASSSQGGYGYTF

SEQ ID NO: 597 - protein, 10 aa, Artificial Sequence
  Description of Artificial Sequence: Synthetic peptide
CAGSGSRLTF

SEQ ID NO: 598 - protein, 13 aa, Artificial Sequence
  Description of Artificial Sequence: Synthetic peptide
CASSFDRGYGYTF

SEQ ID NO: 599 - protein, 8 aa, Artificial Sequence
  Description of Artificial Sequence: Synthetic peptide
VQIISCQY

SEQ ID NO: 600 - protein, 12 aa, Artificial Sequence
  Description of Artificial Sequence: Synthetic peptide
CVVNTNAGKSTF

SEQ ID NO: 601 - protein, 17 aa, Artificial Sequence
  Description of Artificial Sequence: Synthetic peptide
CASSGRVTGGFYNEQFF

SEQ ID NO: 602 - protein, 12 aa, Artificial Sequence
  Description of Artificial Sequence: Synthetic peptide
CASSGGNTPLVF

SEQ ID NO: 603 - protein, 13 aa, Artificial Sequence
  Description of Artificial Sequence: Synthetic peptide
CASSFGGAYEQYF

SEQ ID NO: 604 - protein, 9 aa, Artificial Sequence
  Description of Artificial Sequence: Synthetic peptide
ALSPVIPHI

SEQ ID NO: 605 - protein, 14 aa, Artificial Sequence
  Description of Artificial Sequence: Synthetic peptide
CALNPFNAGNMLTF

SEQ ID NO: 606 - protein, 13 aa, Artificial Sequence
  Description of Artificial Sequence: Synthetic peptide
CSARVGDTGELFF

SEQ ID NO: 607 - protein, 14 aa, Artificial Sequence
  Description of Artificial Sequence: Synthetic peptide
CAVGEAGGGNKLTF

SEQ ID NO: 608 - protein, 14 aa, Artificial Sequence
  Description of Artificial Sequence: Synthetic peptide
CASSYDSTTGELFF

SEQ ID NO: 609 - protein, 25 aa, Artificial Sequence
  Description of Artificial Sequence: Synthetic peptide
TGLFGQTNTGFGDVGSTLFGNNKLT

SEQ ID NO: 610 - protein, 16 aa, Artificial Sequence
  Description of Artificial Sequence: Synthetic peptide
CASTAGPNFGNEKLTF

SEQ ID NO: 611 - protein, 15 aa, Artificial Sequence
  Description of Artificial Sequence: Synthetic peptide
CASSANRGPDTEAFF

SEQ ID NO: 612 - protein, 9 aa, Artificial Sequence
  Description of Artificial Sequence: Synthetic peptide
HMTEVVRHC

SEQ ID NO: 613 - protein, 13 aa, Artificial Sequence
  Description of Artificial Sequence: Synthetic peptide
CVVQPGGYQKVTF

SEQ ID NO: 614 - protein, 16 aa, Artificial Sequence
  Description of Artificial Sequence: Synthetic peptide
CASSEGLWQVGDEQYF

SEQ ID NO: 615 - protein, 12 aa, Artificial Sequence
  Description of Artificial Sequence: Synthetic peptide
CALDIYPHDMRF

SEQ ID NO: 616 - protein, 15 aa, Artificial Sequence
  Description of Artificial Sequence: Synthetic peptide
CASSLDPGDTGELFF

SEQ ID NO: 617 - protein, 16 aa, Artificial Sequence
  Description of Artificial Sequence: Synthetic peptide
CAFMGYSGAGSYQLTF

SEQ ID NO: 618 - protein, 15 aa, Artificial Sequence
  Description of Artificial Sequence: Synthetic peptide
CAISELVTGDSPLHF

SEQ ID NO: 619 - protein, 18 aa, Artificial Sequence
  Description of Artificial Sequence: Synthetic peptide
CAASKSAIMVVLQTSSSL

SEQ ID NO: 620 - protein, 14 aa, Artificial Sequence
  Description of Artificial Sequence: Synthetic peptide
CASSIQQGADTQYF

SEQ ID NO: 621 - protein, 13 aa, Artificial Sequence
  Description of Artificial Sequence: Synthetic peptide
CALITGGGNKLTF

SEQ ID NO: 622 - protein, 14 aa, Artificial Sequence
  Description of Artificial Sequence: Synthetic peptide
CASRLQGWNSPLHF

SEQ ID NO: 623 - protein, 14 aa, Artificial Sequence
  Description of Artificial Sequence: Synthetic peptide
CAWNSGGSNYKLTF

SEQ ID NO: 624 - protein, 15 aa, Artificial Sequence
  Description of Artificial Sequence: Synthetic peptide
CASSYSQAWGQPQHF

SEQ ID NO: 625 - protein, 12 aa, Artificial Sequence
  Description of Artificial Sequence: Synthetic peptide
CAVRVWDYKLSF

SEQ ID NO: 626 - protein, 14 aa, Artificial Sequence
  Description of Artificial Sequence: Synthetic peptide
CASSISAGGDGYTF

SEQ ID NO: 627 - protein, 11 aa, Artificial Sequence
  Description of Artificial Sequence: Synthetic peptide
CAVKGDYKLSF

SEQ ID NO: 628 - protein, 12 aa, Artificial Sequence
  Description of Artificial Sequence: Synthetic peptide
CASSLVNTEAFF

SEQ ID NO: 629 - protein, 12 aa, Artificial Sequence
  Description of Artificial Sequence: Synthetic peptide
CAVYTGGFKTIF

SEQ ID NO: 630 - protein, 15 aa, Artificial Sequence
  Description of Artificial Sequence: Synthetic peptide
CASNLGGGSTDTQYF

SEQ ID NO: 631 - protein, 14 aa, Artificial Sequence
  Description of Artificial Sequence: Synthetic peptide
CAFYYGGSQGNLIF

SEQ ID NO: 632 - protein, 15 aa, Artificial Sequence
  Description of Artificial Sequence: Synthetic peptide
CASSFGSGSTDTQYF

SEQ ID NO: 633 - protein, 14 aa, Artificial Sequence
  Description of Artificial Sequence: Synthetic peptide
CAFYYGGSQGNLIF

SEQ ID NO: 634 - protein, 15 aa, Artificial Sequence
  Description of Artificial Sequence: Synthetic peptide
CASSLGTGSTDTQYF

SEQ ID NO: 635 - protein, 10 aa, Artificial Sequence
  Description of Artificial Sequence: Synthetic peptide
FVVPYMIYLL

SEQ ID NO: 636 - protein, 17 aa, Artificial Sequence
  Description of Artificial Sequence: Synthetic peptide
CAFTELNSGGSNYKLTF

SEQ ID NO: 637 - protein, 17 aa, Artificial Sequence
  Description of Artificial Sequence: Synthetic peptide
CASSLSGGLLRTGELFF

SEQ ID NO: 638 - protein, 25 aa, Artificial Sequence
  Description of Artificial Sequence: Synthetic peptide
ILCHQNPVTGLLLASYDQKDAWVRD

SEQ ID NO: 639 - protein, 14 aa, Artificial Sequence
  Description of Artificial Sequence: Synthetic peptide
CVGDFNNAGNMLTF

SEQ ID NO: 640 - protein, 11 aa, Artificial Sequence
  Description of Artificial Sequence: Synthetic peptide
CASSPGREQYF

SEQ ID NO: 641 - protein, 25 aa, Artificial Sequence
  Description of Artificial Sequence: Synthetic peptide
GNISILQENDFGVFGMDDREIMREG

SEQ ID NO: 642 - protein, 13 aa, Artificial Sequence
  Description of Artificial Sequence: Synthetic peptide
CAARMEYGNKLVF

SEQ ID NO: 643 - protein, 16 aa, Artificial Sequence
  Description of Artificial Sequence: Synthetic peptide
CASSFGTGRGDTEAFF

SEQ ID NO: 644 - protein, 14 aa, Artificial Sequence
  Description of Artificial Sequence: Synthetic peptide
CAVRSSPTGNQFYF

SEQ ID NO: 645 - protein, 16 aa, Artificial Sequence
  Description of Artificial Sequence: Synthetic peptide
CASSFGTGRGDTEAFF

SEQ ID NO: 646 - protein, 14 aa, Artificial Sequence
  Description of Artificial Sequence: Synthetic peptide
CAESRMDSSYKLIF

SEQ ID NO: 647 - protein, 15 aa, Artificial Sequence
  Description of Artificial Sequence: Synthetic peptide
CASSLVAGGNTEAFF

SEQ ID NO: 648 - protein, 14 aa, Artificial Sequence
  Description of Artificial Sequence: Synthetic peptide
CAVRSAAAGNKLTF

SEQ ID NO: 649 - protein, 16 aa, Artificial Sequence
  Description of Artificial Sequence: Synthetic peptide
CASSRSTGGSGNTIYF

SEQ ID NO: 650 - protein, 14 aa, Artificial Sequence
  Description of Artificial Sequence: Synthetic peptide
CAETRGGATNKLIF

SEQ ID NO: 651 - protein, 14 aa, Artificial Sequence
  Description of Artificial Sequence: Synthetic peptide
CASSQAGGTGELFF

SEQ ID NO: 652 - protein, 16 aa, Artificial Sequence
  Description of Artificial Sequence: Synthetic peptide
CAMSPLLETGANNLFF

SEQ ID NO: 653 - protein, 17 aa, Artificial Sequence
  Description of Artificial Sequence: Synthetic peptide
CASSTSTGQGWHYGYTF

SEQ ID NO: 654 - protein, 9 aa, Artificial Sequence
  Description of Artificial Sequence: Synthetic peptide
MLFSHGLVK

SEQ ID NO: 655 - protein, 13 aa, Artificial Sequence
  Description of Artificial Sequence: Synthetic peptide
CAPSYSSASKIIF

SEQ ID NO: 656 - protein, 14 aa, Artificial Sequence
  Description of Artificial Sequence: Synthetic peptide
CASSPGTVKETQYF

SEQ ID NO: 657 - protein, 14 aa, Artificial Sequence
  Description of Artificial Sequence: Synthetic peptide
CAMSGYTNAGKSTF

SEQ ID NO: 658 - protein, 16 aa, Artificial Sequence
  Description of Artificial Sequence: Synthetic peptide
CAWSAASGGAQDTQYF

SEQ ID NO: 659 - protein, 13 aa, Artificial Sequence
  Description of Artificial Sequence: Synthetic peptide
DPPALASTNAEVT

SEQ ID NO: 660 - protein, 14 aa, Artificial Sequence
  Description of Artificial Sequence: Synthetic peptide
CVVSAAQAGTALIF

SEQ ID NO: 661 - protein, 15 aa, Artificial Sequence
  Description of Artificial Sequence: Synthetic peptide
CASSASTGRNQPQHF

SEQ ID NO: 662 - protein, 15 aa, Artificial Sequence
  Description of Artificial Sequence: Synthetic peptide
CAMSAGANTGNQFYF

SEQ ID NO: 663 - protein, 14 aa, Artificial Sequence
  Description of Artificial Sequence: Synthetic peptide
CASSLARRQYGYTF

SEQ ID NO: 664 - protein, 14 aa, Artificial Sequence
  Description of Artificial Sequence: Synthetic peptide
CILRAPSGNTPLVF

SEQ ID NO: 665 - protein, 14 aa, Artificial Sequence
  Description of Artificial Sequence: Synthetic peptide
CASSSQGFYNIQYF

SEQ ID NO: 666 - protein, 14 aa, Artificial Sequence
  Description of Artificial Sequence: Synthetic peptide
CVVSPSGGSYIPTF

SEQ ID NO: 667 - protein, 15 aa, Artificial Sequence
  Description of Artificial Sequence: Synthetic peptide
CASSPGSPSSYEQYF

SEQ ID NO: 668 - protein, 17 aa, Artificial Sequence
  Description of Artificial Sequence: Synthetic peptide
CALSDPQIKAAGNKLTF

SEQ ID NO: 669 - protein, 15 aa, Artificial Sequence
  Description of Artificial Sequence: Synthetic peptide
CASSIRNYSNQPQHF

SEQ ID NO: 670 - protein, 14 aa, Artificial Sequence
  Description of Artificial Sequence: Synthetic peptide
CAVAPSQAGTALIF

SEQ ID NO: 671 - protein, 15 aa, Artificial Sequence
  Description of Artificial Sequence: Synthetic peptide
CASSLRTGQNTEAFF

SEQ ID NO: 672 - protein, 15 aa, Artificial Sequence
  Description of Artificial Sequence: Synthetic peptide
CAFMKRTNRDDKIIF

SEQ ID NO: 673 - protein, 15 aa, Artificial Sequence
  Description of Artificial Sequence: Synthetic peptide
CASSGGTPYNSPLHF

SEQ ID NO: 674 - protein, 10 aa, Artificial Sequence
  Description of Artificial Sequence: Synthetic peptide
FLVYGVRPGM

SEQ ID NO: 675 - protein, 10 aa, Artificial Sequence
  Description of Artificial Sequence: Synthetic peptide
CAFMQFDMRF

SEQ ID NO: 676 - protein, 12 aa, Artificial Sequence
  Description of Artificial Sequence: Synthetic peptide
CASTPGGYEQYF

SEQ ID NO: 677 - protein, 12 aa, Artificial Sequence
  Description of Artificial Sequence: Synthetic peptide
CAVDTNTDKLIF

SEQ ID NO: 678 - protein, 11 aa, Artificial Sequence
  Description of Artificial Sequence: Synthetic peptide
CASSQGYEQYF

SEQ ID NO: 679 - protein, 25 aa, Artificial Sequence
  Description of Artificial Sequence: Synthetic peptide
RKTVRARSRTPSCRSRSHTPSRRRR

SEQ ID NO: 680 - protein, 15 aa, Artificial Sequence
  Description of Artificial Sequence: Synthetic peptide
CALQISSGSARQLTF

SEQ ID NO: 681 - protein, 12 aa, Artificial Sequence
  Description of Artificial Sequence: Synthetic peptide
CASSLIRSEAFF

SEQ ID NO: 682 - protein, 13 aa, Artificial Sequence
  Description of Artificial Sequence: Synthetic peptide
CALRESGYSTLTF

SEQ ID NO: 683 - protein, 13 aa, Artificial Sequence
  Description of Artificial Sequence: Synthetic peptide
CASSRDRSNEQFF

SEQ ID NO: 684 - protein, 17 aa, Artificial Sequence
  Description of Artificial Sequence: Synthetic peptide
CAGQDNPASGNTGKLIF

SEQ ID NO: 685 - protein, 12 aa, Artificial Sequence
  Description of Artificial Sequence: Synthetic peptide
CASSFIRTGQYF

SEQ ID NO: 686 - protein, 14 aa, Artificial Sequence
  Description of Artificial Sequence: Synthetic peptide
CAAPARGGSYIPTF

SEQ ID NO: 687 - protein, 15 aa, Artificial Sequence
  Description of Artificial Sequence: Synthetic peptide
CASSLVDRRGEKLFF

SEQ ID NO: 688 - protein, 9 aa, Artificial Sequence
  Description of Artificial Sequence: Synthetic peptide
TLWCSPIKV

SEQ ID NO: 689 - protein, 15 aa, Artificial Sequence
  Description of Artificial Sequence: Synthetic peptide
CAASLSGGGADGLTF

SEQ ID NO: 690 - protein, 11 aa, Artificial Sequence
  Description of Artificial Sequence: Synthetic peptide
CASSLDRKAFF

SEQ ID NO: 691 - protein, 15 aa, Artificial Sequence
  Description of Artificial Sequence: Synthetic peptide
CALSDSGGSNYKLTF

SEQ ID NO: 692 - protein, 17 aa, Artificial Sequence
  Description of Artificial Sequence: Synthetic peptide
CASRFVAPSDGYNEQFF

SEQ ID NO: 693 - protein, 16 aa, Artificial Sequence
  Description of Artificial Sequence: Synthetic peptide
CALSDPQDSGYSTLTF

SEQ ID NO: 694 - protein, 14 aa, Artificial Sequence
  Description of Artificial Sequence: Synthetic peptide
CASSLGQGRVEQYF

SEQ ID NO: 695 - protein, 15 aa, Artificial Sequence
  Description of Artificial Sequence: Synthetic peptide
CAVSPGAGGSYIPTF

SEQ ID NO: 696 - protein, 17 aa, Artificial Sequence
  Description of Artificial Sequence: Synthetic peptide
CAISEVGTGSSGNTIYF

SEQ ID NO: 697 - protein, 13 aa, Artificial Sequence
  Description of Artificial Sequence: Synthetic peptide
CAVRPSGYSTLTF

SEQ ID NO: 698 - protein, 14 aa, Artificial Sequence
  Description of Artificial Sequence: Synthetic peptide
CAITSGQGNNEQYF

SEQ ID NO: 699 - protein, 14 aa, Artificial Sequence
  Description of Artificial Sequence: Synthetic peptide
CAVQAWGFGNVLHC

SEQ ID NO: 700 - protein, 14 aa, Artificial Sequence
  Description of Artificial Sequence: Synthetic peptide
CASSDRNTNYGYTF

SEQ ID NO: 701 - protein, 21 aa, Artificial Sequence
  Description of Artificial Sequence: Synthetic peptide
CAVKSWSGPGWGNQAGTALIF

SEQ ID NO: 702 - protein, 16 aa, Artificial Sequence
  Description of Artificial Sequence: Synthetic peptide
CASSMRVQSNTGELFF

SEQ ID NO: 703 - protein, 14 aa, Artificial Sequence
  Description of Artificial Sequence: Synthetic peptide
CVVNTPPNTDKLIF

SEQ ID NO: 704 - protein, 17 aa, Artificial Sequence
  Description of Artificial Sequence: Synthetic peptide
CASTPFTSGSGWDEQFF

SEQ ID NO: 705 - protein, 13 aa, Artificial Sequence
  Description of Artificial Sequence: Synthetic peptide
CAVPSGSARQLTF

SEQ ID NO: 706 - protein, 15 aa, Artificial Sequence
  Description of Artificial Sequence: Synthetic peptide
CASSLDFSRQETQYF

SEQ ID NO: 707 - protein, 12 aa, Artificial Sequence
  Description of Artificial Sequence: Synthetic peptide
CAVIKGYSTLTF

SEQ ID NO: 708 - protein, 16 aa, Artificial Sequence
  Description of Artificial Sequence: Synthetic peptide
CASSLYLASNTGELFF

SEQ ID NO: 709 - protein, 141 aa, Artificial Sequence
  Description of Artificial Sequence: Synthetic polypeptide
YIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 710 - protein, 179 aa, Artificial Sequence
  Description of Artificial Sequence: Synthetic polypeptide
LEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSR

SEQ ID NO: 711 - protein, 27 aa, Artificial Sequence
  Description of Artificial Sequence: Synthetic peptide
YPYDVPDYAYPYDVPDYAYPYDVPDYA

SEQ ID NO: 712 - protein, 30 aa, Artificial Sequence
  Description of Artificial Sequence: Synthetic polypeptide
YPYDVPDYASYPYDVPDYASYPYDVPDYAS

SEQ ID NO: 713 - protein, 24 aa, Artificial Sequence
  Description of Artificial Sequence: Synthetic peptide
DYKDDDDKDYKDDDDKDYKDDDDK

SEQ ID NO: 714 - protein, 30 aa, Artificial Sequence
  Description of Artificial Sequence: Synthetic polypeptide
EQKLISEEDLEQKLISEEDLEQKLISEEDL

SEQ ID NO: 715 - protein, 5 aa, Artificial Sequence
  Description of Artificial Sequence: Synthetic peptide
  See specification as filed for detailed description of substitutions and preferred embodiments
GGGGS

(19) United States
(12) Patent Application Publication
(10) Pub. No.: US 2023/0293687 A1
(43) Pub. Date: Sep. 21, 2023

(54) T CELLS AND CHIMERIC STIMULATING RECEPTORS AND USES THEREOF

(71) Applicant: Eureka Therapeutics, Inc., Emeryville, CA (US)

(72) Inventors: Cheng Liu, Emeryville, CA (US); Hongbing Zhang, Emeryville, CA (US); Zhiyuan Yang, Emeryville, CA (US); Pengbo Zhang, Emeryville, CA (US); Yixiang Xu, Emeryville, CA (US); Guangyuan Xiong, Emeryville, CA (US); Ziyou Cui, Emeryville, CA (US)

(21) Appl. No.: 18/017,628

(22) PCT Filed: Jul. 29, 2021
(86) PCT No.: PCT/US2021/043776

§ 371 Date: Jan. 23, 2023

Related U.S. Application Data
(60) Provisional application No. 63/058,046, filed on Jul. 29, 2020.

(51) Int. Cl. A61K 39/00; C12N 15/86; C07K 14/725; C07K 14/705; C07K 16/28; C07K 14/47; C07K 14/005; C07K 16/30; C07K 16/08; C07K 16/18; C07K 14/435; C07K 16/32; C07K 16/40; C07K 14/78; C07K 14/82; C07K 16/44; C07K 14/71; C07K 14/73; C07K 14/735; A61P 35/00

(52) U.S. Cl. CPC A61K 39/4622; C12N 15/86; C07K 14/7051; C07K 14/70578; A61K 39/4632; A61K 39/464417; C07K 14/70521; C07K 14/70553; C07K 14/70507; C07K 14/70503; C07K 14/7056; C07K 14/705; A61K 39/464411; A61K 39/464402; A61K 39/4644; A61K 39/4633; C07K 16/28; C07K 14/4748; C07K 14/005; A61K 39/464489; A61K 39/464481; A61K 39/464453; A61K 39/464414; A61K 39/464406; C07K 16/3038; C07K 16/303; C07K 16/30; C07K 16/2833; C07K 16/084; A61K 39/464838; C07K 16/18; A61K 39/4643; C07K 14/435; C07K 16/3069; A61K 39/464494; C07K 16/32; C07K 16/40; C07K 14/78; A61K 39/464454; A61K 39/464462; C07K 16/3053; C07K 14/70596; C07K 16/2896; A61K 39/46432; A61K 39/464488; A61K 39/464486; A61K 39/464449; A61K 39/464451; C07K 14/4746; C07K 16/3015; C07K 14/82; C07K 16/44; A61K 39/4645; C07K 14/4727; C07K 16/3092; C07K 14/71; C07K 16/2863; A61K 39/464431; A61K 39/464495; A61K 39/46441; C07K 14/70589; A61K 39/464413; C07K 14/70514; C07K 14/70535; C07K 14/70532; A61K 39/4611; A61K 39/4613; A61P 35/00; C12N 2740/15043; C07K 2319/03; C07K 2319/033; C07K 2319/035; C07K 2317/569; C07K 2317/31; C07K 2317/622; C07K 2317/565

(57) ABSTRACT

Described herein are immune cells comprising: a T-cell receptor (TCR) and a chimeric stimulating receptor (CSR) that comprises (i) a ligand-binding module that is capable of binding or interacting with a target ligand; (ii) a transmembrane domain; and (iii) a CD30 costimulatory domain, in which the CSR in the immune cells lacks a functional primary signaling domain. Also provided herein are methods of using the same or components thereof (e.g., the CSR) for therapeutic treatment of cancers (e.g., solid tumor cancers).

### CROSS-REFERENCE TO RELATED APPLICATION

This application claims priority to U.S. Provisional Application No. 63/058,046, filed Jul. 29, 2020, the disclosure of which is hereby incorporated by reference in its entirety for all purposes.

### BACKGROUND

Adoptive T cell immunotherapy, in which a patient's own T lymphocytes are engineered to express various recombinant antigen receptors such as chimeric antigen receptors (CARs), has shown great promise in treating hematological malignancies, but not so much in solid tumors. In addition, CAR by itself is generally not efficacious enough, especially for solid tumors, even with the commonly used costimulatory fragments such as CD28, 4-1BB, or DAP10, no matter if expressed in cis or in trans. Therefore, more efficacious and longer-lasting T cell immunotherapies are needed.

Immunotherapy of cancer is becoming one of the frontline approaches to cancer therapy due to the recent success of check-point inhibitors and adoptive T cell therapy (ACT) of cancer in the clinic. Although ACT therapy with tumor infiltrating lymphocytes (TIL), CAR T cells, or TCR T cells from peripheral blood has shown some clinical results (Phan and Rosenberg, Cancer Control 20(4): 289-297, 2013; and Schuster et al., N Engl J Med. 380(1):45-56, 2019), there is still a great need for improvement of efficacy in treating solid malignancies. One of the major formidable hurdles is the T cell infiltration inhibition or tumor infiltration inhibition (Gajewski, Semin. Oncol. 42:663-671, 2015), which is caused by T cell inhibitory tumor microenvironment and hinders therapeutic effect of TCR T cells. Therefore. T cell immunotherapies with higher tumor infiltration efficacy are needed.

CD30 is a member of the TNF receptor superfamily of receptor proteins. Most of the homology between TNF receptor family members occurs in the extracellular domain, with little homology in the cytoplasmic domain. This suggested that different members of the TNF receptor family might utilize distinct signaling pathways. Consistent with this hypothesis, the TNF receptor type 1 and Fas have been shown to interact with a set of intracellular signaling molecules through a 65-amino acid domain termed a death domain, whereas the TNF receptor type 2 and CD40 have been found to associate with members of the tumor necrosis factor receptor-associated factor (TRAF) family of signal transducing molecules.

The membrane bound form of CD30 is a 120-kDa, 595-amino acid glycoprotein with a 188-amino acid cytoplasmic domain. Cross-linking of CD30 with either antibodies or with CD30 ligand produces a variety of effects in cells, including augmenting the proliferation of primary T cells following T-cell receptor engagement and induction of the NF-kB transcription factor. CD30 was originally identified as an antigen expressed on the surface of Hodgkin's lymphoma cells. Subsequently, CD30 was shown to be expressed by lymphocytes with an activated phenotype, cells on the periphery of germinal centers, and CD45RO1 (memory) T cells. CD30 may also play a role in the development of T helper 2 type cells. The T cell activation properties of the TNF receptor family member 4-1BB have been shown to involve the specific ability of its cytoplasmic domain to associate with the tyrosine kinase p56lck. The sequence of the cytoplasmic domain of CD30 shows little sequence similarity to any of these receptors; CD30 lacks an obvious death domain or a p56lck-binding site.

### SUMMARY

The present invention provides, among other things, chimeric stimulating receptors (CSRs) that use a costimulatory domain from CD30 (also referred to herein as a CD30 costimulatory domain). As described in detail herein, T cells with CSRs containing a costimulatory domain from CD30 express far less PD-1, an inhibitor of T cell activation, than Tcells with CSRs containing a costimulatory domain from, e.g., CD28, 4-1BB, or DAP10, and at the same time demonstrate equal cytotoxic potential. The examples suggest that the costimulatory domain from CD30 ameliorates the functional unresponsiveness that leads to T cell exhaustion, also called anergy, and subsequently, provides superior persistence of tumor cell killing and increased tumor infiltration as compared to the commonly used costimulatory domains such as CD28. It is unexpected since CD30 lacks a p56lck-binding site that is thought to be crucial for CSR costimulation.

In one aspect, the disclosure features an immune cell comprising: (a) an as T-cell receptor (TCR), and (b) a chimeric stimulating receptor (CSR) comprising: (i) a ligand-binding module that is capable of binding or interacting with a target ligand; (ii) a transmembrane domain (a CSR transmembrane domain); and (iii) a CD30 costimulatory domain, wherein the CSR lacks a functional primary signaling domain (e.g., a functional primary signaling domain derived from the intracellular signaling sequence of CD3ζ).

In some embodiments, the CD30 costimulatory domain comprises a sequence that can bind to an intracellular TRAF signaling protein. In some embodiments, the sequence that can bind to an intracellular TRAF signaling protein corresponds to residues 561-573 or 578-586 of a full-length CD30 having the sequence of SEQ ID NO:228. In some embodiments, the CD30 costimulatory domain comprises a sequence that is at least 80%, 85%, 90%, 95%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to residues 561-573 or 578-586 of SEQ ID NO:228. In some embodiments, the CD30 costimulatory domain comprises a sequence that is at least 70%, 75%, 80%, 85%, 90%, 95%, or 100% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identical to the sequence of SEQ ID NO:238.

In some embodiments of this aspect, the CSR comprises more than one CD30 costimulatory domain. In some embodiments, the CSR further comprises at least one costimulatory domain which comprises the intracellular sequence of a costimulatory molecule that is different from CD30. The costimulatory molecule that is different from CD30 can be selected from the group consisting of CD27, CD28, 4-1BB (CD137), OX40, CD40, PD-1, ICOS, lymphocyte function-associated antigen-1 (LFA-1), CD2, CD7, LIGHT, NKG2C, B7-H3, and a ligand that specifically binds with CD83.

In some embodiments, the ligand-binding module of the CSR is derived from the extracellular domain of a receptor. In some embodiments, the ligand-binding module of the CSR comprises an antibody moiety (a CSR antibody moiety). The CSR antibody moiety can be a single chain antibody fragment. In some embodiments, the CSR antibody moiety is a single chain Fv (scFv), a single chain Fab, a single chain Fab′, a single domain antibody fragment, a single domain multispecific antibody, an intrabody, a nanobody, or a single chain immunokine. In some embodiments, the CSR antibody moiety is a single domain multispecific antibody. In some embodiments, the single domain multispecific antibody is a single domain bispecific antibody. In some embodiments, the CSR antibody moiety is a single chain Fv (scFv). In some embodiments, the scFv is a tandem scFv.

In some embodiments, the CSR antibody moiety specifically binds to a disease-related antigen. The disease-related antigen is a cancer-related antigen. The disease-related antigen is a virus-related antigen. In some embodiments, the CSR antibody moiety specifically binds to a cell surface antigen. The cell surface antigen can be selected from the group consisting of protein, carbohydrate, and lipid. The cell surface antigen can be CD19, CD20, CD22, CD47, CD158e, GPC3, ROR1, ROR2, BCMA, GPRC5D, FcRL5, MUC16, MCT4, PSMA, or a variant or mutant thereof.

In some embodiments, the TCR and the CSR antibody moiety specifically bind to the same antigen. In particular embodiments, the TCR and the CSR antibody moiety specifically bind to different epitopes on the same antigen. In some embodiments, the TCR and the CSR antibody moiety specifically bind to different antigens.

In some embodiments, the CSR antibody moiety specifically binds to a MHC-restricted antigen. In some embodiments, the MHC-restricted antigen is a complex comprising a peptide and an MHC protein, and the peptide is derived from a protein selected from the group consisting of WT-1, AFP, HPV16-E7, NY-ESO-1, PRAME, EBV-LMP2A, HIV-1, KRAS, FoxP3, Histone H3.3, PSA, and a variant or mutant thereof.

In some embodiments of this aspect, the TCR specifically binds to a complex comprising an alpha-fetoprotein (AFP) peptide and an MHC class I protein. In certain embodiments, the AFP peptide comprises an amino acid sequence of any one of SEQ ID NOS:26-36. In some embodiments, the TCR comprises: (1) an anti-AFP-TCRα chain comprising sequences of CDR1, CDR2, and CDR3 of SEQ ID NOS:305-307, respectively; and/or (2) an anti-AFP-TCRβ chain comprising sequences of CDR1, CDR2, and CDR3 of SEQ ID NOS:308-310, respectively. In some embodiments, the TCR comprises: (1) an anti-AFP-TCRα chain comprising sequences of CDR1, CDR2, and CDR3 of SEQ ID NOS:311-313, respectively; and/or (2) an anti-AFP-TCRβ chain comprising sequences of CDR1, CDR2, and CDR3 of SEQ ID NOS:308-310, respectively. In some embodiments, the TCR comprises: (1) an anti-AFP-TCRα chain variable region comprising a sequence of SEQ ID NO:314; and/or (2) an anti-AFP-TCRβ chain variable region comprising a sequence of SEQ ID NO:315. In some embodiments, the TCR comprises: (1) an anti-AFP-TCRα chain variable region comprising a sequence of SEQ ID NO:316; and/or (2) an anti-AFP-TCRβ chain variable region comprising a sequence of SEQ ID NO:315. In some embodiments, the TCR comprises a sequence of any one of SEQ ID NOS:1-3. In some embodiments, the TCR comprises the sequences of SEQ ID NOS:1 and 2. In some embodiments, the TCR comprises the sequences of SEQ ID NOS:2 and 3. In some embodiments, the TCR comprises a sequence of any one of SEQ ID NOS:6-19.

In some embodiments, the ligand-binding module of the CSR specifically binds to glypican 3 (GPC3). In some embodiments, the TCR binds to a complex comprising an AFP peptide and an MHC class I protein, and the ligand-binding module of the CSR binds to GPC3. In some embodiments, the anti-GPC3 CSR comprises: (1) sequences of HCDR1, HCDR2, HCDR3, LCDR1, LCDR2, and LCDR3 of SEQ ID NOS:317-322, respectively; or (2) sequences of HCDR1, HCDR2, HCDR3, LCDR1, LCDR2, and LCDR3 of SEQ ID NOS:323-328, respectively; or (3) sequences of HCDR1, HCDR2, HCDR3, LCDR1, LCDR2, and LCDR3 of SEQ ID NOS:329-334, respectively; or (4) sequences of HCDR1, HCDR2, HCDR3, LCDR1, LCDR2, and LCDR3 of SEQ ID NOS:335-340, respectively; or (5) sequences of HCDR1, HCDR2, HCDR3, LCDR1, LCDR2, and LCDR3 of SEQ ID NOS:341-346, respectively; or (6) sequences of HCDR1, HCDR2, HCDR3, LCDR1, LCDR2, and LCDR3 of SEQ ID NOS:347-352, respectively; or (7) sequences of HCDR1, HCDR2, HCDR3, LCDR1, LCDR2, and LCDR3 of SEQ ID NOS:353-358, respectively. In some embodiments, the anti-GPC3 CSR comprises a heavy chain variable region having the sequence of any one of SEQ ID NOS:274, 276, 278, 280, 282, 284, and 286, and a light chain variable region having the sequence of any one of SEQ ID NOS:275, 277, 279, 281, 283, 285, and 287. In some embodiments, the anti-GPC3 CSR comprises an scFv having the sequence of any one of SEQ ID NOS:212-213 and 269-273. In some embodiments, the anti-GPC3 CSR comprises an amino acid sequence of any one of SEQ ID NOS:181-211 and 288-293. In some embodiments, the anti-GPC3 CSR comprises a heavy chain variable region and a light chain variable region that compete with at least one of the anti-GPC3 molecule described above with the recited sequences for its specific binding to GPC3.

In some embodiments of this aspect, the TCR specifically binds to a complex comprising a KRAS, p53, or MSLN peptide and an MHC class I protein. For example, TCRs that specifically bind to a complex comprising an MSLN peptide and an MHC claims I protein are described in, e.g., Stromnes et al., Cancer Cell. 28(5):638-652, 2015. In some embodiments, the CSR specifically binds to MSLN, such as a cell-surface MSLN protein. In some embodiments, the anti-MSLN CSR comprises sequences of HCDR1, HCDR2, and HCDR3 of SEQ ID NOS:71-73, respectively, and optionally a heavy chain variable region having the sequence of SEQ ID NO:70. In some embodiments, the anti-MSLN CSR comprises sequences of LCDR1, LCDR2, and LCDR3 of SEQ ID NOS:75-77, respectively, and optionally a light chain variable region having the sequence of SEQ ID NO:74. In some embodiments, the anti-MSLN CSR comprises a heavy chain variable region and a light chain variable region that compete with at least one of the anti-MSLN molecule described above with the recited sequences for its specific binding to MSLN. In some embodiments, the CSR specifically binds to ROR1. In some embodiments, the anti-ROR1 CSR specifically binds to a ROR1 epitope having a sequence of any one of SEQ ID NOS:443-446. In some embodiments, the anti-ROR1 CSR comprises sequences of HCDR1, HCDR2, and HCDR3 of SEQ ID NOS:447-449, respectively, and optionally a heavy chain variable region having the sequence of SEQ ID NO:450; and/or sequences of LCDR1, LCDR2, and LCDR3 of SEQ ID NOS:451-453, respectively, and optionally a light chain variable region having the sequence of SEQ ID NO:454; and/or optionally an scFv having the sequence of SEQ ID NO:441. In other embodiments, the anti-ROR1 CSR comprises sequences of HCDR1, HCDR2, and HCDR3 of SEQ ID NOS:455-457, respectively, and optionally a heavy chain variable region having the sequence of SEQ ID NO:458; and/or sequences of LCDR1, LCDR2, and LCDR3 of SEQ ID NOS:459-461, respectively, and optionally a light chain variable region having the sequence of SEQ ID NO:462; and/or optionally an scFv having the sequence of SEQ ID NO:442. In some embodiments, the anti-ROR1 CSR comprises a heavy chain variable region and a light chain variable region that compete with at least one of the anti-ROR1 molecule described above with the recited sequences for its specific binding to ROR1.

In some embodiments of this aspect, the TCR specifically binds to a complex comprising a PSA peptide and an MHC class I protein. An PSA peptide can comprise a sequence of any one of SEQ ID NOS:38-40. An anti-PSA TCR can comprise a sequence of any one of SEQ ID NOS:20-25. In some embodiments, the TCR comprises a sequence of any one of SEQ ID NOS:20-25. In some embodiments, the anti-PSMA CSR comprises sequences of HCDR1, HCDR2, and HCDR3 of SEQ ID NOS:373-375, respectively, and optionally a heavy chain variable region having the sequence of SEQ ID NO:376. In some embodiments, the CSR specifically binds to PSMA. In some embodiments, the anti-PSMA CSR comprises sequences of LCDR1, LCDR2, and LCDR3 of SEQ ID NOS:377-379, respectively, and optionally a light chain variable region having the sequence of SEQ ID NO:380. In further embodiments, the anti-PSMA CSR comprises a sequence of SEQ ID NO:214. In some embodiments, the anti-PSMA CSR comprises sequences of HCDR1, HCDR2, and HCDR3 of SEQ ID NOS:381-383, respectively, and optionally a heavy chain variable region having the sequence of SEQ ID NO:384. In some embodiments, the anti-PSMA CSR comprises sequences of LCDR1, LCDR2, and LCDR3 of SEQ ID NOS:385-387, respectively, and optionally a light chain variable region having the sequence of SEQ ID NO:388. In further embodiments, the anti-PSMA CSR comprises a sequence of SEQ ID NO:215. In some embodiments, the anti-PSMA CSR comprises a heavy chain variable region and a light chain variable region that compete with at least one of the anti-PSMA molecule described above with the recited sequences for its specific binding to PSMA. In some embodiments, the CSR specifically binds to ROR1. Specific embodiments of anti-ROR1 CSR are described herein, e.g., in paragraph [0015].

In some embodiments of this aspect, the TCR specifically binds to a complex comprising a COL18A1, SRPX, KIF16B, TFDP2, KIAA1279, XPNPEP1, UGGT2, PHKA1, KIF16B, SON, GNB5, FBXO21, CORO7, RECQL5, TFDP2, KIAA1967, KIF16B, MAGEA6, PDS5A, MED13, ASTN1, CDK4, MLL2, SMARCD3, NY-ESO-1, or PRA ME peptide and an MHC class I protein. In some embodiments, the CSR specifically binds to ROR2. An NY-ESO-1 peptide can comprise a sequence of SEQ ID NO:37. In some embodiments, the TCR specifically binds to a complex comprising NY-ESO-1 and the MHC claims I protein and comprises: (1) sequences of CDR1, CDR2, and CDR3 of SEQ ID NOS:359-361, respectively, and optionally a variable region having the sequence of SEQ ID NO:362, and further optionally the sequence of SEQ ID NO:4; or (2) sequences of CDR1, CDR2, and CDR3 of SEQ ID NOS:363-365, respectively, and optionally a variable region having the sequence of SEQ ID NO:366, and further optionally the sequence of SEQ ID NO:5. In some embodiments, the anti-ROR2 CSR comprises: (1) sequences of HCDR1, HCDR2, and HCDR3 of SEQ ID NOS:91-93, respectively, and optionally a heavy chain variable region having the sequence of SEQ ID NO:90; or (2) sequences of HCDR1, HCDR2, and HCDR3 of SEQ ID NOS:95-97, respectively, and optionally a heavy chain variable region having the sequence of SEQ ID NO:94; or (3) sequences of HCDR1, HCDR2, and HCDR3 of SEQ ID NOS:99-101, respectively, and optionally a heavy chain variable region having the sequence of SEQ ID NO:98; or (4) sequences of HCDR1, HCDR2, and HCDR3 of SEQ ID NOS:103-105, respectively, and optionally a heavy chain variable region having the sequence of SEQ ID NO:102; or (5) sequences of HCDR1, HCDR2, and HCDR3 of SEQ ID NOS:107-109, respectively, and optionally a heavy chain variable region having the sequence of SEQ ID NO:106. In some embodiments, the anti-ROR2 CSR comprises: (1) sequences of LCDR1, LCDR2, and LCDR3 of SEQ ID NOS:111-113, respectively, and optionally a light chain variable region having the sequence of SEQ ID NO:110; or (2) sequences of LCDR1, LCDR2, and LCDR3 of SEQ ID NOS:115-117, respectively, and optionally a light chain variable region having the sequence of SEQ ID NO:114; or (3) sequences of LCDR1, LCDR2, and LCDR3 of SEQ ID NOS:119-121, respectively, and optionally a light chain variable region having the sequence of SEQ ID NO:118; or (4) sequences of LCDR1, LCDR2, and LCDR3 of SEQ ID NOS: 123-125, respectively, and optionally a light chain variable region having the sequence of SEQ ID NO:122; or (5) sequences of LCDR1, LCDR2, and LCDR3 of SEQ ID NOS:127-129, respectively, and optionally a light chain variable region having the sequence of SEQ ID NO:126. In some embodiments, the anti-ROR2 CSR comprises a heavy chain variable region and a light chain variable region that compete with at least one of the anti-ROR2 molecule described above with the recited sequences for its specific binding to ROR2.

In some embodiments of this aspect, the TCR specifically binds to a complex comprising a NUP98, GPD2, CASP8, KRAS, SKIV2L, H3F3B, RAD21, or PRAME peptide and an MHC class I protein. In some embodiments, the CSR specifically binds to ROR2. Specific embodiments of anti-ROR2 CSR are described herein, e.g., in paragraph [0017].

In some embodiments of this aspect, the TCR specifically binds to a complex comprising a SLC3A2, KIAA0368, CADPS2, CTSB, PRAME, p53, or PSA peptide and an MHC class I protein. In some embodiments, the CSR specifically binds to HER2, EpCAM, or ROR1. In some embodiments, the anti-PSA TCR comprises a sequence of any one of SEQ ID NOS:20-25. In some embodiments, the CSR specifically binds to HER2 and comprises sequences of HCDR1, HCDR2, and HCDR3 of SEQ ID NOS:389-391, respectively, and optionally a heavy chain variable region having the sequence of SEQ ID NO:41. In some embodiments, the CSR binds to HER2 and comprises sequences of LCDR1, LCDR2, and LCDR3 of SEQ ID NOS:392-394, respectively, and optionally a light chain variable region having the sequence of SEQ ID NO:42. In some embodiments, the CSR specifically binds to EpCAM and comprises sequences of HCDR1, HCDR2, and HCDR3 of SEQ ID NOS:403-405, respectively, and optionally a heavy chain variable region having the sequence of SEQ ID NO:60. In some embodiments, the CSR binds to EpCAM and comprises sequences of LCDR1, LCDR2, and LCDR3 of SEQ ID NOS:406-408, respectively, and optionally a light chain variable region having the sequence of SEQ ID NO:61. In some embodiments, the anti-HER2 CSR comprises a heavy chain variable region and a light chain variable region that compete with at least one of the anti-HER2 molecule described above with the recited sequences for its specific binding to HER2. In some embodiments, the anti-EpCAM CSR comprises a heavy chain variable region and a light chain variable region that compete with at least one of the anti-EpCAM molecule described above with the recited sequences for its specific binding to EpCAM. In some embodiments, the CSR specifically binds to ROR1. Specific embodiments of anti-ROR1 CSR are described herein, e.g., in paragraph [0015]. In some embodiments, the anti-ROR1 CSR comprises a heavy chain variable region and a light chain variable region that compete with at least one of the anti-ROR1 molecule described above with the recited sequences for its specific binding to ROR1.

In some embodiments of this aspect, the TCR specifically binds to a complex comprising a WT1, NY-ESO-1, p53, DPY19L4, or RNF19B peptide and an MHC class I protein. In some embodiments, the CSR specifically binds to MUC1, MUC16, FRα, or ROR1. In some embodiments, the TCR specifically binds to a complex comprising NY-ESO-1 and the MHC claims I protein and comprises: (1) sequences of CDR1, CDR2, and CDR3 of SEQ ID NOS:359-361, respectively, and optionally a variable region having the sequence of SEQ ID NO:362, and further optionally the sequence of SEQ ID NO:4; or (2) sequences of CDR1, CDR2, and CDR3 of SEQ ID NOS:363-365, respectively, and optionally a variable region having the sequence of SEQ ID NO:366, and further optionally the sequence of SEQ ID NO:5. In some embodiments, the CSR specifically binds to MUC1 and comprises sequences of HCDR1, HCDR2, and HCDR3 of SEQ ID NOS:417-419, respectively, and optionally a heavy chain variable region having the sequence of SEQ ID NO:367. In some embodiments, the CSR specifically binds to MUC1 and comprises sequences of LCDR1, LCDR2, and LCDR3 of SEQ ID NOS:420-422, respectively, and optionally a light chain variable region having the sequence of SEQ ID NO:368. In some embodiments, the CSR specifically binds to MUC16 and comprises: (1) sequences of HCDR1, HCDR2, and HCDR3 of SEQ ID NOS:131-133, respectively, and optionally a heavy chain variable region having the sequence of SEQ ID NO:130; or (2) sequences of HCDR1, HCDR2, and HCDR3 of SEQ ID NOS:135-137, respectively, and optionally a heavy chain variable region having the sequence of SEQ ID NO:134; (3) sequences of HCDR1, HCDR2, and HCDR3 of SEQ ID NOS:429-431, respectively, and optionally a heavy chain variable region having the sequence of any one of SEQ ID NOS:146-147; or (4) sequences of HCDR1, HCDR2, and HCDR3 of SEQ ID NOS:435-437, respectively, and optionally a heavy chain variable region having the sequence of any one of SEQ ID NOS:148-149. In some embodiments, the CSR specifically binds to MUC16 and comprises: (1) sequences of LCDR1, LCDR2, and LCDR3 of SEQ ID NOS:139-141, respectively, and optionally a light chain variable region having the sequence of SEQ ID NO:138; or (2) sequences of LCDR1, LCDR2, and LCDR3 of SEQ ID NOS:143-145, respectively, and optionally a light chain variable region having the sequence of SEQ ID NO:142; (3) sequences of LCDR1, LCDR2, and LCDR3 of SEQ ID NOS:432-434, respectively, and optionally a light chain variable region having the sequence of any one of SEQ ID NOS:150-151; or (4) sequences of LCDR1, LCDR2, and LCDR3 of SEQ ID NOS:438-440, respectively, and optionally a light chain variable region having the sequence of any one of SEQ ID NOS:152-153. In some embodiments, the CSR specifically binds to FRα and comprises sequences of HCDR1, HCDR2, and HCDR3 of SEQ ID NOS:423-425, respectively, and optionally a heavy chain variable region having the sequence of SEQ ID NO:369 and further optionally a heavy chain having the sequence of SEQ ID NO:370. In some embodiments, the CSR specifically binds to FRα and comprises sequences of LCDR1, LCDR2, and LCDR3 of SEQ ID NOS:426-428, respectively, and optionally a light chain variable region having the sequence of SEQ ID NO:371 and further optionally a light chain having the sequence of SEQ ID NO:372. In some embodiments, the anti-MUC1 CSR comprises a heavy chain variable region and a light chain variable region that compete with at least one of the anti-MUC1 molecule described above with the recited sequences for its specific binding to MUC1. In some embodiments, the anti-MUC16 CSR comprises a heavy chain variable region and a light chain variable region that compete with at least one of the anti-MUC16 molecule described above with the recited sequences for its specific binding to MUC16. In some embodiments, the anti-FRα CSR comprises a heavy chain variable region and a light chain variable region that compete with at least one of the anti-FRα molecule described above with the recited sequences for its specific binding to FRα. In some embodiments, the CSR specifically binds to ROR1. Specific embodiments of anti-ROR1 CSR are described herein, e.g., in paragraph [0015]. In some embodiments, the anti-ROR1 CSR comprises a heavy chain variable region and a light chain variable region that compete with at least one of the anti-ROR1 molecule described above with the recited sequences for its specific binding to ROR1.

In some embodiments of this aspect, the TCR specifically binds to a complex comprising a p53 or KRAS peptide and an MHC class I protein. In some embodiments, the CSR specifically binds to EGFR. In some embodiments, the anti-EGFR CSR comprises sequences of HCDR1, HCDR2, and HCDR3 of SEQ ID NOS:79-81, respectively, and optionally a heavy chain variable region having the sequence of SEQ ID NO:78. In some embodiments, the anti-EGFR CSR comprises sequences of LCDR1, LCDR2, and LCDR3 of SEQ ID NOS:83-85, respectively, and optionally a light chain variable region having the sequence of SEQ ID NO:82. In some embodiments, the anti-EGFR CSR comprises a heavy chain variable region and a light chain variable region that compete with at least one of the anti-EGFR molecule described above with the recited sequences for its specific binding to EGFR.

In some embodiments of this aspect, the TCR specifically binds to a complex comprising a ARHGAP35 or Histone H3.3 peptide and an MHC class I protein. In some embodiments, the CSR specifically binds to EGFR or EGFRvIII. In some embodiments, the CSR specifically binds to EGFR and comprises sequences of HCDR1, HCDR2, and HCDR3 of SEQ ID NOS:79-81, respectively, and optionally a heavy chain variable region having the sequence of SEQ ID NO:78. In some embodiments, the CSR specifically binds to EGFR comprises sequences of LCDR1, LCDR2, and LCDR3 of SEQ ID NOS:83-85, respectively, and optionally a light chain variable region having the sequence of SEQ ID NO:82. In some embodiments, the CSR specifically binds to EGFRvIII and comprises sequences of HCDR1, HCDR2, and HCDR3 of SEQ ID NOS:409-411, respectively, and optionally a heavy chain variable region having the sequence of SEQ ID NO:412. In some embodiments, the CSR specifically binds to EGFRvIII and comprises sequences of LCDR1, LCDR2, and LCDR3 of SEQ ID NOS:413-415, respectively, and optionally a light chain variable region having the sequence of SEQ ID NO:416. In further embodiments, the CSR specifically binds to EGFRvIII and comprises comprises the sequence of SEQ ID NO:86. In some embodiments, the anti-EGFR CSR comprises a heavy chain variable region and a light chain variable region that compete with at least one of the anti-EGFR molecule described above with the recited sequences for its specific binding to EGFR. In some embodiments, the anti-EGFRvIII CSR comprises a heavy chain variable region and a light chain variable region that compete with at least one of the anti-EGFRvAII molecule described above with the recited sequences for its specific binding to EGFRvIII.

In some embodiments of this aspect, the TCR specifically binds to a complex comprising a KRAS, HER2, NY-ESO-1, or p53 peptide and an MHC class I protein. In some embodiments, the CSR specifically binds to HER3, DLL3, c-Met, or ROR1. In some embodiments, the TCR specifically binds to a complex comprising NY-ESO-1 and the MHC claims I protein and comprises: (1) sequences of CDR1, CDR2, and CDR3 of SEQ ID NOS:359-361, respectively, and optionally a variable region having the sequence of SEQ ID NO:362, and further optionally the sequence of SEQ ID NO:4; or (2) sequences of CDR1, CDR2, and CDR3 of SEQ ID NOS:363-365, respectively, and optionally a variable region having the sequence of SEQ ID NO:366, and further optionally the sequence of SEQ ID NO:5. In some embodiments, the CSR specifically binds to HER3 and comprises sequences of HCDR1, HCDR2, and HCDR3 of SEQ ID NOS:395-397, respectively, and optionally a heavy chain having the sequence of SEQ ID NO:398. In some embodiments, the CSR specifically binds to HER3 and comprises sequences of LCDR1, LCDR2, and LCDR3 of SEQ ID NOS:399-401, respectively, and optionally a light chain having the sequence of SEQ ID NO:402. In further embodiments, the CSR specifically binds to HER3 and comprises a sequence of SEQ ID NO:43. In some embodiments, the CSR specifically binds to DLL3 and comprises sequences of HCDR1, HCDR2, and HCDR3 of SEQ ID NOS:45-47, respectively, and optionally a heavy chain having the sequence of SEQ ID NO:44. In some embodiments, the CSR specifically binds to DLL3 and comprises sequences of LCDR1, LCDR2, and LCDR3 of SEQ ID NOS:49-51, respectively, and optionally a light chain having the sequence of SEQ ID NO:48. In some embodiments, the anti-HER3 CSR comprises a heavy chain variable region and a light chain variable region that compete with at least one of the anti-HER3 molecule described above with the recited sequences for its specific binding to HER3. In some embodiments, the anti-DLL3 CSR comprises a heavy chain variable region and a light chain variable region that compete with at least one of the anti-DLL3 molecule described above with the recited sequences for its specific binding to DLL3. In some embodiments, the CSR specifically binds to ROR1. Specific embodiments of anti-ROR1 CSR are described herein, e.g., in paragraph [0015]. In some embodiments, the anti-ROR1 CSR comprises a heavy chain variable region and a light chain variable region that compete with at least one of the anti-ROR1 molecule described above with the recited sequences for its specific binding to ROR1.

In some embodiments of this aspect, the TCR specifically binds to a complex comprising a 5T4 or PRAME peptide and an MHC class I protein. In some embodiments, the CSR specifically binds to ROR2, CD70, or MCT4. In some embodiments, the CSR specifically binds to ROR2, and specific embodiments of anti-ROR2 CSR are described herein, e.g., in paragraph [0017]. In some embodiments, the CSR specifically binds to CD70 and comprises sequences of HCDR1, HCDR2, and HCDR3 of SEQ ID NOS:63-65, respectively, and optionally a heavy chain having the sequence of SEQ ID NO:62. In some embodiments, the CSR specifically binds to CD70 and comprises sequences of LCDR1, LCDR2, and LCDR3 of SEQ ID NOS:67-69, respectively, and optionally a light chain having the sequence of SEQ ID NO:66. In some embodiments, the CSR specifically binds to MCT4 and comprises: (1) sequences of HCDR1, HCDR2, and HCDR3 of SEQ ID NOS:155-157, respectively, and optionally a heavy chain having the sequence of SEQ ID NO:154; or (2) sequences of HCDR1, HCDR2, and HCDR3 of SEQ ID NOS:159-161, respectively, and optionally a heavy chain having the sequence of SEQ ID NO:158; or (3) sequences of HCDR1, HCDR2, and HCDR3 of SEQ ID NOS:163-165, respectively, and optionally a heavy chain having the sequence of SEQ ID NO:162. In some embodiments, the CSR specifically binds to MCT4 and comprises: (1) sequences of LCDR1, LCDR2, and LCDR3 of SEQ ID NOS: 167-169, respectively, and optionally a light chain having the sequence of SEQ ID NO:166; or (2) sequences of LCDR1. LCDR2, and LCDR3 of SEQ ID NOS:171-173, respectively, and optionally a light chain having the sequence of SEQ ID NO:170; or (3) sequences of LCDR1, LCDR2, and LCDR3 of SEQ ID NOS:175-177, respectively, and optionally a light chain having the sequence of SEQ ID NO:174. In some embodiments, the anti-ROR2 CSR comprises a heavy chain variable region and a light chain variable region that compete with at least one of the anti-ROR2 molecule described above with the recited sequences for its specific binding to ROR2. In some embodiments, the anti-CD70 CSR comprises a heavy chain variable region and a light chain variable region that compete with at least one of the anti-CD70 molecule described above with the recited sequences for its specific binding to CD70. In some embodiments, the anti-MCT4 CSR comprises a heavy chain variable region and a light chain variable region that compete with at least one of the anti-MCT4 molecule described above with the recited sequences for its specific binding to MCT4.

In some embodiments, the ligand-binding module of the CSR binds to GPC3. In particular embodiments, the ligand-binding module of the CSR specifically binds to an epitope on GPC3.

In some embodiments, the CSR transmembrane domain is derived from the transmembrane domain of a TCR co-receptor or a T cell costimulatory molecule. The TCR co-receptor or T cell costimulatory molecule can be selected from the group consisting of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, and CD154. In certain embodiments, the TCR co-receptor or T cell costimulatory molecule is CD30 or CD8. In some embodiments, the T cell costimulatory molecule can be CD30. In some embodiments, the TCR co-receptor is CD8.

In some embodiments, the CSR transmembrane domain is the transmembrane domain of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, or CD154. In certain embodiments, the CSR transmembrane domain is the transmembrane domain of CD30 or CD8. In certain embodiments, the CSR transmembrane domain is the transmembrane domain of CD30. In certain embodiments, the CSR transmembrane domain is the transmembrane domain of CD8. In certain embodiments, the CSR transmembrane domain comprises an amino acid sequence selected from the group consisting of SEQ ID NOS:229-234.

In some embodiments of this aspect, the CSR lacks a functional primary signaling domain derived from the intracellular signaling sequence of a molecule selected from the group consisting of CD3ζ, TCRζ, FcRγ, FcRβ, CD3γ, CD3δ, CD3ε, CD5, CD22, CD79a, CD79b, and CD66d. In some embodiments, the CSR lacks a functional primary signaling domain derived from the intracellular signaling sequence of CD3ζ. In certain embodiments, the CSR lacks a functional primary signaling domain having a sequence that is at least 80%, 85%, 90%, 95%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to the sequence of SEQ ID NO:241.

In some embodiments, the CSR in the immune cell further comprises a peptide linker between the ligand-binding module and the transmembrane domain of the CSR. In some embodiments, the CSR in the immune cell further comprises a peptide linker between the transmembrane domain and the CD30 costimulatory domain of the CSR.

In some embodiments of this aspect, the expression of the CSR is inducible. In some embodiments, the expression of the CSR is inducible upon activation of the immune cell. In some embodiments, the immune cell is selected from the group consisting of a cytotoxic T cell, a helper T cell, a natural killer T cell, a tumor infiltrating T cell (TIL T cell), and a suppressor T cell.

In another aspect, the disclosure features one or more nucleic acids encoding the TCR and CSR comprised by the immune cell described herein. In some embodiments, the TCR and CSR each consist of one or more polypeptide chains encoded by the one or more nucleic acids.

In another aspect, the disclosure features one or more vectors comprising the one or more nucleic acids described above.

In another aspect, the disclosure features a pharmaceutical composition comprising: (a) the immune cell described herein, the nucleic acid(s) described herein, or the vector(s) described herein, and (b) a pharmaceutically acceptable carrier or diluent.

In another aspect, the disclosure features a method of killing target cells, comprising: contacting one or more target cells with the immune cell described herein under conditions and for a time sufficient so that the immune cells mediate killing of the target cells, wherein the target cells express an antigen specific to the immune cell, and wherein the immune cell expresses a low cell exhaustion level upon contacting the target cells. In some embodiments, the immune cell expresses a low cell exhaustion level of an exhaustion marker selected from the group consisting of PD-1, TIM-3, TIGIT, and LAG-3. In certain embodiments, the immune cell is a T cell. In certain embodiments, the immune cell expresses a low cell exhaustion level of PD-1. In certain embodiments, the immune cell expresses a low cell exhaustion level of TIM-3. In certain embodiments, the immune cell expresses a low cell exhaustion level of TIGIT. In certain embodiments, the immune cell expresses a low cell exhaustion level of LAG-3.

In some embodiments, the immune cell expresses a lower level of PD-1, TIM-3, TIGIT, or LAG-3 than corresponding immune cell expressing a CSR comprising a CD28 costimulatory domain. In some embodiments, the immune cell expresses a lower level of PD-1 than the corresponding CD28 CSR immune cell, and wherein the ratio of PD-1 expression level of the immune cell to the corresponding CD28 CSR immune cell is 0.9, 0.8, 0.7, 0.6, 0.5, 0.4, 0.3, 0.2, 0.1 or lower. In some embodiments, the immune cell expresses a lower level of TIM-3 than the corresponding CD28 CSR immune cell, and wherein the ratio of TIM-3 expression level of the immune cell to the corresponding CD28 CSR immune cell is 0.9, 0.8, 0.7, 0.6, 0.5, 0.4, 0.3, 0.2, 0.1 or lower. In some embodiments, the immune cell expresses a lower level of LAG-3 than the corresponding CD28 CSR immune cell, and wherein the ratio of LAG-3 expression level of the immune cell to the corresponding CD28 CSR immune cell is 0.9, 0.8, 0.7, 0.6, 0.5, 0.4, 0.3, 0.2, 0.1 or lower. In some embodiments, the immune cell expresses a lower level of TIGIT than the corresponding CD28 CSR immune cell, and wherein the ratio of TIGIT expression level of the immune cell to the corresponding CD28 CSR immune cell is 0.9, 0.8, 0.7, 0.6, 0.5, 0.4, 0.3, 0.2, 0.1 or lower.

In some embodiments, the immune cell expresses a lower level of PD-1, TIM-3, TIGIT, or LAG-3 than corresponding immune cell expressing a CSR comprising a 4-1BB costimulatory domain. In some embodiments, the immune cell expresses a lower level of PD-1 than the corresponding 4-1BB CSR immune cell, and wherein the ratio of PD-1 expression level of the immune cell to the corresponding 4-1BB CSR immune cell is 0.9, 0.8, 0.7, 0.6, 0.5, 0.4, 0.3, 0.2, 0.1 or lower. In some embodiments, the immune cell expresses a lower level of TIM-3 than the corresponding 4-1BB CSR immune cell, and wherein the ratio of TIM-3 expression level of the immune cell to the corresponding 4-1BB CSR immune cell is 0.9, 0.8, 0.7, 0.6, 0.5, 0.4, 0.3, 0.2, 0.1 or lower. In some embodiments, the immune cell expresses a lower level of LAG-3 than the corresponding 4-1BB CSR immune cell, and wherein the ratio of LAG-3 expression level of the immune cell to the corresponding 4-1BB CSR immune cell is 0.9, 0.8, 0.7, 0.6, 0.5, 0.4, 0.3, 0.2, 0.1 or lower. In some embodiments, the immune cell expresses a lower level of TIGIT than the corresponding 4-1BB CSR immune cell, and wherein the ratio of TIGIT expression level of the immune cell to the corresponding 4-1BB CSR immune cell is 0.9, 0.8, 0.7, 0.6, 0.5, 0.4, 0.3, 0.2, 0.1 or lower.

In some embodiments of this aspect, the target cells are cancer cells. The cancer cells can be from a cancer selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer. The cancer cells can be hematological cancer cells. The cancer cells can be solid tumor cells.

In some embodiments, the target cells are virus-infected cells.

In another aspect, the disclosure features a method of treating a disease, the method comprising a step of administering to a subject the immune cell described herein, the nucleic acid(s) described herein, or the vector(s) described herein, or the pharmaceutical composition described herein to the subject. In some embodiments, the disease is a viral infection. In some embodiments, the disease is cancer. The cancer can be a hematological cancer. The cancer can be a solid tumor cancer.

In some embodiments, the subject has a higher density of the immune cell described herein in the solid tumor cancer than in the rest of the subject's body.

In some embodiments, the cancer is selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer.

In another aspect, the disclosure features a method for preventing and/or reversing T cell exhaustion in a subject, comprising administering to the subject the nucleic acid(s) described herein, the vector(s) described herein, or the pharmaceutical composition described herein comprising the nucleic acid(s) or the vector(s) to the subject. In some embodiments, the method decreases the expression of an exhaustion marker in a T cell. The exhaustion marker can be selected from the group consisting of PD-1, TIM-3, TIGIT, and LAG-3.

In another aspect, the disclosure features a method of treating a solid tumor cancer in a subject with increased tumor infiltration as compared to treating the same type of solid tumor cancer with immune cells expressing a CSR comprising a CD28, 4-1BB, or DAP10 costimulatory domain, wherein the method comprises administering to the subject corresponding immune cells expressing the same TCR and a corresponding CSR comprising a CD30 costimulatory domain, and wherein the corresponding immune cells comprise the immune cell described herein. In some embodiments, experiments can be conducted in animals, e.g., mice, to compare the effects of the immune cells in treating a solid tumor cancer by using one group of immune cells comprising a TCR and a CSR with a CD30 costimulatory domain and another group of immune cells comprising the same TCR and a corresponding CSR with a non-CD30 costimulatory domain, e.g., a 4-1BB costimulatory domain or a CD28 costimulatory domain.

In another aspect, the disclosure features a method of treating a solid tumor cancer in a subject with increased tumor regression as compared to treating the same type of solid tumor cancer with immune cells expressing a TCR and a CSR comprising a CD28, 4-1BB, or DAP10 costimulatory domain, wherein the method comprises administering to the subject corresponding immune cells expressing the same TCR and a corresponding CSR comprising a CD30 costimulatory domain, and wherein the corresponding immune cells comprise the immune cell described herein. In some embodiments, experiments can be conducted in animals, e.g., mice, to compare the effects of the immune cells on tumor regression by using one group of immune cells comprising a TCR and a CSR with a CD30 costimulatory domain and another group of immune cells comprising the same TCR and a corresponding CSR with a non-CD30 costimulatory domain, e.g., a 4-1BB costimulatory domain or a CD28 costimulatory domain.

In another aspect, the disclosure features a method of treating a solid tumor cancer in a subject, the method comprising the steps of: (a) transducing tumor infiltrating T cells (TIL T cells) obtained from the subject, or progenies of the TIL T cells, with a nucleic acid encoding, or a vector comprising a nucleic acid encoding, a chimeric stimulating receptor (CSR) comprising: (i) a ligand-binding module that is capable of binding or interacting with a target ligand; (ii) a transmembrane domain (a CSR transmembrane domain); and (iii) a CD30 costimulatory domain, wherein the CSR lacks a functional primary signaling domain (e.g., a functional primary signaling domain derived from the intracellular signaling sequence of CD3ζ); and (b) administering to the subject transduced TIL T cells or progenies thereof.

In some embodiments, the ligand-binding module of the CSR comprises an antibody moiety (a CSR antibody moiety). In some embodiments, the CD30 costimulatory domain comprises a sequence that is at least 80%, 85%, 90%, 95%, or 100% identical to residues 561-573 or 578-586 of SEQ ID NO:228. In some embodiments, the CD30 costimulatory domain comprises a sequence that is at least 70%, 75%, 80%, 85%, 90%, 95%, or 100% identical to the sequence of SEQ ID NO:238.

In some embodiments of this aspect, the target ligand is a cell surface antigen on a solid tumor. In particular embodiments, the cell surface antigen is Glypican 3 (GPC3), HER2/ERBB2, EpCAM, MUC16, folate receptor alpha (FRα), MUC1, EGFR, EGFRvIII, HER3, DLL3, c-Met, ROR2, CD70, MCT4, MSLN, PSMA, or a variant or mutant thereof.

In some embodiments of this aspect, the TIL T cells comprise an αβ TCR. In some embodiments, the TCR specifically binds to a disease-related MHC-restricted antigen. In some embodiments, the disease-related MHC-restricted antigen is expressed on cell surface of the solid tumor cancer.

In some embodiments, the TCR does not specifically bind to a disease-related MHC-restricted antigen on cell surface of the solid tumor cancer.

In some embodiments of this aspect, the method further comprises a step of obtaining TIL T cells from the subject prior to the transducing step. In some embodiments, the subject has a higher density of the transduced TIL T cells in the solid tumor cancer than in the rest of the subject's body.

In some embodiments of this aspect, the cancer is selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer.

In another aspect, the disclosure features a method for generating central memory T cells in a subject, comprising administering to the subject the nucleic acid(s) described herein, the vector(s) described herein, or the pharmaceutical composition described herein comprising the nucleic acid(s) or the vector(s) to the subject.

In some embodiments, the method increases the number of central memory T cells and/or the percentage of central memory T cells among all T cells in the subject.

In another aspect, the disclosure provides a method for generating central memory T cells in vitro comprising: contacting one or more target cells with the immune cell described herein under conditions and for a time sufficient so that the immune cell develops into central memory T cells, wherein the target cells express an antigen specific to the immune cell.

In some embodiments, the method increases the number of central memory T cells and/or the percentage of central memory T cells among all T cells descended from the immune cell.

In some embodiments, the method generates higher number of central memory T cells and/or higher percentage of central memory T cells than corresponding immune cell expressing a CSR comprising a CD28 costimulatory domain.

In some embodiments, the method generates at least 10%, 20%, 30%, 40%, 50%, 60%, 70%, 80%, 90%, 100%, 200%, 300%, 400%, or 500% higher number of central memory T cells and/or percentage of central memory T cells than corresponding immune cell expressing a CSR comprising a CD28 costimulatory domain.

In some embodiments, the central memory T cells express high levels of CCR7 and low levels of CD45RA.

In some embodiments, the central memory T cells are CD8⁺ T cells.

In another aspect, the disclosure provides a method of treating a solid tumor cancer in a subject with increased tumor infiltration or immune cell expansion as compared to treating the same type of solid tumor cancer with immune cells expressing a TCR and a CSR comprising a control costimulatory domain, wherein the method comprises administering to the subject corresponding immune cells expressing the same TCR and a corresponding CSR comprising a CD30 costimulatory domain, and wherein the corresponding immune cells comprise the immune cell described herein. In some embodiments, the control costimulatory domain is a CD28, 4-1BB, or DAP10 costimulatory domain.

In another aspect, the disclosure provides a method of treating a solid tumor cancer in a subject with increased tumor regression as compared to treating the same type of solid tumor cancer with immune cells expressing a TCR and a CSR comprising a CD28, 4-1 BB, or DAP10 costimulatory domain, wherein the method comprises administering to the subject corresponding immune cells expressing the same TCR and a corresponding CSR comprising a CD30 costimulatory domain, and wherein the corresponding immune cells comprise the immune cell described herein.

In yet another aspect, the disclosure provides a method for generating central memory T cells in a subject, comprising administering to the subject the nucleic acid(s) described herein, the vector(s) described herein, or the pharmaceutical composition described herein that comprises the nucleic acid(s) or the vector(s) to the subject. In some embodiments, the method increases the number of central memory T cells and/or the percentage of central memory T cells among all T cells in the subject.

In yet another aspect, the disclosure provides a method for generating central memory T cells in vitro comprising: contacting one or more target cells with the immune cell described herein under conditions and for a time sufficient so that the immune cell develops into central memory T cells, wherein the target cells express an antigen specific to the immune cell. In some embodiments, the method increases the number of central memory T cells and/or the percentage of central memory T cells among all T cells descended from the immune cell. In some embodiments, the method generates higher number of central memory T cells and/or higher percentage of central memory T cells than corresponding immune cell expressing a CSR comprising a CD28 or DAP10 costimulatory domain. In certain embodiments, the method generates at least 10%, 20%, 30%, 40%, 50%, 60%, 70%, 80%, 90%, 100%, 200%, 300%, 400%, or 500% higher number of central memory T cells and/or percentage of central memory T cells than corresponding immune cell expressing a CSR comprising a CD28 or DAP10 costimulatory domain. In certain embodiments, the central memory T cells express high levels of CCR7 and low levels of CD45RA. In particular embodiments, the central memory T cells are CD8⁺ T cells.

### Illustrative Embodiments of the Disclosure

The following embodiments serve to illustrate various features of the present disclosure. The scope of the disclosure is not limited to the illustrative embodiments or particular features presented in the illustrative embodiments and encompasses embodiments and features as detailed in the present applications that are not specifically articulated in this section. Thus, in some aspects, the disclosure provides:

Embodiment 1: An immune cell comprising:
(a) an αβ T-cell receptor (TCR), and
(b) a chimeric stimulating receptor (CSR) comprising:
(i) a ligand-binding module that is capable of binding or interacting with a target ligand;
(ii) a transmembrane domain (a CSR transmembrane domain); and
(iii) a CD30 costimulatory domain,
   wherein the CSR lacks a functional primary signaling domain derived from the intracellular signaling sequence of CD3ζ.
   Embodiment 2: The immune cell of embodiment 1, wherein the CD30 costimulatory domain comprises a sequence that can bind to an intracellular TRAF signaling protein.
   Embodiment 3: The immune cell of embodiment 2, wherein the sequence that can bind to an intracellular TRAF signaling protein corresponds to residues 561-573 or 578-586 of a full-length CD30 having the sequence of SEQ ID NO:228.
   Embodiment 4: The immune cell of any one of embodiments 1 to 3, wherein the CD30 costimulatory domain comprises a sequence that is at least 80%, 85%, 90%, 950, or 100% identical to residues 561-573 or 578-586 of SEQ ID NO:228.
   Embodiment 5: The immune cell of any one of embodiments 1 to 4, wherein the CD30 costimulatory domain comprises a sequence that is at least 70%, 75%, 80%, 85%, 90%, 95%, or 100% identical to the sequence of SEQ ID NO:238.
   Embodiment 6: The immune cell of any one of embodiments 1 to 5, wherein the CSR comprises more than one CD30 costimulatory domain.
   Embodiment 7: The immune cell of any one of embodiments 1 to 6, wherein the CSR further comprises at least one costimulatory domain which comprises the intracellular sequence of a costimulatory molecule that is different from CD30.
   Embodiment 8: The immune cell of embodiment 7, wherein the costimulatory molecule that is different from CD30 is selected from the group consisting of CD27, CD28, 4-1BB (CD137), OX40, CD40, PD-1, ICOS, lymphocyte function-associated antigen-1 (LFA-1), CD2, CD7, LIGHT, NKG2C, B7-H3, and a ligand that specifically binds with CD83.
   Embodiment 9: The immune cell of any one of embodiments 1 to 8, wherein the ligand-binding module of the CSR is derived from the extracellular domain of a receptor.
   Embodiment 10: The immune cell of any one of embodiments 1 to 8, the ligand-binding module of the CSR comprises an antibody moiety (a CSR antibody moiety).
   Embodiment 11: The immune cell of embodiment 10, wherein the CSR antibody moiety is a single chain antibody fragment.
   Embodiment 12: The immune cell of embodiment 10 or 11, wherein the CSR antibody moiety is a single chain Fv (scFv), a single chain Fab, a single chain Fab′, a single domain antibody fragment, a single domain multispecific antibody, an intrabody, a nanobody, or a single chain immunokine.
   Embodiment 13: The immune cell of embodiment 12, wherein the CSR antibody moiety is a single domain multispecific antibody.
   Embodiment 14: The immune cell of embodiment 13, wherein the single domain multispecific antibody is a single domain bispecific antibody.
   Embodiment 15: The immune cell of any one of embodiments 10 to 14, wherein the CSR antibody moiety is a single chain Fv (scFv).
   Embodiment 16: The immune cell of embodiment 15, wherein the scFv is a tandem scFv.
   Embodiment 17: The immune cell of any one of embodiments 1 to 16, wherein the TCR and/or the CSR antibody moiety specifically binds to a disease-related MHC-restricted antigen.
   Embodiment 18: The immune cell of embodiment 17, wherein the disease-related antigen is a cancer-related antigen.
   Embodiment 19: The immune cell of any one of embodiments 10 to 18, wherein both the TCR and the CSR antibody moiety specifically bind to a MHC-restricted antigen.
   Embodiment 20: The immune cell of any one of embodiments 10 to 19, wherein the TCR and the CSR antibody moiety specifically bind to the same antigen.
   Embodiment 21: The immune cell of embodiment 20, wherein the TCR and the CSR antibody moiety specifically bind to different peptides from the same antigen.
   Embodiment 22: The immune cell of any one of embodiments 10 to 19, wherein the TCR and the CSR antibody moiety specifically bind to different antigens.
   Embodiment 23: The immune cell of any one of embodiments 1 to 22, wherein the TCR and/or the CSR antibody moiety specifically binds to a complex comprising a peptide and an MHC protein, and wherein the peptide is derived from a protein selected from the group consisting of WT-1, AFP, HPV16-E7, NY-ESO-1, PRAME, EBV-LMP2A, KRAS, FoxP3, Histone H3.3, PSA, COL18A1, SRPX, KIF16B, TFDP2, KIAA1279, XPNPEP1, UGGT2, PHKA1, KIF16B, SON, GNB5, FBXO21, CORO7, RECQL5, TFDP2, KIAA1967, KIF16B, NUP98, GPD2, CASP8, SKIV2L, H3F3B, MAGE-A4, MAGEA6, PDS5A, MED13, SLC3A2, KIAA0368, CADPS2, CTSB, DPY19L4, RNF19B, ASTN1, CDK4, MLL2, SMARCD3, p53, RAD21. RUSC2, VPS16, MGA, ARHGAP35, HER2, 5T4, and a variant or mutant thereof.
   Embodiment 24: The immune cell of embodiment 23, wherein the TCR specifically binds to the complex.
   Embodiment 25: The immune cell of any one of embodiments 10 to 22 and 24, wherein the CSR antibody moiety specifically binds to a cell surface antigen.
   Embodiment 26: The immune cell of embodiment 25, wherein the cell surface antigen is selected from the group consisting of protein, carbohydrate, and lipid.
   Embodiment 27: The immune cell of embodiment 25 or 26, wherein the TCR specifically binds to a complex comprising an MHC protein and a peptide derived from a cell surface antigen, and wherein the CSR antibody moiety specifically bind to the same cell surface antigen.
   Embodiment 28: The immune cell of any one of embodiments 25 to 27, wherein the cell surface antigen is Glypican 3 (GPC3), HER2/ERBB2, EpCAM, MUC16, folate receptor alpha (FRα), MUC1, EGFR, EGFRvIII, HER3, DLL3, c-Met, ROR2, CD70, MCT4, MSLN, PSMA, or a variant or mutant thereof.
   Embodiment 29: The immune cell of any one of embodiments 1 to 28, wherein the TCR specifically binds to a complex comprising an alpha-fetoprotein (AFP) peptide and an MHC class I protein.
   Embodiment 30: The immune cell of embodiment 29, wherein the AFP peptide comprises an amino acid sequence of any one of SEQ ID NOS:26-36.
   Embodiment 31: The immune cell of any one of embodiments 1 to 30, wherein the TCR comprises: (1) an anti-AFP-TCRα chain comprising sequences of CDR1, CDR2, and CDR3 of SEQ ID NOS:305-307, respectively; or (2) an anti-AFP-TCRβ chain comprising sequences of CDR1, CDR2, and CDR3 of SEQ ID NOS:308-310, respectively; or (3) an anti-AFP-TCRα chain comprising sequences of CDR1, CDR2, and CDR3 of SEQ ID NOS:311-313, respectively.
   Embodiment 32: The immune cell of embodiment 31, wherein the TCR comprises: (1) an anti-AFP-TCRα chain variable region comprising a sequence of SEQ ID NO:314; or (2) an anti-AFP-TCRβ chain variable region comprising a sequence of SEQ ID NO:315; or (3) an anti-AFP-TCRα chain variable region comprising a sequence of SEQ ID NO:316.
   Embodiment 33: The immune cell of embodiment 31 or 32, wherein the TCR comprises a sequence of any one of SEQ ID NOS:1-3.
   Embodiment 34: The immune cell of any one of embodiments 1 to 30, wherein the TCR comprises a sequence of any one of SEQ ID NOS:6-19 and 178-180.
   Embodiment 35: The immune cell of any one of embodiments 1 to 34, wherein the CSR specifically binds to glypican 3 (GPC3).
   Embodiment 36: The immune cell of embodiment 35, wherein the CSR comprises: (1) sequences of HCDR1, HCDR2, HCDR3, LCDR1, LCDR2, and LCDR3 of SEQ ID NOS:317-322, respectively; or (2) sequences of HCDR1, HCDR2, HCDR3, LCDR1, LCDR2, and LCDR3 of SEQ ID NOS:323-328, respectively; or (3) sequences of HCDR1, HCDR2. HCDR3, LCDR1, LCDR2, and LCDR3 of SEQ ID NOS:329-334, respectively; or (4) sequences of HCDR1, HCDR2, HCDR3, LCDR1, LCDR2, and LCDR3 of SEQ ID NOS:335-340, respectively; or (5) sequences of HCDR1, HCDR2, HCDR3, LCDR1, LCDR2, and LCDR3 of SEQ ID NOS:341-346, respectively; or (6) sequences of HCDR1, HCDR2, HCDR3, LCDR1, LCDR2, and LCDR3 of SEQ ID NOS:347-352, respectively; or (7) sequences of HCDR1, HCDR2, HCDR3, LCDR1, LCDR2, and LCDR3 of SEQ ID NOS:353-358, respectively.
   Embodiment 37: The immune cell of embodiment 35 or 36, wherein the CSR comprises a heavy chain variable region having the sequence of any one of SEQ ID NOS:274, 276, 278, 280, 282, 284, and 286, and a light chain variable region having the sequence of any one of SEQ ID NOS:275, 277, 279, 281, 283, 285, and 287.
   Embodiment 38: The immune cell of any one of embodiments 35 to 37, wherein the CSR comprises an scFv having the sequence of any one of SEQ ID NOS:212-213 and 269-273.
   Embodiment 39: The immune cell of any one of embodiments 35 to 38, wherein the CSR comprises an amino acid sequence of any one of SEQ ID NOS:181-211 and 288-293.
   Embodiment 40: The immune cell of any one of embodiments 1 to 34, wherein the CSR specifically binds to MSLN.
   Embodiment 41: The immune cell of any one of embodiments 1 to 23, wherein the TCR specifically binds to a complex comprising a KRAS, p53, or MSLN peptide and an MHC class I protein.
   Embodiment 42: The immune cell of any one of embodiments 1 to 23 and 41, wherein the CSR specifically binds to MSLN.
   Embodiment 43: The immune cell of embodiment 42, wherein the CSR comprises sequences of HCDR1, HCDR2, and HCDR3 of SEQ ID NOS:71-73, respectively, and optionally a heavy chain variable region having the sequence of SEQ ID NO:70.
   Embodiment 44: The immune cell of embodiment 42 or 43, wherein the CSR comprises sequences of LCDR1, LCDR2, and LCDR3 of SEQ ID NOS:75-77, respectively, and optionally a light chain variable region having the sequence of SEQ ID NO:74.
   Embodiment 45: The immune cell of any one of embodiments 1 to 23 and 41, wherein the CSR specifically binds to ROR1.
   Embodiment 46: The immune cell of embodiment 45, wherein the CSR specifically binds to a ROR1 peptide having a sequence of any one of SEQ ID NOS:443-446.
   Embodiment 47: The immune cell of embodiment 45 or 46, wherein the CSR comprises sequences of HCDR1, HCDR2, and HCDR3 of SEQ ID NOS:447-449, respectively, and optionally a heavy chain variable region having the sequence of SEQ ID NO:450; and/or sequences of LCDR1, LCDR2, and LCDR3 of SEQ ID NOS:451-453, respectively, and optionally a light chain variable region having the sequence of SEQ ID NO:454; and/or optionally an scFv having the sequence of SEQ ID NO:441.
   Embodiment 48: The immune cell of embodiment 45 or 46, wherein the CSR comprises sequences of HCDR1, HCDR2, and HCDR3 of SEQ ID NOS:455-457, respectively, and optionally a heavy chain variable region having the sequence of SEQ ID NO:458; and/or sequences of LCDR1, LCDR2, and LCDR3 of SEQ ID NOS:459-461, respectively, and optionally a light chain variable region having the sequence of SEQ ID NO:462; and/or optionally an scFv having the sequence of SEQ ID NO:442.
   Embodiment 49: The immune cell of any one of embodiments 1 to 23, wherein the TCR specifically binds to a complex comprising a PSA peptide and an MHC class I protein.
   Embodiment 50: The immune cell of any one of embodiments 1 to 23 and 49, wherein the CSR specifically binds to PSMA.
   Embodiment 51: The immune cell of any one of embodiments 1 to 23 and 49, wherein the CSR specifically binds to ROR1.
   Embodiment 52: The immune cell of embodiment 51, wherein the CSR specifically binds to a ROR1 peptide having a sequence of any one of SEQ ID NOS:443-446.
   Embodiment 53: The immune cell of embodiment 51 or 52, wherein the CSR comprises sequences of HCDR1, HCDR2, and HCDR3 of SEQ ID NOS:447-449, respectively, and optionally a heavy chain variable region having the sequence of SEQ ID NO:450; and/or sequences of LCDR1, LCDR2, and LCDR3 of SEQ ID NOS:451-453, respectively, and optionally a light chain variable region having the sequence of SEQ ID NO:454; and/or optionally an scFv having the sequence of SEQ ID NO:441.
   Embodiment 54: The immune cell of embodiment 51 or 52, wherein the CSR comprises sequences of HCDR1, HCDR2, and HCDR3 of SEQ ID NOS:455-457, respectively, and optionally a heavy chain variable region having the sequence of SEQ ID NO:458; and/or sequences of LCDR1, LCDR2, and LCDR3 of SEQ ID NOS:459-461, respectively, and optionally a light chain variable region having the sequence of SEQ ID NO:462; and/or optionally an scFv having the sequence of SEQ ID NO:442.
   Embodiment 55: The immune cell of embodiment 49 or 50, wherein the TCR comprises a sequence of any one of SEQ ID NOS:20-25.
   Embodiment 56: The immune cell of any one of embodiments 49 to 55, wherein the CSR comprises sequences of HCDR1, HCDR2, and HCDR3 of SEQ ID NOS:373-375, respectively, and optionally a heavy chain variable region having the sequence of SEQ ID NO:376.
   Embodiment 57: The immune cell of any one of embodiments 49 to 56, wherein the CSR comprises sequences of LCDR1, LCDR2, and LCDR3 of SEQ ID NOS:377-379, respectively, and optionally a light chain variable region having the sequence of SEQ ID NO:380.
   Embodiment 58: The immune cell of any one of embodiments 49 to 57, wherein the CSR comprises a sequence of SEQ ID NO:214.
   Embodiment 59: The immune cell of any one of embodiments 49 to 55, wherein the CSR comprises sequences of HCDR1, HCDR2, and HCDR3 of SEQ ID NOS:381-383, respectively, and optionally a heavy chain variable region having the sequence of SEQ ID NO:384.
   Embodiment 60: The immune cell of any one of embodiments 49 to 55 and 59, wherein the CSR comprises sequences of LCDR1, LCDR2, and LCDR3 of SEQ ID NOS:385-387, respectively, and optionally a light chain variable region having the sequence of SEQ ID NO:388.
   Embodiment 61: The immune cell of any one of embodiments 49 to 55, 59, and 60, wherein the CSR comprises a sequence of SEQ ID NO:215.
   Embodiment 62: The immune cell of any one of embodiments 1 to 23, wherein the TCR specifically binds to a complex comprising a COL18A1, SRPX, KIF16B, TFDP2, KIAA1279. XPNPEP1, UGGT2, PHKA1, KIF16B, SON, GNB5, FBXO21, CORO7, RECQL5, TFDP2, KIAA1967, KIF16B, MAGEA6, PDS5A, MED13, ASTN1, CDK4, MLL2, SMARCD3, NY-ESO-1, or PRAME peptide and an MHC class I protein.
   Embodiment 63: The immune cell of any one of embodiments 1 to 23 and 62, wherein the CSR specifically binds to ROR2.
   Embodiment 64: The immune cell of embodiment 62 or 63, wherein the TCR specifically binds to a complex comprising NY-ESO-1 and the MHC I protein and comprises: (1) sequences of CDR1, CDR2, and CDR3 of SEQ ID NOS:359-361, respectively, and optionally a variable region having the sequence of SEQ ID NO:362, and further optionally the sequence of SEQ ID NO:4; or (2) sequences of CDR1, CDR2, and CDR3 of SEQ ID NOS:363-365, respectively, and optionally a variable region having the sequence of SEQ ID NO:366, and further optionally the sequence of SEQ ID NO:5.
   Embodiment 65: The immune cell of any one of embodiments 62 to 64, wherein the CSR comprises: (1) sequences of HCDR1, HCDR2, and HCDR3 of SEQ ID NOS:91-93, respectively, and optionally a heavy chain variable region having the sequence of SEQ ID NO:90; or (2) sequences of HCDR1, HCDR2, and HCDR3 of SEQ ID NOS:95-97, respectively, and optionally a heavy chain variable region having the sequence of SEQ ID NO:94; or (3) sequences of HCDR1, HCDR2, and HCDR3 of SEQ ID NOS:99-101, respectively, and optionally a heavy chain variable region having the sequence of SEQ ID NO:98; or (4) sequences of HCDR1, HCDR2, and HCDR3 of SEQ ID NOS:103-105, respectively, and optionally a heavy chain variable region having the sequence of SEQ ID NO:102; or (5) sequences of HCDR1, HCDR2, and HCDR3 of SEQ ID NOS:107-109, respectively, and optionally a heavy chain variable region having the sequence of SEQ ID NO:106.
   Embodiment 66: The immune cell of any one of embodiments 62 to 65, wherein the CSR comprises: (1) sequences of LCDR1, LCDR2, and LCDR3 of SEQ ID NOS:111-113, respectively, and optionally a light chain variable region having the sequence of SEQ ID NO:110; or (2) sequences of LCDR1, LCDR2, and LCDR3 of SEQ ID NOS:115-117, respectively, and optionally a light chain variable region having the sequence of SEQ ID NO:114; or (3) sequences of LCDR1, LCDR2, and LCDR3 of SEQ ID NOS:119-121, respectively, and optionally a light chain variable region having the sequence of SEQ ID NO:118; or (4) sequences of LCDR1, LCDR2, and LCDR3 of SEQ ID NOS:123-125, respectively, and optionally a light chain variable region having the sequence of SEQ ID NO:122; or (5) sequences of LCDR1, LCDR2, and LCDR3 of SEQ ID NOS:127-129, respectively, and optionally a light chain variable region having the sequence of SEQ ID NO:126.
   Embodiment 67: The immune cell of any one of embodiments 1 to 23, wherein the TCR specifically binds to a complex comprising a NUP98, GPD2, CASP8, KRAS, SKIV2L, H3F3B, RAD21, or PRAME peptide and an MHC class I protein.
   Embodiment 68: The immune cell of any one of embodiments 1 to 23 and 67, wherein the CSR specifically binds to ROR2.
   Embodiment 69: The immune cell of any one of embodiments 67 or 68, wherein the CSR comprises: (1) sequences of HCDR1, HCDR2, and HCDR3 of SEQ ID NOS:91-93, respectively, and optionally a heavy chain variable region having the sequence of SEQ ID NO:90; or (2) sequences of HCDR1, HCDR2, and HCDR3 of SEQ ID NOS:95-97, respectively, and optionally a heavy chain variable region having the sequence of SEQ ID NO:94; or (3) sequences of HCDR1, HCDR2, and HCDR3 of SEQ ID NOS:99-101, respectively, and optionally a heavy chain variable region having the sequence of SEQ ID NO:98; or (4) sequences of HCDR1, HCDR2, and HCDR3 of SEQ ID NOS:103-105, respectively, and optionally a heavy chain variable region having the sequence of SEQ ID NO:102; or (5) sequences of HCDR1, HCDR2, and HCDR3 of SEQ ID NOS:107-109, respectively, and optionally a heavy chain variable region having the sequence of SEQ ID NO:106.
   Embodiment 70: The immune cell of any one of embodiments 67 to 69, wherein the CSR comprises: (1) sequences of LCDR1, LCDR2, and LCDR3 of SEQ ID NOS:111-113, respectively, and optionally a light chain variable region having the sequence of SEQ ID NO:110; or (2) sequences of LCDR1, LCDR2, and LCDR3 of SEQ ID NOS: 15-117, respectively, and optionally a light chain variable region having the sequence of SEQ ID NO:114; or (3) sequences of LCDR1, LCDR2, and LCDR3 of SEQ ID NOS:119-121, respectively, and optionally a light chain variable region having the sequence of SEQ ID NO:118; or (4) sequences of LCDR1, LCDR2, and LCDR3 of SEQ ID NOS:123-125, respectively, and optionally a light chain variable region having the sequence of SEQ ID NO:122; or (5) sequences of LCDR1, LCDR2, and LCDR3 of SEQ ID NOS:127-129, respectively, and optionally a light chain variable region having the sequence of SEQ ID NO:126.
   Embodiment 71: The immune cell of any one of embodiments 1 to 23, wherein the TCR specifically binds to a complex comprising a SLC3A2, KIAA0368, CADPS2, CTSB, PRAME, Embodiment p53, or PSA peptide and an MHC class I protein.
   Embodiment 72: The immune cell of any one of embodiments 1 to 23 and 71, wherein the CSR specifically binds to HER2, EpCAM, or ROR1.
   Embodiment 73: The immune cell of embodiment 71 or 72, wherein the TCR comprises a sequence of any one of SEQ ID NOS:20-25.
   Embodiment 74: The immune cell of any one of embodiments 71 to 73, wherein the CSR binds to HER2 and comprises sequences of HCDR1, HCDR2, and HCDR3 of SEQ ID NOS:389-391, respectively, and optionally a heavy chain variable region having the sequence of SEQ ID NO:41.
   Embodiment 75: The immune cell of any one of embodiments 71 to 74, wherein the CSR binds to HER2 and comprises sequences of LCDR1, LCDR2, and LCDR3 of SEQ ID NOS:392-394, respectively, and optionally a light chain variable region having the sequence of SEQ ID NO:42.
   Embodiment 76: The immune cell of any one of embodiments 71 to 73, wherein the CSR specifically binds to EpCAM and comprises sequences of HCDR1, HCDR2, and HCDR3 of SEQ ID NOS:403-405, respectively, and optionally a heavy chain variable region having the sequence of SEQ ID NO:60.
   Embodiment 77: The immune cell of any one of embodiments 71 to 76, wherein the CSR binds to EpCAM and comprises sequences of LCDR1, LCDR2, and LCDR3 of SEQ ID NOS:406-408, respectively, and optionally a light chain variable region having the sequence of SEQ ID NO:61.
   Embodiment 78: The immune cell of any one of embodiments 71 to 73, wherein the CSR binds to ROR1 and comprises sequences of HCDR1, HCDR2, and HCDR3 of SEQ ID NOS:447-449, respectively, and optionally a heavy chain variable region having the sequence of SEQ ID NO:450; and/or sequences of LCDR1, LCDR2, and LCDR3 of SEQ ID NOS:451-453, respectively, and optionally a light chain variable region having the sequence of SEQ ID NO:454; and/or optionally an scFv having the sequence of SEQ ID NO:441.
   Embodiment 79: The immune cell of any one of embodiments 71 to 73, wherein the CSR comprises sequences of HCDR1, HCDR2, and HCDR3 of SEQ ID NOS:455-457, respectively, and optionally a heavy chain variable region having the sequence of SEQ ID NO:458; and/or sequences of LCDR1, LCDR2, and LCDR3 of SEQ ID NOS:459-461, respectively, and optionally a light chain variable region having the sequence of SEQ ID NO:462; and/or optionally an scFv having the sequence of SEQ ID NO:442.
   Embodiment 80: The immune cell of embodiment 78 or 79, wherein the CSR specifically binds to a ROR1 peptide having a sequence of any one of SEQ ID NOS:443-446.
   Embodiment 81: The immune cell of any one of embodiments 1 to 23, wherein the TCR specifically binds to a complex comprising a WT1, NY-ESO-1, p53, DPY19L4, or RNF19B peptide and an MHC class I protein.
   Embodiment 82: The immune cell of any one of embodiments 1 to 23 and 81, wherein the CSR specifically binds to MUC1, MUC16, FRα, or ROR1.
   Embodiment 83: The immune cell of embodiment 81 or 82, wherein the TCR specifically binds to a complex comprising NY-ESO-1 and the MHC embodiments 1 protein and comprises: (1) sequences of CDR1, CDR2, and CDR3 of SEQ ID NOS:359-361, respectively, and optionally a variable region having the sequence of SEQ ID NO:362, and further optionally the sequence of SEQ ID NO:4; or (2) sequences of CDR1, CDR2, and CDR3 of SEQ ID NOS:363-365, respectively, and optionally a variable region having the sequence of SEQ ID NO:366, and further optionally the sequence of SEQ ID NO:5.
   Embodiment 84: The immune cell of any one of embodiments 81 to 83, wherein the CSR specifically binds to MUC1 and comprises sequences of HCDR1, HCDR2, and HCDR3 of SEQ ID NOS:417-419, respectively, and optionally a heavy chain variable region having the sequence of SEQ ID NO:367.
   Embodiment 85: The immune cell of any one of embodiments 81 to 84, wherein the CSR specifically binds to MUC1 and comprises sequences of LCDR1, LCDR2, and LCDR3 of SEQ ID NOS:420-422, respectively, and optionally a light chain variable region having the sequence of SEQ ID NO:368.
   Embodiment 86: The immune cell of any one of embodiments 81 to 83, wherein the CSR specifically binds to MUC16 and comprises: (1) sequences of HCDR1, HCDR2, and HCDR3 of SEQ ID NOS:131-133, respectively, and optionally a heavy chain variable region having the sequence of SEQ ID NO:130; or (2) sequences of HCDR1, HCDR2, and HCDR3 of SEQ ID NOS:135-137, respectively, and optionally a heavy chain variable region having the sequence of SEQ ID NO:134; (3) sequences of HCDR1, HCDR2, and HCDR3 of SEQ ID NOS:429-431, respectively, and optionally a heavy chain variable region having the sequence of any one of SEQ ID NOS:146-147; or (4) sequences of HCDR1, HCDR2, and HCDR3 of SEQ ID NOS:435-437, respectively, and optionally a heavy chain variable region having the sequence of any one of SEQ ID NOS:148-149.
   Embodiment 87: The immune cell of any one of embodiments 81 to 86, wherein the CSR specifically binds to MUC16 and comprises: (1) sequences of LCDR1, LCDR2, and LCDR3 of SEQ ID NOS:139-141, respectively, and optionally a light chain variable region having the sequence of SEQ ID NO:138; or (2) sequences of LCDR1, LCDR2, and LCDR3 of SEQ ID NOS:143-145, respectively, and optionally a light chain variable region having the sequence of SEQ ID NO:142; (3) sequences of LCDR1, LCDR2, and LCDR3 of SEQ ID NOS:432-434, respectively, and optionally a light chain variable region having the sequence of any one of SEQ ID NOS:150-151; or (4) sequences of LCDR1, LCDR2, and LCDR3 of SEQ ID NOS:438-440, respectively, and optionally a light chain variable region having the sequence of any one of SEQ ID NOS: 152-153.
   Embodiment 88: The immune cell of any one of embodiments 81 to 83, wherein the CSR specifically binds to FRα and comprises sequences of HCDR1, HCDR2, and HCDR3 of SEQ ID NOS:423-425, respectively, and optionally a heavy chain variable region having the sequence of SEQ ID NO:369 and further optionally a heavy chain having the sequence of SEQ ID NO:370.
   Embodiment 89: The immune cell of any one of embodiments 81 to 83 and 88, wherein the CSR specifically binds to FRα and comprises sequences of LCDR1, LCDR2, and LCDR3 of SEQ ID NOS:426-428, respectively, and optionally a light chain variable region having the sequence of SEQ ID NO:371 and further optionally a light chain having the sequence of SEQ ID NO:372.
   Embodiment 90: The immune cell of any one of embodiments 81 to 83, wherein the CSR binds to ROR1 and comprises sequences of HCDR1, HCDR2, and HCDR3 of SEQ ID NOS:447-449, respectively, and optionally a heavy chain variable region having the sequence of SEQ ID NO:450; and/or sequences of LCDR1, LCDR2, and LCDR3 of SEQ ID NOS:451-453, respectively, and optionally a light chain variable region having the sequence of SEQ ID NO:454; and/or optionally an scFv having the sequence of SEQ ID NO:441.
   Embodiment 91: The immune cell of any one of embodiments 81 to 83, wherein the CSR comprises sequences of HCDR1, HCDR2, and HCDR3 of SEQ ID NOS:455-457, respectively, and optionally a heavy chain variable region having the sequence of SEQ ID NO:458; and/or sequences of LCDR1, LCDR2, and LCDR3 of SEQ ID NOS:459-461, respectively, and optionally a light chain variable region having the sequence of SEQ ID NO:462; and/or optionally an scFv having the sequence of SEQ ID NO:442.
   Embodiment 92: The immune cell of embodiment 90 or 91, wherein the CSR specifically binds to a ROR1 peptide having a sequence of any one of SEQ ID NOS:443-446.
   Embodiment 93: The immune cell of any one of embodiments 1 to 23, wherein the TCR specifically binds to a complex comprising a WT1 peptide and an MHC class I protein Embodiment 94: The immune cell of embodiment 93, wherein the CSR specifically binds to MUC1.
   Embodiment 95: The immune cell of any one of embodiments 1 to 23, wherein the TCR specifically binds to a complex comprising a p53 or KRAS peptide and an MHC class I protein.
   Embodiment 96: The immune cell of any one of embodiments 1 to 23 and 95, wherein the CSR specifically binds to EGFR.
   Embodiment 97: The immune cell of embodiment 95 or 96, wherein the CSR comprises sequences of HCDR1, HCDR2, and HCDR3 of SEQ ID NOS:79-81, respectively, and optionally a heavy chain variable region having the sequence of SEQ ID NO:78.
   Embodiment 98: The immune cell of any one of embodiments 95 to 97, wherein the CSR comprises sequences of LCDR1, LCDR2, and LCDR3 of SEQ ID NOS:83-85, respectively, and optionally a light chain variable region having the sequence of SEQ ID NO:82.
   Embodiment 99: The immune cell of any one of embodiments 1 to 23, wherein the TCR specifically binds to a complex comprising a ARHGAP35 or Histone H3.3 peptide and an MHC class I protein.
   Embodiment 100: The immune cell of any one of embodiments 1 to 23 and 99, wherein the CSR specifically binds to EGFR or EGFRvIII.
   Embodiment 101: The immune cell of embodiment 99 or 100, wherein the CSR specifically binds to EGFR and comprises sequences of HCDR1, HCDR2, and HCDR3 of SEQ ID NOS:79-81, respectively, and optionally a heavy chain variable region having the sequence of SEQ ID NO:78.
   Embodiment 102: The immune cell of any one of embodiments 99 to 101, wherein the CSR specifically binds to EGFR comprises sequences of LCDR1, LCDR2, and LCDR3 of SEQ ID NOS:83-85, respectively, and optionally a light chain variable region having the sequence of SEQ ID NO:82.
   Embodiment 103: The immune cell of embodiment 99 or 100, wherein the CSR specifically binds to EGFRvIII and comprises sequences of HCDR1, HCDR2, and HCDR3 of SEQ ID NOS:409-411, respectively, and optionally a heavy chain variable region having the sequence of SEQ ID NO:412.
   Embodiment 104: The immune cell of any one of embodiments 99, 100, and 103, wherein the CSR specifically binds to EGFRvIII and comprises sequences of LCDR1, LCDR2, and LCDR3 of SEQ ID NOS:413-415, respectively, and optionally a light chain variable region having the sequence of SEQ ID NO:416.
   Embodiment 105: The immune cell of any one of embodiments 99, 100, 103, and 104, wherein the CSR specifically binds to EGFRvIII and comprises comprises the sequence of SEQ ID NO:86.
   Embodiment 106: The immune cell of any one of embodiments 1 to 23, wherein the TCR specifically binds to a complex comprising a KRAS, HER2, NY-ESO-1, or p53 peptide and an MHC class I protein.
   Embodiment 107: The immune cell of any one of embodiments 1 to 23 and 106, wherein the CSR specifically binds to HER3, DLL3, c-Met, or ROR1.
   Embodiment 108: The immune cell of embodiment 106 or 107, wherein the TCR specifically binds to a complex comprising NY-ESO-1 and the MHC embodiments 1 protein and comprises: (1) sequences of CDR1, CDR2, and CDR3 of SEQ ID NOS:359-361, respectively, and optionally a variable region having the sequence of SEQ ID NO:362, and further optionally the sequence of SEQ ID NO:4; or (2) sequences of CDR1, CDR2, and CDR3 of SEQ ID NOS:363-365, respectively, and optionally a variable region having the sequence of SEQ ID NO:366, and further optionally the sequence of SEQ ID NO:5.
   Embodiment 109: The immune cell of any one of embodiments 106 to 108, wherein the CSR specifically binds to HER3 and comprises sequences of HCDR1, HCDR2, and HCDR3 of SEQ ID NOS:395-397, respectively, and optionally a heavy chain having the sequence of SEQ ID NO:398.
   Embodiment 110: The immune cell of any one of embodiments 106 to 109, wherein the CSR specifically binds to HER3 and comprises sequences of LCDR1, LCDR2, and LCDR3 of SEQ ID NOS:399-401, respectively, and optionally a light chain having the sequence of SEQ ID NO:402.
   Embodiment 111: The immune cell of any one of embodiments 106 to 110, wherein the CSR specifically binds to HER3 and comprises the sequence of SEQ ID NO:43.
   Embodiment 112: The immune cell of any one of embodiments 106 to 108, wherein the CSR specifically binds to DLL3 and comprises sequences of HCDR1, HCDR2, and HCDR3 of SEQ ID NOS:45-47, respectively, and optionally a heavy chain having the sequence of SEQ ID NO:44.
   Embodiment 113: The immune cell of any one of embodiments 106 to 108 and 112, wherein the CSR specifically binds to DLL3 and comprises sequences of LCDR1, LCDR2, and LCDR3 of SEQ ID NOS:49-51, respectively, and optionally a light chain having the sequence of SEQ ID NO:48.
   Embodiment 114: The immune cell of any one of embodiments 106 to 108, wherein the CSR binds to ROR1 and comprises sequences of HCDR1, HCDR2, and HCDR3 of SEQ ID NOS:447-449, respectively, and optionally a heavy chain variable region having the sequence of SEQ ID NO:450; and/or sequences of LCDR1, LCDR2, and LCDR3 of SEQ ID NOS:451-453, respectively, and optionally a light chain variable region having the sequence of SEQ ID NO:454; and/or optionally an scFv having the sequence of SEQ ID NO:41.
   Embodiment 115: The immune cell of any one of embodiments 106 to 108, wherein the CSR comprises sequences of HCDR1 HCDR2, and HCDR3 of SEQ ID NOS:455-457, respectively, and optionally a heavy chain variable region having the sequence of SEQ ID NO:458; and/or sequences of LCDR1, LCDR2, and LCDR3 of SEQ ID NOS:459-461, respectively, and optionally a light chain variable region having the sequence of SEQ ID NO:462; and/or optionally an scFv having the sequence of SEQ ID NO:442.
   Embodiment 116: The immune cell of embodiment 114 or 115, wherein the CSR specifically binds to a ROR1 peptide having a sequence of any one of SEQ ID NOS:443-446.
   Embodiment 117: The immune cell of any one of embodiments 1 to 23, wherein the TCR specifically binds to a complex comprising a 5T4 or PRAME peptide and an MHC class I protein.
   Embodiment 118: The immune cell of any one of embodiments 1 to 23 and 117, wherein the CSR specifically binds to ROR2, CD70, or MCT4.
   Embodiment 119: The immune cell of embodiment 117 or 118, wherein the CSR specifically binds to ROR2 and comprises: (1) sequences of HCDR1, HCDR2, and HCDR3 of SEQ ID NOS:91-93, respectively, and optionally a heavy chain variable region having the sequence of SEQ ID NO:90; or (2) sequences of HCDR1, HCDR2, and HCDR3 of SEQ ID NOS:95-97, respectively, and optionally a heavy chain variable region having the sequence of SEQ ID NO:94; or (3) sequences of HCDR1, HCDR2, and HCDR3 of SEQ ID NOS:99-101, respectively, and optionally a heavy chain variable region having the sequence of SEQ ID NO:98; or (4) sequences of HCDR1, HCDR2, and HCDR3 of SEQ ID NOS:103-105, respectively, and optionally a heavy chain variable region having the sequence of SEQ ID NO:102; or (5) sequences of HCDR1, HCDR2, and HCDR3 of SEQ ID NOS:107-109, respectively, and optionally a heavy chain variable region having the sequence of SEQ ID NO:106.
   Embodiment 120: The immune cell of any one of embodiments 117 to 119, wherein the CSR specifically binds to ROR2 and comprises: (1) sequences of LCDR1, LCDR2, and LCDR3 of SEQ ID NOS: 111-113, respectively, and optionally a light chain variable region having the sequence of SEQ ID NO:110; or (2) sequences of LCDR1, LCDR2, and LCDR3 of SEQ ID NOS: 115-117, respectively, and optionally a light chain variable region having the sequence of SEQ ID NO:114; or (3) sequences of LCDR1, LCDR2, and LCDR3 of SEQ ID NOS:119-121, respectively, and optionally a light chain variable region having the sequence of SEQ ID NO:118; or (4) sequences of LCDR1, LCDR2, and LCDR3 of SEQ ID NOS:123-125, respectively, and optionally a light chain variable region having the sequence of SEQ ID NO:122; or (5) sequences of LCDR1, LCDR2, and LCDR3 of SEQ ID NOS:127-129, respectively, and optionally a light chain variable region having the sequence of SEQ ID NO:126.
   Embodiment 121: The immune cell of embodiment 117 or 118, wherein the CSR specifically binds to CD70 and comprises sequences of HCDR1, HCDR2, and HCDR3 of SEQ ID NOS:63-65, respectively, and optionally a heavy chain having the sequence of SEQ ID NO:62.
   Embodiment 122: The immune cell of any one of embodiments 117, 118, and 121, wherein the CSR specifically binds to CD70 and comprises sequences of LCDR1, LCDR2, and LCDR3 of SEQ ID NOS:67-69, respectively, and optionally a light chain having the sequence of SEQ ID NO:66.
   Embodiment 123: The immune cell of embodiment 117 or 118, wherein the CSR specifically binds to MCT4 and comprises: (1) sequences of HCDR1, HCDR2, and HCDR3 of SEQ ID NOS:155-157, respectively, and optionally a heavy chain having the sequence of SEQ ID NO:154; or (2) sequences of HCDR1, HCDR2, and HCDR3 of SEQ ID NOS:159-161, respectively, and optionally a heavy chain having the sequence of SEQ ID NO:158; or (3) sequences of HCDR1, HCDR2, and HCDR3 of SEQ ID NOS:163-165, respectively, and optionally a heavy chain having the sequence of SEQ ID NO:162.
   Embodiment 124: The immune cell of any one of embodiments 117, 118, and 123, wherein the CSR specifically binds to MCT4 and comprises: (1) sequences of LCDR1, LCDR2, and LCDR3 of SEQ ID NOS:167-169, respectively, and optionally a light chain having the sequence of SEQ ID NO:166; or (2) sequences of LCDR1, LCDR2, and LCDR3 of SEQ ID NOS:171-173, respectively, and optionally a light chain having the sequence of SEQ ID NO:170; or (3) sequences of LCDR1, LCDR2, and LCDR3 of SEQ ID NOS:175-177, respectively, and optionally a light chain having the sequence of SEQ ID NO:174.
   Embodiment 125: The immune cell of any one of embodiments 1 to 23, wherein the TCR specifically binds to a complex comprising a MAGE-A4 peptide and an MHC class I protein.
   Embodiment 126: The immune cell of any one of embodiments 1 to 23 and embodiment 125, wherein the CSR specifically binds to MSLN, MUC16, EGFR, or RORA.
   Embodiment 127: The immune cell of any one of embodiments 1 to 23 and embodiment 125, wherein the CSR specifically binds to EGFR Embodiment 128: The immune cell of any one of embodiments 1 to 124, wherein the CSR transmembrane domain is derived from the transmembrane domain of a TCR co-receptor or a T cell costimulatory molecule.
   Embodiment 129: The immune cell of embodiment 128, wherein the TCR co-receptor or T cell costimulatory molecule is selected from the group consisting of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, and CD154.
   Embodiment 130: The immune cell of embodiment 128 or 129, wherein the TCR co-receptor or T cell costimulatory molecule is CD30, CD28, or CD8.
   Embodiment 131: The immune cell of embodiment 130, wherein the T cell costimulatory molecule is CD30.
   Embodiment 132: The immune cell of embodiment 130, wherein the TCR co-receptor is CD8 or CD28.
   Embodiment 133: The immune cell of any one of embodiments 1 to 132, wherein the CSR transmembrane domain is the transmembrane domain of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, or CD154.
   Embodiment 134: The immune cell of embodiment 133, wherein the CSR transmembrane domain is the transmembrane domain of CD30, CD28, or CD8.
   Embodiment 135: The immune cell of embodiment 134, wherein the CSR transmembrane domain is the transmembrane domain of CD30.
   Embodiment 136: The immune cell of embodiment 134, wherein the CSR transmembrane domain is the transmembrane domain of CD8 or CD28.
   Embodiment 137: The immune cell of any one of embodiments 1 to 136, wherein the CSR transmembrane domain comprises an amino acid sequence selected from the group consisting of SEQ ID NOS:66-71.
   Embodiment 138: The immune cell of any one of embodiments 1 to 137, wherein the CSR lacks a functional primary signaling domain derived from the intracellular signaling sequence of a molecule selected from the group consisting of FcRγ, FcRβ, CD3γ, CD3δ, CD3ε, CD5, CD22, CD79a, CD79b, and CD66d.
   Embodiment 139: The immune cell of any one of embodiments 1 to 138, further comprises a peptide linker between the ligand-binding module and the transmembrane domain of the CSR.
   Embodiment 140: The immune cell of any one of embodiments 1 to 139, further comprises a peptide linker between the transmembrane domain and the CD30 costimulatory domain of the CSR.
   Embodiment 141: The immune cell of any one of embodiments 1 to 140, wherein the expression of the CSR is inducible.
   Embodiment 142: The immune cell of embodiment 141, wherein the expression of the CSR is inducible upon activation of the immune cell.
   Embodiment 143: The immune cell of any one of embodiments 1 to 142, wherein the immune cell is selected from the group consisting of a cytotoxic T cell, a helper T cell, a natural killer T cell, a tumor infiltrating T cell (TIL T cell), and a suppressor T cell.
   Embodiment 144: One or more nucleic acids encoding the TCR and CSR comprised by the immune cell of any one of embodiments 1 to 143.
   Embodiment 145: One or more vectors comprising the one or more nucleic acids of embodiment 144.
   Embodiment 146: A pharmaceutical composition comprising. (a) the immune cell of any one of embodiments 1 to 143, the nucleic acid(s) of embodiment 144, or the vector(s) of embodiment 145, and (b) a pharmaceutically acceptable carrier or diluent.
   Embodiment 147: A method of killing target cells, comprising:
contacting one or more target cells with the immune cell of any one of embodiments 1 to 143 under conditions and for a time sufficient so that the immune cells mediate killing of the target cells,
wherein the target cells express an antigen specific to the immune cell, and
wherein the immune cell does not express a cell exhaustion marker upon contacting the target cells.
   Embodiment 148: The method of embodiment 147, wherein the immune cell is capable of developing into a population of immune cells that have a low percentage of cells expressing the cell exhaustion marker upon contacting the target cells
   Embodiment 149: The method of embodiment 148, wherein the immune cell is capable of developing into a population of immune cells that have a lower percentage of cells expressing the cell exhaustion marker compared to a population of immune cells that develops from a corresponding immune cell expressing a CSR comprising a CD28, 4-1BB, or DAP10 costimulatory domain, optionally wherein the ratio of the exhaustion marker expression level of the immune cell to the corresponding CD28 CSR immune cell is 0.9, 0.8, 0.7, 0.6, 0.5, 0.4, 0.3, 0.2, 0.1 or lower.
   Embodiment 150: The method of any one of embodiments 147-149, wherein the exhaustion marker is selected from the group consisting of PD-1, TIM-3, TIGIT, and LAG-3; and/or the immune cell is a T cell.
   Embodiment 151: A method of killing target cells, comprising:
contacting one or more target cells with the immune cell of any one of embodiments 1 to 143 under conditions and for a time sufficient so that the immune cells mediate killing of the target cells,
wherein the target cells express an antigen specific to the immune cell, and
wherein the immune cell expresses a low cell exhaustion level upon contacting the target cells.
   Embodiment 152: The method of embodiment 151, wherein the immune cell expresses a low cell exhaustion level of an exhaustion marker selected from the group consisting of PD-1, TIM-3, TIGIT, and LAG-3.
   Embodiment 153: The method of embodiment 151 or 152, wherein the immune cell is a T cell.
   Embodiment 154: The method of any one of embodiments 151 to 153, wherein the immune cell expresses a low cell exhaustion level of PD-1.
   Embodiment 155: The method of any one of embodiments 151 to 153, wherein the immune cell expresses a low cell exhaustion level of TIM-3.
   Embodiment 156: The method of any one of embodiments 151 to 153, wherein the immune cell expresses a low cell exhaustion level of LAG-3.
   Embodiment 157: The method of any one of embodiments 151 to 153, wherein the immune cell expresses a low cell exhaustion level of TIGIT.
   Embodiment 158: The method of any one of embodiments 151 to 157, wherein the immune cell expresses a lower level of PD-1, TIM-3, TIGIT, or LAG-3 than corresponding immune cell expressing a CSR comprising a CD28 costimulatory domain.
   Embodiment 159: The method of embodiment 158, wherein the immune cell expresses a lower level of PD-1 than the corresponding CD28 CSR immune cell, and wherein the ratio of PD-1 expression level of the immune cell to the corresponding CD28 CSR immune cell is 0.9, 0.8, 0.7, 0.6, 0.5, 0.4, 0.3, 0.2, 0.1 or lower.
   Embodiment 160: The method of embodiment 158, wherein the immune cell expresses a lower level of TIM-3 than the corresponding CD28 CSR immune cell, and wherein the ratio of TIM-3 expression level of the immune cell to the corresponding CD28 CSR immune cell is 0.9, 0.8, 0.7, 0.6, 0.5, 0.4, 0.3, 0.2, 0.1 or lower.
   Embodiment 161: The method of embodiment 158, wherein the immune cell expresses a lower level of LAG-3 than the corresponding CD28 CSR immune cell, and wherein the ratio of LAG-3 expression level of the immune cell to the corresponding CD28 CSR immune cell is 0.9, 0.8, 0.7, 0.6, 0.5, 0.4, 0.3, 0.2, 0.1 or lower.
   Embodiment 162: The method of embodiment 158, wherein the immune cell expresses a lower level of TIGIT than the corresponding CD28 CSR immune cell, and wherein the ratio of TIGIT expression level of the immune cell to the corresponding CD28 CSR immune cell is 0.9, 0.8, 0.7, 0.6, 0.5, 0.4, 0.3, 0.2, 0.1 or lower.
   Embodiment 163: The method of any one of embodiments 151 to 157, wherein the immune cell expresses a lower level of PD-1, TIM-3, TIGIT, or LAG-3 than corresponding immune cell expressing a CSR comprising a 4-1BB costimulatory domain.
   Embodiment 164: The method of embodiment 163, wherein the immune cell expresses a lower level of PD-1 than the corresponding 4-1BB CSR immune cell, and wherein the ratio of PD-1 expression level of the immune cell to the corresponding 4-1BB CSR immune cell is 0.9, 0.8, 0.7, 0.6, 0.5, 0.4, 0.3, 0.2, 0.1 or lower.
   Embodiment 165: The method of embodiment 163, wherein the immune cell expresses a lower level of TIM-3 than the corresponding 4-1BB CSR immune cell, and wherein the ratio of TIM-3 expression level of the immune cell to the corresponding 4-1BB CSR immune cell is 0.9, 0.8, 0.7, 0.6, 0.5, 0.4, 0.3, 0.2, 0.1 or lower.
   Embodiment 166: The method of embodiment 163, wherein the immune cell expresses a lower level of LAG-3 than the corresponding 4-1BB CSR immune cell, and wherein the ratio of LAG-3 expression level of the immune cell to the corresponding 4-1BB CSR immune cell is 0.9, 0.8, 0.7, 0.6, 0.5, 0.4, 0.3, 0.2, 0.1 or lower.
   Embodiment 167: The method of embodiment 163, wherein the immune cell expresses a lower level of TIGIT than the corresponding 4-1BB CSR immune cell, and wherein the ratio of TIGIT expression level of the immune cell to the corresponding 4-1BB CSR immune cell is 0.9, 0.8, 0.7, 0.6, 0.5, 0.4, 0.3, 0.2, 0.1 or lower.
   Embodiment 168: The method of any one of embodiments 151 to 157, wherein the immune cell expresses a lower level of PD-1, TIM-3, TIGIT, or LAG-3 than corresponding immune cell expressing a CSR comprising a DAP10 costimulatory domain.
   Embodiment 169: The method of embodiment 168, wherein the immune cell expresses a lower level of PD-1 than the corresponding DAP10 CSR immune cell, and wherein the ratio of PD-1 expression level of the immune cell to the corresponding DAP10 CSR immune cell is 0.9, 0.8, 0.7, 0.6, 0.5, 0.4, 0.3, 0.2, 0.1 or lower.
   Embodiment 170: The method of embodiment 168, wherein the immune cell expresses a lower level of TIM-3 than the corresponding DAP10 CSR immune cell, and wherein the ratio of TIM-3 expression level of the immune cell to the corresponding DAP10 CSR immune cell is 0.9, 0.8, 0.7, 0.6, 0.5, 0.4, 0.3, 0.2, 0.1 or lower.
   Embodiment 171: The method of embodiment 168, wherein the immune cell expresses a lower level of LAG-3 than the corresponding DAP10 CSR immune cell, and wherein the ratio of LAG-3 expression level of the immune cell to the corresponding DAP10 CSR immune cell is 0.9, 0.8, 0.7, 0.6, 0.5, 0.4, 0.3, 0.2, 0.1 or lower.
   Embodiment 172: The method of embodiment 168, wherein the immune cell expresses a lower level of TIGIT than the corresponding DAP10 CSR immune cell, and wherein the ratio of TIGIT expression level of the immune cell to the corresponding DAP10 CSR immune cell is 0.9, 0.8, 0.7, 0.6, 0.5, 0.4, 0.3, 0.2, 0.1 or lower.
   Embodiment 173: The method of any one of embodiments 151 to 172, wherein the target cells are cancer cells.
   Embodiment 174: The method of embodiment 173, wherein the cancer cells are from a cancer selected from the group consisting of liver cancer, gastrointestinal cancer, bile duct cancer, renal cell carcinoma, adrenocortical carcinoma, bladder cancer, breast cancer, cervical cancer, cholangiocarcinoma, colorectal cancer, esophageal cancer, glioblastoma, glioma, hepatocellular carcinoma, head and neck cancer, kidney cancer, lung cancer, melanoma, mesothelioma, myeloma, pancreatic cancer, pheochromocytoma, plasmacytoma, neuroblastoma, ovarian cancer, prostate cancer, sarcoma, stomach cancer, uterine cancer, and thyroid cancer.
   Embodiment 175: The method of embodiment 173 or 174, wherein the cancer cells are solid tumor cells.
   Embodiment 176: A method of treating a disease, the method comprising a step of administering to a subject the immune cell of any one of embodiments 1 to 143, the nucleic acid(s) of embodiment 144, or the vector(s) of embodiment 145, or the pharmaceutical composition of embodiment 146 to the subject.
   Embodiment 177: The method of embodiment 176, wherein the disease is cancer.
   Embodiment 178: The method of embodiment 177, wherein the cancer is a solid tumor cancer.
   Embodiment 179: The method of embodiment 178, wherein the subject has a higher density of the immune cell of any one of embodiments 1 to 143 in the solid tumor cancer than in the rest of the subject's body.
   Embodiment 180: The method of any one of claims 176 to 179, wherein administration of the immune cell results in a population of immune cells in the subject that arise from the immune cell.
   Embodiment 181: The method of claim 180, wherein the population of immune cells arising from the immune cell in the subject is larger than a population of immune cells that can arise from administration of a corresponding immune cell expressing a CSR comprising a CD28 costimulatory domain, if the corresponding immune cell is administered to the same subject.
   Embodiment 182: A method of treating a solid tumor cancer in a subject, the method comprising the steps of:
(a) transducing tumor infiltrating T cells (TIL T cells) obtained from the subject, or progenies of the TIL T cells, with a nucleic acid encoding, or a vector comprising a nucleic acid encoding, a chimeric stimulating receptor (CSR) comprising:
(i) a ligand-binding module that is capable of binding or interacting with a target ligand;
(ii) a transmembrane domain (a CSR transmembrane domain); and
(iii) a CD30 costimulatory domain, wherein the CSR lacks a functional primary signaling domain; and
(b) administering to the subject transduced TIL T cells or progenies thereof.
   Embodiment 183: The method of claim 182, wherein the ligand-binding module of the CSR comprises an antibody moiety (a CSR antibody moiety).
   Embodiment 184: The method of claim 182 or 183, wherein the CD30 costimulatory domain comprises a sequence that is at least 80%, 85%/6, 90%, 95%, or 100% identical to residues 561-573 or 578-586 of SEQ ID NO:228.
   Embodiment 185: The method of claim 182 or 183, wherein the CD30 costimulatory domain comprises a sequence that is at least 70%, 75%, 80%, 85%, 90%, 95%, or 100% identical to the sequence of SEQ ID NO:238.
   Embodiment 186: The method of any one of claims 182 to 185, wherein the target ligand is a cell surface antigen on a solid tumor.
   Embodiment 187: The method of claim 186, wherein the cell surface antigen is Glypican 3 (GPC3), HER2/ERBB2, EpCAM, MUC16, folate receptor alpha (FRα), MUC1, EGFR, EGFRvIII, HER3, DLL3, c-Met, ROR2, CD70, MCT4, MSLN, PSMA, or a variant or mutant thereof.
   Embodiment 188: The method of any one of claims 182 to 187, wherein the TIL T cells comprise an αβ TCR.
   Embodiment 189: The method of claim 188, wherein the TCR specifically binds to a disease-related MHC-restricted antigen.
   Embodiment 190: The method of claim 189, wherein the disease-related MHC-restricted antigen is expressed on cell surface of the solid tumor cancer.
   Embodiment 191: The method of claim 188, wherein the TCR does not specifically bind to a disease-related MHC-restricted antigen on cell surface of the solid tumor cancer.
   Embodiment 192: The method of any one of claims 182 to 191, further comprising a step of obtaining TIL T cells from the subject prior to the transducing step.
   Embodiment 193: The method of any one of claims 182 to 192, wherein the subject has a higher density of the transduced TIL T cells in the solid tumor cancer than in the rest of the subject's body.
   Embodiment 194: The method of any one of claims 182 to 193, wherein the cancer is selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer.
   Embodiment 195: A method for preventing and/or reversing T cell exhaustion in a subject, comprising administering to the subject the nucleic acid(s) of claim 144, the vector(s) of claim 145, or the pharmaceutical composition of claim 146 comprising the nucleic acid(s) or the vector(s) to the subject.
   Embodiment 196: The method of claim 195, wherein the method decreases the expression of an exhaustion marker in a T cell.
   Embodiment 197: The method of claim 195 or 196, wherein the exhaustion marker is selected from the group consisting of PD-1, TIM-3, TIGIT, and LAG-3.
   Embodiment 198: A method of treating a solid tumor cancer in a subject with increased tumor infiltration or immune cell expansion as compared to treating the same type of solid tumor cancer with immune cells expressing a TCR and a CSR comprising a control costimulatory domain, wherein the method comprises administering to the subject corresponding immune cells expressing the same TCR and a corresponding CSR comprising a CD30 costimulatory domain, and wherein the corresponding immune cells comprise the immune cell of any one of claims 1 to 143.
   Embodiment 199: The method of claim 198, wherein the control costimulatory domain is a CD28, 4-1BB, or DAP10 costimulatory domain.
   Embodiment 200: A method of treating a solid tumor cancer in a subject with increased tumor regression as compared to treating the same type of solid tumor cancer with immune cells expressing a TCR and a CSR comprising a CD28, 4-1BB, or DAP10 costimulatory domain, wherein the method comprises administering to the subject corresponding immune cells expressing the same TCR and a corresponding CSR comprising a CD30 costimulatory domain, and wherein the corresponding immune cells comprise the immune cell of any one of claims 1 to 143.
   Embodiment 201: A method for generating central memory T cells in a subject, comprising administering to the subject the nucleic acid(s) of claim 144, the vector(s) of claim 145, or the pharmaceutical composition of claim 146 comprising the nucleic acid(s) or the vector(s) to the subject.
   Embodiment 202: The method of claim 201, wherein the method increases the number of central memory T cells and/or the percentage of central memory T cells among all T cells in the subject.
   Embodiment 203: A method for generating central memory T cells in vitro comprising: contacting one or more target cells with the immune cell of any one of claims 1 to 143 under conditions and for a time sufficient so that the immune cell develops into central memory T cells, wherein the target cells express an antigen specific to the immune cell.
   Embodiment 204: The method of claim 203, wherein the method increases the number of central memory T cells and/or the percentage of central memory T cells among all T cells descended from the immune cell.
   Embodiment 205: The method of claim 203 or 204, wherein the method generates higher number of central memory T cells and/or higher percentage of central memory T cells than corresponding immune cell expressing a CSR comprising a CD28, 4-1BB, or DAP10 costimulatory domain.
   Embodiment 206: The method of claim 205, wherein the method generates at least 10%, 20%, 30%, 40%, 50%, 60%, 70%, 80%, 90%, 100%, 200%, 300%, 400%, or 500% higher number of central memory T cells and/or percentage of central memory T cells than corresponding immune cell expressing a CSR comprising a CD28 or DAP10 costimulatory domain.
   Embodiment 207: The method of any one of claims 203 to 206, wherein the central memory T cells express high levels of CCR7 and low levels of CD45RA.
   Embodiment 208: The method of any one of claims 203 to 207, wherein the central memory T cells are CD8+ T cells.

### Definitions

The scope of present invention is defined by the claims appended hereto and is not limited by particular embodiments described herein: those skilled in the art, reading the present disclosure, will be aware of various modifications that may be equivalent to such described embodiments, or otherwise within the scope of the claims.

In general, terminology used herein is in accordance with its understood meaning in the art, unless clearly indicated otherwise. Explicit definitions of certain terms are provided below; meanings of these and other terms in particular instances throughout this specification will be clear to those skilled in the art from context.

In order that the present invention may be more readily understood, certain terms are first defined below. Additional definitions for the following terms and other terms are set forth throughout the specification.

Administration: As used herein, the term “administration” refers to the administration of a composition to a subject or system (e.g., to a cell, organ, tissue, organism, or relevant component or set of components thereof). Those of ordinary skill will appreciate that route of administration may vary depending, for example, on the subject or system to which the composition is being administered, the nature of the composition, the purpose of the administration, etc. For example, in certain embodiments, administration to an animal subject (e.g., to a human) may be bronchial (including by bronchial instillation), buccal, enteral, interdermal, intra-arterial, intradermal, intragastric, intrahepatic, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal and/or vitreal. In some embodiments, administration may involve intermittent dosing. In some embodiments, administration may involve continuous dosing (e.g., perfusion) for at least a selected period of time.

Affinity: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Affinity matured (or affinity matured antibody): As used herein, refers to an antibody with one or more alterations in one or more CDRs (or, in some embodiments, framework regions) thereof which result an improvement in the affinity of the antibody for antigen, compared to a parent antibody which does not possess those alteration(s). In some embodiments, affinity matured antibodies will have nanomolar or even picomolar affinities for a target antigen. Affinity matured antibodies may be produced by any of a variety of procedures known in the art. Marks et al., 1992, BioTechnology 10:779-783 describes affinity maturation by V_{H} and V_{L} domain shuffling. Random mutagenesis of CDR and/or framework residues is described by: Barbas et al., 1994, Proc. Nat. Acad. Sci., U.S.A. 91:3809-3813; Schier et al., 1995, Gene 169: 147-155; Yelton et al., 1995. J. Immunol. 155:1994-2004; Jackson et al., 1995, J. Immunol. 154(7):3310-9; and Hawkins et al., 1992, J. Mol. Biol. 226:889-896. Selection of binders with improved binding properties is described by Thie et al., 2009, Methods Mol. Bio. 525:309-22.

Agent: As used herein may refer to a compound or entity of any chemical class including, for example, polypeptides, nucleic acids, saccharides, lipids, small molecules, metals, or combinations thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, aptamers, nucleic acids (e.g., siRNAs, shRNAs, DNA/RNA hybrids, antisense oligonucleotides, ribozymes), peptides, peptide mimetics, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or post-translational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a mouse, a rat, a rabbit, a pig, a cow, a deer, a sheep, a goat, a cat, a dog, or a monkey). In some embodiments, animals include, but are not limited to, mammals, birds, reptiles, amphibians, fish, insects, and/or worms. In some embodiments, an animal may be a transgenic animal, genetically engineered animal, and/or a clone.

Antibody moiety: As used herein, this term encompasses full-length antibodies and antigen-binding fragments thereof. A full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as lgG1 (γ1 heavy chain), lgG2 (γ2 heavy chain), lgG3 (γ3 heavy chain), lgG4 (γ4 heavy chain), IgA1 (α1 heavy chain), or lgA2 (α2 heavy chain).

Antigen-binding fragment or Antigen-binding portion: The term “antigen-binding fragment” or “antigen-binding portion,” as used herein, refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

Biological activity: As used herein, refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Bispecific antibody: As used herein, refers to a bispecific binding agent in which at least one, and typically both, of the binding moieties is or comprises an antibody moiety. A variety of different bispecific antibody structures are known in the art. In some embodiments, each binding moiety in a bispecific antibody that is or comprises an antibody moiety includes V_{H} and/or V_{L} regions; in some such embodiments, the V_{H} and/or V_{L} regions are those found in a particular monoclonal antibody. In some embodiments, where the bispecific antibody contains two antibody moieties, each includes V_{H} and/or V_{L} regions from different monoclonal antibodies.

The term “bispecific antibody” as used herein also refers to a polypeptide with two discrete binding moieties, each of which binds a distinct target. In some embodiments, a bispecific binding antibody is a single polypeptide; in some embodiments, a bispecific binding antibody is or comprises a plurality of peptides which, in some such embodiments may be covalently associated with one another, for example by cross-linking. In some embodiments, the two binding moieties of a bispecific binding antibody recognize different sites (e.g., epitopes) of the same target (e.g., antigen); in some embodiments, they recognize different targets. In some embodiments, a bispecific binding antibody is capable of binding simultaneously to two targets, which are of different structure.

Carrier: As used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

CDR: As used herein, the term “CDR” or “complementarity determining region” is intended to mean the non-contiguous antigen combining sites found within a variable region, such as the variable region of a heavy chain of an antibody, the variable region of a light chain of an antibody, or the variable region of a polypeptide chain in a TCR (e.g., a TCRα chain, a TCRβ chain, a TCRγ chain, or a TCRS chain). There are three CDRs in a variable region, which are designated CDR1, CDR2 and CDR3, for each of the variable regions. A “set of CDRs” or “CDR set” refers to a group of three or six CDRs that occur in either a single variable region capable of binding the antigen or the CDRs of two variable regions (e.g., two variable regions in a heavy chain and a light chain of an antibody, two variable regions in the two polypeptides of an αβ TCR, or two variable regions in the two polypeptides of a γδ TCR). These particular regions have been described by Kabat et al., J. Biol. Chem. 252:6609-6616 (1977); Kabat et al., U.S. Dept. of Health and Human Services, “Sequences of proteins of immunological interest” (1991); Chothia et al. J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mot. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol. 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present invention and for possible inclusion in one or more claims herein.

TABLE 1

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

T-cell receptor (TCR): As used herein, refers to a protein heterodimer found on the surface of T cells that is responsible for antigen recognition. There are two types of TCRs naturally: alpha beta TCR (αβ TCR, present on αβ T cells naturally) and gamma delta TCR (γδ TCR, present on γδ T cells naturally). An αβ TCR comprises a TCRα polypeptide chain and a TCRβ polypeptide chain, while a γδ TCR comprises a TCRγ polypeptide chain and a TCRδ polypeptide chain. αβ TCRs recognize fragments of antigens as peptides bound to major histocompatibility complex (MHC) molecules. γδ TCRs do not recognize antigen peptides presented by MHC, although some can recognize MHC class Ib molecules. The antigenic molecules that can activate γδ T cells are mostly unknown, but it is believed that γδ T cells play an important role in recognition of lipid antigens. αβ TCRs usually display more specific antigen binding capabilities (to peptide/MHC) than γδ TCRs. In some embodiments of the disclosure, the TCR comprises a TCRα polypeptide chain and a TCRβ polypeptide chain. In other embodiments, the TCR comprises a TCRγ polypeptide chain and a TCRδ polypeptide chain. The TCR of the disclosure can be a naturally occurring TCR or an engineered TCR. A detailed description of TCRs is provided further herein.

Adoptive cell therapy: Adoptive cell therapy is a therapeutic approach that typically includes isolation and ex vivo expansion and/or manipulation of immune cells (e.g., NK cells or T cells) and subsequent administration of these cells to a patient, for example for the treatment of cancer. Administered cells may be autologous or allogeneic. Cells may be manipulated to express engineered receptors (including TCR, CSR, CAR, and antibody-TCR) in any one of the known ways, including, for example, by using RNA and DNA transfection, viral transduction, electroporation, all of which are technologies known in the art.

The term “adoptive cell therapeutic composition” refers to any composition comprising cells suitable for adoptive cell transfer. In exemplary embodiments, the adoptive cell therapeutic composition comprises a cell type selected from a group consisting of a tumor infiltrating lymphocyte (TIL) and TCR and/or CSR modified lymphocytes. In another embodiment, the adoptive cell therapeutic composition comprises a cell type selected from a group consisting of T cells, CD8⁺ cells, CD4⁺ cells, NK-cells, delta-gamma T cells, regulatory T cells, and peripheral blood mononuclear cells. In another embodiment, TILs, T cells, CD8⁺ cells, CD4⁺ cells, NK-cells, delta-gamma T cells, regulatory T cells, or peripheral blood mononuclear cells form the adoptive cell therapeutic composition. In one embodiment, the adoptive cell therapeutic composition comprises T cells.

Comparable: As used herein, refers to two or more agents, entities, situations, sets of conditions, etc. that may not be identical to one another but that are sufficiently similar to permit comparison there between so that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Control: As used herein, refers to the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. As used herein, a “control” may refer to a “control antibody”. A “control antibody” may be a human, chimeric, humanized, CDR-grafted, multispecific, or bispecific antibody as described herein, an antibody that is different as described herein, or a parental antibody. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

The term “costimulatory domain”, or “costimulatory signaling sequence” or “costimulatory fragment”, as used herein refers to a polypeptide fragment comprising all or a portion of the intracellular domain, or intracellular signaling domain, of an immune cell costimulatory molecule that enhances cytokine production by the immune cell upon ligand-engagement (such as CD27, CD28, 4-1BB (CD137), OX40, CD30, CD40, ICOS, lymphocyte function-associated antigen-1 (LFA-1), CD2, CD7, LIGHT, NKG2C, B7-H3, a ligand that specifically binds with CD83, and the like). Such costimulatory molecules act in an antigen-independent manner in their native forms, and they themselves do not provide immune cell primary signaling activities as CD3ζ does.

Corresponding to: As used herein designates the position/identity of an amino acid residue in a polypeptide of interest. Those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190th amino acid in a particular amino acid chain but rather corresponds to the residue found at 190 in the reference polypeptide; those of ordinary skill in the art readily appreciate how to identify “corresponding” amino acids.

Detection entity/agent: As used herein, refers to any element, molecule, functional group, compound, fragment or moiety that is detectable. In some embodiments, a detection entity is provided or utilized alone. In some embodiments, a detection entity is provided and/or utilized in association with (e.g., joined to) another agent. Examples of detection entities include, but are not limited to: various ligands, radionuclides (e.g., 3H, 14C, 18F, 19F, 32P, 35S, 135I, 125I, 123I, 64Cu, 187Re, 111In, 90Y, 99mTc, 177Lu, 89Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

Effector function: As used herein refers a biochemical event that results from the interaction of an antibody Fc region with an Fc receptor or ligand. Effector functions include but are not limited to antibody-dependent cell-mediated cytotoxicity (ADCC), antibody-dependent cell-mediated phagocytosis (ADCP), and complement-mediated cytotoxicity (CMC). In some embodiments, an effector function is one that operates after the binding of an antigen, one that operates independent of antigen binding, or both.

Effector cell: As used herein refers to a cell of the immune system that mediates one or more effector functions. In some embodiments, effector cells may include, but may not be limited to, one or more of monocytes, macrophages, neutrophils, dendritic cells, eosinophils, mast cells, platelets, large granular lymphocytes, Langerhans' cells, natural killer (NK) cells, T-lymphocytes, B-lymphocytes and may be from any organism including but not limited to humans, mice, rats, rabbits, and monkeys.

Engineered: As used herein refers, in general, to the aspect of having been manipulated by the hand of man. For example, in some embodiments, a polynucleotide may be considered to be “engineered” when two or more sequences that are not linked together in that order in nature are manipulated by the hand of man to be directly linked to one another in the engineered polynucleotide. In some particular such embodiments, an engineered polynucleotide may comprise a regulatory sequence that is found in nature in operative association with a first coding sequence but not in operative association with a second coding sequence, is linked by the hand of man so that it is operatively associated with the second coding sequence. Alternatively or additionally, in some embodiments, first and second nucleic acid sequences that each encode polypeptide elements or domains that in nature are not linked to one another may be linked to one another in a single engineered polynucleotide. Comparably, in some embodiments, a cell or organism may be considered to be “engineered” if it has been manipulated so that its genetic information is altered (e.g., new genetic material not previously present has been introduced, or previously present genetic material has been altered or removed). As is common practice and is understood by those in the art, progeny of an engineered polynucleotide or cell are typically still referred to as “engineered” even though the actual manipulation was performed on a prior entity. Furthermore, as will be appreciated by those skilled in the art, a variety of methodologies are available through which “engineering” as described herein may be achieved. For example, in some embodiments, “engineering” may involve selection or design (e.g., of nucleic acid sequences, polypeptide sequences, cells, tissues, and/or organisms) through use of computer systems programmed to perform analysis or comparison, or otherwise to analyze, recommend, and/or select sequences, alterations, etc.). Alternatively or additionally, in some embodiments, “engineering” may involve use of in vitro chemical synthesis methodologies and/or recombinant nucleic acid technologies such as, for example, nucleic acid amplification (e.g., via the polymerase chain reaction) hybridization, mutation, transformation, transfection, etc., and/or any of a variety of controlled mating methodologies. As will be appreciated by those skilled in the art, a variety of established such techniques (e.g., for recombinant DNA, oligonucleotide synthesis, and tissue culture and transformation (e.g., electroporation, lipofection, etc.) are well known in the art and described in various general and more specific references that are cited and/or discussed throughout the present specification. See e.g., Sambrook et al., Molecular Cloning: A Laboratory Manual (2d ed., Cold Spring Harbor Laboratory Press, Cold Spring Harbor, N.Y., 1989).

Epitope: As used herein, includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are exposed on the surface when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized). An antibody moiety described herein may bind to an epitope comprising between 7 and 50 amino acids (e.g., between 7 and 50 contiguous amino acids), e.g., between 7 and 45, between 7 and between 7 and 40, between 7 and 35, between 7 and 30, between 7 and 25, between 7 and 20, between 7 and 15, between 7 and 10, between 10 and 50, between 15 and 50, between 20 and 50, between 25 and 50, between 30 and 50, between 35 and 50, between 40 and 50, between 45 and 50, between 10 and 45, between 15 and 40, between 20 and 35, or between 25 and 30 amino acids.

Excipient: As used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression cassette: As used herein, refers to a nucleic acid construct that, when introduced into a host cell, results in transcription and/or translation of an RNA or polypeptide, respectively.

Heterologous: As used herein, refers to a polynucleotide or polypeptide that does not naturally occur in a host cell or a host organism. A heterologous polynucleotide or polypeptide may be introduced into the host cell or host organism using well-known recombinant methods. e.g., using an expression cassette comprising the heterologous polynucleotide optionally linked to a promoter.

Framework or framework region: As used herein, refers to the sequences of a variable region minus the CDRs. Because a CDR sequence can be determined by different systems, likewise a framework sequence is subject to correspondingly different interpretations. The six CDRs divide the framework regions on the heavy and light chains into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, FR1, for example, represents the first framework region closest to the amino terminal end of the variable region and 5′ with respect to CDR1, and FRs represents two or more of the sub-regions constituting a framework region.

Host cell: As used herein, refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli. Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, a host cell is a human, monkey, ape, hamster, rat, or mouse cell. In some embodiments, a host cell is eukaryotic and is selected from the following cells: CHO (e.g., CHO K1, DXB-1 1 CHO, Veggie-CHO), COS (e.g., COS-7), retinal cell, Vero, CV1, kidney (e.g., HEK293, 293 EBNA, MSR 293, MDCK, HaK, BHK), HeLa. HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, and a cell line derived from an aforementioned cell. In some embodiments, a host cell comprises one or more viral genes, e.g., a retinal cell that expresses a viral gene (e.g., a PER.C6™ cell).

Human antibody: As used herein, is intended to include antibodies having variable and constant regions generated (or assembled) from human immunoglobulin sequences. In some embodiments, antibodies (or antibody moieties) may be considered to be “human” even though their amino acid sequences include residues or elements not encoded by human germline immunoglobulin sequences (e.g., include sequence variations, for example, that may (originally) have been introduced by random or site-specific mutagenesis in vitro or by somatic mutation in vivo), for example in one or more CDRs and in particular CDR3. Human antibodies, human antibody moieties, and their fragments can be isolated from human immune cells or generated recombinantly or synthetically, including semi-synthetically.

Humanized: As is known in the art, the term “humanized” is commonly used to refer to antibodies (or moieties) whose amino acid sequence includes V_{H} and V_{L} region sequences from a reference antibody raised in a non-human species (e.g., a mouse), but also includes modifications in those sequences relative to the reference antibody intended to render them more “human-like”, i.e., more similar to human germline variable sequences. In some embodiments, a “humanized” antibody (or antibody moiety) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′. F(ab′)₂, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a C_{H}1, hinge, C_{H}2, C_{H}3, and, optionally, a C_{H}4 region of a heavy chain constant region. In some embodiments, a humanized antibody only contains a humanized V_{L} region. In some embodiments, a humanized antibody only contains a humanized V_{H} region. In some certain embodiments, a humanized antibody contains humanized V_{H} and V_{L} regions. In some embodiments, the V_{H} region is also called H_{V} (heavy chain variable region). In some embodiments, the V_{L} region is also called L_{V} (light chain variable region). As used herein, the terms V_{H} and H_{V} are interchangeable. The terms V_{L} and L_{V} are interchangeable.

Hydrophilic: As used herein, the term “hydrophilic” and/or “polar” refers to a tendency to mix with, or dissolve easily in, water.

Hydrophobic: As used herein, the term “hydrophobic” and/or “non-polar”, refers to a tendency to repel, not combine with, or an inability to dissolve easily in, water.

Improve, increase, or reduce: As used herein, or grammatical equivalents thereof, indicate values that are relative to a baseline measurement, such as a measurement in the same individual prior to initiation of a treatment described herein, or a measurement in a control individual (or multiple control individuals) in the absence of the treatment described herein. A “control individual” is an individual afflicted with the same form of disease or injury as the individual being treated. In some embodiments, the methods for treating a cancer (e.g., a hematological cancer or a solid tumor cancer) using an immune cell described herein may increase cell apoptosis (e.g., increase tumor cell apoptosis) in an individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% compared to the individual prior to receiving treatment or to a control individual. In some embodiments, the methods for treating a cancer (e.g., a hematological cancer or a solid tumor cancer) using an immune cell described herein may reduce tumor size (e.g., reduce tumor size) in an individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% compared to the individual prior to receiving treatment or to a control individual.

In vitro: As used herein refers to events that occur in an artificial environment, e.g., in a test tube or reaction vessel, in cell culture, etc., rather than within a multi-cellular organism.

In vivo: As used herein refers to events that occur within a multi-cellular organism, such as a human and a non-human animal. In the context of cell-based systems, the term may be used to refer to events that occur within a living cell (as opposed to, for example, in vitro systems).

Isolated: As used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature, and/or b) with which it was associated when initially produced.

K_{D}: As used herein, refers to the dissociation constant of a binding agent (e.g., an antibody agent or binding component thereof) from a complex with its partner (e.g., the epitope to which the antibody agent or binding component thereof binds).

k_{off}: As used herein, refers to the off-rate constant for dissociation of a binding agent (e.g., an antibody agent or binding component thereof) from a complex with its partner (e.g., the epitope to which the antibody agent or binding component thereof binds).

kₒₙ: As used herein, refers to the on-rate constant for association of a binding agent (e.g., an antibody agent or binding component thereof) with its partner (e.g., the epitope to which the antibody agent or binding component thereof binds).

Linker: As used herein, is used to refer to that portion of a multi-element polypeptide that connects different elements to one another. For example, those of ordinary skill in the art appreciate that a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker. In some embodiments, a linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker has between 3 and 7 amino acids, between 7 and 15 amino acids, or between 20 and 30 (e.g., between 20 and 25 or between 25 and 30) amino acids. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) known in the art (see e.g., Holliger, P., et al., 1993, Proc. Natl. Acad. S. U.S.A. 90:6444-6448; Poljak, R. J. et al., 1994, Structure 2:1121-1123).

Multivalent binding antibody (or multispecific antibody): As used herein, refers an antibody capable of binding to two or more antigens, which can be on the same molecule or on different molecules. Multivalent binding antibodies as described herein are, in some embodiments, engineered to have the two or more antigen binding sites, and are typically not naturally occurring proteins. Multivalent binding antibodies as described herein refer to antibodies capable of binding two or more related or unrelated targets. Multivalent binding antibodies may be composed of multiple copies of a single antibody moiety or multiple copies of different antibody moieties. Such antibodies are capable of binding to two or more antigens and may be tetravalent or multivalent. Multivalent binding antibodies may additionally comprise a therapeutic agent, such as, for example, an immunomodulator, toxin or an RNase. Multivalent binding antibodies as described herein are, in some embodiments, capable of binding simultaneously to at least two targets that are of different structure, e.g., two different antigens, two different epitopes on the same antigen, or a hapten and/or an antigen or epitope. Multivalent binding antibodies of the present invention may be monospecific (capable of binding one antigen) or multispecific (capable of binding two or more antigens) and may be composed of two heavy chain polypeptides and two light chain polypeptides. Each binding site, in some embodiments, is composed of a heavy chain variable domain and a light chain variable domain with a total of six CDRs involved in antigen binding per antigen binding site.

Neoantigen: As used herein, refers to newly formed antigens that have not been previously recognized by the immune system. Neoantigens can arise from altered tumor proteins formed as a result of tumor mutations or from foreign proteins, such as bacterial or viral proteins.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds.

In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine. C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments a nucleic acid has a nucleotide sequence comprising at least one element that encodes, or is the complement of a sequence that encodes, a polypeptide. In some embodiments, a nucleic acid has enzymatic activity.

Operably linked: As used herein, refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with a gene of interest and expression control sequences that act in trans or at a distance to control said gene of interest. The term “expression control sequence” as used herein refers to polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism. For example, in prokaryotes, such control sequences generally include promoter, ribosomal binding site, and transcription termination sequence, while in eukaryotes, typically, such control sequences include promoters and transcription termination sequence. The term “control sequences” is intended to include components whose presence is essential for expression and processing, and can also include additional components whose presence is advantageous, for example, leader sequences and fusion partner sequences.

Physiological conditions: As used herein, has its art-understood meaning referencing conditions under which cells or organisms live and/or reproduce. In some embodiments, the term refers to conditions of the external or internal milieu that may occur in nature for an organism or cell system. In some embodiments, physiological conditions are those conditions present within the body of a human or non-human animal, especially those conditions present at and/or within a surgical site. Physiological conditions typically include, e.g., a temperature range of 20-40° C., atmospheric pressure of 1, pH of 6-8, glucose concentration of 1-20 mM, oxygen concentration at atmospheric levels, and gravity as it is encountered on earth. In some embodiments, conditions in a laboratory are manipulated and/or maintained at physiologic conditions. In some embodiments, physiological conditions are encountered in an organism.

Polypeptide: As used herein, refers to any polymeric chain of amino acids. In some embodiments, the amino acids are joined to each other by peptide bonds or modified peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is synthetically designed and/or produced. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids.

In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class.

In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30 to 40%, and is often greater than about 50%, 60%, 70%, 80%, 90%, 91%, 92%, 93%, 94%, 95%, 96%, 97%, 98%, 99% or more and/or includes at least one region (i.e., a conserved region that may in some embodiments may be or comprise a characteristic sequence element) that shows very high sequence identity, often greater than 90% or even 95%, 96%, 97%, 98%, or 99%. Such a conserved region usually encompasses at least three to four and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice-versa, and/or fragments may be present in a different order in the polypeptide of interest than in the parent), so that the polypeptide of interest is a derivative of its parent polypeptide.

Prevent or prevention: As used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Recombinant: As used herein, is intended to refer to polypeptides (e.g., antibodies or antibody moieties) that are designed, engineered, prepared, expressed, created or isolated by recombinant means, such as polypeptides expressed using a recombinant expression vector transfected into a host cell, polypeptides isolated from a recombinant, combinatorial human polypeptide library (Hoogenboom H. R., 1997, TIB Tech. 15:62-70; Azzazy H., and Highsmith W. E., 2002, Clin. Biochem. 35:425-45; Gavilondo J. V., and Larrick J. W., 2002, BioTechniques 29:128-45; Hoogenboom H., and Chames P., 2000, Immunol. Today 21:371-8), antibodies isolated from an animal (e.g., a mouse) that is transgenic for human immunoglobulin genes (see e.g., Taylor. L. D., et al., 1992, Nucl. Acids Res. 20:6287-95; Kellermann S-A., and Green L. L., 2002, Curr. Opin. Biotech. 13:593-7; Little, M. et al., 2000, Immunol. Today 21:364-70; Murphy, A. J. et al., 2014, Proc. Natl. Acad. Sci. U.S.A. 111(14):5153-8) or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source. For example, in some embodiments, a recombinant antibody is comprised of sequences found in the germline of a source organism of interest (e.g., human, mouse, etc.). In some embodiments, a recombinant antibody has an amino acid sequence that resulted from mutagenesis (e.g., in vitro or in vivo, for example in a transgenic animal), so that the amino acid sequences of the V_{H} and V_{L} regions of the recombinant antibodies are sequences that, while originating from and related to germline V_{H} and V_{L} sequences, may not naturally exist within the germline antibody repertoire in vivo.

Reference: As used herein describes a standard, control, or other appropriate reference against which a comparison is made as described herein. For example, in some embodiments, a reference is a standard or control agent, animal, individual, population, sample, sequence, series of steps, set of conditions, or value against which an agent, animal, individual, population, sample, sequence, series of steps, set of conditions, or value of interest is compared. In some embodiments, a reference is tested and/or determined substantially simultaneously with the testing or determination of interest. In some embodiments, a reference is a historical reference, optionally embodied in a tangible medium. Typically, as would be understood by those skilled in the art, a reference is determined or characterized under conditions comparable to those utilized in the assessment of interest.

Specific binding: As used herein, refers to a binding agent's ability to discriminate between possible partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations. In some embodiments, specific binding is assessed by determining the difference in binding affinity between cognate and non-cognate targets. For example, a binding agent may have a binding affinity for a cognate target that is about 3-fold, 4-fold, 5-fold, 6-fold, 7-fold, 8-fold, 9-fold, 10-fold or more than binding affinity for a non-cognate target. As used herein, the terms “specific binding,” “specifically binds,” “can specifically bind,” “specifically binding,” and “capable of specific binding” have the same meaning.

Specificity: As is known in the art, “specificity” is a measure of the ability of a particular ligand to distinguish its binding partner from other potential binding partners.

Subject: As used herein, means any mammal, including humans. In certain embodiments of the present invention the subject is an adult, an adolescent or an infant. In some embodiments, terms “individual” or “patient” are used and are intended to be interchangeable with “subject.” Also contemplated by the present invention are the administration of the pharmaceutical compositions and/or performance of the methods of treatment in utero.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial sequence homology: As used herein, the phrase “substantial homology” to refers to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues with appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “homologous” substitution. Typical amino acid categorizations are summarized as follows:

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., 1990, J. Mol. Biol., 215(3):403-410; Altschul et al., 1996, Meth. Enzymology 266:460-480; Altschul et al., 1997, Nucleic Acids Res. 25:3389-3402; Baxevanis et al, Bioinformatics: A Practical Guide to the Analysis of Genes and Proteins, Wiley, 1998; and Misener, et al, (eds.), Bioinformatics Methods and Protocols (Methods in Molecular Biology. Vol. 132), Humana Press, 1999. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Surface plasmon resonance: As used herein, refers to an optical phenomenon that allows for the analysis of specific binding interactions in real-time, for example through detection of alterations in protein concentrations within a biosensor matrix, such as by using a BIAcore system (Pharmacia Biosensor AB, Uppsala, Sweden and Piscataway, N.J.). For further descriptions, see Jonsson, U. et al., 1993, Ann. Biol. Clin. 51:19-26; Jonsson, U. et al., 1991, Biotechniques 11:620-627; Johnsson, B. et al., 1995, J. Mol. Recognit. 8:125-131; and Johnsson. B. et al., 1991, Anal. Biochem. 198:268-277.

Therapeutic agent: As used herein, generally refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically effective amount: As used herein, is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”), in its broadest sense, refers to any administration of a substance (e.g., provided compositions) that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be administered to a subject who does not exhibit signs of the relevant disease, disorder and/or condition and/or of a subject who exhibits only early signs of the disease, disorder, and/or condition. Alternatively or additionally, in some embodiments, treatment may be administered to a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Tumor infiltrating lymphocytes (TILs) refer to lymphocytes such as T cells or B cells that have migrated from the blood into tumors. In Adoptive T cell transfer therapy, TILs are isolated from surgically resected tumors and then expanded ex vivo. Multiple individual cell lines are often established, grown separately and assayed for specific tumor/cancer cell recognition. TIL cell lines with high tumor reactivities are then further expanded, and TIL T cells are activated with anti-CD3 antibodies. The final TIL T cells are infused back into the same patient to kill the cancer cells.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide.

In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 insertions or deletions, and often has no insertions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature. As will be understood by those of ordinary skill in the art, a plurality of variants of a particular polypeptide of interest may commonly be found in nature, particularly when the polypeptide of interest is an infectious agent polypeptide.

Vector: As used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Wild type: As used herein, the term “wild type” has its art-understood meaning that refers to an entity having a structure and/or activity as found in nature in a “normal” (as contrasted with mutant, variant, diseased, altered, etc.) state or context. Those of ordinary skill in the art will appreciate that wild type genes and polypeptides often exist in multiple different forms (e.g., alleles).

### BRIEF DESCRIPTION OF THE DRAWINGS

FIG. 1: T cell-mediated short-term target cell killing by mock-transduced T cells or T cells expressing (1) anti-AFP-TCR1 (“anti-AFP-TCR1”) or (2) anti-AFP-TCR1+anti-GPC3-CD30-CSR (“anti-AFP-TCR1+CD30”).

FIG. 2: Number of TCR T cells remaining after long-term engagement. T cells expressing anti-AFP-TCR+anti-GPC3-CD30-CSR survived much better than mock-transduced T cells and T cells expressing only anti-AFP-TCR.

FIG. 3: Long-term killing of HepG2 cells by TCR T cells. T cells expressing anti-AFP-TCR together with anti-GPC3-CD30-CSR killed more target cells than T cells expressing anti-AFP-TCR alone.

FIG. 4: T cell-mediated short-term target cell killing by T cells expressing (1) anti-AFP-TCR1 (“AFP-TCR”), (2) anti-AFP-TCR1+anti-GPC3-CD30-CSR (“AFP-TCR+GPC3-CD30-CSR”), (3) anti-AFP-TCR1+anti-GPC3-CD30T-CD28-CSR (“AFP-TCR+GPC3-CD30T-CD28-CSR”), (4) anti-AFP-TCR1+anti-GPC3-CD28T-CD30-CSR (“AFP-TCR+GPC3-CD28T-CD30-CSR”), (5) anti-AFP-TCR1+anti-GPC3-CD28T-41BB-CSR (“AFP-TCR+GPC3-CD28T-41BB-CSR”), or (6) anti-AFP-TCR1+anti-GPC3-CD28T-DAP10-CSR (“AFP-TCR+GPC3-CD28T-DAP10-CSR”) using an LDH-based cytotoxicity assay at two different effector:target (E:T) ratios.

FIG. 5: Cytokine IFNγ released from T cells expressing AFP-TCR or T cells expressing the various AFP-TCR plus GPC3-CSR combinations upon short-term engagement with target HepG2 cells. The results of E:T ratio of 2:1 and 10:1 are shown.

FIG. 6: Cytokines released from T cells expressing AFP-TCR or T cells expressing the various AFP-TCR plus GPC3-CSR combinations upon short-term engagement with target HepG2 cells using a 4-plex assay. The cytokines measured are IFNγ, TNFα, GM-CSF and IL-2.

FIG. 7: Long-term killing of HepG2 target cells by T cells expressing AFP-TCR or AFP TCR+GPC3-CSR combinations. E1D3 represents 3 days after the first engagement, while E2D4 and E2D10 represent 4 and 10 days after the second engagement.

FIG. 8: T cell survival in a long-term killing assay. Cell counts of T cells expressing AFP-TCR or AFP TCR+GPC3-CSR combinations. E1D3 represents 3 days after the first engagement, while E2D4 and E2D10 represent 4 and 10 days after the second engagement.

FIG. 9: Persistence of central memory T cells during a long-term killing assay. The graph shows the percentage of Tcm in the receptor-positive, CD8-positive population across time in T cells expressing an AFP-TCR alone or an AFP-TCR+GPC3-CSR combination.

FIG. 10: Persistence of central memory T cells during a long-term killing assay. The graph shows the number of Tcm present in the receptor-positive, CD8-positive population across time in T cells expressing an AFP-TCR alone or an AFP TCR+GPC3-CSR combination.

FIG. 11: Expression of T cell exhaustion marker PD1 during a long-term killing assay in T cells expressing AFP-TCR or AFP TCR+GPC3-CSR combinations. The percentage of T cells expressing PD1 is shown.

FIG. 12: Expression of T cell exhaustion marker TIM-3 during a long-term killing assay in T cells expressing AFP-TCR or AFP-TCR+GPC3-CSR combinations. The percentage of T cells expressing TIM-3 is shown.

FIG. 13: LDH-based cytotoxicity assay of short-term target cell killing by T cells expressing (1) anti-AFP-TCR1 (“AFP-TCR”), (2) anti-AFP-TCR1+anti-MSLN-CD28-CSR (“AFP-TCR+CD28-CSR”) or (3) anti-AFP-TCR1+anti-MSLN-CD30-CSR (“AFP-TCR+CD30-CSR”). R68 and R74 represent the two donor T cell sources that were engineered to express these three TCR or TCR+CSR constructs. The results of E:T (Effector:Target) ratio of 1:1 and 5:1 are shown.

FIG. 14: Cytokine IFNγ released from T cells expressing (1) anti-AFP-TCR1 (“AFP-TCR”), (2) anti-AFP-TCR1+anti-MSLN-CD28-CSR (“AFP-TCR+CD28-CSR”) or (3) anti-AFP-TCR1+anti-MSLN-CD30-CSR (“AFP-TCR+CD30-CSR”) upon short-term engagement with target cells. The results of E:T (Effector:Target) ratio of 1:1 and 5:1 are shown.

FIG. 15: Long-term killing of HepG2-MSLN target cells by T cells expressing (1) anti-AFP-TCR1 (“AFP-TCR”), (2) anti-AFP-TCR1+anti-MSLN-CD28-CSR (“AFP-TCR+CD28-CSR”), (3) anti-AFP-TCR1+anti-MSLN-CD30-CSR (“AFP-TCR+CD30-CSR”), or (4) no TCR (“Mock”). E1 represents 3 days after the first engagement, while E2 represents 4 days after the second engagement.

FIG. 16: Long-term killing of HepG2-MSLN target cells by T cells expressing (1) anti-MSLN-TCR (“MSLN-TCR”), (2) anti-MSLN-TCR+anti-MSLN-CD30-CSR (“MSLN-TCR+MSLN-CD30-CSR”), or (3) anti-MSLN-TCR+anti-MSLN-CD28-CSR (“MSLN-TCR+MSLN-CD28-CSR”). The data was collected 1 week after the engagement between the target cells and the T cells.

FIG. 17: MSLN-TCR T cell survival in a long-term killing assay. Cell counts of T cells expressing (1) anti-MSLN-TCR, (2) anti-MSLN-TCR+anti-MSLN-CD30-CSR, or (3) anti-MSLN-TCR+anti-MSLN-CD28-CSR. The data was collected 1 week after the engagement between the HepG2-MSLN target cells and the T cells.

FIG. 18: Expression of T cell exhaustion marker PD1 during a long-term killing assay in T cells expressing (1) anti-MSLN-TCR. (2) anti-MSLN-TCR+anti-MSLN-CD30-CSR, (3) anti-MSLN-TCR+anti-MSLN-CD28-CSR. The percentage of T cells expressing PD1 is shown. The data was collected 1 week after the engagement between the HepG2-MSLN target cells and the T cells.

FIG. 19: Persistence of MSLN-TCR T cells measured by central memory T cell (Tcm) percentage during a long-term killing assay. The graph shows the percentages of Tcm in the receptor-positive, CD8-positive T cell population 4 days after the engagement between the HepG2-MSLN target cells and T cells expressing (1) anti-MSLN-TCR, (2) anti-MSLN-TCR+anti-MSLN-CD30-CSR, or (3) anti-MSLN-TCR+anti-MSLN-CD28-CSR.

FIG. 20: Long-term killing of A375-Muc16 target cells by T cells expressing (1) anti-NY-ESO-1-TCR (“NY-ESO-1-TCR”), (2) anti-NY-ESO-1-TCR+anti-Muc16-CD30-CSR (“NY-ESO-1-TCR+Muc16-CD30-CSR”), or (3) anti-NY-ESO-1-TCR+anti-Muc16-41BB-CSR (“NY-ESO-1-TCR+Muc16-41BB-CSR”).

FIG. 21: Persistence of NY-ESO-1-TCR T cells measured by central memory T cell (Tem) percentage during a long-term killing assay. The graph shows the percentages of Tcm in the receptor-positive, CD8-positive T cell population 6 days after the engagement between the A375-Muc16 target cells and T cells expressing (1) anti-NY-ESO-1-TCR, (2) anti-NY-ESO-1-TCR+anti-Muc16-CD30-CSR, or (3) anti-NY-ESO-1-TCR+anti-Muc16-41BB-CSR.

### DETAILED DESCRIPTION OF THE EMBODIMENTS

Adoptive T cell immunotherapy (ACT), in which a patient's own T lymphocytes are engineered to express various recombinant antigen receptors such as chimeric antigen receptors (CARs), has shown great promise in treating hematological malignancies, but not so much in solid tumors. The same with ACT therapies with tumor infiltrating lymphocytes (TIL) or T cells expressing engineered TCRs. Therefore, more efficacious and longer-lasting T cell immunotherapies are needed.

We disclose herein that co-expression of TCR and CSR, in particular a CSR comprising a CD30 costimulatory fragment, will benefit any TCR T cell that targets a low-density antigen. Most MHC-restricted peptide antigens and solid tumor antigens are of low-density. However, even some blood cancer related cell surface antigens. e.g., CD22, are of low-density. When used to treat solid tumors, T cells expressing TCR and the CD30-CSR have increased tumor infiltration. As described herein, increased tumor infiltration by immune cells also includes increased immune cell expansion in tumors.

The present invention relates to the discovery of CSRs that use a costimulatory domain from CD30 (also referred to herein as a CD30 costimulatory domain) and T cells expressing these CSRs and TCRs have far less expression of PD-1, an inhibitor of T cell activation, than T cells with the same TCRs and CSRs containing a costimulatory domain from, e.g., CD28, 4-1BB, or DAP10. The T cells with TCRs and CSRs comprising a CD30 costimulatory domain provide superior persistence of tumor cell killing. The invention also provides the use of such T cells to treat cancer (e.g., a hematological cancer or a solid tumor cancer).

### I. T-Cell Receptors (TCRS)

The disclosure provides immune cells comprising: a T-cell receptor (TCR) and a chimeric stimulating receptor (CSR). The TCR comprises two different polypeptide chains (e.g., a heterodimer). In some embodiments, the TCR is an αβ TCR and comprises a TCRα chain and a TCRβ chain. In other embodiments, the TCR is a γδ TCR and comprises a TCRγ chain and a TCRδ chain. The two polypeptide chains in a TCR are linked by disulfide bonds. The extracellular portion of each polypeptide chain in the TCR is composed of a variable region and a constant region. The variable region of each polypeptide chain contains three complementarity-determining regions (CDR1, CDR3, and CDR3). The constant region is proximal to the cell membrane. The constant region is followed by a transmembrane region and a short cytoplasmic tail.

The TCR forms a complex with cluster of differentiation 3 (CD3) in order to carry out signal transduction inside cells. CD3, or the “CD3 complex”, is composed of six distinct chains—a CD3γ chain, a CD3δ chain, two CD3ε chains, and two CD3ζ chains (CD3ζ is also called zeta chain, ζ chain, or TCR ζ sometimes, and this application uses the term CD3ζ to refer to this molecule). These six chains of the CD3 complex associate with the TCR upon the binding of TCR to its antigen to generate an activation signal in T cells. The TCR and the CD3 chains together constitute the TCR complex, which is often an octameric complex. The TCR-CD3 complex contains both polypeptide chains of the TCR, forming the ligand-binding site, and the signaling modules CD3δ chain, CD3γ chain, two CD3ε chains, and two CD3ζ chains.

The αβ TCR recognizes and binds to an antigen fragment or peptide that is bound to a major histocompatibility complex (MHC) (a peptide/MHC complex). An antigen fragment or peptide can be bound to an MHC via the MHC class I or class II pathway. In MHC class I pathway, any nucleated cell normally presents cytosolic peptides, mostly self peptides derived from protein turnover and defective ribosomal products. During an infection or other diseases (e.g., cancer), such proteins degraded in the proteasome, as well as foreign antigens, are loaded onto MHC class I molecules and displayed on the cell surface. In MHC class II, phagocytes, such as macrophages, fuse with lysosomes whose acidic enzymes cleave the uptaken protein into many different peptides. These peptides are loaded onto MHC class II molecules. These complexes are then trafficked to and externalized on the cell surface.

In some embodiments of the present disclosure, the TCR can be a naturally occurring TCR. In other embodiments, the TCR can be an engineered TCR. Table 2 further lists exemplary proteins whose fragments or peptides can be targeted by the TCR.

In some embodiments of the compositions and methods described herein, a TCR is an as TCR. In particular embodiments, the disclosure features an αβ TCR co-expressed with a chimeric stimulating receptor (CSR) comprising a ligand-binding module, a transmembrane domain, and a CD30 costimulatory domain.

### II. Chimeric Stimulating Receptors (CSRS)

The disclosure provides a chimeric stimulating receptor (CSR), also called chimeric signaling receptor by us, comprising: (i) a ligand-binding module that is capable of binding or interacting with a target ligand; (ii) a transmembrane domain (a CSR transmembrane domain); and (iii) a CD30 costimulatory domain, wherein the CSR lacks a functional primary signaling domain (e.g., a functional primary signaling domain derived from the intracellular signaling sequence of CD3ζ). The CSRs described herein specifically binds to a target ligand (such as a cell surface antigen or a peptide/MHC complex) and is capable of stimulating an immune cell on the surface of which it is functionally expressed upon target ligand binding. The CSR comprises a ligand-binding module that provides the ligand-binding specificity, a transmembrane module, and a CD30 costimulatory immune cell signaling module that allows for stimulating the immune cell. The CSR lacks a functional primary immune cell signaling sequence. In some embodiments, the CSR lacks any primary immune cell signaling sequence. In some embodiments, the CSR comprises a single polypeptide chain comprising the ligand-binding module, transmembrane module, and CD30 costimulatory signaling module. In some embodiments, the CSR comprises a first polypeptide chain and a second polypeptide chain, wherein the first and second polypeptide chains together form the ligand-binding module, transmembrane module, and CD30 costimulatory signaling module. In some embodiments, the first and second polypeptide chains are separate polypeptide chains, and the CSR is a multimer, such as a dimer. In some embodiments, the first and second polypeptide chains are covalently linked, such as by a peptide linkage, or by another chemical linkage, such as a disulfide linkage. In some embodiments, the first polypeptide chain and the second polypeptide chain are linked by at least one disulfide bond. In some embodiments, the expression of the CSR in the TCR plus CSR immune cell is inducible. In some embodiments, the expression of the CSR in the TCR plus CSR immune cell is inducible upon signaling through the TCR. Exemplary sequences of CSRs described herein can be found in the Informal Sequence Listing table, e.g., SEQ ID NOS:181-211. In some embodiments, the CSRs with myc-tags are used in in vitro and pre-clinical assays. For in vivo use, i.e., in vivo use in humans, the corresponding CSR constructs without myc-tags are used.

The CD30 costimulatory domain of the CSR can comprise a sequence that can bind to an intracellular TRAF signaling protein. In some embodiments, the sequence that can bind to an intracellular TRAF signaling protein corresponds to residues 561-573 or 578-586 of a full-length CD30 having the sequence of SEQ ID NO:228. In certain embodiments, the CD30 costimulatory domain comprises a sequence that is at least 80%, 85%, 90%, 95%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to residues 561-573 or 578-586 of SEQ ID NO:228. In certain embodiments, the CD30 costimulatory domain comprises a sequence that is at least 70%, 75%, 80%, 85%, 90%, 95%, or 100% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identical to the sequence of SEQ ID NO:238. As described herein, immune T cells with a TCR and a CSR that comprises a costimulatory domain from CD30 express far less PD-1, an inhibitor of T cell activation, than T cells with the same TCR and a corresponding CSR that does not have a CD30 costimulatory domain, e.g., a costimulatory domain from, e.g., CD28, 4-1BB, or DAP10. T cells with a CSR containing a costimulatory domain from CD30 also demonstrate persistence in cytotoxic potential. The costimulatory domain from CD30 may ameliorate the functional unresponsiveness that leads to T cell exhaustion, i.e., anergy. The ability of a CD30 costimulatory domain to provide T cells with superior persistence of tumor cell killing is unexpected since CD30 lacks a p56lck-binding site that is thought to be crucial for costimulation.

The CSR can comprise more than one CD30 costimulatory domain. In addition to the CD30 costimulatory domain, in some embodiments, the CSR further comprises at least one costimulatory domain which comprises the intracellular sequence of a costimulatory molecule that is different from CD30. In particular embodiments, the costimulatory molecule that is different from CD30 is selected from the group consisting of CD27, CD28, 4-1BB (CD137), OX40, CD40, PD-1, ICOS, lymphocyte function-associated antigen-1 (LFA-1), CD2, CD7, LIGHT, NKG2C, B7-H3, and a ligand that specifically binds with CD83.

In some embodiments, a spacer domain may be present between the ligand-binding module and the transmembrane domain of the CSR. In some embodiments, a spacer domain may be present between the transmembrane domain and the CD30 costimulatory domain of the CSR. The spacer domain can be any oligo- or polypeptide that functions to link two parts of the TCR. A spacer domain may comprise up to about 300 amino acids, including for example about 10 to about 100, or about 25 to about 50 amino acids.

Target Antigen

In some embodiments, the TCR and the ligand-binding module of the CSR can target the same target antigen. In other embodiments, the TCR and the ligand-binding module of the CSR can target different target antigens. In some embodiments, the ligand-binding module of the CSR is derived from the extracellular domain of a receptor. The ligand-binding module of the CSR can comprise an antibody moiety (a CSR antibody moiety). The CSR antibody moiety can be a single chain antibody fragment. In some embodiments, the CSR antibody moiety is a single chain Fv (scFv), a single chain Fab, a single chain Fab′, a single domain antibody fragment, a single domain multispecific antibody, an intrabody, a nanobody, or a single chain immunokine. In certain embodiments, the CSR antibody moiety is a single domain multispecific antibody. e.g., a single domain bispecific antibody. In certain embodiments, the CSR antibody moiety is a single chain Fv (scFv), e.g., a tandem scFv. In some embodiments, the CSR antibody moiety specifically binds to a disease-related antigen. The disease-related antigen can be a cancer-related antigen or a virus-related antigen. In some embodiments, the disease-related antigen is a cancer-related neoantigen.

The TCR variable region/domain specifically binds to an MHC-restricted antigen, while the CSR antibody moiety can specifically bind to an MHC-restricted antigen or a cell surface antigen.

The MHC-restricted antigen can be any complex comprising a peptide and an MHC protein. In some embodiments, the peptide can be derived from a protein selected from the group consisting of WT-1, AFP, HPV16-E7, NY-ESO-1, PRAME, EBV-LMP2A, of WT-1, AFP, HPV16-E7, NY-ESO-1, PRAME, EBV-LMP2A, HIV-1, KRAS, FoxP3, Histone H3.3, PSA, COL18A1, SRPX, KIF16B, TFDP2, KIAA1279, XPNPEP1, UGGT2, PHKA1, KIF16B, SON, GNB5, FBXO21, CORO7, RECQL5, TFDP2, KIAA1967, KIF16B, NUP98, GPD2, CASP8, SKIV2L, H3F3B, MAGEA6, PDS5A, MED13, SLC3A2, KIAA0368, CADPS2, CTSB, DPY19L4, RNF19B, ASTN1, CDK4, MLL2, SMARCD3, p53, RAD21, RUSC2, VPS16, MGA, ARHGAP35, HER2, 5T4, and a variant or mutant thereof. In some embodiments, the peptide can be derived from a protein selected from the group consisting of KRAS, FoxP3, Histone H3.3, PSA, COL18A1, SRPX, KIF16B, TFDP2, KIAA1279. XPNPEP1, UGGT2, PHKA1, KIF16B, SON, GNB5, FBXO21, CORO7, RECQL5, TFDP2, KIAA1967, KIF16B, NUP98, GPD2, CASP8, SKIV2L, H3F3B, MAGEA6, PDS5A, MED13, SLC3A2, KIAA0368, CADPS2, CTSB, DPY19L4, RNF19B, ASTN1, CDK4, MLL2, SMARCD3, p53, RAD21, RUSC2, VPS16, MGA, ARHGAP35, HER2, 5T4, and a variant or mutant thereof.

In some embodiments, the TCR variable region/domain comprises naturally occurring or wild-type TCR sequences. In some other embodiments, the TCR variable region comprises mutant TCR sequences, such as affinity enhanced TCR sequences.

Various MHC-restricted antigen peptides and specific TCRs targeting them are disclosed in the references cited herein, the contents of which incorporated herein by reference in their entirety.

In some embodiments, the TCR variable region/domain specifically binds to a complex comprising an MHC protein and a peptide derived from AFP (see, e.g., as described in WO2015/0011450; an AFP peptide can comprise a sequence of any one of SEQ ID NOS:26-36). In some embodiments, the TCR variable region specifically binds to a complex comprising an MHC protein and a peptide derived from NY-ESO-1, e.g., SLLMWITQC (SEQ ID NO:37) (see, e.g., US20180010095; Robbins et al., J Immunol. 180(9):6116-31, 2008; Baghel et al., Oncoimmunology 5(7):e1196299, 2016; and Tan et al., Clin Exp Immunol. 180(2):255-70, 2015). In some embodiments, the TCR variable region specifically binds to a complex comprising an MHC protein and a peptide derived from PRAME (see, e.g., Amir et al., Clin Cancer Res 17(17):5615-25, 2011 and US 2016/0263155). In some embodiments, the TCR variable region specifically binds to a complex comprising an MHC protein and a peptide derived from p53 (see, e.g., Lo et al., Cancer Immunol Res 7(4):534-543, 2019; Malckzadeh et al., J Clin Invest 129(3):1109-1114, 2019). In some embodiments, the TCR variable region specifically binds to a complex comprising an MHC protein and a peptide derived from KRAS (see, e.g., Veatch et al., Cancer Immunol Res 7(6):910-922, 2019; Tran et al., N Engl J Med 375(23):2255-2262, 2016). In some embodiments, the TCR variable region specifically binds to a complex comprising an MHC protein and a peptide derived from PSA (see, e.g., EP1572929B1 and US2018/339028A1; a PSA peptide can comprise a sequence of any one of SEQ ID NOS:38-40).

In some embodiments, the TCR variable region specifically binds to a complex comprising an MHC protein and a peptide derived from a protein selected from the group consisting of COL18A1, SRPX, KIF16B, TFDP2, KIAA1279, XPNPEP1, UGGT2, PHKA1, KIF16B, SON, GNB5, FBXO21, CORO7, RECQL5, TFDP2, KIAA1967, and KIF16B (see, e.g., Parkhurst et al., Clin Cancer Res 23(10):2491-2505, 2017). In some embodiments, the TCR variable region specifically binds to a complex comprising an MHC protein and a peptide derived from a protein selected from the group consisting of MAGEA6, PDS5A, and MED13 (see, e.g., Gros et al., Nat Med. 22(4):433-8, 2016). In some embodiments, the TCR variable region specifically binds to a complex comprising an MHC protein and a peptide derived from a protein selected from the group consisting of ASTN1, CDK4, MLL2, and SMARCD3 (see, e.g., Strønen et al., Science 352(6291):1337-41, 2016).

In some embodiments, the TCR variable region specifically binds to a complex comprising an MHC protein and a peptide derived from a protein selected from the group consisting of NUP98, GPD2, CASP8, KRAS, SKIV2L, and H3F3B (see, e.g., Tran et al., Science 350(6266):1387-90, 2015). In some embodiments, the TCR variable region specifically binds to a complex comprising an MHC protein and a peptide derived from RAD21 (see, e.g., Parkhurst et al., Cancer Discov. 9(8):1022-1035, 2019).

In some embodiments, the TCR variable region specifically binds to a complex comprising an MHC protein and a peptide derived from a protein selected from the group consisting of SLC3A2, KIAA0368, CADPS2, and CTSB (see, e.g., Zacharakis et al., Nat Med. 24(6):724-730, 2018).

In some embodiments, the TCR variable region specifically binds to a complex comprising an MHC protein and a peptide derived from DPY19L4 or RNF19B protein (see, e.g., Parkhurst et al., Cancer Discov. 9(8):1022-1035, 2019). In some embodiments, the TCR variable region specifically binds to a complex comprising an MHC protein and a peptide derived from WT1 (see, e.g., Jaigirdar et al., J Immunother. 39(3):105-16, 2016).

In some embodiments, the TCR variable region specifically binds to a complex comprising an MHC protein and a peptide derived from ARHGAP35 (see, e.g., Keskin et al., Nature 565(7738):234-239, 2019). In some embodiments, the TCR variable region specifically binds to a complex comprising an MHC protein and a peptide derived from Histone H3.3. e.g., a mutated H3.3 peptide (see, e.g., WO2016/179326).

In some embodiments, the TCR variable region specifically binds to a complex comprising an MHC protein and a peptide derived from HER2/ERBB2 (see, e.g., Veatch et al., Cancer Immunol Res. 7(6):910-922, 2019).

In some embodiments, the TCR variable region specifically binds to a complex comprising an MHC protein and a peptide derived from 5T4 (see, e.g., Xu et al., Cancer Immunol Immunother. 68(12):1979-1993, 2019).

In some embodiments, the CSR antibody moiety that can specifically bind to an MHC-restricted antigen can have antibody variable region sequences or CDR sequences disclosed in the following references, the contents of which incorporated herein by reference in their entirety. For antibody sequences against a WT1 peptide/MHC complex, see, e.g., WO2012/135854. For antibody sequences against an AFP peptide/MHC complex, see, e.g., WO2016/161390. For antibody sequences against a HPV16-E7 peptide/MHC complex, see, e.g., WO2016/182957. For antibody sequences against a NY-ESO-1 peptide/MHC complex, see, e.g., WO2016/210365. For antibody sequences against a PRAME peptide/MHC complex, see, e.g., WO2016/191246. For antibody sequences against an EBV-LMP2A peptide/MHC complex, see, e.g., WO2016/201124. For antibody sequences against a KRAS peptide/MHC complex, see, e.g., WO2016/154047. For antibody sequences against a PSA peptide/MHC complex, see, e.g., WO2017/015634. For antibody sequences against a FoxP3 peptide/MHC complex, see, e.g., WO2017/124001. For antibody sequences against a Histone H3.3 peptide/MHC complex, see, e.g., WO2018/132597.

In some embodiments, the CSR antibody moiety specifically binds to a cell surface antigen. In general, cell surface antigens are more abundant than MHC-restricted antigens, therefore in general cell surface antigens are more preferred targets for the CSRs of the present disclosure. The cell surface antigen can be selected from the group consisting of protein, carbohydrate, and lipid. In certain embodiments, the cell surface antigen is glypican 3 (GPC3), human epidermal growth factor receptor 2 (HER2)/erb-b2 receptor tyrosine kinase 2 (ERBB2), epithelial cell adhesion molecule (EpCAM), mucin 16 (MUC16), folate receptor alpha (FRα), mucin 1 (MUC1), epidermal growth factor receptor (EGFR), EGFRvIII, HER3, delta-like ligand 3 (DLL3), tyrosine-protein kinase Met (c-Met), receptor tyrosine kinase like orphan receptor 2 (ROR2), cluster of differentiation 70 (CD70), monocarboxylate transporter 4 (MCT4), mesothelin (MSLN), prostate-specific membrane antigen (PSMA), or a variant or mutant thereof.

In some embodiments, the CSR antibody moiety that can specifically bind to one of the above listed cell surface antigens can have antibody variable region sequences or CDR sequences disclosed in the following references, the contents of which incorporated herein by reference in their entirety. For antibody sequences against GPC3, see, e.g., WO2018/200586. For antibody sequences against HER2, see, e.g., EP1210372B1. For antibody sequences against EpCAM, see, e.g., EP1629013B1. For antibody sequences against MUC16, see, e.g., WO2020/102555, and PCT/US2020/031886, filed May 7, 2020. For antibody sequences against FRα, see, e.g., U.S. Pat. No. 9,950,077B2. For antibody sequences against MUC1, see, e.g., U.S. Pat. No. 7,183,388B2. For antibody sequences against EGFR, see, e.g., U.S. Pat. No. 7,060,808B1. For antibody sequences against EGFRvIII, see, e.g., Lorimer et al., Proc Natl Acad Sci USA 93(25):14815-20, 1996 and U.S. Pat. No. 7,129,332B2. For antibody sequences against HER3, see, e.g., U.S. Pat. No. 7,332,585B2. For antibody sequences against DLL3, see, e.g., U.S. Pat. No. 9,127,071B2. For antibody sequences against c-Met, see, e.g., U.S. Pat. No. 8,163,280B2. For antibody sequences against ROR2, see, e.g., US2018/0127503A1. For antibody sequences against CD70, see, e.g., U.S. Pat. No. 7,662,387B2. For antibody sequences against MCT4, see, e.g., WO2019/183375. For antibody sequences against MSLN, see, e.g., U.S. Ser. No. 10/100,121B2. For antibody sequences against PSMA, see, e.g., WO2019/245991.

T cells of the current disclosure can comprise or express anyone of the TCRs and any one of the CSRs described herein.

Table 2 lists some specific embodiments of the Tcells of the current disclosure, which comprise the specific combinations of TCR and CSRs. Also listed are possible diseases, specifically possible cancers that such T cells can treat.

TABLE 2
Exemplary TCR-CSR Combinations and Cancers to be Treated

| TCR Target | | |
|---|---|---|
| (Peptide-MHC Complex, | CSR Target | Exemplary Cancers to |
| including mutant peptide) | (Cell Surface Protein) | be Treated |
| AFP | GPC3 or MSLN | Liver Cancer (e.g., |
| | | hepatocellular |
| | | carcinoma); |
| | | Bile Duct Cancer (e.g., |
| | | cholangiocarcinoma) |
| KRAS, p53, MSLN | MSLN or ROR1 | Pancreatic Cancer |
| MSLN | MSLN OR ROR1 | Liver Cancer (MSLN- |
| | | TCR + MSLN-CSR), |
| | | Pancreatic Cancer |
| | | (MSLN and ROR1 |
| | | CSRs) |
| PSA | PSMA or ROR1 | Prostate Cancer |
| COL18A1, SRPX, KIF16B, TFDP2, | ROR2 | Melanoma |
| KIAA1279, XPNPEP1, UGGT2, | | |
| PHKA1, KIF16B, SON, GNB5, | | |
| FBXO21, CORO7, RECQL5, | | |
| TFDP2, KIAA1967, KIF16B, | | |
| MAGEA6, PDS5A, MED13, | | |
| ASTN1, CDK4, MLL2, SMARCD3, | | |
| NY-ESO-1, or PRAME | | |
| NUP98, GPD2, CASP8, KRAS, | ROR2 | Gastrointestinal Cancers |
| SKIV2L, H3F3B, RAD21, or | | |
| PRAME | | |
| SLC3A2, KIAA0368, CADPS2, | HER2, ROR1, or EpCAM | Breast Cancers |
| CTSB, PRAME, p53, or PSA | | (including Metastatic |
| | | Breast Cancer) |
| WT1, NY-ESO-1, p53, DPY19L4 or | MUC1, MUC16, ROR1, | Ovarian Cancer |
| RNF19B | or FRα | |
| WT1 | MUC1¹ | Colon Cancer |
| p53, KRAS | EGFR | Colorectal Cancers |
| | | (including Metastatic |
| | | Colorectal Cancer) |
| ARHGAP35, Histone H3.3 | EGFR or EGFRvIII | Glioblastoma |
| KRAS, HER2, NY-ESO-1, p53 | HER3, DLL3, ROR1, or | Lung Cancer |
| | c-Met | |
| 5T4 or PRAME | ROR2, CD70, or MCT4 | Renal Cell Carcinoma |
| MAGE-A4 | MSLN, MUC16, EGFR, | Ovarian Cancer² |
| | RORA | |
| MAGE-A4 | EGFR | Lung Cancer³ |
| MAGE-A4 | EGFR | Melanoma⁴ |
| MAGE-A4 | EGFR | Myeloma⁵ |

¹Naitch, Anticancer Res. 36: 3715-24, 2016
²Coles et al., J. Biol. Chem 295: 11486-11595, 2020
³Holland et al., Immunotherapy of Cancer 2021; 9: e002035
⁴Sanderson et al., Oncoimmunology. 2020; 9(1): 1682381
⁵Clin Cancer Res. 2005 Aug. 1; 11(15): 5581-9; Gene Ther. 2008 May; 15(9): 695-9; Sun et al. Cell Death and Disease (2019) 10: 475)

In some embodiments, the antibody moiety comprises the CDRs or variable domains (V_{H} and/or V_{L} domains) of an antibody moiety specific for GPC3 (see, e.g., WO2018/200586A1, the contents of which are incorporated herein by reference in their entirety). In some embodiments, the antibody moiety comprises the CDRs or variable domains (V_{H} and/or V_{L} domains) of an antibody moiety specific for GPC3 (e.g., V_{H} domain comprising, consisting essentially of, or consisting of the amino acid sequence of SEQ ID NO:262 and/or V_{L} domain comprising, consisting essentially of, or consisting of the amino acid sequence of SEQ ID NO:263, or CDRs contained therein). In some embodiments, the antibody moiety comprises the CDRs or variable domains (V_{H} and/or V_{L} domains) of an antibody moiety specific for GPC3 (e.g., V_{H} domain comprising, consisting essentially of, or consisting of the amino acid sequence of SEQ ID NO:264 and/or V_{L} domain comprising, consisting essentially of, or consisting of the amino acid sequence of SEQ ID NO:265, or CDRs contained therein).

In some embodiments, the antibody moiety comprises the CDRs or variable domains (V_{H} and/or V_{L} domains) of an antibody moiety specific for ROR2 (see, e.g., WO2016/142768, the contents of which are incorporated herein by reference in their entirety). In some embodiments, the antibody moiety comprises the CDRs or variable domains (V_{H} and/or V_{L} domains) of an antibody moiety specific for ROR2 (e.g., V_{H} domain comprising, consisting essentially of, or consisting of the amino acid sequence of SEQ ID NO:90, 94, 98, 102, or 106, and/or V_{L} domain comprising, consisting essentially of, or consisting of the amino acid sequence of SEQ ID NO:110, 114, 118, 122, or 126, or CDRs contained therein). Anti-ROR2 V_{H} having SEQ ID NO:90 comprises HCDR1, HCDR2, and HCDR3 sequences of SEQ ID NOS:91-93, respectively. Anti-ROR2 V_{H} having SEQ ID NO:94 comprises HCDR1, HCDR2, and HCDR3 sequences of SEQ ID NOS:95-97, respectively. Anti-ROR2 V_{H} having SEQ ID NO:98 comprises HCDR1, HCDR2, and HCDR3 sequences of SEQ ID NOS:99-101, respectively. Anti-ROR2 V_{H} having SEQ ID NO:102 comprises HCDR1, HCDR2, and HCDR3 sequences of SEQ ID NOS:103-105, respectively. Anti-ROR2 V_{H} having SEQ ID NO:106 comprises HCDR1, HCDR2, and HCDR3 sequences of SEQ ID NOS:107-109, respectively. Anti-ROR2 V_{L} having SEQ ID NO 110 comprises HCDR1, HCDR2, and HCDR3 sequences of SEQ ID NOS:111-113, respectively. Anti-ROR2 V_{L} having SEQ ID NO:114 comprises HCDR1, HCDR2, and HCDR3 sequences of SEQ ID NOS:115-117, respectively. Anti-ROR2 V_{L} having SEQ ID NO:118 comprises HCDR1, HCDR2, and HCDR3 sequences of SEQ ID NOS:119-121, respectively. Anti-ROR2 V_{L} having SEQ ID NO:122 comprises HCDR1, HCDR2, and HCDR3 sequences of SEQ ID NOS:123-125, respectively. Anti-ROR2 V_{L} having SEQ ID NO:126 comprises HCDR1, HCDR2, and HCDR3 sequences of SEQ ID NOS:127-129, respectively. In some embodiments, the antibody moiety comprises the CDRs or variable domains (V_{H} and/or V_{L} domains) of an antibody moiety specific for ROR2 (e.g., V_{H} domain comprising, consisting essentially of, or consisting of the amino acid sequence of SEQ ID NO:90 and/or V_{L} domain comprising, consisting essentially of, or consisting of the amino acid sequence of SEQ ID NO:110, or CDRs contained therein). In some embodiments, the antibody moiety comprises the CDRs or variable domains (V_{H} and/or V_{L} domains) of an antibody moiety specific for ROR2 (e.g., V_{H} domain comprising, consisting essentially of, or consisting of the amino acid sequence of SEQ ID NO:94 and/or V_{L} domain comprising, consisting essentially of, or consisting of the amino acid sequence of SEQ ID NO:114, or CDRs contained therein). In some embodiments, the antibody moiety comprises the CDRs or variable domains (V_{H} and/or V_{L} domains) of an antibody moiety specific for ROR2 (e.g., V_{H} domain comprising, consisting essentially of, or consisting of the amino acid sequence of SEQ ID NO:98 and/or V_{L} domain comprising, consisting essentially of, or consisting of the amino acid sequence of SEQ ID NO:118, or CDRs contained therein). In some embodiments, the antibody moiety comprises the CDRs or variable domains (V_{H} and/or V_{L} domains) of an antibody moiety specific for ROR2 (e.g., V_{H} domain comprising, consisting essentially of, or consisting of the amino acid sequence of SEQ ID NO:102 and/or V_{L} domain comprising, consisting essentially of, or consisting of the amino acid sequence of SEQ ID NO:122, or CDRs contained therein). In some embodiments, the antibody moiety comprises the CDRs or variable domains (V_{H} and/or V_{L} domains) of an antibody moiety specific for ROR2 (e.g., V_{H} domain comprising, consisting essentially of, or consisting of the amino acid sequence of SEQ ID NO:106 and/or V_{L} domain comprising, consisting essentially of, or consisting of the amino acid sequence of SEQ ID NO:126, or CDRs contained therein).

In some embodiments, the antibody moiety comprises the CDRs or variable domains (V_{H} and/or V_{L} domains) of an antibody moiety specific for MUC16 (see, e.g., WO2020/102555, the contents of which are incorporated herein by reference in their entirety). In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:130 or 134, and/or V_{L} domain comprising, consisting essentially of, or consisting of the amino acid sequence of SEQ ID NO:138 or 142, or CDRs contained therein). Anti-MUC16 V_{H} having SEQ ID NO:130 comprises HCDR1, HCDR2, and HCDR3 sequences of SEQ ID NOS:131-133, respectively. Anti-ROR2 V_{H} having SEQ ID NO:134 comprises HCDR1, HCDR2, and HCDR3 sequences of SEQ ID NOS:135-137, respectively. Anti-MUC16 V_{L} having SEQ ID NO:138 comprises HCDR1, HCDR2, and HCDR3 sequences of SEQ ID NOS:139-141, respectively. Anti-ROR2 V_{L} having SEQ ID NO:142 comprises HCDR1, HCDR2, and HCDR3 sequences of SEQ ID NOS: 143-145, respectively. In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:130 and/or V_{L} domain comprising, consisting essentially of, or consisting of the amino acid sequence of SEQ ID NO:138, or CDRs contained therein). In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:134 and/or V_{L} domain comprising, consisting essentially of, or consisting of the amino acid sequence of SEQ ID NO:142, or CDRs contained therein).

In some embodiments, the antibody moiety comprises the CDRs or variable domains (V_{H} and/or V_{L} domains) of an antibody moiety specific for MUC16 (see, e.g., PCT/US2020/031886, the contents of which are incorporated herein by reference in their entirety). In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:146-149, and/or V_{L} domain comprising, consisting essentially of, or consisting of the amino acid sequence of SEQ ID NO:150-153, or CDRs contained therein).

In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:146 and/or V_{L} domain comprising, consisting essentially of, or consisting of the amino acid sequence of SEQ ID NO:150, or CDRs contained therein). In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:146 and/or V_{L} domain comprising, consisting essentially of, or consisting of the amino acid sequence of SEQ ID NO:151, or CDRs contained therein). In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:146 and/or V_{L} domain comprising, consisting essentially of, or consisting of the amino acid sequence of SEQ ID NO:152, or CDRs contained therein). In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:146 and/or V_{L} domain comprising, consisting essentially of or consisting of the amino acid sequence of SEQ ID NO:153, or CDRs contained therein).

In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:147 and/or V_{L} domain comprising, consisting essentially of, or consisting of the amino acid sequence of SEQ ID NO:150, or CDRs contained therein). In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:147 and/or V_{L} domain comprising, consisting essentially of, or consisting of the amino acid sequence of SEQ ID NO:151, or CDRs contained therein). In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:147 and/or V_{L} domain comprising, consisting essentially of, or consisting of the amino acid sequence of SEQ ID NO:152, or CDRs contained therein). In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:147 and/or V_{L} domain comprising, consisting essentially of, or consisting of the amino acid sequence of SEQ ID NO:153, or CDRs contained therein).

In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:148 and/or V_{L} domain comprising, consisting essentially of, or consisting of the amino acid sequence of SEQ ID NO:150, or CDRs contained therein). In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:148 and/or V_{L} domain comprising, consisting essentially of, or consisting of the amino acid sequence of SEQ ID NO:151, or CDRs contained therein). In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:148 and/or V_{L} domain comprising, consisting essentially of, or consisting of the amino acid sequence of SEQ ID NO:152, or CDRs contained therein). In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:148 and/or V_{L} domain comprising, consisting essentially of or consisting of the amino acid sequence of SEQ ID NO:153, or CDRs contained therein).

In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:149 and/or V_{L} domain comprising, consisting essentially of, or consisting of the amino acid sequence of SEQ ID NO:150, or CDRs contained therein). In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:149 and/or V_{L} domain comprising, consisting essentially of, or consisting of the amino acid sequence of SEQ ID NO:151, or CDRs contained therein). In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:149 and/or V_{L} domain comprising, consisting essentially of, or consisting of the amino acid sequence of SEQ ID NO:152, or CDRs contained therein). In some embodiments, the antibody moiety comprises the CDRs or variable domains (V_{H} and/or V_{L} domains) of an antibody moiety specific for MUC16 (e.g., V_{H} domain comprising, consisting essentially of, or consisting of the amino acid sequence of SEQ ID NO:149 and/or V_{L} domain comprising, consisting essentially of, or consisting of the amino acid sequence of SEQ ID NO:153, or CDRs contained therein).

In some embodiments, the antibody moiety comprises the CDRs or variable domains (V_{H} and/or V_{L} domains) of an antibody moiety specific for MCT4 (see, e.g., WO2020/102555, the contents of which are incorporated herein by reference in their entirety). In some embodiments, the antibody moiety comprises the CDRs or variable domains (V_{H} and/or V_{L} domains) of an antibody moiety specific for MCT4 (e.g., V_{H} domain comprising, consisting essentially of, or consisting of the amino acid sequence of SEQ ID NO:154, 158, or 162, and/or V_{L} domain comprising, consisting essentially of, or consisting of the amino acid sequence of SEQ ID NO:166, 170, or 174, or CDRs contained therein). Anti-MCT4 Vu having SEQ ID NO:154 comprises HCDR1, HCDR2, and HCDR3 sequences of SEQ ID NOS:155-157, respectively. Anti-ROR2 V_{H} having SEQ ID NO:158 comprises HCDR1, HCDR2, and HCDR3 sequences of SEQ ID NOS:159-161, respectively. Anti-ROR2 V_{H} having SEQ ID NO:162 comprises HCDR1, HCDR2, and HCDR3 sequences of SEQ ID NOS:163-165, respectively. Anti-MCT4 V_{L} having SEQ ID NO:166 comprises HCDR1, HCDR2, and HCDR3 sequences of SEQ ID NOS:167-169, respectively. Anti-ROR2 V_{L} having SEQ ID NO:170 comprises HCDR1, HCDR2, and HCDR3 sequences of SEQ ID NOS:171-173, respectively. Anti-ROR2 V_{L} having SEQ ID NO:174 comprises HCDR1, HCDR2, and HCDR3 sequences of SEQ ID NOS:175-177, respectively. In some embodiments, the antibody moiety comprises the CDRs or variable domains (V_{H} and/or V_{L} domains) of an antibody moiety specific for MCT4 (e.g., V_{H} domain comprising, consisting essentially of, or consisting of the amino acid sequence of SEQ ID NO:154 and/or V_{L} domain comprising, consisting essentially of, or consisting of the amino acid sequence of SEQ ID NO:166, or CDRs contained therein). In some embodiments, the antibody moiety comprises the CDRs or variable domains (V_{H} and/or V_{L} domains) of an antibody moiety specific for MCT4 (e.g., V_{H} domain comprising, consisting essentially of, or consisting of the amino acid sequence of SEQ ID NO:158 and/or V_{L} domain comprising, consisting essentially of, or consisting of the amino acid sequence of SEQ ID NO:170, or CDRs contained therein). In some embodiments, the antibody moiety comprises the CDRs or variable domains (V_{H} and/or V_{L} domains) of an antibody moiety specific for MCT4 (e.g., V_{H} domain comprising, consisting essentially of, or consisting of the amino acid sequence of SEQ ID NO:162 and/or V_{L} domain comprising, consisting essentially of, or consisting of the amino acid sequence of SEQ ID NO:174, or CDRs contained therein).

In some embodiments, the antibody moiety comprises the CDRs or variable domains (V_{H} and/or V_{L} domains) of an antibody moiety specific for ROR1 (see, e.g., WO2016/187220 and WO2016/187216).

In some embodiments, the antibody moiety comprises the CDRs or variable domains (V_{H} and/or V_{L} domains) of an antibody moiety specific for BCMA (see, e.g., WO2016/090327 and WO2016/090320).

In some embodiments, the antibody moiety comprises the CDRs or variable domains (V_{H} and/or V_{L} domains) of an antibody moiety specific for GPRC5D (see, e.g., WO2016/090329 and WO2016/090312).

In some embodiments, the antibody moiety comprises the CDRs or variable domains (V_{H} and/or V_{L} domains) of an antibody moiety specific for FCRL5 (see, e.g., WO2016/090337).

In some embodiments, the antibody moiety comprises the CDRs or variable domains (V_{H} and/or V_{L} domains) of an antibody moiety specific for PSMA (see, e.g., WO 2019/245991, the contents of which are incorporated herein by reference in their entirety).

In some embodiments, the antibody moiety comprises the CDRs or variable domains (V_{H} and/or V_{L} domains) of an antibody moiety specific for a WT-1 peptide/MHC complex (see, e.g., WO2012/135854, WO2015/070078, and WO2015/070061).

In some embodiments, the antibody moiety comprises the CDRs or variable domains (V_{H} and/or V_{L} domains) of an antibody moiety specific for an AFP peptide/MHC complex (see, e.g., WO2016/161390). In some embodiments, the AFP peptide comprises the sequence of any one of SEQ ID NOS:26-36.

In some embodiments, the antibody moiety comprises the CDRs or variable domains (V_{H} and/or V_{L} domains) of an antibody moiety specific for a HPV16-E7 peptide/MHC complex (see, e.g., WO2016/182957).

In some embodiments, the antibody moiety comprises the CDRs or variable domains (V_{H} and/or V_{L} domains) of an antibody moiety specific for a NY-ESO-1 peptide/MHC complex (see, e.g., WO2016/210365). In some embodiments, the NY-ESO-1 peptide comprises the sequence of SEQ ID NO:37.

In some embodiments, the antibody moiety comprises the CDRs or variable domains (V_{H} and/or V_{L} domains) of an antibody moiety specific for a PRAME peptide/MHC complex (see, e.g., WO2016/191246).

In some embodiments, the antibody moiety comprises the CDRs or variable domains (V_{H} and/or V_{L} domains) of an antibody moiety specific for a EBV-LMP2A peptide/MHC complex (see, e.g., WO2016/201124).

In some embodiments, the antibody moiety comprises the CDRs or variable domains (V_{H} and/or V_{L} domains) of an antibody moiety specific for a KRAS peptide/MHC complex (see, e.g., WO2016/154047).

In some embodiments, the antibody moiety comprises the CDRs or variable domains (V_{H} and/or V_{L} domains) of an antibody moiety specific for a PSA peptide/MHC complex (see, e.g., WO2017/015634). In some embodiments, the PSA peptide comprises the sequence of SEQ ID NO:38-40.

In some embodiments, the antibody moiety comprises the CDRs or variable domains (V_{H} and/or V_{L} domains) of an antibody moiety specific for a FoxP3 peptide/MHC complex (see, e.g., WO2019/161133, the contents of which are incorporated herein by reference in their entirety).

In some embodiments, the antibody moiety comprises the CDRs or variable domains (V_{H} and/or V_{L} domains) of an antibody moiety specific for a Histone H3.3 peptide/MHC complex (see, e.g., WO2018/132597).

In some embodiments, the antibody moiety comprises the CDRs or variable domains (V_{H} and/or V_{L} domains) of an antibody moiety specific for a HIV-1 peptide/MHC complex (see, e.g., WO2018057967).

In some embodiments, the antibody moiety is a scFv (single chain variable fragment) comprising a V_{H} domain and a V_{L} domain. In some embodiments, the scFv comprises an antigen-binding module that specifically binds to a complex comprising a peptide and an MHC protein, known as a peptide/MHC complex.

Ligand-Binding Module

A ligand-binding module of a CSR described herein may comprise an antibody moiety or an antigen-binding fragment thereof. In certain embodiments, the extracellular target-binding domain can be a single-chain variable fragment derived from an antibody (scFv), a tandem scFv, a single-domain antibody fragment (V_{H}Hs or sdAbs), a single domain bispecific antibody (BsAbs), an intrabody, a nanobody, an immunokine in a single chain format, and Fab, Fab′, or (Fab′)₂ in a single chain format. In other embodiments, the extracellular target-binding domain can be an antibody moiety that comprises covalently bound multiple chains of variable fragments. The extracellular target-binding domain can be joined to the TM domain via a flexible hinge/spacer region.

scFv and Tandem scFv

The ligand-binding module of a CSR described herein may comprise an antibody moiety that is a single chain Fv (scFv) antibody. An scFv antibody may comprise a light chain variable region and a heavy chain variable region, in which the light chain variable region and the heavy chain variable region may be joined using recombinant methods by a synthetic linker to make a single polypeptide chain. In some embodiments, the scFv may have the structure “(N-terminus) light chain variable region-linker-heavy chain variable region (C-terminus),” in which the heavy chain variable region is joined to the C-terminus of the light chain variable region by way of a linker. In other embodiments, the scFv may have the structure “(N-terminus) heavy chain variable region-linker-light chain variable region (C-terminus),” in which the light chain variable region is joined to the C-terminus of the heavy chain variable region by way of a linker. A linker may be a polypeptide including 2 to 200 (e.g., 2, 3, 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200) amino acids. Suitable linkers may contain flexible amino acid residues such as glycine and serine.

The ligand-binding module of a CSR may comprise an antibody moiety that is a tandem scFv comprising a first scFv and a second scFv (also referred to herein as a “tandem scFv multispecific antibody”). In some embodiments, the tandem scFv multispecific antibody further comprises at least one (such as at least about any of 2, 3, 4, 5, or more) additional scFv.

In some embodiments, there is provided a tandem scFv multispecific (e.g., bispecific) antibody comprising a) a first scFv that specifically binds to an extracellular region of a target ligand, and b) a second scFv. In some embodiments, the target ligand is CD22 and the first scFv specifically binds to an extracellular region of CD22. In some embodiments, the target ligand is CD19 and the first scFv specifically binds to an extracellular region of CD19. In some embodiments, the target ligand is an alpha-fetoprotein (AFP) peptide and the first scFv specifically binds to an extracellular region of the AFP peptide.

In some embodiments, the second scFv specifically binds to another antigen. In some embodiments, the second scFv specifically binds to an antigen on the surface of a cancer cell. In some embodiments, the second scFv specifically binds to an antigen on the surface of a cell that does not express CD22. In some embodiments, the second scFv specifically binds to an antigen on the surface of a cell that does not express CD19. In some embodiments, the second scFv specifically binds to an antigen on the surface of a cell that does not express AFP peptide. In some embodiments, the second scFv specifically binds to an antigen on the surface of a cytotoxic cell. In some embodiments, the second scFv specifically binds to an antigen on the surface of a lymphocyte, such as a T cell, an NK cell, a neutrophil, a monocyte, a macrophage, or a dendritic cell. In some embodiments, the second scFv specifically binds to an antigen on the surface of an effector T cell, such as a cytotoxic T cell. In some embodiments, the second scFv specifically binds to an antigen on the surface of an effector cell, including for example CD3γ, CD3δ, CD3ε, CD3ζ, CD28, CD16a, CD56, CD68, GDS2D, OX40, GITR, CD137. CD27, CD40L and HVEM.

In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic. In some embodiments, the tandem scFv multispecific antibody further comprises at least one (such as at least about any of 2, 3, 4, 5, or more) additional scFv.

In some embodiments, there is provided a tandem scFv multispecific (e.g., bispecific) antibody comprising a) a first scFv that specifically binds to an extracellular region of a target antigen, and b) a second scFv, wherein the tandem scFv multispecific antibody is a tandem di-scFv or a tandem tri-scFv. In some embodiments, the tandem scFv multispecific antibody is a tandem di-scFv. In some embodiments, the tandem scFv multispecific antibody is a bispecific T-cell engager.

In some embodiments, the tandem di-scFv bispecific antibody binds to an extracellular region of a target antigen or a portion thereof with a Kd between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values). In some embodiments, the tandem di-scFv bispecific antibody binds to an extracellular region of a target antigen or a portion thereof with a Kd between about 1 nM to about 500 nM (such as about any of 1, 10, 25, 50, 75, 100, 150, 200, 250, 300, 350, 400, 450, or 500 nM, including any ranges between these values).

A variety of technologies are known in the art for designing, constructing, and/or producing multispecific antibodies. Multispecific antibodies may be constructed that either utilize the full immunoglobulin framework (e.g., IgG), single chain variable fragment (scFv), or combinations thereof. Bispecific antibodies may be composed of two scFv units in tandem as described above. In the case of anti-tumor immunotherapy, bispecific antibodies that comprise two single chain variable fragments (scFvs) in tandem may be designed such that an scFv that binds a tumor antigen is linked with an scFv that engages T cells, i.e., by binding CD3 on the T cells. Thus, T cells are recruited to a tumor site to mediate killing of the tumor cells. Bispecific antibodies can be made, for example, by combining heavy chains and/or light chains that recognize different epitopes of the same or different antigen. In some embodiments, by molecular function, a bispecific binding agent binds one antigen (or epitope) on one of its two binding arms (one V_{H}/V_{L} pair), and binds a different antigen (or epitope) on its second arm (a different V_{H}/V_{L} pair). By this definition, a bispecific binding agent has two distinct antigen binding arms (in both specificity and CDR sequences), and is monovalent for each antigen to which it binds. In certain embodiments, a bispecific binding agent according to the present invention comprises a first and a second scFv. In some certain embodiments, a first scFv is linked to the C-terminal end of a second scFv. In some certain embodiments, a second scFv is linked to the C-terminal end of a first scFv. In some certain embodiments, scFvs are linked to each other via a linker (e.g., SRGGGGSGGGGSGGGGSLEMA (SEQ ID NO:242)). In some certain embodiments, scFvs are linked to each other without a linker.

A linker may be a polypeptide including 2 to 200 (e.g., 2, 3, 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200) amino acids. Suitable linkers may contain flexible amino acid residues such as glycine and serine. In certain embodiments, a linker may contain motifs, e.g., multiple or repeating motifs, of GS, GGS, GGGGS (SEQ ID NO:243), GGSG (SEQ ID NO:244), or SGGG (SEQ ID NO:245). In some embodiments, a linker may have the sequence GSGS (SEQ ID NO:246), GSGSGS (SEQ ID NO:247), GSGSGSGS (SEQ ID NO:248), GSGSGSGSGS (SEQ ID NO:249), GGSGGS (SEQ ID NO:250), GGSGGSGGS (SEQ ID NO:251), GGSGGSGGSGGS (SEQ ID NO:252), GGSG (SEQ ID NO:253), GGSGGGSG (SEQ ID NO:254), or GGSGGGSGGGSG (SEQ ID NO:255). In other embodiments, a linker may also contain amino acids other than glycine and serine, e.g., SRGGGGSGGGGSGGGGSLEMA (SEQ ID NO:242).

### Transmembrane Domain (TM)

The transmembrane domain of the CSR may be derived either from a natural or from a synthetic source. Where the source is natural, the domain may be derived from any membrane-bound or transmembrane protein. Transmembrane regions of particular use in this invention may be derived from (i.e., comprise at least the transmembrane region(s) of) the α, β, δ, γ, or ζ chain of the T-cell receptor, CD28, CD3ε, CD3ζ, CD45, CD4, CD5, CD8, CD9, CD16, CD22, CD30, CD33, CD37, CD64, CD80, CD86, CD134, CD137, or CD154. In some embodiments, a transmembrane domain can be chosen based on, for example, the nature of the various other proteins or trans-elements that bind the transmembrane domain or the cytokines induced by the transmembrane domain. For example, the transmembrane domain derived from CD30 lacks a binding site for the p56lck kinase, a common motif in the TNF receptor family. In some embodiments, a transmembrane region of particular use in this invention may be derived from (i.e., comprise at least the transmembrane region(s) of) CD8, e.g., a transmembrane region comprising a sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the sequence of SEQ ID NO:229. In some embodiments, a transmembrane region of particular use in this invention may be derived from (i.e., comprise at least the transmembrane region(s) of) CD30, e.g., a transmembrane region comprising a sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the sequence of SEQ ID NO:233.

In some embodiments, the transmembrane domain may be synthetic, in which case it may comprise predominantly hydrophobic residues such as leucine and valine. In some embodiments, a triplet of phenylalanine, tryptophan, and valine may be found at each end of a synthetic transmembrane domain. In some embodiments, a short oligo- or polypeptide linker, having a length of, for example, between about 2 and about 10 (such as about any of 2, 3, 4, 5, 6, 7, 8, 9, or 10) amino acids in length may form the linkage between the transmembrane domain and the intracellular signaling domain of a CSR described herein. In some embodiments, the linker is a glycine-serine doublet. In some embodiments, the linker between the CSR's ligand-binding module and the transmembrane domain comprises a partial extracellular domain (ECD) of a molecule such as the same as or a different molecule from the transmembrane domain's original molecule. For example, the linker connecting a transmembrane domain derived from or comprising CD8 or CD30 can comprise an ECD of CD8 or CD30, respectively or alternatively.

In some embodiments, the transmembrane domain that naturally is associated with one of the sequences in the intracellular signaling domain of the CSR is used. In some embodiments, the transmembrane domain can be selected or modified by amino acid substitution to avoid binding of such domains to the transmembrane domains of the same or different surface membrane proteins to minimize interactions with other members of the receptor complex.

Intracellular Signaling Domain

The intracellular signaling domain of the CSR is responsible for activation of at least one of the normal effector functions of the immune cell in which the TCR and CSR have been placed in. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such a truncated portion may be used in place of the intact chain as long as it transduces the effector function signal. The term “intracellular signaling sequence” is thus meant to include any truncated portion of the intracellular signaling domain sufficient to transduce the effector function signal.

Examples of intracellular signaling domains for use in a CSR include the cytoplasmic sequences of the T-cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or costimulatory signal is also required. Thus, T cell activation can be said to be mediated by two distinct classes of intracellular signaling sequence: those that initiate antigen-dependent primary activation through the TCR (primary signaling sequences) and those that act in an antigen-independent manner to provide a secondary or costimulatory signal (costimulatory signaling sequences).

Primary signaling sequences or primary signaling domain regulate primary activation of the TCR complex either in a stimulatory way, or in an inhibitory way. Primary signaling sequences that act in a stimulatory manner may contain signaling motifs which are known as immunoreceptor tyrosine-based activation motifs or ITAMs.

Examples of ITAM containing primary signaling sequences that are of particular use in the invention include those derived from TCRζ, FcRγ, FcRβ, CD3γ, CD3δ, CD3ε, CD3ζ, CD5, CD22, CD79a, CD79b, and CD66d. In some embodiments, an ITAM containing primary signaling sequence is derived from CD3ζ.

In some embodiments, the intracellular signaling domain is capable of activating an immune cell. In some embodiments, the intracellular signaling domain comprises a primary signaling sequence and a costimulatory signaling sequence. In some embodiments, the primary signaling sequence comprises a CD3ζ intracellular signaling sequence. In some embodiments, the costimulatory signaling sequence comprises a CD30 intracellular signaling sequence.

### III. Multispecific Antibodies

A ligand-binding module of a CSR may comprise an antibody moiety that is a multispecific antibody. A multispecific antibody may comprise a first binding moiety and a second binding moiety (such as a second antigen-binding moiety). Multispecific antibodies are antibodies that have binding specificities for at least two different antigens or epitopes (e.g., bispecific antibodies have binding specificities for two antigens or epitopes). Multispecific antibodies with more than two specificities are also contemplated. For example, trispecific antibodies can be prepared (see, e.g., Tutt et al., J Immunol. 147: 60 (1991)). It is to be appreciated that one of skill in the art could select appropriate features of individual multispecific antibodies described herein to combine with one another to form a multispecific antibodies of the invention.

Thus, for example, in some embodiments, there is provided a multispecific (e.g., bispecific) antibody comprising a) a first binding moiety that specifically binds to an extracellular region of a first target antigen, and b) a second binding moiety (such as an antigen-binding moiety). In some embodiments, the second binding moiety specifically binds to a different target antigen. In some embodiments, the second binding moiety specifically binds to an antigen on the surface of a cell, such as a cytotoxic cell. In some embodiments, the second binding moiety specifically binds to an antigen on the surface of a lymphocyte, such as a T cell, an NK cell, a neutrophil, a monocyte, a macrophage, or a dendritic cell. In some embodiments, the second binding moiety specifically binds to an effector T cell, such as a cytotoxic T cell (also known as cytotoxic T lymphocyte (CTL) or T killer cell).

In some embodiments, the second binding moiety specifically binds to a tumor antigen. Examples of tumor antigens include, but are not limited to, alpha fetoprotein (AFP), CA15-3, CA27-29, CA19-9, CA-125, calretinin, carcinoembryonic antigen, CD34, CD99, CD117, chromogranin, cytokeratin, desmin, epithelial membrane protein (EMA), Factor VIII, CD31 FL1, glial fibrillary acidic protein (GFAP), gross cystic disease fluid protein (GCDFP-15), HMB-45, human chorionic gonadotropin (hCG), inhibin, keratin, CD45, a lymphocyte marker, MART-1 (Melan-A), Myo Dl, muscle-specific actin (MSA), neurofilament, neuron-specific enolase (NSE), placental alkaline phosphatase (PLAP), prostate-specific antigen, S100 protein, smooth muscle actin (SMA), synaptophysin, thyroglobulin, thyroid transcription factor-1, tumor M2-PK, and vimentin.

In some embodiments, the second antigen-binding moiety in a bispecific antibody binds to CD3. In some embodiments, the second antigen-binding moiety specifically binds to CD3ε. In some embodiments, the second antigen-binding moiety specifically binds to an agonistic epitope of CD3ε. The term “agonistic epitope”, as used herein, means (a) an epitope that, upon binding of the multispecific antibody, optionally upon binding of several multispecific antibodies on the same cell, allows said multispecific antibodies to activate T-cell receptor (TCR) signaling and induce T cell activation, and/or (b) an epitope that is solely composed of amino acid residues of the epsilon chain of CD3 and is accessible for binding by the multispecific antibody, when presented in its natural context on T cells (i.e., surrounded by the TCR, the CD3γ chain, etc.), and/or (c) an epitope that, upon binding of the multispecific antibody, does not lead to stabilization of the spatial position of CD3ε relative to CD3γ.

In some embodiments, the second antigen-binding moiety binds specifically to an antigen on the surface of an effector cell, including for example CD3γ, CD3δ, CD3ε, CD3ζ, CD28, CD16a, CD56, CD68, GDS2D, OX40, GITR, CD137, CD27, CD40L and HVEM. In other embodiments, the second antigen-binding moiety binds to a component of the complement system, such as C1q. C1q is a subunit of the C1 enzyme complex that activates the serum complement system. In other embodiments, the second antigen-binding moiety specifically binds to an Fc receptor. In some embodiments, the second antigen-binding moiety specifically binds to an Fcγ receptor (FcγR). The FcγR may be an FcγRIII present on the surface of natural killer (NK) cells or one of FcγRI, FcγRIIA, FcγRIIBI, FcγRIIB2, and FcγRIIIB present on the surface of macrophages, monocytes, neutrophils and/or dendritic cells. In some embodiments, the second antigen-binding moiety is an Fc region or functional fragment thereof. A “functional fragment” as used in this context refers to a fragment of an antibody Fc region that is still capable of binding to an FcR, in particular to an FcγR, with sufficient specificity and affinity to allow an FcγR bearing effector cell, in particular a macrophage, a monocyte, a neutrophil and/or a dendritic cell, to kill the target cell by cytotoxic lysis or phagocytosis. A functional Fc fragment is capable of competitively inhibiting the binding of the original, full-length Fc portion to an FcR such as the activating FcγRI. In some embodiments, a functional Fc fragment retains at least 30%, 40%, 50%, 60%, 70%, 80%, 90% or 95% of its affinity to an activating FcγR. In some embodiments, the Fc region or functional fragment thereof is an enhanced Fc region or functional fragment thereof. The term “enhanced Fc region”, as used herein, refers to an Fc region that is modified to enhance Fc receptor-mediated effector-functions, in particular antibody-dependent cell-mediated cytotoxicity (ADCC), complement-dependent cytotoxicity (CDC), and antibody-mediated phagocytosis. This can be achieved as known in the art, for example by altering the Fc region in a way that leads to an increased affinity for an activating receptor (e.g. FcγRIIIA (CD16A) expressed on natural killer (NK) cells) and/or a decreased binding to an inhibitory receptor (e.g., FcγRIIB1/B2 (CD32B)).

In some embodiments, the multispecific antibodies allow killing of antigen-presenting target cells and/or can effectively redirect CTLs to lyse target-presenting target cells. In some embodiments, the multispecific (e.g., bispecific) antibodies of the present invention show an in vitro EC50 ranging from 10 to 500 ng/ml and is able to induce redirected lysis of about 50% of the target cells through CTLs at a ratio of CTLs to target cells of from about 1:1 to about 50:1 (such as from about 1:1 to about 15:1, or from about 2:1 to about 10:1).

In some embodiments, the multispecific (e.g., bispecific) antibody is capable of cross-linking a stimulated or unstimulated CTL and the target cell in such a way that the target cell is lysed. This offers the advantage that no generation of target-specific T cell clones or common antigen presentation by dendritic cells is required for the multispecific antibody to exert its desired activity. In some embodiments, the multispecific antibody of the present invention is capable of redirecting CTLs to lyse the target cells in the absence of other activating signals. In some embodiments, the second antigen-binding moiety specifically binds to CD3 (e.g., specifically binds to CD3ε), and signaling through CD28 and/or IL-2 is not required for redirecting CTLs to lyse the target cells.

Methods for measuring the preference of the multispecific antibody to simultaneously bind to two antigens (e.g., antigens on two different cells) are within the normal capabilities of a person skilled in the art. For example, when the second binding moiety specifically binds to the second antigen, the multispecific antibody may be contacted with a mixture of first antigen⁺/second antigen⁻ cells and first antigen-/second antigen⁺ cells. The number of multispecific antibody-positive single cells and the number of cells cross-linked by multispecific antibodies may then be assessed by microscopy or fluorescence-activated cell sorting (FACS) as known in the art.

In some embodiments, the multispecific antibody is, for example, a diabody (db), a single-chain diabody (scDb), a tandem scDb (Tandab), a linear dimeric scDb (LD-scDb), a circular dimeric scDb (CD-scDb), a di-diabody, a tandem scFv, a tandem di-scFv (e.g. a bispecific T cell engager), a tandem tri-scFv, a tri(a)body, a bispecific Fab2, a di-miniantibody, a tetrabody, an scFv-Fc-scFv fusion, a dual-affinity retargeting (DART) antibody, a dual variable domain (DVD) antibody, an IgG-scFab, an scFab-ds-scFv, an Fv2-Fe, an IgG-scFv fusion, a dock and lock (DNL) antibody, a knob-into-hole (KiH) antibody (bispecific IgG prepared by the KiH technology), a DuoBody (bispecific IgG prepared by the Duobody technology), a single-domain antibody fragment (VHHs or sdAbs), a single domain bispecific antibody (BsAbs), an intrabody, a nanobody, an immunokine in a single chain format, a heteromultimeric antibody, or a heteroconjugate antibody. In some embodiments, the multispecific antibody is a single chain antibody fragment. In some embodiments, the multispecific antibody is a tandem scFv (e.g., a tandem di-scFv, such as a bispecific T cell engager).

### IV. Antibody-Drug Conjugates

In some embodiments, there is provided an immunoconjugate comprising an antibody moiety and a therapeutic agent (also referred to herein as an “antibody-drug conjugate”, or “ADC”). In some embodiments, therapeutic agent is a toxin that is either cytotoxic, cytostatic, or otherwise prevents or reduces the ability of the target cells to divide. The use of ADCs for the local delivery of cytotoxic or cytostatic agents, i.e., drugs to kill or inhibit tumor cells in the treatment of cancer (Syrigos and Epenetos, Anticancer Research 19:605-614 (1999); Niculescu-Duvaz and Springer, Adv. Drg. Del. Rev. 26:151-172 (1997); U.S. Pat. No. 4,975,278) allows targeted delivery of the drug moiety to target cells, and intracellular accumulation therein, where systemic administration of these unconjugated therapeutic agents may result in unacceptable levels of toxicity to normal cells as well as the target cells sought to be eliminated (Baldwin et al., Lancet (Mar. 15, 1986):603-605 (1986); Thorpe, (1985) “Antibody Carriers Of Cytotoxic Agents In Cancer Therapy: A Review,” in Monoclonal Antibodies '84: Biological And Clinical Applications. A. Pinchera et al. (eds.), pp. 475-506). Maximal efficacy with minimal toxicity is sought thereby.

Therapeutic agents used in immunoconjugates (e.g., an ADC) include, for example, daunomycin, doxorubicin, methotrexate, and vindesine (Rowland et al., Cancer Immunol. Immunother. 21:183-187 (1986)). Toxins used in immunoconjugates include bacterial toxins such as diphtheria toxin, plant toxins such as ricin, small molecule toxins such as geldanamycin (Mandler et al., J. Nat. Cancer Inst. 92(19):1573-1581 (2000); Mandler et al., Bioorganic & Med. Chem. Letters 10:1025-1028 (2000); Mandler et al., Bioconjugate Chem. 13:786-791 (2002)), maytansinoids (EP 1391213; Liu et al., Proc. Natl. Acad. Sci. USA 93:8618-8623 (1996)), and calicheamicin (Lode et al., Cancer Res. 58:2928 (1998); Hinman et al., Cancer Res. 53:3336-3342 (1993)). The toxins may exert their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

Enzymatically active toxins and fragments thereof that can be used include, for example, diphtheria A chain, nonbinding active fragments of diphtheria toxin, exotoxin A chain (from Pseudomonas aeruginosa), ricin A chain, abrin A chain, modeccin A chain, α-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. See, e.g., WO 93/21232 published Oct. 28, 1993.

Immunoconjugates (e.g., an ADC) of an antibody moiety and one or more small molecule toxins, such as a calicheamicin, maytansinoids, dolastatins, aurostatins, a trichothecene, and CC1065, and the derivatives of these toxins that have toxin activity, are also contemplated herein.

In some embodiments, there is provided an immunoconjugate (e.g., an ADC) comprising a therapeutic agent that has an intracellular activity. In some embodiments, the immunoconjugate is internalized and therapeutic agent is a cytotoxin that blocks the protein synthesis of the cell, therein leading to cell death. In some embodiments, therapeutic agent is a cytotoxin comprising a polypeptide having ribosome-inactivating activity including, for example, gelonin, bouganin, saporin, ricin, ricin A chain, bryodin, diphtheria toxin, restrictocin, Pseudomonas exotoxin A and variants thereof. In some embodiments, where therapeutic agent is a cytotoxin comprising a polypeptide having a ribosome-inactivating activity, the immunoconjugate must be internalized upon binding to the target cell in order for the protein to be cytotoxic to the cells.

In some embodiments, there is provided an immunoconjugate (e.g., an ADC) comprising a therapeutic agent that acts to disrupt DNA. In some embodiments, therapeutic agent that acts to disrupt DNA is, for example, selected from the group consisting of enediyne (e.g. calicheamicin and esperamicin) and non-enediyne small molecule agents (e.g., bleomycin, methidiumpropyl-EDTA-Fe(II)).

The present invention further contemplates an immunoconjugate (e.g., an ADC) formed between the antibody moiety and a compound with nucleolytic activity (e.g., a ribonuclease or a DNA endonuclease such as a deoxyribonuclease; DNase).

In some embodiments, the immunoconjugate comprises an agent that acts to disrupt tubulin. Such agents may include, for example, rhizoxin/maytansine, paclitaxel, vincristine and vinblastine, colchicine, auristatin dolastatin 10 MMAE, and peloruside A.

In some embodiments, the immunoconjugate (e.g., an ADC) comprises an alkylating agent including, for example, Asaley NSC 167780, AZQ NSC 182986, BCNU NSC 409962, Busulfan NSC 750, carboxyphthalatoplatinum NSC 271674, CBDCA NSC 241240, CCNU NSC 79037, CHIP NSC 256927, chlorambucil NSC 3088, chlorozotocin NSC 178248, cis-platinum NSC 119875, clomesone NSC 338947, cyanomorpholinodoxorubicin NSC 357704, cyclodisone NSC 348948, dianhydrogalactitol NSC 132313, fluorodopan NSC 73754, hepsulfam NSC 329680, hycanthone NSC 142982, melphalan NSC 8806, methyl CCNU NSC 95441, mitomycin C NSC 26980, mitozolamide NSC 353451, nitrogen mustard NSC 762, PCNU NSC 95466, piperazine NSC 344007, piperazinedione NSC 135758, pipobroman NSC 25154, porfiromycin NSC 56410, spirohydantoin mustard NSC 172112, teroxirone NSC 296934, tetraplatin NSC 363812, thio-tepa NSC 6396, triethylenemelamine NSC 9706, uracil nitrogen mustard NSC 34462, and Yoshi-864 NSC 102627.

In some embodiments, the immunoconjugate (e.g., an ADC) comprises a highly radioactive atom. A variety of radioactive isotopes are available for the production of radioconjugated antibodies. Examples include 211At, 131I, 125I, 90Y, 186Re, 188Re, 153Sm, 212Bi, 32P, 212Pb and radioactive isotopes of Lu.

In some embodiments, the antibody moiety can be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

In some embodiments, an immunoconjugate (e.g., an ADC) may comprise an antibody moiety conjugated to a prodrug-activating enzyme. In some such embodiments, a prodrug-activating enzyme converts a prodrug to an active drug, such as an anti-viral drug. Such immunoconjugates are useful, in some embodiments, in antibody-dependent enzyme-mediated prodrug therapy (“ADEPT”). Enzymes that may be conjugated to an antibody include, but are not limited to, alkaline phosphatases, which are useful for converting phosphate-containing prodrugs into free drugs; arylsulfatases, which are useful for converting sulfate-containing prodrugs into free drugs; proteases, such as serratia protease, thermolysin, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as β-galactosidase and neuraminidase, which are useful for converting glycosylated prodrugs into free drugs; p-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. In some embodiments, enzymes may be covalently bound to antibody moieties by recombinant DNA techniques well known in the art. See, e.g., Neuberger et al., Nature 312:604-608 (1984).

In some embodiments, therapeutic portion of the immunoconjugates (e.g., an ADC) may be a nucleic acid. Nucleic acids that may be used include, but are not limited to, antisense RNA, genes or other polynucleotides, including nucleic acid analogs such as thioguanine and thiopurine.

The present application further provides immunoconjugates (e.g., an ADC) comprising an antibody moiety attached to an effector molecule, wherein the effector molecule is a label, which can generate a detectable signal, indirectly or directly. These immunoconjugates can be used for research or diagnostic applications, such as for the in vivo detection of cancer. The label is preferably capable of producing, either directly or indirectly, a detectable signal. For example, the label may be radio-opaque or a radioisotope, such as 3H, 14C, 32P, 35S, 123I, 125I, 131I; a fluorescent (fluorophore) or chemiluminescent (chromophore) compound, such as fluorescein isothiocyanate, rhodamine or luciferin; an enzyme, such as alkaline phosphatase, β-galactosidase or horseradish peroxidase; an imaging agent; or a metal ion. In some embodiments, the label is a radioactive atom for scintigraphic studies, for example 99Tc or 123I, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983).

In some embodiments, the immunoconjugate is detectable indirectly. For example, a secondary antibody that is specific for the immunoconjugate and contains a detectable label can be used to detect the immunoconjugate.

### V. Immune Cells

The present invention provides immune cells comprising: a T-cell receptor (TCR) and a chimeric stimulating receptor (CSR) that comprises (i) a ligand-binding module that is capable of binding or interacting with a target ligand; (ii) a transmembrane domain; and (iii) a CD30 costimulatory domain, in which the CSR in the immune cells lacks a functional primary signaling domain (e.g., a functional primary signaling domain derived from the intracellular signaling sequence of CD3ζ). In some embodiments, the immune cell comprises one or more nucleic acids encoding the TCR and CSR, wherein the TCR and CSR are expressed from the nucleic acid and localized to the immune cell surface. In some embodiments, the immune cell is a T cell. In some embodiments, the immune cell is selected from the group consisting of a cytotoxic T cell, a helper T cell, a natural killer T cell, a tumor infiltrating T cell (TIL T cell), and a suppressor T cell. In some embodiments, the immune cell is modified to block or decrease the expression of one or more of the endogenous TCR subunits of the immune cell. For example, in some embodiments, the immune cell is an αβ T cell modified to block or decrease the expression of the TCR α and/or β chains or the immune cell is a γδ T cell modified to block or decrease the expression of the TCR γ and/or δ chains. Modifications of cells to disrupt gene expression include any such techniques known in the art, including for example RNA interference (e.g., siRNA, shRNA, miRNA), gene editing (e.g., CRISPR- or TALEN-based gene knockout), and the like.

In exemplary embodiments, the cell of the present disclosure is an immune cell or a cell of the immune system. Accordingly, the cell may be a B-lymphocyte, T-lymphocyte, thymocyte, dendritic cell, natural killer (NK) cell, monocyte, macrophage, granulocyte, cosinophil, basophil, neutrophil, myelomonocytic cell, megakaryocyte, peripheral blood mononuclear cell, myeloid progenitor cell, or a hematopoietic stem cell. In exemplary aspects, the cell is a T lymphocyte. In exemplary aspects, the T lymphocyte is CD8⁺, CD4⁺, CD8⁺/CD4⁺, or a T-regulatory (T-reg) cell. In exemplary embodiments, the T lymphocyte is genetically engineered to silence the expression of an endogenous TCR. In exemplary aspects, the cell is a natural killer (NK) cell.

For example, in some embodiments, there is provided an immune cell (such as a T cell) comprising one or more nucleic acids encoding a TCR and a CSR according to any of the TCRs and CSRs described herein, wherein the TCR and CSR are expressed from the nucleic acid and localized to the immune cell surface. In some embodiments, the nucleic acid sequence is contained in a vector. Vectors may be selected, for example, from the group consisting of mammalian expression vectors and viral vectors (such as those derived from retroviruses, adenoviruses, adeno-associated viruses, herpes viruses, and lentiviruses). In some embodiments, one or more of the vectors is integrated into the host genome of the immune cell. In some embodiments, the nucleic acid sequence is under the control of a promoter. In some embodiments, the promoter is inducible. In some embodiments, the promoter is operably linked to the 5′ end of the nucleic acid sequence. In some embodiments, the immune cell is selected from the group consisting of a cytotoxic T cell, a helper T cell, a natural killer T cell, a tumor infiltrating T cell (TIL T cell), and a suppressor T cell.

Thus, in some embodiments, there is provided a immune cell (such as a T cell) expressing on its surface a TCR and CSR described herein, wherein the immune cell comprises: a nucleic acid sequence encoding a TCR polypeptide chain of the TCR and a CSR polypeptide chain of the CSR, wherein the TCR polypeptide chain and the CSR polypeptide chain are expressed from the nucleic acid sequence as a single polypeptide chain. The single polypeptide chain is then cleaved to form a TCR polypeptide chain and a CSR polypeptide chain, and the TCR polypeptide chain and the CSR polypeptide chain localize to the surface of the immune cell.

In other embodiments, there is provided a immune cell (such as a T cell) expressing on its surface a TCR and CSR described herein, wherein the immune cell comprises: a TCR nucleic acid sequence encoding a TCR polypeptide chain of the TCR, and a CSR nucleic acid sequence encoding a CSR polypeptide chain of the CSR, wherein the TCR polypeptide chain is expressed from the TCR nucleic acid sequence to form the TCR, wherein the CSR polypeptide chain is expressed from the CSR nucleic acid sequence to form the CSR, and wherein the TCR and CSR localize to the surface of the immune cell.

### VI. Fc Variants

In some embodiments, CSRs described herein may comprise a variant Fc region, wherein the variant Fc region may comprise at least one amino acid modification relative to a reference Fc region (or parental Fc region or a wild-type Fc region). Amino acid modifications may be made in an Fc region to alter effector function and/or to increase serum stability of the CSR. CSRs comprising variant Fc regions may demonstrate an altered affinity for an Fc receptor (e.g., an FcγR), provided that the variant Fc regions do not have a substitution at positions that make a direct contact with Fc receptor based on crystallographic and structural analysis of Fc-Fc receptor interactions such as those disclosed by Sondermann et al., 2000, Nature, 406:267-273. Examples of positions within the Fc region that make a direct contact with an Fc receptor such as an FcγR are amino acids 234-239 (hinge region), amino acids 265-269 (B/C loop), amino acids 297-299 (C′/E loop), and amino acids 327-332 (F/G) loop. In some embodiments, CSRs comprising variant Fc regions may comprise a modification of at least one residue that makes a direct contact with an FcγR based on structural and crystallographic analysis.

Amino acid modifications in Fc regions to create variant Fc regions that, e.g., alter affinity for activating and/or inhibitory receptors, lead to improved effector function such as, e.g., Antibody-Dependent Cell-Mediated Cytotoxicity (ADCC) and Complement Dependent Cytotoxicity (CDC), increase binding affinity for C1q, reduce or eliminate FcR binding, increase half-life are known in the art (see, e.g., U.S. Pat. Nos. 9,051,373, 9,040,041, 8,937,158, 8,883,973, 8,883,147, 8,858,937, 8,852,586, 8,809,503, 8,802,823, 8,802,820, 8,795,661, 8,753,629, 8,753,628, 8,735,547, 8,735,545, 8,734,791, 8,697,396, 8,546,543, 8,475,792, 8,399,618, 8,394,925, 8,388,955, 8,383,109, 8,367,805, 8,362,210, 8,338,574, 8,324,351, 8,318,907, 8,188,231, 8,124,731, 8,101,720, 8,093,359, 8,093,357, 8,088,376, 8,084,582, 8,039,592, 8,012,476, 7,799,900, 7,790,858, 7,785,791, 7,741,072, 7,704,497, 7,662,925, 7,416,727, 7,371,826, 7,364,731, 7,335,742, 7,332,581, 7,317,091, 7,297,775, 7,122,637, 7,083,784, 6,737,056, 6,538,124, 6,528,624 and 6,194,551).

In some embodiments, a variant Fc region may have different glycosylation patterns as compared to a parent Fc region (e.g., aglycosylated). In some embodiments, different glycosylation patterns may arise from expression in different cell lines, e.g., an engineered cell line.

CSRs described herein may comprise variant Fc regions that bind with a greater affinity to one or more FcγRs. Such CSRs preferably mediate effector function more effectively as discussed infra. In some embodiments, CSRs described herein may comprise variant Fc regions that bind with a weaker affinity to one or more FcγRs. Reduction or elimination of effector function may be desirable in certain cases, for example, in the case of TCRs and/or CSRs whose mechanism of action involves blocking or antagonism but not killing of the cells bearing a target antigen. In some embodiments, increased effector function may be directed to tumor cells and cells expressing foreign antigens.

### VII. Nucleic Acids

Nucleic acid molecules encoding the TCRs and CSRs described herein are also contemplated. In some embodiments, according to any of the TCRs and CSRs described herein, there is provided a nucleic acid (or a set of nucleic acids) encoding the TCRs and CSRs.

The present invention also provides vectors in which a nucleic acid of the present invention is inserted.

In brief summary, the expression of a TCR and/or CSR described herein by a nucleic acid encoding the TCR and/or CSR can be achieved by inserting the nucleic acid into an appropriate expression vector, such that the nucleic acid is operably linked to 5′ and 3′ regulatory elements, including for example a promoter (e.g., a lymphocyte-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequence.

The nucleic acids of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the invention provides a gene therapy vector.

The nucleic acid can be cloned into a number of types of vectors. For example, the nucleic acid can be cloned into a vector including, but not limited to, a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Further, the invention should not be limited to the use of constitutive promoters. Inducible promoters are also contemplated as part of the invention. The use of an inducible promoter provides a molecular switch capable of turning on expression of the polynucleotide sequence to which it is operatively linked when such expression is desired or turning off the expression when expression is not desired. Exemplary inducible promoter systems for use in eukaryotic cells include, but are not limited to, hormone-regulated elements (e.g., see Mader, S. and White, J. H. (1993) Proc. Natl. Acad. Sci. USA 90:5603-5607), synthetic ligand-regulated elements (see, e.g., Spencer, D. M. et al 1993) Science 262: 1019-1024) and ionizing radiation-regulated elements (e.g., see Manome, Y. et al. (1993) Biochemistry 32: 10607-10613; Datta, R. et al. (1992) Proc. Natl. Acad. Sci. USA 89: 1014-10153). Further exemplary inducible promoter systems for use in in vitro or in vivo mammalian systems are reviewed in Gingrich et al. (1998) Annual Rev. Neurosci 21:377-405.

An exemplary inducible promoter system for use in the present invention is the Tet system. Such systems are based on the Tet system described by Gossen et al. (1993). In an exemplary embodiment, a polynucleotide of interest is under the control of a promoter that comprises one or more Tet operator (TetO) sites. In the inactive state, Tet repressor (TetR) will bind to the TetO sites and repress transcription from the promoter. In the active state, e.g., in the presence of an inducing agent such as tetracycline (Tc), anhydrotetracycline, doxycycline (Dox), or an active analog thereof, the inducing agent causes release of TetR from TetO, thereby allowing transcription to take place. Doxycycline is a member of the tetracycline family of antibiotics having the chemical name of 1-dimethylamino-2,4a,5,7,12-pentahydroxy-11-methyl-4,6-dioxo-1,4a,11,11a,12,12a-hexahydrotetracene-3-carboxamide.

In one embodiment, a TetR is codon-optimized for expression in mammalian cells, e.g., murine or human cells. Most amino acids are encoded by more than one codon due to the degeneracy of the genetic code, allowing for substantial variations in the nucleotide sequence of a given nucleic acid without any alteration in the amino acid sequence encoded by the nucleic acid. However, many organisms display differences in codon usage, also known as “codon bias” (i.e., bias for use of a particular codon(s) for a given amino acid). Codon bias often correlates with the presence of a predominant species of tRNA for a particular codon, which in turn increases efficiency of mRNA translation. Accordingly, a coding sequence derived from a particular organism (e.g., a prokaryote) may be tailored for improved expression in a different organism (e.g., a eukaryote) through codon optimization.

Other specific variations of the Tet system include the following “Tet-Off” and “Tet-On” systems. In the Tet-Off system, transcription is inactive in the presence of Tc or Dox. In that system, a tetracycline-controlled transactivator protein (tTA), which is composed of TetR fused to the strong transactivating domain of VP16 from Herpes simplex virus, regulates expression of a target nucleic acid that is under transcriptional control of a tetracycline-responsive promoter element (TRE). The TRE is made up of TetO sequence concatamers fused to a promoter (commonly the minimal promoter sequence derived from the human cytomegalovirus (hCMV) immediate-early promoter). In the absence of Tc or Dox, tTA binds to the TRE and activates transcription of the target gene. In the presence of Tc or Dox, tTA cannot bind to the TRE, and expression from the target gene remains inactive.

Conversely, in the Tet-On system, transcription is active in the presence of Tc or Dox. The Tet-On system is based on a reverse tetracycline-controlled transactivator, rtTA. Like tTA, rtTA is a fusion protein comprised of the TetR repressor and the VP16 transactivation domain. However, a four amino acid change in the TetR DNA binding moiety alters rtTA's binding characteristics such that it can only recognize the tetO sequences in the TRE of the target transgene in the presence of Dox. Thus, in the Tet-On system, transcription of the TRE-regulated target gene is stimulated by rtTA only in the presence of Dox.

Another inducible promoter system is the lac repressor system from E. coli. (See, Brown et al., Cell 49:603-612 (1987). The lac repressor system functions by regulating transcription of a polynucleotide of interest operably linked to a promoter comprising the lac operator (lacO). The lac repressor (lacR) binds to LacO, thus preventing transcription of the polynucleotide of interest. Expression of the polynucleotide of interest is induced by a suitable inducing agent, e.g., isopropyl-β-D-thiogalactopyranoside (IPTG).

Another exemplary inducible promoter system for use in the present invention is the nuclear-factor of the activated T-cell (NFAT) system. The NFAT family of transcription factors are important regulators of T cell activation. NFAT response elements are found, for example, in the IL-2 promoter (see for example Durand, D. et. al., Molec. Cell. Biol. 8, 1715-1724 (1988); Clipstone, N A, Crabtree, G R. Nature. 1992 357(6380): 695-7; Chmielewski, M., et al. Cancer research 71.17 (2011): 5697-5706; and Zhang, L., et al. Molecular therapy 19.4 (2011): 751-759). In some embodiments, an inducible promoter described herein comprises one or more (such as 2, 3, 4, 5, 6, or more) NFAT response elements. In some embodiments, the inducible promoter comprises 6 NFAT response elements, for example, comprising the nucleotide sequence of SEQ ID NO:266. In some embodiments, an inducible promoter described herein comprises one or more (such as 2, 3, 4, 5, 6, or more) NFAT response elements linked to a minimal promoter, such as a minimal TA promoter. In some embodiments, the minimal TA promoter comprises the nucleotide sequence of SEQ ID NO:267. In some embodiments, the inducible promoter comprises the nucleotide sequence of SEQ ID NO:268.

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, p-galactosidase, chloramphenicol acetyl transferase, secreted alkaline phosphatase, or the green fluorescent protein gene (e.g., Ui-Tel et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, there is provided nucleic acid encoding a TCR and/or CSR according to any of the TCRs and CSRs described herein. In some embodiments, the nucleic acid comprises one or more nucleic acid sequences encoding all of the polypeptide chains of the TCR. In some embodiments, the nucleic acid comprises one or more nucleic acid sequences encoding all of the polypeptide chains of the CSR. In some embodiments, the nucleic acid comprises one or more nucleic acid sequences encoding all of the polypeptide chains of the TCR and the CSR. In some embodiments, each of the one or more nucleic acid sequences is contained in separate vectors. In some embodiments, at least some of the nucleic acid sequences are contained in the same vector. In some embodiments, all of the nucleic acid sequences are contained in the same vector. Vectors may be selected, for example, from the group consisting of mammalian expression vectors and viral vectors (such as those derived from retroviruses, adenoviruses, adeno-associated viruses, herpes viruses, and lentiviruses).

For example, in some embodiments, the CSR is a monomer comprising a single CSR polypeptide chain. In some embodiments, the nucleic acid comprises one or more nucleic acid sequences encoding all of the polypeptide chains of the TCR and the CSR. In some embodiments, the nucleic acid sequences are contained in multiple vectors. In some embodiments, the nucleic acid sequences are contained in one vector. In some embodiments, one or more nucleic acid sequences are under the control of one promoter. In some embodiments, each nucleic acid sequence is under the control of a promoter. In some embodiments, two or more promoters have the same sequence. In some embodiments, the nucleic acid sequences are expressed as a single transcript under the control of a single promoter in a multicistronic vector. See for example Kim, J H, et al., PLoS One 6(4):e18556, 2011. In some embodiments, one or more of the promoters are inducible. In some embodiments, the nucleic acid sequence encoding the CSR polypeptide chain is operably linked to an inducible promoter. In some embodiments, the inducible promoter comprises one or more elements responsive to immune cell activation, such as NFAT response elements.

In some embodiments, the nucleic acid sequences have similar (such as substantially or about the same) expression levels in a host cell (such as a T cell). In some embodiments, the nucleic acid sequences have expression levels in a host cell (such as a T cell) that differ by at least about two (such as at least about any of 2, 3, 4, 5, or more) times. Expression can be determined at the mRNA or protein level. The level of mRNA expression can be determined by measuring the amount of mRNA transcribed from the nucleic acid using various well-known methods, including Northern blotting, quantitative RT-PCR, microarray analysis and the like. The level of protein expression can be measured by known methods including immunocytochemical staining, enzyme-linked immunosorbent assay (ELISA), western blot analysis, luminescent assays, mass spectrometry, high performance liquid chromatography, high-pressure liquid chromatography-tandem mass spectrometry, and the like.

Thus, in some embodiments, there is provided a nucleic acid encoding a) two TCR polypeptide chains according to any of the TCRs described herein; and b) a CSR polypeptide chain according to any of the CSRs described herein. In some embodiments, the nucleic acid sequence is contained in a vector (such as a lentiviral vector). In some embodiments, the portion of the nucleic acid encoding the first TCR polypeptide chain is under the control of a first promoter, the portion of the nucleic acid encoding the second TCR polypeptide chain is under the control of a second promoter, and the portion of the nucleic acid encoding the CSR polypeptide chain is under the control of a third promoter. In some embodiments, the first promoter is operably linked to the 5′ end of the TCR nucleic acid sequence encoding the first TCR polypeptide chain. In some embodiments, the second promoter is operably linked to the 5′ end of the TCR nucleic acid sequence encoding the second TCR polypeptide chain. In some embodiments, the third promoter is operably linked to the 5′ end of the CSR nucleic acid sequence. In some embodiments, only one promoter is used. In some embodiments, there is nucleic acid linker selected from the group consisting of an internal ribosomal entry site (IRES) and a nucleic acid encoding a self-cleaving 2A peptide (such as P2A, T2A, E2A, or F2A) linking the 3′ end of a first and/or second TCR polypeptide chain nucleic acid sequence to the 5′ end of the CSR nucleic acid sequence, or the 5′ end of the promoter that is linked to the CSR, if the promoter specific to the CSR is present. In some embodiments, there is nucleic acid linker selected from the group consisting of an internal ribosomal entry site (IRES) and a nucleic acid encoding a self-cleaving 2A peptide (such as P2A, T2A, E2A, or F2A) linking the 3′ end of the CSR nucleic acid sequence to the 5′ end of a first and/or second TCR polypeptide chain nucleic acid sequence, or the 5′ end of the promoter that is linked to the TCR, if the promoter specific to the TCR is present. In some embodiments, the first and/or second TCR polypeptide chain nucleic acid sequence and the CSR nucleic acid sequence are transcribed as a single RNA under the control of one promoter.

Thus, in some embodiments, there is provided there nucleic acids, wherein a first nucleic acid encodes a first TCR polypeptide chain according to any of the TCRs described herein; a second nucleic acid encodes a second TCR polypeptide chain according to any of the TCRs described herein; and a third nucleic acid encodes a CSR polypeptide chain according to any of the CSRs described herein. In some embodiments, the three nucleic acids are contained in three vectors (such as lentiviral vectors).

In some embodiments, the first, second, and/or third promoters are inducible. In some embodiments, the first, second, and/or third vectors are viral vectors (such as lentiviral vectors). It is to be appreciated that embodiments where any of the nucleic acid sequences are swapped are also contemplated, such as where the first and/or second TCR polypeptide chain nucleic acid sequence is swapped with the CSR nucleic acid sequence.

### VIII. TCR and CSR Production

Provided TCRs and/or CSRs or portions thereof, or nucleic acids encoding them, may be produced by any available means. Methods for production are well-known in the art. Technologies for generating antibodies (e.g., scFv antibodies, monoclonal antibodies, and/or polyclonal antibodies) are available in the art. It will be appreciated that a wide range of animal species can be used for the production of antisera, e.g., mouse, rat, rabbit, pig, cow, deer, sheep, goat, cat, dog, monkey, and chicken. The choice of animal may be decided upon the ease of manipulation, costs or the desired amount of sera, as would be known to one of skill in the art. It will be appreciated that antibodies can also be produced transgenically through the generation of a mammal or plant that is transgenic for the immunoglobulin heavy and light chain sequences of interest (e.g., a transgenic rodent transgenic for human immunoglobulin heavy and light chain genes). In connection with the transgenic production in mammals, antibodies can be produced in, and recovered from, the milk of goats, cows, or other mammals (see, e.g., U.S. Pat. Nos. 5,827,690, 5,756,687, 5,750,172, and 5,741,957; herein incorporated by reference in their entireties). Alternatively, antibodies may be made in chickens, producing IgY molecules (Schade et al., 1996, ALTEX 13(5):80-85).

Although embodiments employing CSRs that contain human antibodies having, i.e., human heavy and light chain variable region sequences including human CDR sequences, are extensively discussed herein, the present invention also provides CSRs that contain non-human antibodies. In some embodiments, non-human antibodies comprise human CDR sequences from an antibody as described herein and non-human framework sequences. Non-human framework sequences include, in some embodiments, any sequence that can be used for generating synthetic heavy and/or light chain variable regions using one or more human CDR sequences as described herein, including, e.g., sequences generated from mouse, rat, rabbit, pig, cow, deer, sheep, goat, cat, dog, monkey, chicken, etc. In some embodiments, a provided CSR includes an antibody generated by grafting one or more human CDR sequences as described herein onto a non-human framework sequence (e.g., a mouse or chicken framework sequence). In many embodiments, provided CSRs comprise or are human antibodies (e.g., a human monoclonal antibody or fragment thereof, human antigen-binding protein or polypeptide, human multispecific antibody (e.g., a human bispecific antibody), a human polypeptide having one or more structural components of a human immunoglobulin polypeptide).

In some embodiments, antibodies suitable for the present invention are subhuman primate antibodies. For example, general techniques for raising therapeutically useful antibodies in baboons may be found, for example, in International Patent Application Publication No. 1991/11465 and in Losman et al., 1990, Int. J. Cancer 46:310. In some embodiments, antibodies (e.g., monoclonal antibodies) may be prepared using hybridoma methods (Milstein and Cuello, 1983, Nature 305(5934):537-40). In some embodiments, antibodies (e.g., monoclonal antibodies) may also be made by recombinant methods (see, e.g., U.S. Pat. No. 4,166,452).

Many of the difficulties associated with generating antibodies by B-cell immortalization can be overcome by engineering and expressing CSR components in E. coli or yeast using phage display. To ensure the recovery of high affinity antibodies a combinatorial immunoglobulin library must typically contain a large repertoire size. A typical strategy utilizes mRNA obtained from lymphocytes or spleen cells of immunized mice to synthesize cDNA using reverse transcriptase. The heavy and light chain genes are amplified separately by PCR and ligated into phage cloning vectors. Two different libraries may be produced, one containing the heavy chain genes and one containing the light chain genes. The libraries can be naïve or they can be semi-synthetic, i.e., with all amino acids (with the exception of cysteine) equally likely to be present at any given position in a CDR. Phage DNA is isolated from each library, and the heavy and light chain sequences are ligated together and packaged to form a combinatorial library. Each phage contains a random pair of heavy and light chain cDNAs and upon infection of E. coli directs the expression of the polypeptides in a CSR in infected cells. To identify a CSR that recognizes the antigen of interest, the phage library is plated, and the CSR molecules present in the plaques are transferred to filters. The filters are incubated with radioactively labeled antigen and then washed to remove excess unbound ligand. A radioactive spot on the autoradiogram identifies a plaque that contains a CSR that binds the antigen. Alternatively, identification of a CSR that recognizes the antigen of interest may be achieved by iterative binding of phage to the antigen, which is bound to a solid support, for example, beads or mammalian cells followed by removal of non-bound phage and by elution of specifically bound phage. In such embodiments, antigens are first biotinylated for immobilization to, for example, streptavidin-conjugated Dynabeads M-280. The phage library is incubated with the cells, beads or other solid support and non-binding phage is removed by washing. CSR phage clones that bind the antigen of interest are selected and tested for further characterization.

Once selected, positive clones may be tested for their binding to the antigen of interest expressed on the surface of live cells by flow cytometry. Briefly, phage clones may be incubated with cells (e.g., engineered to express the antigen of interest, or those that naturally express the antigen) that either do or do not express the antigen. The cells may be washed and then labeled with a mouse anti-M13 coat protein monoclonal antibody. Cells may be washed again and labeled with a fluorescent-conjugated secondary antibody (e.g., FITC-goat (Fab)₂ anti-mouse IgG) prior to flow cytometry. Cloning and expression vectors that are useful for producing a human immunoglobulin phage library can be obtained, for example, from Stratagene Cloning Systems (La Jolla, CA).

A similar strategy may be employed to obtain high affinity scFv clones. A library with a large repertoire may be constructed by isolating V-genes from non-immunized human donors using PCR primers corresponding to all known V_{H}, Vκ and Vλ gene families. Following amplification, the Vκ and Vλ pools may be combined to form one pool. These fragments may be ligated into a phagemid vector. An scFv linker (e.g., (G₄S)n) may be ligated into the phagemid upstream of the V_{L} fragment (or upstream of the V_{H} fragment as so desired). The V_{H} and linker-V_{L} fragments (or V_{L} and linker-V_{H} fragments) may be amplified and assembled on the JH region. The resulting V_{H}-linker-V_{L} (or V_{L}-linker-V_{H}) fragments may be ligated into a phagemid vector. The phagemid library may be panned using filters, as described above, or using immunotubes (Nunc; Maxisorp). Similar results may be achieved by constructing a combinatorial immunoglobulin library from lymphocytes or spleen cells of immunized rabbits and by expressing the scFv in P. pastoris (see, e.g., Ridder et al., 1995, Biotechnology, 13:255-260). Additionally, following isolation of appropriate scFv antibodies, higher binding affinities and slower dissociation rates may be obtained through affinity maturation processes such as mutagenesis and chain-shuffling (see, e.g., Jackson et al., 1998, Br. J. Cancer. 78:181-188); Osbourn et al., 1996, Immunotechnology 2:181-196).

Human antibodies may be produced using various techniques, i.e., introducing human Ig genes into transgenic animals in which the endogenous Ig genes have been partially or completely inactivated can be exploited to synthesize human antibodies. In some embodiments, human antibodies may be made by immunization of non-human animals engineered to make human antibodies in response to antigen challenge with human antigen.

Provided TCRs and CSRs may be also produced, for example, by utilizing a host cell system engineered to express a TCR- or CSR-encoding nucleic acid. Alternatively or additionally, provided TCRs may be partially or fully prepared by chemical synthesis (e.g., using an automated peptide synthesizer or gene synthesis of TCR- or CSR-encoding nucleic acids). TCRs and/or CSRs described herein may be expressed using any appropriate vector or expression cassette. A variety of vectors (e.g., viral vectors) and expression cassettes are known in the art and cells into which such vectors or expression cassettes may be introduced may be cultured as known in the art (e.g., using continuous or fed-batch culture systems). In some embodiments, cells may be genetically engineered; technologies for genetically engineering cells to express engineered polypeptides are well known in the art (see, e.g., Ausabel et al., eds., 1990, Current Protocols in Molecular Biology (Wiley, New York)).

TCRs and/or CSRs described herein may be purified, i.e., using filtration, centrifugation, and/or a variety of chromatographic technologies such as HPLC or affinity chromatography. In some embodiments, fragments of provided TCRs and/or CSRs are obtained by methods that include digestion with enzymes, such as pepsin or papain, and/or by cleavage of disulfide bonds by chemical reduction.

It will be appreciated that provided TCRs and/or CSRs may be engineered, produced, and/or purified in such a way as to improve characteristics and/or activity of the TCRs and/or CSRs. For example, improved characteristics include, but are not limited to, increased stability, improved binding affinity and/or avidity, increased binding specificity, increased production, decreased aggregation, decreased nonspecific binding, among others. In some embodiments, provided TCRs and/or CSRs may comprise one or more amino acid substitutions (e.g., in a framework region in the context of an immunoglobulin or fragment thereof (e.g., an scFv antibody) in the case of CSRs) that improve protein stability, antigen binding, expression level, or provides a site or location for conjugation of a therapeutic, diagnostic or detection agent.

Purification Tag

In some embodiments, a purification tag may be joined to a TCR and/or CSR described herein. A purification tag refers to a peptide of any length that can be used for purification, isolation, or identification of a polypeptide. A purification tag may be joined to a polypeptide (e.g., joined to the N- or C-terminus of the polypeptide) to aid in purifying the polypeptide and/or isolating the polypeptide from, e.g., a cell lysate mixture. In some embodiments, the purification tag binds to another moiety that has a specific affinity for the purification tag. In some embodiments, such moieties which specifically bind to the purification tag are attached to a solid support, such as a matrix, a resin, or agarose beads. Examples of a purification tag that may be joined to a TCR or CSR include, but are not limited to, a hexa-histidine peptide, a hemagglutinin (HA) peptide, a FLAG peptide, and a myc peptide. In some embodiments, two or more purification tags may be joined to a TCR or CSR, e.g., a hexa-histidine peptide and a HA peptide. A hexa-histidine peptide (HHHHHH (SEQ ID NO:257)) binds to nickel-functionalized agarose affinity column with micromolar affinity. In some embodiments, an HA peptide includes the sequence YPYDVPDYA (SEQ ID NO:258) or YPYDVPDYAS (SEQ ID NO:259). In some embodiments, an HA peptide includes integer multiples of the sequence YPYDVPDYA (SEQ ID NO:258) or YPYDVPDYAS (SEQ ID NO:259) in tandem series, e.g., 3×YPYDVPDYA or 3×YPYDVPDYAS. In some embodiments, a FLAG peptide includes the sequence DYKDDDDK (SEQ ID NO:260). In some embodiments, a FLAG peptide includes integer multiples of the sequence DYKDDDDK (SEQ ID NO:260) in tandem series, e.g., 3×DYKDDDDK. In some embodiments, a myc peptide includes the sequence EQKLISEEDL (SEQ ID NO:261). In some embodiments, a myc peptide includes integer multiples of the sequence EQKLISEEDL in tandem series, e.g., 3×EQKLISEEDL.

### IX. Therapeutic and Detection Agents

A therapeutic agent or a detection agent may be attached to a TCR or CSR described herein. Therapeutic agents may be any class of chemical entity including, for example, but not limited to, proteins, carbohydrates, lipids, nucleic acids, small organic molecules, non-biological polymers, metals, ions, radioisotopes, etc. In some embodiments, therapeutic agents for use in accordance with the present invention may have a biological activity relevant to the treatment of one or more symptoms or causes of cancer. In some embodiments, therapeutic agents for use in accordance with the present invention may have a biological activity relevant to modulation of the immune system and/or enhancement of T-cell mediated cytotoxicity. In some embodiments, therapeutic agents for use in accordance with the present invention have one or more other activities.

A detection agent may comprise any moiety that may be detected using an assay, for example due to its specific functional properties and/or chemical characteristics. Non-limiting examples of such agents include enzymes, radiolabels, haptens, fluorescent labels, phosphorescent molecules, chemiluminescent molecules, chromophores, luminescent molecules, photoaffinity molecules, colored particles or ligands, such as biotin.

Many detection agents are known in the art, as are systems for their attachment to proteins and peptides (see, for e.g., U.S. Pat. Nos. 5,021,236; 4,938,948; and 4,472,509). Examples of such detection agents include paramagnetic ions, radioactive isotopes, fluorochromes, NMR-detectable substances, X-ray imaging agents, among others. For example, in some embodiments, a paramagnetic ion is one or more of chromium (111), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), erbium (III), lanthanum (III), gold (III), lead (II), and/or bismuth (Ill).

The radioactive isotope may be one or more of actinium-225, astatine-211, bismuth-212, carbon-14, chromium-51, chlorine-36, cobalt-57, cobalt-58, copper-67, Europium-152, gallium-67, hydrogen-3, iodine-123, iodine-124, iodine-125, iodine-131, indium-111, iron-59, lead-212, lutetium-177, phosphorus-32, radium-223, radium-224, rhenium-186, rhenium-188, selenium-75, sulphur-35, technicium-99m, thorium-227, yttrium-90, and zirconium-89. Radioactively labeled TCRs or CSRs may be produced according to well-known technologies in the art.

A fluorescent label may be or may comprise one or more of Alexa 350, Alexa 430, AMCA, BODIPY 630/650, BODIPY 650/665, BODIPY-FL, BODIPY-R6G, BODIPY-TMR, BODIPY-TRX. Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red, among others.

### X. Methods of Treatment

The compositions of the invention can be administered to individuals (e.g., mammals such as humans) to treat diseases including viral infections and cancers (e.g., a hematological cancer or a solid tumor cancer).

Cancers that may be treated using any of the methods described herein include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated include, but are not limited to, carcinoma, blastoma, sarcoma, melanoma, neuroendocrine tumors, and glioma, and certain leukemia or lymphoid malignancies, benign and malignant tumors, and malignancies e.g., sarcomas, carcinomas, melanomas, and gliomas. Adult tumors/cancers and pediatric tumors/cancers are also included.

Solid tumors contemplated for treatment by any of the methods described herein include CNS tumors, such as glioma (e.g., brainstem glioma and mixed gliomas), glioblastoma (also known as glioblastoma multiforme), astrocytoma (such as high-grade astrocytoma), pediatric glioma or glioblastoma (such as pediatric high-grade glioma (HGG) and diffuse intrinsic pontine glioma (DIPG)), CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

In some embodiments, the cancer is pediatric glioma. In some embodiments, the pediatric glioma is a low-grade glioma. In some embodiments, the pediatric glioma is a high-grade glioma (HGG). In some embodiments, the pediatric glioma is glioblastoma multiforme. In some embodiments, the pediatric glioma is diffuse intrinsic pontine glioma (DIPG). In some embodiments, the DIPG is grade II. In some embodiments, the DIPG is grade III. In some embodiments, the DIPG is grade IV.

Additional solid tumors contemplated for treatment include fibrosarcoma, myxosarcoma, liposarcoma, chondrosarcoma (such as clear-cell chondrosarcoma), chondroblastoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer (e.g., cervical carcinoma and pre-invasive cervical dysplasia), cancer of the anus, anal canal, or anorectum, vaginal cancer, cancer of the vulva (e.g., squamous cell carcinoma, intraepithelial carcinoma, adenocarcinoma, and fibrosarcoma), penile cancer, oropharyngeal cancer, head cancers (e.g., squamous cell carcinoma), neck cancers (e.g., squamous cell carcinoma), testicular cancer (e.g., seminoma, teratoma, embryonal carcinoma, teratocarcinoma, choriocarcinoma, sarcoma, Leydig cell tumor, fibroma, fibroadenoma, adenomatoid tumors, and lipoma), bladder carcinoma, melanoma, cancer of the uterus (e.g., endometrial carcinoma), and urothelial cancers (e.g., squamous cell carcinoma, transitional cell carcinoma, adenocarcinoma, ureter cancer, and urinary bladder cancer).

Hematologic cancers contemplated for treatment by any of the methods described herein include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma. Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of other cancers include, without limitation, acute lymphoblastic leukemia (ALL), Hodgkin's lymphoma, non-Hodgkin's lymphoma, B cell chronic lymphocytic leukemia (CLL), multiple myeloma, follicular lymphoma, mantle cell lymphoma, pro-lymphocytic leukemia, hairy cell leukemia, common acute lymphocytic leukemia, and null-acute lymphoblastic leukemia.

Cancer treatments can be evaluated, for example, by tumor regression, tumor weight or size shrinkage, time to progression, duration of survival, progression free survival, overall response rate, duration of response, quality of life, protein expression and/or activity. Approaches to determining efficacy of therapy can be employed, including for example, measurement of response through radiological imaging.

In some embodiments of any of the methods for treating cancer (e.g., a hematological cancer or a solid tumor cancer), the TCR and CSR are conjugated to a cell (such as an immune cell, e.g., a T cell) prior to being administered to the individual. Thus, for example, there is provided a method of treating cancer (e.g., a hematological cancer or a solid tumor cancer) in an individual comprising a) conjugating a TCR and CSR described herein or an antibody moiety thereof to a cell (such as an immune cell, e.g., a T cell) to form a TCR+CSR/cell conjugate, and b) administering to the individual an effective amount of a composition comprising the TCR+CSR/cell conjugate. In some embodiments, the cell is derived from the individual. In some embodiments, the cell is not derived from the individual. In some embodiments, the TCR and CSR are conjugated to the cell by covalent linkage to a molecule on the surface of the cell. In some embodiments, the TCR and CSR are conjugated to the cell by non-covalent linkage to a molecule on the surface of the cell. In some embodiments, the TCR and CSR are conjugated to the cell by insertion of a portion of the TCR and a portion of the CSR into the outer membrane of the cell.

Treatments can be evaluated, for example, by tumor regression, tumor weight or size shrinkage, time to progression, duration of survival, progression free survival, overall response rate, duration of response, quality of life, protein expression and/or activity. Approaches to determining efficacy of therapy can be employed, including for example, measurement of response through radiological imaging.

In some embodiments, the efficacy of treatment may be measured as the percentage tumor growth inhibition (% TGI), which may be calculated using the equation 100−(T/C×100), where T is the mean relative tumor volume of the treated tumor, and C is the mean relative tumor volume of a non-treated tumor. In some embodiments, the % TGI is about 2%, about 4%, about 6, about 8%, 10%, about 20%, about 30%, about 40%, about 50%, about 60%, about 70%, about 80%, about 90%, about 91%, about 92%, about 93%, about 94%, about 95%, or more than 95%.

### XI. Preparation of TCR Plus CSR Immune Cells

The present invention in one aspect provides immune cells (such as lymphocytes, for example T cells) expressing a TCR and a CSR according to any of the embodiments described herein. Exemplary methods of preparing immune cells (such as T cells) expressing a TCR and a CSR (TCR plus CSR immune cells, such as TCR plus CSR T cells) are provided herein.

In some embodiments, a TCR plus CSR immune cell (such as a TCR plus CSR T cell) can be generated by introducing one or more nucleic acids (including for example a lentiviral vector) encoding a TCR (such as any of the TCRs described herein) that specifically binds to a target antigen (such as a disease-associated antigen) and a CSR (such as any of the CSRs described herein) that specifically binds to a target ligand into the immune cell. The introduction of the one or more nucleic acids into the immune cell can be accomplished using techniques known in the art, such as those described herein for Nucleic Acids. In some embodiments, the TCR plus CSR immune cells (such as TCR plus CSR T cells) of the invention are able to replicate in vivo, resulting in long-term persistence that can lead to sustained control of a disease associated with expression of the target antigen (such as cancer or viral infection).

In some embodiments, the invention relates to administering a genetically modified T cell expressing a TCR that specifically binds to a target antigen according to any of the TCRs described herein and a CSR that specifically binds to a target ligand according to any of the CSRs described herein for the treatment of a patient having or at risk of developing a disease and/or disorder associated with expression of the target antigen (also referred to herein as a “target antigen-positive” or “TA-positive” disease or disorder), including, for example, cancer or viral infection, using lymphocyte infusion. In some embodiments, autologous lymphocyte infusion is used in the treatment. Autologous PBMCs are collected from a patient in need of treatment and T cells are activated and expanded using the methods described herein and known in the art and then infused back into the patient.

In some embodiments, there is provided a T cell expressing a TCR that specifically binds to a target antigen according to any of the TCRs described herein and a CSR that specifically binds to a target ligand according to any of the CSRs described herein (also referred to herein as an “TCR plus CSR T cell”). The TCR plus CSR T cells of the invention can undergo robust in vivo T cell expansion and can establish target antigen-specific memory cells that persist at high levels for an extended amount of time in blood and bone marrow. In some embodiments, the TCR plus CSR T cells of the invention infused into a patient can eliminate target antigen-presenting cells, such as target antigen-presenting cancer or virally infected cells, in vivo in patients having a target antigen-associated disease. In some embodiments, the TCR plus CSR T cells of the invention infused into a patient can eliminate target antigen-presenting cells, such as target antigen-presenting cancer or virally infected cells, n vivo in patients having a target antigen-associated disease that is refractory to at least one conventional treatment.

Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments of the present invention, any number of T cell lines available in the art may be used. In some embodiments of the present invention. T cells can be obtained from a unit of blood collected from a subject using any number of techniques known to the skilled artisan, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as Ca²⁺-free, Mg²⁺-free PBS, PlasmaLyte A, or other saline solutions with or without buffer. Alternatively, the undesirable components of the apheresis sample may be removed, and the cells directly resuspended in culture media.

In some embodiments. T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In some embodiments, the time period ranges from 30 minutes to 36 hours or longer (including all ranges between these values). In some embodiments, the time period is at least one, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such as in isolating tumor infiltrating lymphocytes (TIL) from tumor tissue or from immune-compromised individuals. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In some embodiments, it may be desirable to enrich for or positively select for regulatory T cells which typically express CD4⁺, CD25⁺, CD62Lhi, GITR⁺, and FoxP3⁺. Alternatively, in some embodiments, T regulatory cells are depleted by anti-CD25 conjugated beads or other similar methods of selection.

For isolation of a desired population of cells by positive or negative selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/ml is used. In some embodiments, a concentration of about 1 billion cells/ml is used. In some embodiments, greater than about 100 million cells/ml is used. In some embodiments, a concentration of cells of about any of 10, 15, 20, 25, 30, 35, 40, 45, or 50 million cells/ml is used. In some embodiments, a concentration of cells of about any of 75, 80, 85, 90, 95, or 100 million cells/ml is used. In some embodiments, a concentration of about 125 or about 150 million cells/ml is used. Using high concentrations can result in increased cell yield, cell activation, and cell expansion or proliferation. As used herein, the terms “expansion” and “proliferation” are used synonymously. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In some embodiments of the present invention. T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in some embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

Whether prior to or after genetic modification of the T cells to express a desirable TCR, CSR and optionally SSE, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a costimulatory molecule on the surface of the T cells. In particular, T cell populations may be stimulated, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besançon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

### XII. Genetic Modification

In some embodiments, the TCR plus CSR immune cells (such as TCR plus CSR T cells) of the invention are generated by transducing immune cells (such as T cells prepared by the methods described herein) with one or more viral vectors encoding a TCR as described herein and a CSR as described herein. Viral vector delivery systems include DNA and RNA viruses, which have either episomal or integrated genomes after delivery to the immune cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1 154 (1988); Vigne, Restorative Neurology and Neuroscience 8:35-36 (1995); Kremer & Perricaudet, British Medical Bulletin 51(l):31-44 (1995); and Yu et al., Gene Therapy 1:13-26 (1994). In some embodiments, the TCR plus CSR immune cell comprises the one or more vectors integrated into the TCR plus CSR immune cell genome. In some embodiments, the one or more viral vectors are lentiviral vectors. In some embodiments, the TCR plus CSR immune cell is a TCR plus CSR T cell comprising the lentiviral vectors integrated into its genome.

In some embodiments, the TCR plus CSR immune cell is a T cell modified to block or decrease the expression of one or both of its endogenous TCR chains. For example, in some embodiments, the TCR plus CSR immune cell is an αβ T cell modified to block or decrease the expression of the TCR α and/or β chains, or the TCR plus CSR immune cell is a γδ T cell modified to block or decrease the expression of the TCR γ and/or δ chains. Modifications of cells to disrupt gene expression include any such techniques known in the art, including for example RNA interference (e.g., siRNA, shRNA, miRNA), gene editing (e.g., CRISPR- or TALEN-based gene knockout), and the like.

In some embodiments, TCR plus CSR T cells with reduced expression of one or both of the endogenous TCR chains of the T cell are generated using the CRISPR/Cas system. For a review of the CRISPR/Cas system of gene editing, see for example Jian W & Marraffini L A, Annu. Rev. Microbiol. 69, 2015; Hsu P D et al., Cell, 157(6):1262-1278, 2014; and O'Connell M R et al., Nature 516: 263-266, 2014. In some embodiments, TCR plus CSR T cells with reduced expression of one or both of the endogenous TCR chains of the T cell are generated using TALEN-based genome editing.

### XIII. Enrichment

In some embodiments, there is provided a method of enriching a heterogeneous cell population for a TCR plus CSR immune cell according to any of the TCR plus CSR immune cells described herein.

A specific subpopulation of TCR plus CSR immune cells (such as TCR plus CSR T cells) that specifically bind to a target antigen and target ligand can be enriched for by positive selection techniques. For example, in some embodiments, TCR plus CSR immune cells (such as TCR plus CSR T cells) are enriched for by incubation with target antigen-conjugated beads and/or target ligand-conjugated beads for a time period sufficient for positive selection of the desired TCR plus CSR immune cells. In some embodiments, the time period is about 30 minutes. In some embodiments, the time period ranges from 30 minutes to 36 hours or longer (including all ranges between these values). In some embodiments, the time period is at least one, 2, 3, 4, 5, or 6 hours. In some embodiments, the time period is 10 to 24 hours. In some embodiments, the incubation time period is 24 hours. For isolation of TCR plus CSR immune cells present at low levels in the heterogeneous cell population, use of longer incubation times, such as 24 hours, can increase cell yield. Longer incubation times may be used to isolate TCR plus CSR immune cells in any situation where there are few TCR plus CSR immune cells as compared to other cell types. The skilled artisan would recognize that multiple rounds of selection can also be used in the context of this invention.

For isolation of a desired population of TCR plus CSR immune cells by positive selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/ml is used. In some embodiments, a concentration of about 1 billion cells/ml is used. In some embodiments, greater than about 100 million cells/ml is used. In some embodiments, a concentration of cells of about any of 10, 15, 20, 25, 30, 35, 40, 45, or 50 million cells/ml is used. In some embodiments, a concentration of cells of about any of 75, 80, 85, 90, 95, or 100 million cells/ml is used. In some embodiments, a concentration of about 125 or about 150 million cells/ml is used. Using high concentrations can result in increased cell yield, cell activation, and cell expansion. Further, use of high cell concentrations allows more efficient capture of TCR plus CSR immune cells that may weakly express the TCR and/or CSR.

In some of any such embodiments described herein, enrichment results in minimal or substantially no exhaustion of the TCR plus CSR immune cells. For example, in some embodiments, enrichment results in fewer than about 50% (such as fewer than about any of 45, 40, 35, 30, 25, 20, 15, 10, or 5%) of the TCR plus CSR immune cells becoming exhausted. Immune cell exhaustion can be determined by any means known in the art, including any means described herein.

In some of any such embodiments described herein, enrichment results in minimal or substantially no terminal differentiation of the TCR plus CSR immune cells. For example, in some embodiments, enrichment results in fewer than about 50% (such as fewer than about any of 45, 40, 35, 30, 25, 20, 15, 10, or 5%) of the TCR plus CSR immune cells becoming terminally differentiated. Immune cell differentiation can be determined by any means known in the art, including any means described herein.

In some of any such embodiments described herein, enrichment results in minimal or substantially no internalization of TCRs and/or CSRs on the TCR plus CSR immune cells. For example, in some embodiments, enrichment results in less than about 50% (such as less than about any of 45, 40, 35, 30, 25, 20, 15, 10, or 5%) of TCRs and/or CSRs on the TCR plus CSR immune cells becoming internalized. Internalization of TCRs and/or CSRs on TCR plus CSR immune cells can be determined by any means known in the art, including any means described herein.

In some of any such embodiments described herein, enrichment results in increased proliferation of the TCR plus CSR immune cells. For example, in some embodiments, enrichment results in an increase of at least about 10% (such as at least about any of 20, 30, 40, 50, 60, 70, 80, 90, 100, 200, 300, 400, 500, 1000% or more) in the number of TCR plus CSR immune cells following enrichment.

Thus, in some embodiments, there is provided a method of enriching a heterogeneous cell population for TCR plus CSR immune cells expressing a TCR that specifically binds to a target antigen and a CSR that specifically binds to a target ligand comprising: a) contacting the heterogeneous cell population with a first molecule comprising the target antigen or one or more epitopes contained therein and/or a second molecule comprising the target ligand or one or more epitopes contained therein to form complexes comprising the TCR plus CSR immune cell bound to the first molecule and/or complexes comprising the TCR plus CSR immune cell bound to the second molecule; and b) separating the complexes from the heterogeneous cell population, thereby generating a cell population enriched for the TCR plus CSR immune cells. In some embodiments, the first and/or second molecules are immobilized, individually, to a solid support. In some embodiments, the solid support is particulate (such as beads). In some embodiments, the solid support is a surface (such as the bottom of a well). In some embodiments, the first and/or second molecules are labelled, individually, with a tag. In some embodiments, the tag is a fluorescent molecule, an affinity tag, or a magnetic tag. In some embodiments, the method further comprises eluting the TCR plus CSR immune cells from the first and/or second molecules and recovering the eluate.

### XIV. Effector Cell Therapy

The present application also provides methods of using immune cells as described herein to redirect the specificity of an effector cell (such as a primary T cell) to a cancer cell. Thus, the present invention also provides a method of stimulating an effector cell-mediated response (such as a T cell-mediated immune response) to a target cell population or tissue comprising cancer cells in a mammal, comprising the step of administering to the mammal an effector cell (such as a T cell) that expresses a TCR and a CSR as described herein. In some embodiments, “stimulating” an immune cell refers to eliciting an effector cell-mediated response (such as a T cell-mediated immune response), which is different from activating an immune cell.

Effector cells (such as T cells) expressing a TCR and a CSR as described herein can be infused to a recipient in need thereof. The infused cell is able to kill cancer cells in the recipient. In some embodiments, unlike antibody therapies, effector cells (such as T cells) are able to replicate in vivo resulting in long-term persistence that can lead to sustained tumor control.

In some embodiments, the effector cells are T cells that can undergo robust in vivo T cell expansion and can persist for an extended amount of time. In some embodiments, the T cells of the invention develop into specific memory T cells that can be reactivated to inhibit any additional tumor formation or growth.

The effector cells (such as T cells) of the invention may also serve as a type of vaccine for ex vivo immunization and/or in vivo therapy in a mammal. In some embodiments, the mammal is a human.

With respect to ex vivo immunization, at least one of the following occurs in vitro prior to administering the cell into a mammal: i) expansion of the cells, ii) introducing nucleic acid(s) encoding a TCR and a CSR to the cells, and/or iii) cryopreservation of the cells. Er vivo procedures are well-known in the art. Briefly, cells are isolated from a mammal (preferably a human) and genetically modified (i.e., transduced or transfected in vitro) with vector(s) expressing a TCR and a CSR disclosed herein. The cell can be administered to a mammalian recipient to provide a therapeutic benefit. The mammalian recipient may be a human and the cell can be autologous with respect to the recipient. Alternatively, the cells can be allogeneic, syngeneic or xenogeneic with respect to the recipient. The procedure for ex vivo expansion of hematopoietic stem and progenitor cells is described in U.S. Pat. No. 5,199,942, incorporated herein by reference, can be applied to the cells of the present invention. Other suitable methods are known in the art; therefore, the present invention is not limited to any particular method of ex vivo expansion of the cells. Briefly, ex vivo culture and expansion of T cells comprises: (1) collecting T cells from peripheral blood mononuclear cells (PBMC); and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient. The effector cells (such as T cells) of the present invention may be administered either alone, or as a pharmaceutical composition in combination with diluents and/or with other components such as IL-2 or other cytokines or cell populations. Briefly, pharmaceutical compositions of the present invention may comprise effector cells (such as T cells), in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, effector cell (such as T cell) compositions are formulated for administration by intravenous, intrathecal, intracranial, intracerebral, or intracerebroventricular route.

The precise amount of the effector cell (such as TCR T cell) compositions of the present invention to be administered can be determined by a physician with consideration of individual differences in age, weight, tumor size, extent of infection or metastasis, and condition of the patient (subject). In some embodiments, a pharmaceutical composition comprising the effector cells (such as T cells) is administered at a dosage of about 10⁴ to about 10⁹ cells/kg body weight, such any of about 10⁴ to about 10⁵, about 10⁵ to about 10⁶, about 10⁶ to about 10⁷, about 10⁷ to about 10⁸, or about 10⁸ to about 10⁹ cells/kg body weight, including all integer values within those ranges. Effect cell (such as T cell) compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regimen for a particular patient can readily be determined by one skilled in the art of medicine by monitoring the patient for signs of disease and adjusting the treatment accordingly.

In some embodiments, it may be desired to administer activated effector cells (such as T cells) to a subject and then subsequently redraw blood (or have an apheresis performed), activate T cells therefrom according to the present invention, and reinfuse the patient with these activated and expanded T cells. This process can be carried out multiple times every few weeks. In some embodiments, T cells can be activated from blood draws of from 10 cc to 400 cc. In some embodiments, T cells are activated from blood draws of 20 cc, 30 cc, 40 cc, 50 cc, 60 cc, 70 cc, 80 cc, 90 cc, or 100 cc.

The administration of the effector cells (such as T cells) may be carried out in any convenient manner, including by injection, ingestion, transfusion, implantation or transplantation. The compositions described herein may be administered to a patient subcutaneously, intradermally, intratumorally, intranodally, intramedullary, intramuscularly, intrathecally, intracranially, intracerebrally, intracerebroventricularly, by intravenous (i.v.) injection, or intraperitoneally. In some embodiments, the effector cell (such as T cell) compositions of the present invention are administered to a patient by intradermal or subcutaneous injection. In some embodiments, the effector cell (such as T cell) compositions of the present invention are administered by i.v. injection. In some embodiments, the effector cell (such as T cell) compositions of the present invention are administered by intrathecal injection. In some embodiments, the effector cell (such as T cell) compositions of the present invention are administered by intracranial injection. In some embodiments, the effector cell (such as T cell) compositions of the present invention are administered by intracerebral injection. In some embodiments, the effector cell (such as T cell) compositions of the present invention are administered by intracerebroventricular injection. The compositions of effector cell (such as T cell) may be injected directly into a tumor, lymph node, or site of infection.

### XV. Methods of Diagnosis and Imaging Using TCRS and CSRS

Labeled TCRs and CSRs can be used for diagnostic purposes to detect, diagnose, or monitor a cancer. For example, the TCRs and CSRs described herein can be used in in situ, in vivo, ex vivo, and in vitro diagnostic assays or imaging assays.

Additional embodiments of the invention include methods of diagnosing a cancer (e.g., a hematological cancer or a solid tumor cancer) in an individual (e.g., a mammal such as a human). The methods comprise detecting antigen-presenting cells in the individual. In some embodiments, there is provided a method of diagnosing a cancer (e.g., a hematological cancer or a solid tumor cancer) in an individual (e.g., a mammal, such as a human) comprising (a) administering an effective amount of a labeled antibody moiety according to any of the embodiments described above to the individual; and (b) determining the level of the label in the individual, such that a level of the label above a threshold level indicates that the individual has the cancer. The threshold level can be determined by various methods, including, for example, by detecting the label according to the method of diagnosing described above in a first set of individuals that have the cancer and a second set of individuals that do not have the cancer, and setting the threshold to a level that allows for discrimination between the first and second sets. In some embodiments, the threshold level is zero, and the method comprises determining the presence or absence of the label in the individual. In some embodiments, the method further comprises waiting for a time interval following the administering of step (a) to permit the labeled antibody moiety to preferentially concentrate at sites in the individual where the antigen is expressed (and for unbound labeled antibody moiety to be cleared). In some embodiments, the method further comprises subtracting a background level of the label. Background level can be determined by various methods, including, for example, by detecting the label in the individual prior to administration of the labeled antibody moiety, or by detecting the label according to the method of diagnosing described above in an individual that does not have the cancer.

Antibody moieties of the invention can be used to assay levels of antigen-presenting cell in a biological sample using methods known to those of skill in the art. Suitable antibody labels are known in the art and include enzyme labels, such as, glucose oxidase; radioisotopes, such as iodine (131I, 125I, 123I, 121I), carbon (14C), sulfur (35S), tritium (3H), indium (115mIn, 113mIn, 112In, 111In), technetium (99Tc, 99mTc), thallium (201Ti), gallium (68Ga, 67Ga), palladium (103Pd), molybdenum (99Mo), xenon (133Xe), fluorine (18F), samarium (153Sm), lutetium (177Lu), gadolinium (159Gd), promethium (149Pm), lanthanum (140La), ytterbium (175Yb), holmium (166Ho), yttrium (90Y), scandium (47Sc), rhenium (186Re, 188Re), praseodymium (142Pr), rhodium (105Rh), and ruthenium (97Ru); luminol; fluorescent labels, such as fluorescein and rhodamine; and biotin.

Techniques known in the art may be applied to labeled antibody moieties of the invention. Such techniques include, but are not limited to, the use of bifunctional conjugating agents (see e.g., U.S. Pat. Nos. 5,756,065; 5,714,631; 5,696,239; 5,652,361; 5,505,931; 5,489,425; 5,435,990; 5,428,139; 5,342,604; 5,274,119; 4,994,560; and 5,808,003). Aside from the above assays, various in vivo and ex vivo assays are available to the skilled practitioner. For example, one can expose cells within the body of the subject to an antibody moiety which is optionally labeled with a detectable label, e.g., a radioactive isotope, and binding of the antibody moiety to the cells can be evaluated, e.g., by external scanning for radioactivity or by analyzing a sample (e.g., a biopsy or other biological sample) derived from a subject previously exposed to the antibody moiety.

### XVI. Pharmaceutical Compositions

Also provided herein are TCR plus CSR immune cell compositions (such as pharmaceutical compositions, also referred to herein as formulations) comprising an immune cell (such as a T cell) presenting on its surface a TCR according to any of the TCRs described herein and a CSR according to any of the CSRs described herein. In some embodiments, the TCR plus CSR immune cell composition is a pharmaceutical composition.

The composition may comprise a homogenous cell population comprising TCR plus CSR immune cells of the same cell type and expressing the same TCR and CSR, or a heterogeneous cell population comprising a plurality of TCR plus CSR immune cell populations comprising TCR plus CSR immune cells of different cell types, expressing different TCRs, and/or expressing different CSRs. The composition may further comprise cells that are not TCR plus CSR immune cells.

Thus, in some embodiments, there is provided a TCR plus CSR immune cell composition comprising a homogeneous cell population of TCR plus CSR immune cells (such as TCR plus CSR T cells) of the same cell type and expressing the same TCR and CSR. In some embodiments, the TCR plus CSR immune cell is a T cell. In some embodiments, the TCR plus CSR immune cell is selected from the group consisting of a cytotoxic T cell, a helper T cell, a natural killer T cell, a tumor infiltrating T cell (TIL T cell), and a suppressor T cell. In some embodiments, the TCR plus CSR immune cell composition is a pharmaceutical composition.

In some embodiments, there is provided a TCR plus CSR immune cell composition comprising a heterogeneous cell population comprising a plurality of TCR plus CSR immune cell populations comprising TCR plus CSR immune cells of different cell types, expressing different TCRs, and/or expressing different CSRs. In some embodiments, the TCR plus CSR immune cells are T cells. In some embodiments, each population of TCR plus CSR immune cells is, independently from one another, of a cell type selected from the group consisting of cytotoxic T cells, helper T cells, natural killer T cells, tumor infiltrating T cells (TIL T cells), and suppressor T cells. In some embodiments, all of the TCR plus CSR immune cells in the composition are of the same cell type (e.g., all of the TCR plus CSR immune cells are cytotoxic T cells). In some embodiments, at least one population of TCR plus CSR immune cells is of a different cell type than the others (e.g., one population of TCR plus CSR immune cells consists of cytotoxic T cells and the other populations of TCR plus CSR immune cells consist of natural killer T cells). In some embodiments, each population of TCR plus CSR immune cells expresses the same TCR. In some embodiments, at least one population of TCR plus CSR immune cells expresses a different TCR than the others. In some embodiments, each population of TCR plus CSR immune cells expresses a different TCR than the others. In some embodiments, each population of TCR plus CSR immune cells expresses a TCR that specifically binds to the same target antigen. In some embodiments, at least one population of TCR plus CSR immune cells expresses a TCR that specifically binds to a different target antigen than the others (e.g., one population of TCR plus CSR immune cells specifically binds to a pMHC complex and the other populations of TCR plus CSR immune cells specifically bind to a cell surface receptor). In some embodiments, where at least one population of TCR plus CSR immune cells expresses a TCR that specifically binds to a different target antigen, each population of TCR plus CSR immune cells expresses a TCR that specifically binds to a target antigen associated with the same disease or disorder (e.g., each of the target antigens are associated with a cancer, such as breast cancer). In some embodiments, each population of TCR plus CSR immune cells expresses the same CSR. In some embodiments, at least one population of TCR plus CSR immune cells expresses a different CSR than the others. In some embodiments, each population of TCR plus CSR immune cells expresses a different CSR than the others. In some embodiments, each population of TCR plus CSR immune cells expresses a CSR that specifically binds to the same target ligand. In some embodiments, at least one population of TCR plus CSR immune cells expresses a CSR that specifically binds to a different target ligand than the others (e.g., one population of TCR plus CSR immune cells specifically binds to a pMHC complex and the other populations of TCR plus CSR immune cells specifically bind to a cell surface receptor). In some embodiments, where at least one population of TCR plus CSR immune cells expresses a CSR that specifically binds to a different target ligand, each population of TCR plus CSR immune cells expresses a CSR that specifically binds to a target ligand associated with the same disease or disorder (e.g., each of the target ligands are associated with a cancer, such as breast cancer). In some embodiments, the TCR plus CSR immune cell composition is a pharmaceutical composition.

Thus, in some embodiments, there is provided a TCR plus CSR immune cell composition comprising a plurality of TCR plus CSR immune cell populations according to any of the embodiments described herein, wherein all of the TCR plus CSR immune cells in the composition are of the same cell type (e.g., all of the TCR plus CSR immune cells are cytotoxic T cells), and wherein each population of TCR plus CSR immune cells expresses a different TCR than the others. In some embodiments, the TCR plus CSR immune cells are T cells. In some embodiments, the TCR plus CSR immune cells are selected from the group consisting of cytotoxic T cells, helper T cells, natural killer T cells, tumor infiltrating T cells (TIL T cells), and suppressor T cells. In some embodiments, each population of TCR plus CSR immune cells expresses a TCR that specifically binds to the same target antigen. In some embodiments, at least one population of TCR plus CSR immune cells expresses a TCR that specifically binds to a different target antigen than the others (e.g., one population of TCR plus CSR immune cells specifically binds to a pMHC complex and the other populations of TCR plus CSR immune cells specifically bind to a cell surface receptor). In some embodiments, where at least one population of TCR plus CSR immune cells expresses a TCR that specifically binds to a different target antigen, each population of TCR plus CSR immune cells expresses a TCR that specifically binds to a target antigen associated with the same disease or disorder (e.g., each of the target antigens are associated with a cancer, such as breast cancer). In some embodiments, the TCR plus CSR immune cell composition is a pharmaceutical composition.

In some embodiments, there is provided a TCR plus CSR immune cell composition comprising a plurality of TCR plus CSR immune cell populations according to any of the embodiments described herein, wherein all of the TCR plus CSR immune cells in the composition are of the same cell type (e.g., all of the TCR plus CSR immune cells are cytotoxic T cells), and wherein each population of TCR plus CSR immune cells expresses a different CSR than the others. In some embodiments, the TCR plus CSR immune cells are T cells. In some embodiments, the TCR plus CSR immune cells are selected from the group consisting of cytotoxic T cells, helper T cells, natural killer T cells, tumor infiltrating T cells (TIL T cells), and suppressor T cells. In some embodiments, each population of TCR plus CSR immune cells expresses a CSR that specifically binds to the same target ligand. In some embodiments, at least one population of TCR plus CSR immune cells expresses a CSR that specifically binds to a different target ligand than the others (e.g., one population of TCR plus CSR immune cells specifically binds to a pMHC complex and the other populations of TCR plus CSR immune cells specifically bind to a cell surface receptor). In some embodiments, where at least one population of TCR plus CSR immune cells expresses a CSR that specifically binds to a different target ligand, each population of TCR plus CSR immune cells expresses a CSR that specifically binds to a target ligand associated with the same disease or disorder (e.g., each of the target ligands are associated with a cancer, such as breast cancer). In some embodiments, the TCR plus CSR immune cell composition is a pharmaceutical composition.

In some embodiments, there is provided a composition comprising a plurality of TCR plus CSR immune cell populations according to any of the embodiments described herein, wherein at least one population of TCR plus CSR immune cells is of a different cell type than the others. In some embodiments, all of the populations of TCR plus CSR immune cells are of different cell types. In some embodiments, the TCR plus CSR immune cells are T cells. In some embodiments, each population of TCR plus CSR immune cells is, independently from one another, of a cell type selected from the group consisting of cytotoxic T cells, helper T cells, natural killer T cells, tumor infiltrating T cells (TIL T cells), and suppressor T cells. In some embodiments, each population of TCR plus CSR immune cells expresses the same TCR. In some embodiments, at least one population of TCR plus CSR immune cells expresses a different TCR than the others. In some embodiments, each population of TCR plus CSR immune cells expresses a different TCR than the others. In some embodiments, each population of TCR plus CSR immune cells expresses a TCR that specifically binds to the same target antigen. In some embodiments, at least one population of TCR plus CSR immune cells expresses a TCR that specifically binds to a different target antigen than the others (e.g., one population of TCR plus CSR immune cells specifically binds to a pMHC complex and the other populations of TCR plus CSR immune cells specifically bind to a cell surface receptor). In some embodiments, where at least one population of TCR plus CSR immune cells expresses a TCR that specifically binds to a different target antigen, each population of TCR plus CSR immune cells expresses a TCR that specifically binds to a target antigen associated with the same disease or disorder (e.g., each of the target antigens are associated with a cancer, such as breast cancer). In some embodiments, each population of TCR plus CSR immune cells expresses the same CSR. In some embodiments, at least one population of TCR plus CSR immune cells expresses a different CSR than the others. In some embodiments, each population of TCR plus CSR immune cells expresses a different CSR than the others. In some embodiments, each population of TCR plus CSR immune cells expresses a CSR that specifically binds to the same target ligand. In some embodiments, at least one population of TCR plus CSR immune cells expresses a CSR that specifically binds to a different target ligand than the others (e.g., one population of TCR plus CSR immune cells specifically binds to a pMHC complex and the other populations of TCR plus CSR immune cells specifically bind to a cell surface receptor). In some embodiments, where at least one population of TCR plus CSR immune cells expresses a CSR that specifically binds to a different target ligand, each population of TCR plus CSR immune cells expresses a CSR that specifically binds to a target ligand associated with the same disease or disorder (e.g., each of the target ligands are associated with a cancer, such as breast cancer). In some embodiments, the TCR plus CSR immune cell composition is a pharmaceutical composition.

At various points during preparation of a composition, it can be necessary or beneficial to cryopreserve a cell. The terms “frozen/freezing” and “cryopreserved/cryopreserving” can be used interchangeably. Freezing includes freeze drying.

As is understood by one of ordinary skill in the art, the freezing of cells can be destructive (see Mazur, P., 1977, Cryobiology 14:251-272) but there are numerous procedures available to prevent such damage. For example, damage can be avoided by (a) use of a cryoprotective agent, (b) control of the freezing rate, and/or (c) storage at a temperature sufficiently low to minimize degradative reactions. Exemplary cryoprotective agents include dimethyl sulfoxide (DMSO) (Lovelock and Bishop, 1959, Nature 183:1394-1395; Ashwood-Smith, 1961, Nature 190:1204-1205), glycerol, polyvinylpyrrolidine (Rinfret, 1960, Ann. N.Y. Acad. Sci. 85:576), polyethylene glycol (Sloviter and Ravdin, 1962, Nature 196:548), albumin, dextran, sucrose, ethylene glycol, i-erythritol, D-ribitol, D-mannitol (Rowe et al., 1962, Fed. Proc. 21:157), D-sorbitol, i-inositol, D-lactose, choline chloride (Bender et al., 1960, J. Appl. Physiol. 15:520), amino acids (Phan The Tran and Bender, 1960, Exp. Cell Res. 20:651), methanol, acetamide, glycerol monoacetate (Lovelock, 1954, Biochem. J. 56:265), and inorganic salts (Phan The Tran and Bender, 1960, Proc. Soc. Exp. Biol. Med. 104:388; Phan The Tran and Bender, 1961, in Radiobiology, Proceedings of the Third Australian Conference on Radiobiology, Ilbery ed., Butterworth, London, p. 59). In particular embodiments, DMSO can be used. Addition of plasma (e.g., to a concentration of 20-25%) can augment the protective effects of DMSO. After addition of DMSO, cells can be kept at 0° C. until freezing, because DMSO concentrations of 1% can be toxic at temperatures above 4° C.

In the cryopreservation of cells, slow controlled cooling rates can be critical and different cryoprotective agents (Rapatz et al., 1968, Cryobiology 5(1): 18-25) and different cell types have different optimal cooling rates (see e.g., Rowe and Rinfret, 1962, Blood 20:636; Rowe, 1966, Cryobiology 3(1):12-18; Lewis, et al., 1967. Transfusion 7(1):17-32; and Mazur, 1970, Science 168:939-949 for effects of cooling velocity on survival of stem cells and on their transplantation potential). The heat of fusion phase where water turns to ice should be minimal. The cooling procedure can be carried out by use of, e.g., a programmable freezing device or a methanol bath procedure. Programmable freezing apparatuses allow determination of optimal cooling rates and facilitate standard reproducible cooling.

In particular embodiments, DMSO-treated cells can be pre-cooled on ice and transferred to a tray containing chilled methanol which is placed, in turn, in a mechanical refrigerator (e.g., Harris or Revco) at −80° C. Thermocouple measurements of the methanol bath and the samples indicate a cooling rate of 1° to 3° C./minute can be preferred. After at least two hours, the specimens can have reached a temperature of −80° C. and can be placed directly into liquid nitrogen (−196° C.).

After thorough freezing, the cells can be rapidly transferred to a long-term cryogenic storage vessel. In a preferred embodiment, samples can be cryogenically stored in liquid nitrogen (−196° C.) or vapor (−1° C.). Such storage is facilitated by the availability of highly efficient liquid nitrogen refrigerators.

Further considerations and procedures for the manipulation, cryopreservation, and long-term storage of cells, can be found in the following exemplary references: U.S. Pat. Nos. 4,199,022; 3,753,357; and 4,559,298; Gorin, 1986, Clinics In Haematology 15(1):19-48; Bone-Marrow Conservation, Culture and Transplantation, Proceedings of a Panel, Moscow, Jul. 22-26, 1968, International Atomic Energy Agency, Vienna, pp. 107-186; Livesey and Linner, 1987, Nature 327:255; Linner et al., 1986, J. Histochem. Cytochem. 34(9):1 123-1 135; Simione, 1992, J. Parenter. Sci. Technol. 46(6):226-32).

Following cryopreservation, frozen cells can be thawed for use in accordance with methods known to those of ordinary skill in the art. Frozen cells are preferably thawed quickly and chilled immediately upon thawing. In particular embodiments, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed on ice.

In particular embodiments, methods can be used to prevent cellular clumping during thawing. Exemplary methods include: the addition before and/or after freezing of DNase (Spitzer et al., 1980, Cancer 45:3075-3085), low molecular weight dextran and citrate, hydroxyethyl starch (Stiff et al., 1983, Cryobiology 20:17-24), etc. [0162] As is understood by one of ordinary skill in the art, if a cryoprotective agent that is toxic to humans is used, it should be removed prior to therapeutic use. DMSO has no serious toxicity.

Exemplary carriers and modes of administration of cells are described at pages 14-15 of U.S. Patent Publication No. 2010/0183564. Additional pharmaceutical carriers are described in Remington: The Science and Practice of Pharmacy, 21 st Edition, David B. Troy, ed., Lippicott Williams & Wilkins (2005).

In particular embodiments, cells can be harvested from a culture medium, and washed and concentrated into a carrier in a therapeutically effective amount. Exemplary carriers include saline, buffered saline, physiological saline, water, Hanks' solution, Ringer's solution. Nonnosol-R (Abbott Labs), Plasma-Lyte A® (Baxter Laboratories, Inc., Morton Grove, IL), glycerol, ethanol, and combinations thereof.

In particular embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, compositions can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within compositions can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In compositions and formulations disclosed herein, cells are generally in a volume of a liter or less, 500 ml or less, 250 ml or less or 100 ml or less. Hence the density of administered cells is typically greater than 10⁴ cells/ml, 10⁷ cells/ml or 10⁸ cells/ml.

Also provided herein are nucleic acid compositions (such as pharmaceutical compositions, also referred to herein as formulations) comprising any of the nucleic acids encoding a TCR and/or CSR and/or SSE described herein. In some embodiments, the nucleic acid composition is a pharmaceutical composition. In some embodiments, the nucleic acid composition further comprises any of an isotonizing agent, an excipient, a diluent, a thickener, a stabilizer, a buffer, and/or a preservative; and/or an aqueous vehicle, such as purified water, an aqueous sugar solution, a buffer solution, physiological saline, an aqueous polymer solution, or RNase free water. The amounts of such additives and aqueous vehicles to be added can be suitably selected according to the form of use of the nucleic acid composition.

The compositions and formulations disclosed herein can be prepared for administration by, for example, injection, infusion, perfusion, or lavage. The compositions and formulations can further be formulated for bone marrow, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous injection.

The formulations to be used for in vivo administration must be sterile. This is readily accomplished by, e.g., filtration through sterile filtration membranes.

### XVII. Dosage and Administration

The dose of the compositions administered to an individual (such as a human) may vary with the particular composition, the mode of administration, and the type of disease being treated. In some embodiments, the amount of the composition is sufficient to result in a complete response in the individual. In some embodiments, the amount of the composition is sufficient to result in a partial response in the individual. In some embodiments, the amount of the composition administered (for example when administered alone) is sufficient to produce an overall response rate of more than about any of 2%, 4%, 6%, 8%, 10%, 15%, 20%, 25%, 30%, 35%, 40%, 45%, 50%, 55%, 60%, 64%, 65%, 70%, 75%, 80%, 85%, or 90% among a population of individuals treated with the composition. Responses of an individual to the treatment of the methods described herein can be determined, for example, based on the percentage tumor growth inhibition (% TGI).

In some embodiments, the amount of the composition is sufficient to prolong overall survival of the individual. In some embodiments, the amount of the composition (for example when administered along) is sufficient to produce clinical benefit of more than about any of 2%, 4%, 6%, 8%, 10%, 15%, 20%, 25%, 30%, 35%, 40%, 45%, 50%, 60%, 70%, or 77% among a population of individuals treated with the composition.

In some embodiments, the amount of the composition is an amount sufficient to decrease the size of a tumor, decrease the number of cancer cells, or decrease the growth rate of a tumor by at least about any of 2%, 4%, 6%, 8%, 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the amount of the composition is below the level that induces a toxicological effect (i.e., an effect above a clinically acceptable level of toxicity) or is at a level where a potential side effect can be controlled or tolerated when the composition is administered to the individual. In some embodiments, the amount of the composition is close to a maximum tolerated dose (MTD) of the composition following the same dosing regimen. In some embodiments, the amount of the composition is more than about any of 80%, 90%, 95%, or 98% of the MTD. In some embodiments, the amount of the composition is included in a range of about 0.001 μg to about 1000 μg. In some embodiments of any of the above aspects, the effective amount of the composition is in the range of about 0.1 μg/kg to about 100 mg/kg of total body weight.

The compositions can be administered to an individual (such as human) via various routes, including, for example, intravenous, intra-arterial, intraperitoneal, intrapulmonary, oral, nasal, inhalation, intravesicular, intramuscular, intra-tracheal, subcutaneous, intraocular, intrathecal, intracranial, intracerebral, intracerebroventricular, transmucosal, and transdermal. In some embodiments, sustained continuous release formulation of the composition may be used. In some embodiments, the composition is administered intravenously. In some embodiments, the composition is administered intraarterially. In some embodiments, the composition is administered intraperitoneally. In some embodiments, the composition is administered intrathecally. In some embodiments, the composition is administered intracranially. In some embodiments, the composition is administered intracerebrally. In some embodiments, the composition is administered intracerebroventricularly. In some embodiments, the composition is administered nasally.

### XVII. Manufacturing

In some embodiments of the invention, there is provided an article of manufacture containing materials useful for the treatment of a target antigen-positive disease such as cancer (for example adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lung cancer, lymphoma, melanoma, mesothelioma, multiple myeloma, pancreatic cancer, pheochromocytoma, plasmacytoma, neuroblastoma, ovarian cancer, prostate cancer, sarcoma, stomach cancer, uterine cancer or thyroid cancer) or viral infection (for example infection by CMV, EBV, HBV, KSHV, HPV, MCV, HTLV-1, HIV-1, or HCV). The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is an immune cell presenting on its surface a TCR and a CSR of the invention. The label or package insert indicates that the composition is used for treating the particular condition. The label or package insert will further comprise instructions for administering the TCR plus CSR immune cell composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a target antigen-positive cancer (such as adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lung cancer, lymphoma, melanoma, mesothelioma, multiple myeloma, pancreatic cancer, pheochromocytoma, plasmacytoma, neuroblastoma, ovarian cancer, prostate cancer, sarcoma, stomach cancer, uterine cancer or thyroid cancer). In other embodiments, the package insert indicates that the composition is used for treating a target antigen-positive viral infection (for example infection by CMV, EBV, HBV, KSHV, HPV, MCV, HTLV-1, HIV-1, or HCV).

Additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a target antigen-positive disease or disorder described herein, optionally in combination with the articles of manufacture. Kits of the invention include one or more containers comprising a TCR plus CSR immune cell composition (or unit dosage form and/or article of manufacture), and in some embodiments, further comprise another agent (such as the agents described herein) and/or instructions for use in accordance with any of the methods described herein. The kit may further comprise a description of selection of individuals suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an immune cell presenting on its surface a TCR and a CSR. In some embodiments, the kit comprises a) a composition comprising an immune cell presenting on its surface a TCR and a CSR, and b) an effective amount of at least one other agent, wherein the other agent increases the expression of MHC proteins and/or enhances the surface presentation of peptides by MHC proteins (e.g., IFNγ, IFNβ, IFNα, or Hsp90 inhibitor). In some embodiments, the kit comprises a) a composition comprising an immune cell presenting on its surface a TCR and a CSR, and b) instructions for administering the TCR plus CSR immune cell composition to an individual for treatment of a target antigen-positive disease (such as cancer or viral infection). In some embodiments, the kit comprises a) a composition comprising an immune cell presenting on its surface a TCR and a CSR, b) an effective amount of at least one other agent, wherein the other agent increases the expression of MHC proteins and/or enhances the surface presentation of peptides by MHC proteins (e.g., IFNγ, IFNβ, IFNα, or Hsp90 inhibitor), and c) instructions for administering the TCR plus CSR immune cell composition and the other agent(s) to an individual for treatment of a target antigen-positive disease (such as cancer or viral infection). The TCR plus CSR immune cell composition and the other agent(s) can be present in separate containers or in a single container. For example, the kit may comprise one distinct composition or two or more compositions wherein one composition comprises the TCR plus CSR immune cell and another composition comprises the other agent.

In some embodiments, the kit comprises a) one or more compositions comprising a TCR and a CSR, and b) instructions for combining the TCR and CSR with immune cells (such as immune cells, e.g., T cells or natural killer cells, derived from an individual) to form a composition comprising the immune cells presenting on their surface the TCR and CSR, and administering the TCR plus CSR immune cell composition to the individual for treatment of a target antigen-positive disease (such as cancer or viral infection). In some embodiments, the kit comprises a) one or more compositions comprising a TCR and a CSR, and b) an immune cell (such as a cytotoxic cell). In some embodiments, the kit comprises a) one or more compositions comprising a TCR and a CSR, b) an immune cell (such as a cytotoxic cell), and c) instructions for combining the TCR and CSR with the immune cell to form a composition comprising the immune cell presenting on its surface the TCR and CSR, and administering the TCR plus CSR immune cell composition to an individual for the treatment of a target antigen-positive disease (such as cancer or viral infection).

In some embodiments, the kit comprises a nucleic acid (or set of nucleic acids) encoding a TCR and a CSR. In some embodiments, the kit comprises a) a nucleic acid (or set of nucleic acids) encoding a TCR and a CSR, and b) a host cell (such as an immune cell) for expressing the nucleic acid (or set of nucleic acids). In some embodiments, the kit comprises a) a nucleic acid (or set of nucleic acids) encoding a TCR and a CSR, and b) instructions for i) expressing the TCR and CSR in a host cell (such as an immune cell, e.g., a T cell), ii) preparing a composition comprising the host cell expressing the TCR and CSR, and iii) administering the composition comprising the host cell expressing the TCR and CSR to an individual for the treatment of a target antigen-positive disease (such as cancer or viral infection). In some embodiments, the host cell is derived from the individual. In some embodiments, the kit comprises a) a nucleic acid (or set of nucleic acids) encoding a TCR and a CSR, b) a host cell (such as an immune cell) for expressing the nucleic acid (or set of nucleic acids), and c) instructions for i) expressing the TCR and CSR in the host cell, ii) preparing a composition comprising the host cell expressing the TCR and CSR, and iii) administering the composition comprising the host cell expressing the TCR and CSR to an individual for the treatment of a target antigen-positive disease (such as cancer or viral infection).

The kits of the invention are in suitable packaging. Suitable packaging includes, but is not limited to, vials, bottles, jars, flexible packaging (e.g. sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the TCR plus CSR immune cell compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of a TCR plus CSR immune cell composition as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the TCR and CSR, and pharmaceutical compositions and instructions for use and packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

### Materials and Methods

### Cell Samples, Cell Lines, Antibodies, TCRs, and CSRs

Various cell lines are used as target cells for various assays testing T cells expressing TCRs with or without co-expressed CSRs and are obtained from the American Type Culture Collection. For example, the cell lines HepG2 (ATCC HB-8065; HLA-A2⁺, AFP⁺, GPC3⁺) and SK-HEP-1 (ATCC HTB-52; HLA-A2⁺, AFP⁻) are used to test T cells expressing anti-AFP/MHC-TCR with or without CSR. The cell line IM9 (ATCC CCL-159; HLA-A2⁺, NY-ESO-1⁺) is used to test T cells expressing anti-NY-ESO-1/MHC-TCR with or without CSR. The cell lines 82.3 (Expasy, CVCL_A7NJ; AFP+; cholangiocarcinoma); and RBE (Expasy, CVCL_4896; AFP+; cholangiocarcinoma) are used to test T cells expressing anti-AFP/MHC-TCR with or without CSR. The cell lines Pa-TU-8988T (DSM ACC 162; KRAS+, MSLN+; pancreatic adenocarcinoma) and AsPC-1 (ATCC CRL-1682; KRAS+, MSLN+, pancreatic adenocarcinoma) can be used to evaluate constructs that target KRAS or MSLN, e.g., for the treatment of pancreatic cancer. The cell lines CFPAC-1 (ATCC CRL-1918; HLA-A2+, MSLN+) and Capan-2 (ATCC HTB-80); HLA-A2+, MSLN+) can be used to evaluate constructs that target MSLN, e.g., for the treatment of pancreatic cancer. The cell line YMB1 (Expasy CVCL_2814; HLA-A2, PSA+, EPCAM+, SLC3A2+, KIAA0368+, CTSB+, may be used to evaluate constructs that target EPCAM, SLC3A2, KTAA0368, or CTSB, e.g., for the treatment of breast cancer. The cell line OVCAR3: (ATCC HTB161; HLA-A0201+ MAGE-A4+, MSLN+, MUC16+, EGFR+, ROR1+, MUC1+, WT1+; ovarian adenocarcinoma) can be used to evaluate constructs that target MAGE-A4, MSLN, MUC16, EGFR, ROR1, MUC1, or WT1, e.g., for the treatment of ovarian cancer. The cell lines COLO 205 (ATCC CCL-222; HLA*A0201, MUC1+, WT1+) and SW480 (ATCC CCL-228; KRAS_G12V+, Tp53+, HLA-A2/A24, EGFR+) can be used to evaluate constructs that target MUC1 or WT1 (COLO 205) or KRAS G12V, p53, or EGFR (SW480), e.g., for the treatment of colon cancer. Cell lines SF7761 (Expasy CVCL_IT45); and SF8628 (Expasy CVCL_IT46); which are brainstem glioma cell lines, can be used to evaluate constructs for the treatment of glioma. Cell line A498 (Expasy CVCL_1056; HLA-A2+, PRAME+, CD70+) can be used to evaluate constructs that target PRAME or CD70, e.g., for the treatment of kidney cancer. Cell line NCIH1755 (ATCC, CRL-5892, non-small cell lung adenocarcinoma; Stage 4, HLA-A0201+MAGE-A4+, EGFR+) can be used to evaluate constructions that target MAGE-A4 or EGFR, e.g., for the treatment of lung cancer. Cell line A375 (ATCC, CRL-1619™, malignant melanoma, HLA-A0201+MAGE-A4+, EGFR+) can be used to evaluate constructs that target MAGE-A4 or EGFR, e.g., for the treatment of melanoma. The cell line OPM2 (Expasy, CVCL_1625, plasma cell myeloma, multiple myeloma, HLA-A0201+ MAGE-A4+, EGFR+) can be used to evaluate constructs that target MAGE-A4 or EGFR, e.g., for the treatment of myeloma. Cell lines are culture using known culture conditions, see, e.g., ATCC entries. For example, cell lines can be cultured in RPMI 1640 or DMEM supplemented with 10% FBS and 2 mM glutamine at 37° C./5% CO₂.

HepG2 is a hepatocellular carcinoma cell line that expresses AFP and GPC3; SK-HEP1 is a liver adenocarcinoma cell line that does not express AFP or GPC3. SK-HEP1-AFP MG was generated by transducing the SK-HEP1 parental cell line with an AFP158 peptide expressing minigene cassette, which results in a high level of cell surface expression of AFP158/HLA-A*02:01 complex in SK-HEP1. SK-HEP1-AFP MG-GPC3 was generated by further transducing the SK-HEP1-AFP MG cell line with an GPC3 expressing cassette, which results in a high level of cell surface expression of AFP158/HLA-A*02:01 complex and GPC3 in SK-HEP1. SK-HEP1-GPC3 is generated by transducing the SK-HEP1 cell line with an GPC3 expressing cassette, which results in a high level of cell surface expression of GPC3 in SK-HEP1.

Antibodies against human or mouse CD3, CD4, CD8, CD28, CCR7, CD45RA or myc tag are purchased from Invitrogen.

Peptides are purchased and synthesized by Elim Biopharma. Peptides are >90% pure. The peptides are dissolved in DMSO or diluted in saline at 10 mg/mL and frozen at −80° C. Biotinylated single chain AFP158/HLA-A*02:01 and control peptides/HLA-A*02:01 complex monomers are generated by refolding the peptides with recombinant HLA-A*02:01 and beta-2 microglobulin (02M). The monomers are biotinylated via the BSP peptide linked to the C-terminal end of HLA-A*02:01 extracellular domain (ECD) by the BirA enzyme. Fluorescence-labelled streptavidin is mixed with biotinylated peptide/HLA-A*02:01 complex monomer to form fluorescence-labelled peptide/HLA-A*02:01 tetramer.

Lentiviruses encoding TCRs or TCR+CSR constructs are produced, for example, by transfection of 293T cells with a lentiviral vector that encodes only TCR or both TCR and CSR, or with two lentiviral vectors, one encoding TCR, one encoding CSR. Examples of various TCR constructs and TCR+CSR constructs (TCR co-expressed with CSR) are disclosed in later examples. Primary human T cells are used for transduction after one-day stimulation with CD3/CD28 beads (Dynabeads®, Invitrogen) in the presence of interleukin-2 (IL-2) at 100 U/ml. Concentrated lentiviruses are applied to T cells in Retronectin- (Takara) coated 6-well plates for 96 hours. In some experiments, primary T cells are mock-transduced (no DNA added) or transduced with lentiviral vectors for seven days.

Transduction efficiencies of the anti-AFP/MHC TCRs (or “anti-AFP TCRs” or “anti-AFP-TCRs”) and anti-AFP/MHC TCR plus anti-GPC3 CSR (or “anti-AFP-TCR+anti-GPC3-CSR”) constructs are assessed by flow cytometry. For anti-AFP TCRs, a biotinylated AFP158/HLA-A*02:01 tetramer (“AFP158 tetramer”) with PE-conjugated streptavidin was used in some experiments. For anti-GPC3 CSR, an anti-myc antibody was used. Repeat flow cytometry analyses are done on day 5 and every 3-4 days thereafter.

Tumor cytotoxicities are assayed by Cytox 96 Non-radioactive LDH Cytotoxicity Assay (Promega). CD3⁺ T cells are prepared from PBMC-enriched whole blood using EasySep Human T Cell Isolation Kit (StemCell Technologies) which negatively depletes CD14, CD16, CD19, CD20, CD36, CD56, CD66b, CD123, glycophorin A expressing cells. Human T cells are activated and expanded with, for example, CD3/CD28 Dynabeads (Invitrogen) according to manufacturer's protocol. Activated T cells (ATC) are cultured and maintained in RPMI 1640 medium with 10% FBS plus 100 U/ml IL-2 and used at day 7-14. Activated T cells (immune cells) and target cells are co-cultured at various effector-to-target ratios (e.g., 2.5:1 or 5:1) for 16-24 hours and assayed for cytotoxicities.

TCR alpha/beta knock outs were generated as follows: sgRNA targeting human TRAC and TRBC locus and Cas9 nuclease V3 (all purchased from Integrated DNA technologies) were combined to form an individual RNP complex. T cells were resuspended in Nucleofectorf® Solution using the Lonza Amaxa® Human T Cell Nucleofector® Kit. Nucleofector® Program T-023 for Nucleofector® I Device was used for electroporation.

### Example 1—Short-Term In Vitro Tumor Cell Killing Assay

A FACS-based assay comparing the short-term killing ability of the various TCR T cells is performed. Effector cells used in this example and the following examples include the following.
1) TCR T cells without CSR;
2) TCR T cells with a CSR that comprises at least the intracellular CD30 costimulatory domain (CD30 IC domain), either with a CD30 transmembrane domain (referred to as “TCR+CD30-CSR T cells”) or a different costimulatory molecule's transmembrane (TM) domain, e.g., CD28 TM (referred to as “TCR+CD28T-CD30-CSR T cells”);
3) TCR T cells with a CSR that comprises at least intracellular CD28 costimulatory domain, typically with a CD28 TM domain (referred to as “TCR+CD28-CSR T cells”);
4) TCR T cells with a CSR that comprises at least intracellular 4-1 BB costimulatory domain, either with a 4-1BB TM domain (referred to as “TCR+41BB-CSR T cells”) or a different costimulatory molecule's TM domain, e.g., CD28 TM (referred to as “TCR+CD28T-41BB-CSR T cells”); and
5) TCR T cells with a CSR that comprises at least intracellular DAP10 costimulatory domain, either with a DAP10 TM domain (referred to as “TCR+DAP10-CSR T cells”) or a different costimulatory molecule's TM domain, e.g., CD28 TM (referred to as “TCR+CD28T-DAP10-CSR T cells”).

Other constructs or more detailed descriptions of constructs/T cells that can be used are disclosed herein, e.g., Example 8.

Activated effector cells and their corresponding target cells are co-cultured at an E:T ratio between 2:1 to 5:1 for 16-24 hours. Specific killing is determined by measuring LDH activity in culture supernatants. Tumor cytotoxicity is assayed by LDH Cytotoxicity Assay (Promega). Human T cells purchased from AllCells are activated and expanded with CD3/CD28 Dynabeads (Invitrogen) according to manufacturer's protocol. Activated T cells (ATC) are cultured and maintained in RPMI 1640 medium with 10% FBS plus 100 U/ml IL-2 and used at day 7-14. The T cells are >99% CD3⁺ by FACS analysis. Activated T cells (Effector cells) and the target cells e.g., HepG2 cells, are co-cultured at a 2:1 to 5:1 ratio 16-24 hours, typically 16 hours. Cytotoxicities are then determined by measuring LDH activities in culture supernatants.

The short-term killing ability of the various TCR T cells is also determined by measuring the amounts/levels of cytokines released from T cells upon engagement with target cells. The levels of cytokine release in the supernatant after 16 hour co-culture are quantified with Luminex Magpix technology using BioRad Bio-Plex kits or with ELISA. T cells with high cytotoxic potency secrete high levels of cytokines that are related to T cell activity, such as TNFα, GM-CSF, IFNγ, and IL-2.

TCR T cells with a CSR comprising at least the CD30 IC domain have higher killing efficacies than corresponding TCR T cells without CSR, and higher than or about the same killing efficacies as corresponding TCR T cells with CSRs that do not have a CD30 IC domain but have a different costimulatory molecule's IC domain, e.g., CD28, 4-1BB, or DAP10's IC domain.

### Example 2—In Vitro T Cell Proliferation and Persistence Assays

The proliferation and persistence of genetically modified T cells are crucial for the success of adoptive T-cell transfer therapies when treating cancers. To assay the effect of the CSR on T-cell proliferation and persistence we label T cells with the intracellular dye CFSE and observe the dilution of the dye as the T cells divide when stimulated with tumor cells. We are also able to measure persistence of the T cells by counting the number of CFSE-positive cells remaining on various days.

Respective T cells are serum starved overnight and labeled with CFSE using CellTrace CFSE (Thermo Fisher C34554). 50,000 to 100,000 T cells are incubated with target cells at an effector cell to target cell ratio (E:T ratio) of 2:1, and flow cytometry is used to observe serial dilution of the CFSE dye as the T cells divide over time. The total number of T cells are counted with FACs.

TCR T cells with a CSR comprising at least the CD30 IC domain proliferate more than corresponding TCR T cells without CSR and proliferate more than or about the same as corresponding TCR T cells with CSRs that do not have a CD30 IC domain but have a different costimulatory molecule's IC domain, e.g., CD28, 4-1BB, or DAP10's IC domain.

### Example 3—Long-Term In Vitro T Cell and Target Cell Counts after Multi-Week Engagements

A FACS based assay for counting T cells and target cells is used to compare the long-term survival and target-cell killing potential of TCR+CD30-CSR T cells with TCR T cells without CSR or with CSRs comprising other costimulatory fragments. Typically, 50,000 to 100,000 T cells are incubated with target cells at an effector cell to target cell ratio (E:T ratio) of 2:1. The cells are rechallenged with target cells on various days, typically every 7 days after the first engagement. The numbers of remaining target cells and total T cells are quantified with FACS on various days after each target cell engagement.

TCR T cells with a CSR comprising at least the CD30 IC domain persist/survive for longer period of time over multiple engagements of tumor target cells and kill more tumor cells than corresponding TCR T cells without CSR do, and survive better and/or kill more tumor cells than or about the same as corresponding TCR T cells with CSRs that do not have a CD30 IC domain but have a different costimulatory molecule's IC domain, e.g., CD28, 4-1BB, or DAP10's IC domain.

### Example 4-Differentiation of T Cell Subsets Over Time (CCR7/CD45RA) and Memory T Cell Quantification

This example shows that TCR+CD30-CSR T cells develop into and maintain a high memory T cell population after target stimulation, including central memory and effector memory T cells. To determine the effect of expressing TCR+CD30-CSR on T cells' ability to develop into and maintain memory T cells as compared to expressing TCR only or TCR co-expressed with a CSR comprising a different costimulatory fragment, e.g., CD28, 4-1BB, or DAP10's IC domain, we measure the cell surface expression of memory T cell markers CCR7 and CD45RA. As known in the field, T cells with high CCR7 expression levels and low CD45RA expression levels are considered as central memory T cells, T cells with low CCR7 and low CD45RA expression levels are effector memory T cells, T cells with low CCR7 and high CD45RA expression levels are effector T cells, while T cells with high CCR7 and high CD45RA are naïve T cells which are the initial type of T cells before target/antigen challenge/recognition (Mahnke et al., Eur J Immunol. 43(11):2797-809, 2013). When in response to antigen encounter, naïve T cells proliferate and differentiate into effector cells, most of which carry out the job of destroying targets and then die, while a small pool of T cells ultimately develops into long-lived memory T cells which can store the T cell immunity against the specific target. Among the memory T cells, the central memory T cells are found to have longer lives than effector memory T cells and be capable of generating effector memory T cells, but not vice versa. Therefore, the ability to develop into and maintain memory T cells, especially central memory T cells, is an important and desired feature for potentially successful T cell therapies.

The effector cells expressing TCR constructs alone are incubated with target cells at an E:T ratio of 2:1 (e.g., 100,000 receptor⁺ T cells and 50,000 target cells in each well on a 96-well plate) for 7 days. The cells are then rechallenged with 50,000-100,000 target cells per well every 7 days.

The TCR+CD30-CSR T cells are incubated with target cells at an E:T ratio of 1:2 (e.g., 25,000 receptor⁺ T cells and 50,000 target cells in each well) for 7 days. The cells are then rechallenged with 50,000-100,000 target cells per well every 7 days.

Each different T cell and target cell mixture sample is made in replicates to ensure at least one mixture to be available for quantification on each selected day. The TCR+CD30-CSR T cell and target cell mixtures are diluted 1:6 before the fourth and fifth target cell engagement (E4 and E5) to avoid the overcrowdedness of T cells due to the significant T cell expansion, so that only one sixth of the previously remaining cells are rechallenged with 50,000-100,000 target cells.

On selected days after each target cell engagement, the entire cell mixture in a well from each sample is stained with antibodies against CCR7 and CD45RA and analyzed by flow cytometry. Receptor⁺ T cell numbers are counted, and cells are grouped into various T cell types based on their CCR7 and CD45RA expression levels: central memory T cells (CD45RA⁻ CCR7⁺), effector memory T cells (CD45RA⁻ CCR7⁻), effector T cells (CD45RA⁺ CCR7⁻), and naïve T cells (CD45RA⁺ CCR7⁺). Percentages of various types of T cells among the total number of receptor⁺ T cells are calculated. In some experiments, the cells are also stained with antibodies against CD8 or CD4 to determine the CD8-CD4 characteristics of the counted T cells.

Proliferation and survival of TCR+CD30-CSR T cells is measured before and after target cell engagement in two independent flow cytometric assays. FACS analysis of TCR+CD30-CSR T cells shows a greater level of expression of the T cell differentiation markers CCR7 and CD45RA compared to TCR+CD28 (or other costimulatory domain)-CSR T cells prior to target engagement.

TCR T cells with a CSR comprising at least the CD30 IC domain are able to develop into and maintain high numbers and high percentages of central memory T cells upon engagement with target calls, higher than T cells expressing TCR alone or co-expressing TCR and a CSR that does not have a CD30 IC domain but has a different costimulatory molecule's IC domain, e.g., CD28, 4-1BB, or DAP10's IC domain.

### Example 5—Expression of T Cell Exhaustion Markers in T Cells after Co-Culture with Target Cells

Molecules such as PD-1, LAG3, TIM-3, and TIGIT are inhibitory receptors that accumulate on T cells as T cells lose function. Because of this phenomenon these molecules' expression is seen as a marker of exhausted T cells. To examine the level of exhaustion markers expressed on TCR+CSR-transduced cells upon antigen stimulation, CD3 T cells are prepared from PBMC-enriched whole blood using EasySep Human T Cell Isolation Kit (StemCell Technologies) and activated with CD3/CD28 Dynabeads as above. The activated and expanded cell population is >99% CD3⁺ by flow cytometry. These cells are then transduced with lentiviral vectors encoding a TCR+CD30-CSR, +other CSR, or no CSR for 7-9 days. The transduced T cells (effector cells) are co-cultured with target cells for 16 hours at an effector-to-target ratio in the range of 1:1 to 2.5:1. Using antibodies to exhaustion marker PD-1, LAG3, TIGIT, or TIM-3, the levels of exhaustion markers. e.g., MFI levels, on the transduced T cells are analyzed by flow cytometry. In some experiments, the cells are incubated for longer times and rechallenged with target cells every 7 days, and exhaustion marker levels are measured on selected days after each target cell engagement.

Over a series of target cell engagements, TCR T cells with a CSR comprising at least the CD30 IC domain have lower levels of T cell exhaustion markers than corresponding TCR T cells without CSR do, and have lower levels of T cell exhaustion markers than corresponding TCR T cells with CSRs that do not have a CD30 IC domain but have a different costimulatory molecule's IC domain, e.g., CD28, 4-1BB, or DAP10's IC domain.

### Example 6—In Vivo Tumor Infiltration/Penetration by T Cells

About 10⁷ tumor cells used for an animal model, e.g., HepG2 cells for liver cancer animal model, are implanted subcutaneously in NSG mice and allowed to form a solid tumor, e.g., a solid tumor with the mass of about 150 mm³. About 5×10⁶ various TCR T cells (e.g., TCR only, TCR+CD30 CSR, TCR+CD28-CSR, TCR+DAP10-CSR, TCR+4-1BB-CSR, or TCR+other costimulatory domain-CSR T cells) are injected i.v. into the tumor bearing mice. 3 weeks after T-cell dosing, the mice are sacrificed and tumors removed, fixed and sectioned onto slides. Tumor sections are stained with an anti-CD3 antibody to visualize the T cells that are present within the solid tumor. Quantification of the number of CD3⁺ cells can be used to score the tumor infiltration ability of the T cells (T-cell/mm²) TCR T cells with a CSR comprising at least the CD30 IC domain have higher in vivo tumor infiltration/penetration rates/levels (i.e., higher numbers of T cells/mm²) than corresponding TCR T cells without CSR or corresponding TCR T cells with CSRs that do not have a CD30 IC domain but have a different costimulatory molecule's IC domain, e.g., CD28, 4-1BB, or DAP10's IC domain.

### Example 7—Tumor Infiltrating Lymphocyte (Til) Engineering and Testing

In this example, tumor infiltrating lymphocytes (TILs) are isolated and then engineered to express CSRs comprising CD30 or other costimulatory domains. TILs expressing CSRs comprising at least the CD30 IC domain have increased tumor infiltration/penetration rates/levels.

TILs in Animal Model

About 10⁷ tumor cells of various cancer types, e.g., HepG2 cells (AFP+GPC3+) from liver cancer, are implanted subcutaneously in NSG mice and allowed to form a solid tumor, e.g., a solid tumor with the mass of about 150 mm³. Then TILs are generated using various methods including the following three:
(1) 5×10⁶ T cells isolated from healthy human PBMCs are injected i.v. into each tumor-bearing mouse. Three weeks after T cell dosing, the mice are sacrificed and tumors removed, and TILs, in particular tumor infiltrating T cells (CD3+ cells) are isolated (e.g., with the method described in Gros et al., J Clin Invest. 129(11):4992-5004, 2019). TIL T cells are cultured and mock-transduced or transduced with vectors encoding CSRs comprising CD30 or other costimulatory domains, e.g., those described in Example 8. Then the engineered TIL T cells are re-introduced into new NSG mice bearing HepG2 tumors but not exposed to human T cells. Quantification of the number of CD3+ cells (i.e., T cells) can be used to score the tumor infiltration ability of the various T cells (T-cell/mm²).
(2) 5×10⁶ anti-AFP/MHC CAR T cells (e.g., as described in WO2016/161390) are injected i.v. into each tumor-bearing mouse. For example, the anti-AFP/MHC CAR T cells can comprise the antibody moiety comprises the CDRs or variable domains (V_{H} and/or V_{L} domains) of an antibody moiety specific for AFP (e.g., (i) V_{H} domain comprising, consisting essentially of, or consisting of the amino acid sequence of SEQ ID NO:295 and/or V_{L} domain comprising, consisting essentially of or consisting of the amino acid sequence of SEQ ID NO:296, or CDRs contained therein; (ii) V_{H} domain comprising, consisting essentially of, or consisting of the amino acid sequence of SEQ ID NO:297 and/or V_{L} domain comprising, consisting essentially of, or consisting of the amino acid sequence of SEQ ID NO:298, or CDRs contained therein; (iii) V_{H} domain comprising, consisting essentially of, or consisting of the amino acid sequence of SEQ ID NO:299 and/or V_{L} domain comprising, consisting essentially of, or consisting of the amino acid sequence of SEQ ID NO:300, or CDRs contained therein; (iv) V_{H} domain comprising, consisting essentially of, or consisting of the amino acid sequence of SEQ ID NO:301 and/or V_{L} domain comprising, consisting essentially of, or consisting of the amino acid sequence of SEQ ID NO:302, or CDRs contained therein; or (v) V_{H} domain comprising, consisting essentially of, or consisting of the amino acid sequence of SEQ ID NO:303 and/or V_{L} domain comprising, consisting essentially of, or consisting of the amino acid sequence of SEQ ID NO:304, or CDRs contained therein). Exemplary anti-AFP/MHC CAR can comprise a scFv comprising any pair of the V_{H} and V_{L} variable region sequences described above, a CD28 TM and costimulatory domain, and a CD3ζ signaling domain. Three weeks after T cell dosing, the mice are sacrificed and tumors removed, and TILs, in particular tumor infiltrating T cells are isolated (e.g., with the method described in Gros et al., J Clin Invest. 129(11):4992-5004, 2019). TIL T cells are cultured and mock-transduced or transduced with vectors encoding CSRs comprising CD30 or other costimulatory domains, e.g., those described in Example 8. Then the engineered TIL T cells are re-introduced into new NSG mice bearing SK-HEP1-GPC3 tumors (AFP− GPC3+, generated by injecting SK-HEP1-GPC3 cells into NSG mice) but not exposed to anti-AFP/MHC CAR T cells. Quantification of the number of CD3+ cells (i.e., T cells) can be used to score the tumor infiltration ability of the various T cells (T cell/mm²).
(3) 5×10⁶ anti-AFP/MHC TCR T cells (e.g., as described herein) are injected i.v. into each tumor-bearing mouse. Three weeks after T cell dosing, the mice are sacrificed and tumors removed, and TILs, in particular tumor infiltrating T cells are isolated (e.g., with the method described in Gros et al., J. Clin Invest. 129(11):4992-5004, 2019). TIL T cells are cultured and mock-transduced or transduced with vectors encoding CSRs comprising CD30 or other costimulatory domains, e.g., those described in Example 8. Then the engineered TIL T cells are re-introduced into new NSG mice bearing SK-HEP1-GPC3 tumors (AFP− GPC3+) but not exposed to anti-AFP/MHC TCR T cells. Quantification of the number of CD3+ cells (i.e., T cells) can be used to score the tumor infiltration ability of the various T cells (T cell/mm²).

TIL T cells with a CSR comprising at least the CD30 IC domain have higher in vivo tumor infiltration/penetration rates/levels (i.e., higher numbers of T cells/mm²) than corresponding TILs without CSR or corresponding TILs with CSRs that do not have a CD30 IC domain but have a different costimulatory molecule's IC domain, e.g., CD28, 4-1BB, or DAP10's IC domain.

TILs for Treating Human

TIL T cells are isolated from human patient tumor specimen (e.g., with the method described in Gros et al., J Clin Invest. 129(11):4992-5004, 2019) and cultured to grow to sufficient numbers. The TIL T cells are then transduced with vectors encoding CSRs comprising CD30 (e.g., those described in Example 8) and infused back to the patient. In clinical trials, the TIL T cells are also mock-transduced or transduced with vectors encoding CSRs comprising other costimulatory domains (e.g., those described in Example 8) as the controls. For liver cancer patients, vectors encoding anti-GPC3 CD30 CSRs, with at least the CD30 IC domain, are used to transduce human TIL T cells and infused back to the patients for the treatment of liver cancer. TIL T cells with a CSR comprising at least the CD30 IC domain have higher in vivo tumor infiltration/penetration rates/levels (i.e., higher numbers of T cells/mm2) than corresponding TIL T cells without CSR or corresponding TIL T cells with CSRs that do not have a CD30 IC domain but have a different costimulatory molecule's IC domain, e.g., CD28, 4-1BB, or DAP10's IC domain. Thus, TIL T cells engineered to express CD30 CSRs can treat cancer patients effectively, especially patients with solid tumors. e.g., liver cancer or other cancers shown in Table 2 or other sections of the current disclosure.

### Example 8—Exemplary Constructs

Nucleic acids encoding the following constructs are made. Representative amino acid sequences of the components/domains/regions of the CSRs and TCRs disclosed in this example are shown in the Informal Sequence Listing and/or in the references cited in the current specification, including various TCR variable regions (CDRs and complete variable regions), TCR constant regions, TCR transmembrane and cytoplasmic regions, various CSR antibody moieties (including CDRs, complete variable regions, and scFv fragments), various CSR transmembrane domains and intracellular costimulatory domains. The CSRs disclosed herein can comprise a myc tag between the scFv and transmembrane domains (for in vitro expression detection) or not (for come clinical uses). There can be an antibody constant region present in some embodiments of CSR, between the antibody variable region (e.g., in the form of an scFv) and the CSR transmembrane domain. When co-expressed, the TCRs and the CSRs can be expressed from the same cloning vector or different vectors.

For Liver Cancers Including HCC:
Constructs: anti-GPC3 CSRs comprising CD30, CD28, 4-1BB, or DAP10 costimulatory fragments and anti-AFP TCRs co-expressed with such anti-GPC3 CSRs. An anti-GPC3 CSR can comprise an anti-GPC3 scFv.
Construct: anti-GPC3-CD30-CSR: a CSR comprising anti-GPC3 scFv extracellular (EC), CD30 transmembrane (TM), and CD30 intracellular (IC) domains
Construct: anti-GPC3-CD28T-CD30-CSR: a CSR comprising anti-GPC3 scFv EC, CD28 TM, and CD30 IC
Construct: anti-GPC3-CD8T-CD30-CSR: a CSR comprising anti-GPC3 scFv EC, CD8 TM, and CD30 IC
Construct: anti-GPC3-CD27T-CD30-CSR: a CSR comprising anti-GPC3 scFv EC, CD27 TM, and CD30 IC
Construct: anti-GPC3-OX40T-CD30-CSR: a CSR comprising anti-GPC3 scFv EC, OX40 TM, and CD30 IC
Construct: anti-GPC3-41BBT-CD30-CSR: a CSR comprising anti-GPC3 scFv EC, 4-1BB TM, and CD30 IC
Construct: anti-GPC3-CD28-CSR: a CSR comprising anti-GPC3 scFv EC, CD28 TM, and CD28 IC Construct: anti-GPC3-CD28T-41BB-CSR: a CSR comprising anti-GPC3 scFv EC, CD28 TM, and 4-1BB IC
Construct: αGPC3-CD28T-DAP10-CSR a CSR comprising anti-GPC3 scFv EC, CD28 TM, and DAP10 IC
Construct: αGPC3-CD30T-OX40-CSR: a CSR comprising anti-GPC3 scFv EC, CD30 TM, and OX40 IC
Construct: αGPC3-CD30T-CD27-CSR: a CSR comprising anti-GPC3 scFv EC, CD30 TM, and CD27 IC
Construct: anti-GPC3-CD27-CSR: a CSR comprising anti-GPC3 scFv EC, CD27 TM, and CD27 IC
Construct: anti-GPC3-OX40-CSR: a CSR comprising anti-GPC3 scFv EC, OX40 TM, and OX40 IC
Construct: anti-GPC3-41BB-CSR: a CSR comprising anti-GPC3 scFv EC, 4-1BB TM, and 4-1BB IC
Construct: αGPC3-DAP10-CSR: a CSR comprising anti-GPC3 scFv EC, DAP10 TM, and DAP10 IC
Construct: anti-AFP-TCR1 (a.k.a. anti-AFP-TCR1 or anti-AFP/MHC-TCR1): alpha beta TCR pair/chains comprising anti-AFP/MHC TCR1 binding domains and alpha beta TCR transmembrane (TM) and intracellular (IC) domains, without CSR.
Construct: anti-AFP-TCR1+anti-GPC3-CD30-CSR-anti-AFP-TCR1 co-expressed with a anti-GPC3-CD30-CSR
Construct: anti-AFP-TCR1+anti-GPC3-CD8T-CD30-CSR
Construct: anti-AFP-TCR1+anti-GPC3-CD28T-CD30-CSR
Construct: anti-AFP-TCR1+anti-GPC3-CD28-CSR
Construct: anti-AFP-TCR1+anti-GPC3-CD28T-41BB-CSR
Construct: anti-AFP-TCR1+anti-GPC3-CD28T-DAP10-CSR
Construct: anti-AFP-TCR2 (a.k.a. anti-AFP-TCR2 or anti-AFP/MHC-TCR2): alpha beta TCR pair/chains comprising anti-AFP/MHC TCR2 binding domains and alpha beta TCR TM and IC domains, without CSR.
Construct: anti-AFP-TCR2+anti-GPC3-CD30-CSR: anti-AFP-TCR2 co-expressed with a anti-GPC3-CD30-CSR
Construct: anti-AFP-TCR2+anti-GPC3-CD8T-CD30-CSR
Construct: anti-AFP-TCR2+anti-GPC3-CD28T-CD30-CSR
Construct: anti-AFP-TCR2+anti-GPC3-CD28-CSR
Construct: anti-AFP-TCR2+anti-GPC3-CD28T-41BB-CSR
Construct: anti-AFP-TCR2+anti-GPC3-CD28T-DAP10-CSR
Constructs: anti-MSLN CSRs comprising CD30, CD28, 4-1BB, or DAP10 costimulatory fragments and anti-MSLN TCR co-expressed with such anti-MSLN CSRs. An anti-MSLN CSR can comprise an anti-MSLN-scFv.
Construct: anti-MSLN-CD30-CSR: a CSR comprising anti-MSLN scFv extracellular (EC). CD30 transmembrane (TM), and CD30 intracellular (IC) domains
Construct: anti-MSLN-CD28T-CD30-CSR: a CSR comprising anti-MSLN scFv EC, CD28 TM, and CD30 IC
Construct: anti-MSLN-CD8T-CD30-CSR: a CSR comprising anti-MSLN scFv EC, CD8 TM, and CD30 IC
Construct: anti-MSLN-CD27T-CD30-CSR: a CSR comprising anti-MSLN scFv EC, CD27 TM, and CD30 IC
Construct: anti-MSLN-OX40T-CD30-CSR: a CSR comprising anti-MSLN scFv EC, OX40 TM, and CD30 IC
Construct: anti-MSLN-41BBT-CD30-CSR: a CSR comprising anti-MSLN scFv EC, 4-1BB TM, and CD30 IC
Construct: anti-MSLN-CD28-CSR: a CSR comprising anti-MSLN scFv EC, CD28 TM, and CD28 IC
Construct: anti-MSLN-CD28T-41BB-CSR: a CSR comprising anti-MSLN scFv EC, CD28 TM, and 4-1BB IC
Construct: anti-MSLN-CD28T-DAP10-CSR: a CSR comprising anti-MSLN scFv EC, CD28 TM, and DAP10 IC
Construct: anti-MSLN-CD27-CSR: a CSR comprising anti-MSLN scFv EC, CD27 TM, and CD27 IC
Construct: anti-MSLN-OX40-CSR: a CSR comprising anti-MSLN scFv EC, OX40 TM, and OX40 IC
Construct: anti-MSLN-41BB-CSR: a CSR comprising anti-MSLN scFv EC, 4-1BB TM, and 4-1BB IC
Construct: anti-MSLN-DAP10-CSR a CSR comprising anti-MSLN scFv EC, DAP10 TM, and DAP10 IC
Construct: anti MSLN-TCR construct: (a.k.a. anti-MSLN-TCR or anti-MSLN/MHC-TCR): alpha beta TCR pair/chains comprising anti-MSLN/MHC TCR binding domains and alpha beta TCR transmembrane (TM) and intracellular (IC) domains, without CSR
Construct: anti-MSLN-TCR+anti-MSLN-CD30-CSR: anti-MSLN-TCR co-expressed with a anti-MSLN-CD30-CSR
Construct: anti-MSLN-TCR+anti-MSLN-CD8T-CD30-CSR
Construct: anti-MSLN-TCR+anti-MSLN-CD28T-CD30-CSR
Construct: anti-MSLN-TCR+anti-MSLN-CD28-CSR
Construct: anti-MSLN-TCR+anti-MSLN-CD28T-41BB-CSR
Construct: anti-MSLN-TCR+anti-MSLN-CD28T-DAP10-CSR

For Pancreatic Cancer:
Constructs: anti-MSLN CSRs comprising CD30, CD28, 4-1BB, or DAP10 costimulatory fragments and anti-KRAS, anti-MSLN, or anti-p53 TCRs co-expressed with such anti-MSLN CSRs. An anti-MSLN CSR can comprise an anti-MSLN-scFv.
Construct: anti-MSLN-CD30-CSR: a CSR comprising anti-MSLN scFv extracellular (EC), CD30 transmembrane (TM), and CD30 intracellular (IC) domains
Construct: anti-MSLN-CD28T-CD30-CSR: a CSR comprising anti-MSLN scFv EC, CD28 TM, and CD30 IC
Construct: anti-MSLN-CD8T-CD30-CSR: a CSR comprising anti-MSLN scFv EC, CD8 TM, and CD30 IC
Construct: anti-MSLN-CD27T-CD30-CSR: a CSR comprising anti-MSLN scFv EC, CD27 TM, and CD30 IC
Construct: anti-MSLN-OX40T-CD30-CSR: a CSR comprising anti-MSLN scFv EC, OX40 TM, and CD30 IC
Construct: anti-MSLN-41BBT-CD30-CSR: a CSR comprising anti-MSLN scFv EC, 4-1BB TM, and CD30 IC
Construct: anti-MSLN-CD28-CSR: a CSR comprising anti-MSLN scFv EC, CD28 TM, and CD28 IC
Construct: anti-MSLN-CD28T-41BB-CSR: a CSR comprising anti-MSLN scFv EC, CD28 TM, and 4-1BB IC
Construct: anti-MSLN-CD28T-DAP10-CSR a CSR comprising anti-MSLN scFv EC, CD28 TM, and DAP10 IC
Construct: anti-MSLN-CD27-CSR: a CSR comprising anti-MSLN scFv EC, CD27 TM, and CD27 IC
Construct: anti-MSLN-OX40-CSR: a CSR comprising anti-MSLN scFv EC, OX40 TM, and OX40 IC
Construct: anti-MSLN-41BB-CSR: a CSR comprising anti-MSLN scFv EC, 4-1BB TM, and 4-1BB IC
Construct: anti-MSLN-DAP10-CSR: a CSR comprising anti-MSLN scFv EC, DAP10 TM, and DAP10 IC
Constructs: anti-ROR1 CSRs comprising CD30, CD28, 4-1BB, or DAP10 costimulatory fragments and anti-KRAS, anti-p53, or anti-MSLN TCRs co-expressed with such anti-ROR1 CSRs. An anti-ROR1 CSR can comprise an anti-ROR1-scFv.
Construct: anti-ROR1-CD30-CSR: a CSR comprising anti-ROR1 scFv extracellular (EC), CD30 transmembrane (TM), and CD30 intracellular (IC) domains
Construct: anti-ROR1-CD28T-CD30-CSR: a CSR comprising anti-ROR1 scFv EC, CD28 TM, and CD30 IC
Construct: anti-ROR1-CD8T-CD30-CSR: a CSR comprising anti-ROR1 scFv EC, CD8 TM, and CD30 IC
Construct: anti-ROR1-CD27T-CD30-CSR: a CSR comprising anti-ROR1 scFv EC, CD27 TM, and CD30 IC
Construct: anti-ROR1-OX40T-CD30-CSR: a CSR comprising anti-ROR1 scFv EC, OX40 TM, and CD30 IC
Construct: anti-ROR1-41BBT-CD30-CSR: a CSR comprising anti-ROR1 scFv EC, 4-1BB TM, and CD30 IC
Construct: anti-ROR1-CD28-CSR: a CSR comprising anti-ROR1 scFv EC, CD28 TM, and CD28 IC
Construct: anti-ROR1-CD28T-41BB-CSR: a CSR comprising anti-ROR1 scFv EC, CD28 TM, and 4-1BB IC
Construct: anti-ROR1-CD28T-DAP10-CSR a CSR comprising anti-ROR1 scFv EC, CD28 TM, and DAP10 IC
Construct: anti-ROR1-CD27-CSR: a CSR comprising anti-ROR1 scFv EC, CD27 TM, and CD27 IC
Construct: anti-ROR1-OX40-CSR: a CSR comprising anti-ROR1 scFv EC, OX40 TM, and OX40 IC
Construct: anti-ROR1-41BB-CSR: a CSR comprising anti-ROR1 scFv EC, 4-1BB TM, and 4-1BB IC
Construct: anti-ROR1-DAP10-CSR: a CSR comprising anti-ROR1 scFv EC, DAP10 TM, and DAP10 IC
Construct: anti-KRAS-TCR (a.k.a. anti-KRAS-TCR or anti-KRAS/MHC-TCR): alpha beta TCR pair/chains comprising anti-KRAS/MHC TCR binding domains and alpha beta TCR transmembrane (TM) and intracellular (IC) domains, without CSR.
Construct: anti-KRAS-TCR+anti-MSLN-CD30-CSR anti-KRAS-TCR co-expressed with a anti-MSLN-CD30-CSR
Construct: anti-KRAS-TCR+anti-MSLN-CD8T-CD30-CSR
Construct: anti-KRAS-TCR+anti-MSLN-CD28T-CD30-CSR
Construct: anti-KRAS-TCR+anti-MSLN-CD28-CSR
Construct: anti-KRAS-TCR+anti-MSLN-CD28T-41BB-CSR
Construct: anti-KRAS-TCR+anti-MSLN-CD28T-DAP10-CSR
Construct: anti-KRAS-TCR+anti-ROR1-CD30-CSR: anti-KRAS-TCR co-expressed with a anti-ROR1-CD30-CSR
Construct: anti-KRAS-TCR+anti-ROR1-CD8T-CD30-CSR
Construct: anti-KRAS-TCR+anti-ROR1-CD28T-CD30-CSR
Construct: anti-KRAS-TCR+anti-ROR1-CD28-CSR
Construct: anti-KRAS-TCR+anti-ROR1-CD28T-41BB-CSR
Construct: anti-KRAS-TCR+anti-ROR1-CD28T-DAP10-CSR
Construct: anti-p53-TCR (a.k.a. anti-p53-TCR or anti-p53/MHC-TCR): alpha beta TCR pair/chains comprising anti-p53/MHC TCR binding domains and alpha beta TCR transmembrane (TM) and intracellular (IC) domains, without CSR.
Construct: anti-p53-TCR+anti-MSLN-CD30-CSR: anti-p53-TCR co-expressed with a anti-MSLN-CD30-CSR
Construct: anti-p53-TCR+anti-MSLN-CD8T-CD30-CSR
Construct: anti-p53-TCR+anti-MSLN-CD28T-CD30-CSR
Construct: anti-p53-TCR+anti-MSLN-CD28-CSR
Construct: anti-p53-TCR+anti-MSLN-CD28T-41BB-CSR
Construct: anti-p53-TCR+anti-MSLN-CD28T-DAP10-CSR
Construct: anti-p53-TCR+anti-ROR1-CD30-CSR: anti-p53-TCR co-expressed with a anti-ROR1-CD30-CSR
Construct: anti-p53-TCR+anti-ROR1-CD8T-CD30-CSR
Construct: anti-p53-TCR+anti-ROR1-CD28T-CD30-CSR
Construct: anti-p53-TCR+anti-ROR1-CD28-CSR
Construct: anti-p53-TCR+anti-ROR1-CD28T-41BB-CSR
Construct: anti-p53-TCR+anti-ROR1-CD28T-DAP10-CSR
Construct: anti-MSLN-TCR (a.k.a. anti-MSLN-TCR or anti-MSLN/MHC-TCR): alpha beta TCR pair/chains comprising anti-MSLN/MHC TCR binding domains and alpha beta TCR transmembrane (TM) and intracellular (IC) domains, without CSR.
Construct: anti-MSLN-TCR+anti-MSLN-CD30-CSR: anti-MSLN-TCR co-expressed with a anti-MSLN-CD30-CSR
Construct: anti-MSLN-TCR+anti-MSLN-CD8T-CD30-CSR
Construct: anti-MSLN-TCR+anti-MSLN-CD28T-CD30-CSR
Construct: anti-MSLN-TCR+anti-MSLN-CD28-CSR
Construct: anti-MSLN-TCR+anti-MSLN-CD28T-41BB-CSR
Construct: anti-MSLN-TCR+anti-MSLN-CD28T-DAP10-CSR
Construct: anti-MSLN-TCR+anti-ROR1-CD30-CSR: anti-MSLN-TCR co-expressed with a anti-ROR1-CD30-CSR
Construct: anti-MSLN-TCR+anti-ROR1-CD8T-CD30-CSR
Construct: anti-MSLN-TCR+anti-ROR1-CD28T-CD30-CSR
Construct: anti-MSLN-TCR+anti-ROR1-CD28-CSR
Construct: anti-MSLN-TCR+anti-ROR1-CD28T-41BB-CSR
Construct: anti-MSLN-TCR+anti-ROR1-CD28T-DAP10-CSR

For Prostate Cancer:
Constructs: anti-PSMA CSRs comprising CD30, CD28, 4-1BB, or DAP10 costimulatory fragments and anti-PSA TCRs co-expressed with such anti-PSMA CSRs. An anti-PSMA CSR can comprise an anti-PSMA scFv.
Construct: anti-PSMA-CD30-CSR: a CSR comprising anti-PSMA scFv extracellular (EC), CD30 transmembrane (TM), and CD30 intracellular (IC) domains
Construct: anti-PSMA-CD28T-CD30-CSR: a CSR comprising anti-PSMA scFv EC, CD28 TM, and CD30 IC
Construct: anti-PSMA-CD8T-CD30-CSR: a CSR comprising anti-PSMA scFv EC, CD8 TM, and CD30 IC
Construct: anti-PSMA-CD27T-CD30-CSR: a CSR comprising anti-PSMA scFv EC, CD27 TM, and CD30 IC
Construct: anti-PSMA-OX40T-CD30-CSR: a CSR comprising anti-PSMA scFv EC, OX40 TM, and CD30 IC
Construct: anti-PSMA-41BBT-CD30-CSR: a CSR comprising anti-PSMA scFv EC, 4-1BB TM, and CD30 IC
Construct: anti-PSMA-CD28-CSR: a CSR comprising anti-PSMA scFv EC, CD28 TM, and CD28 IC
Construct: anti-PSMA-CD28T-41BB-CSR: a CSR comprising anti-PSMA scFv EC, CD28 TM, and 4-1BB IC
Construct: αPSMA-CD28T-DAP10-CSR: a CSR comprising anti-PSMA scFv EC, CD28 TM, and DAP10 IC
Construct: anti-PSMA-CD27-CSR: a CSR comprising anti-PSMA scFv EC, CD27 TM, and CD27 IC
Construct: anti-PSMA-OX40-CSR: a CSR comprising anti-PSMA scFv EC, OX40 TM, and OX40 IC
Construct: anti-PSMA-41BB-CSR: a CSR comprising anti-PSMA scFv EC, 4-1BB TM, and 4-1BB IC
Construct: anti-PSMA-DAP10-CSR: a CSR comprising anti-PSMA scFv EC, DAP10 TM, and DAP10 IC
Constructs: anti-ROR1 CSRs comprising CD30, CD28, 4-1BB, or DAP10 costimulatory fragments (as disclosed above under “For pancreatic cancer”) and anti-PSA TCRs co-expressed with such anti-ROR1 CSRs. An anti-ROR1 CSR can comprise an anti-ROR1 scFv.
Construct: anti-PSA-TCR (a.k.a. anti-PSA-TCR or anti-PSA/MHC-TCR): alpha beta TCR pair/chains comprising anti-PSA/MHC TCR binding domains and alpha beta TCR transmembrane (TM) and intracellular (IC) domains, without CSR.
Construct: anti-PSA-TCR+anti-PSMA-CD30-CSR: anti-PSA-TCR co-expressed with a anti-PSMA-CD30-CSR
Construct: anti-PSA-TCR+anti-PSMA-CD8T-CD30-CSR
Construct: anti-PSA-TCR+anti-PSMA-CD28T-CD30-CSR
Construct: anti-PSA-TCR+anti-PSMA-CD28-CSR
Construct: anti-PSA-TCR+anti-PSMA-CD28T-41BB-CSR
Construct: anti-PSA-TCR+anti-PSMA-CD28T-DAP10-CSR
Construct: anti-PSA-TCR+anti-ROR1-CD30-CSR: anti-PSA-TCR co-expressed with a anti-ROR1-CD30-CSR
Construct: anti-PSA-TCR+anti-ROR1-CD8T-CD30-CSR
Construct: anti-PSA-TCR+anti-ROR1-CD28T-CD30-CSR
Construct: anti-PSA-TCR+anti-ROR1-CD28-CSR
Construct: anti-PSA-TCR+anti-ROR1-CD28T-41BB-CSR
Construct: anti-PSA-TCR+anti-ROR1-CD28T-DAP10-CSR

For Melanoma or Gastrointestinal Cancers:
Constructs: anti-ROR2 CSRs comprising CD30, CD28, 4-1BB, or DAP10 costimulatory fragments and various TCRs (e.g., those targeting COL18A1, SRPX, KIF16B, TFDP2, KIAA1279, XPNPEP1, UGGT2, PHKA1, KIF16B, SON, GNB5, FBXO21, CORO7, RECQL5, TFDP2, KIAA1967, KIF16B, MAGEA6, PDS5A, MED13, ASTN1, CDK4, MLL2, SMARCD3, NY-ESO-1, or PRAME as listed in Table 2) co-expressed with such anti-ROR2 CSRs. An anti-ROR2 CSR can comprise an anti-ROR2 scFv.
Constructs: anti-ROR2 CSRs comprising CD30, CD28, 4-1BB, or DAP10 costimulatory fragments and various TCRs (e.g., those targeting NUP98, GPD2, CASP8, KRAS, SKIV2L, H3F3B, RAD21, or PRAME as listed in Table 2) co-expressed with such anti-ROR2 CSRs. An anti-ROR2 CSR can comprise an anti-ROR2 scFv.
Construct: anti-ROR2-CD30-CSR: a CSR comprising anti-ROR2 scFv extracellular (EC), CD30 transmembrane (TM), and CD30 intracellular (IC) domains
Construct: anti-ROR2-CD28T-CD30-CSR: a CSR comprising anti-ROR2 scFv EC, CD28 TM, and CD30 IC
Construct: anti-ROR2-CD8T-CD30-CSR: a CSR comprising anti-ROR2 scFv EC, CD8 TM, and CD30 IC
Construct: anti-ROR2-CD27T-CD30-CSR: a CSR comprising anti-ROR2 scFv EC, CD27 TM, and CD30 IC
Construct: anti-ROR2-OX40T-CD30-CSR: a CSR comprising anti-ROR2 scFv EC, OX40 TM, and CD30 IC
Construct: anti-ROR2-41BBT-CD30-CSR: a CSR comprising anti-ROR2 scFv EC, 4-1BB TM, and CD30 IC
Construct: anti-ROR2-CD28-CSR: a CSR comprising anti-ROR2 scFv EC, CD28 TM, and CD28 IC
Construct: anti-ROR2-CD28T-41BB-CSR: a CSR comprising anti-ROR2 scFv EC, CD28 TM, and 4-1BB IC
Construct: anti-ROR2-CD28T-DAP10-CSR: a CSR comprising anti-ROR2 scFv EC, CD28 TM, and DAP10 IC
Construct: anti-ROR2-CD27-CSR: a CSR comprising anti-ROR2 scFv EC, CD27 TM, and CD27 IC
Construct: anti-ROR2-OX40-CSR: a CSR comprising anti-ROR2 scFv EC, OX40 TM, and OX40 IC
Construct: anti-ROR2-41BB-CSR: a CSR comprising anti-ROR2 scFv EC, 4-1BB TM, and 4-1BB IC
Construct: anti-ROR2-DAP10-CSR: a CSR comprising anti-ROR2 scFv EC, DAP10 TM, and DAP10 IC
Constructs (for melanoma): anti-EFGR CSR comprising CD30, CD28, 4-1BB, or DAP10 costimulatory fragments and anti-MAGE-A4 TCR co-expressed with such anti-EGFR CSRs. An anti-EGFR CSR can comprise an anti-EGF scFv.
Construct: anti-EGFR-CD30-CSR: a CSR comprising anti-EGFR scFv extracellular (EC), CD30 transmembrane (TM), and CD30 intracellular (IC) domains
Construct: anti-EGFR-CD28T-CD30-CSR: a CSR comprising anti-EGFR scFv EC, CD28 TM, and CD30 IC
Construct: anti-EGFR-CD8T-CD30-CSR: a CSR comprising anti-EGFR scFv EC, CD8 TM, and CD30 IC
Construct: anti-EGFR-CD27T-CD30-CSR: a CSR comprising anti-EGFR scFv EC, CD27 TM, and CD30 IC
Construct: anti-EGFR-OX40T-CD30-CSR: a CSR comprising anti-EGFR scFv EC, OX40 TM, and CD30 IC
Construct: anti-EGFR-41BBT-CD30-CSR: a CSR comprising anti-EGFR scFv EC, 4-1BB TM, and CD30 IC
Construct: anti-EGFR-CD28-CSR: a CSR comprising anti-EGFR scFv EC, CD28 TM, and CD28 IC
Construct: anti-EGFR-CD28T-41BB-CSR: a CSR comprising anti-EGFR scFv EC, CD28 TM, and 4-1BB IC
Construct: anti-EGFR-CD28T-DAP10-CSR: a CSR comprising anti-EGFR scFv EC, CD28 TM, and DAP10 IC
Construct: anti-EGFR-CD27-CSR: a CSR comprising anti-EGFR scFv EC, CD27 TM, and CD27 IC
Construct: anti-EGFR-OX40-CSR: a CSR comprising anti-EGFR scFv EC, OX40 TM, and OX40 IC
Construct: anti-EGFR-41BB-CSR: a CSR comprising anti-EGFR scFv EC, 4-1BB TM, and 4-1BB IC
Construct: anti-EGFR-DAP10-CSR: a CSR comprising anti-EGFR scFv EC, DAP10 TM, and DAP10 IC
Construct: anti-MAGE-A4-TCR (a.k.a. anti-MAGE-A4-TCR or anti-MAGE-A4/MHC-TCR): alpha beta TCR pair/chains comprising anti-MAGE-A4/MHC TCR binding domains and alpha beta TCR transmembrane (TM) and intracellular (IC) domains, without CSR.
Construct: anti-MAGE-A4-TCR+anti-EGFR-CD30-CSR: anti-PSA-TCR co-expressed with a anti-PSMA-CD30-CSR
Construct: anti-MAGE-A4-TCR+anti-EGFR-CD8T-CD30-CSR
Construct: anti-MAGE-A4-TCR+anti-EGFR-CD28T-CD30-CSR
Construct: anti-MAGE-A4-TCR+anti-EGFR-CD28-CSR
Construct: anti-MAGE-A4-TCR+anti-EGFR-CD28T-41BB-CSR
Construct: anti-MAGE-A4-TCR+anti-EGFR-CD28T-DAP10-CSR

For Breast Cancers:
Constructs: anti-HER2 or anti-EpCAM CSRs comprising CD30, CD28, 4-1BB, or DAP10 costimulatory fragments and various TCRs (e.g., those targeting SLC3A2, KIAA0368. CADPS2, CTSB, PRAME, p53, or PSA as listed in Table 2) co-expressed with such CSRs. An anti-HER2 CSR can comprise an anti-HER2 scFv. An anti-EpCAM CSR can comprise an anti-EpCAM scFv.
Construct: anti-HER2-CD30-CSR: a CSR comprising anti-HER2 scFv extracellular (EC), CD30 transmembrane (TM), and CD30 intracellular (IC) domains
Construct: anti-HER2-CD28T-CD30-CSR a CSR comprising anti-HER2 scFv EC, CD28 TM, and CD30 IC
Construct: anti-HER2-CD8T-CD30-CSR: a CSR comprising anti-HER2 scFv EC, CD8 TM, and CD30 IC
Construct: anti-HER2-CD27T-CD30-CSR: a CSR comprising anti-HER2 scFv EC, CD27 TM, and CD30 IC
Construct: anti-HER2-OX40T-CD30-CSR: a CSR comprising anti-HER2 scFv EC, OX40 TM, and CD30 IC
Construct: anti-HER2-41BBT-CD30-CSR: a CSR comprising anti-HER2 scFv EC, 4-1BB TM, and CD30 IC
Construct: anti-HER2-CD28-CSR: a CSR comprising anti-HER2 scFv EC, CD28 TM, and CD28 IC
Construct: anti-HER2-CD28T-41BB-CSR: a CSR comprising anti-HER2 scFv EC, CD28 TM, and 4-1BB IC
Construct: anti-HER2-CD28T-DAP10-CSR a CSR comprising anti-HER2 scFv EC, CD28 TM, and DAP10 IC
Construct: anti-HER2-CD27-CSR: a CSR comprising anti-HER2 scFv EC, CD27 TM, and CD27 IC
Construct: anti-HER2-OX40-CSR: a CSR comprising anti-HER2 scFv EC, OX40 TM, and OX40 IC
Construct: anti-HER2-41BB-CSR: a CSR comprising anti-HER2 scFv EC, 4-1BB TM, and 4-1BB IC
Construct: anti-HER2-DA P10-CSR: a CSR comprising anti-HER2 scFv EC, DAP10 TM, and DAP10 IC
Construct: anti-EpCAM-CD30-CSR: a CSR comprising anti-EpCAM scFv extracellular (EC), CD30 transmembrane (TM), and CD30 intracellular (IC) domains
Construct: anti-EpCAM-CD28T-CD30-CSR: a CSR comprising anti-EpCAM scFv EC, CD28 TM, and CD30 IC
Construct: anti-EpCAM-CD8T-CD30-CSR: a CSR comprising anti-EpCAM scFv EC, CD8 TM, and CD30 IC
Construct: anti-EpCAM-CD27T-CD30-CSR: a CSR comprising anti-EpCAM scFv EC, CD27 TM, and CD30 IC
Construct: anti-EpCAM-OX40T-CD30-CSR: a CSR comprising anti-EpCAM scFv EC, OX40 TM, and CD30 IC
Construct: anti-EpCAM-41BBT-CD30-CSR: a CSR comprising anti-EpCAM scFv EC, 4-1BB TM, and CD30 IC
Construct: anti-EpCAM-CD28-CSR: a CSR comprising anti-EpCAM scFv EC, CD28 TM, and CD28 IC
Construct: anti-EpCAM-CD28T-41BB-CSR: a CSR comprising anti-EpCAM scFv EC, CD28 TM, and 4-1BB IC
Construct: anti-EpCAM-CD28T-DAP10-CSR: a CSR comprising anti-EpCAM scFv EC, CD28 TM, and DAP10 IC
Construct: anti-EpCAM-CD27-CSR: a CSR comprising anti-EpCAM scFv EC, CD27 TM, and CD27 IC
Construct: anti-EpCAM-OX40-CSR: a CSR comprising anti-EpCAM scFv EC, OX40 TM, and OX40 IC
Construct: anti-EpCAM-41BB-CSR: a CSR comprising anti-EpCAM scFv EC, 4-1BB TM, and 4-1BB IC
Construct: anti-EpCAM-DAP10-CSR: a CSR comprising anti-EpCAM scFv EC, DAP10 TM, and DAP10 IC
Constructs: anti-ROR1 CSRs comprising CD30, CD28, 4-1BB, or DAP10 costimulatory fragments (as disclosed above under “For pancreatic cancer”) and various TCRs (e.g., those targeting SLC3A2, KAA0368, CADPS2, CTSB, PRAME, p53, or PSA as listed in Table 2) co-expressed with such CSRs. An anti-ROR1 CSR can comprise an anti-ROR1 scFv.

For Ovarian Cancer:
Constructs: anti-MUC1 CSR comprising CD30, CD28, 4-1BB, or DAP10 costimulatory fragments and various TCRs (e.g., those targeting WT1, NY-ESO-1, p53, DPY19L4, or RNF19B as listed in Table 2) co-expressed with such CSRs. The anti-MUC1 CSR can comprise an anti-MUC1 scFv.
Construct: anti-MUC1-CD30-CSR: a CSR comprising anti-MUC1 scFv extracellular (EC), CD30 transmembrane (TM), and CD30 intracellular (IC) domains
Construct: anti-MUC1-CD28T-CD30-CSR: a CSR comprising anti-MUC1 scFv EC, CD28 TM, and CD30 IC
Construct: anti-MUC1-CD8T-CD30-CSR: a CSR comprising anti-MUC1 scFv EC, CD8 TM, and CD30 IC
Construct: anti-MUC1-CD27T-CD30-CSR: a CSR comprising anti-MUC1 scFv EC, CD27 TM, and CD30 IC
Construct: anti-MUC1-OX40T-CD30-CSR: a CSR comprising anti-MUC1 scFv EC, OX40 TM, and CD30 IC
Construct: anti-MUC1-41BBT-CD30-CSR: a CSR comprising anti-MUC scFv EC, 4-1BB TM, and CD30 IC
Construct: anti-MUC-CD28-CSR: a CSR comprising anti-MUC1 scFv EC, CD28 TM, and CD28 IC
Construct: anti-MUC1-CD28T-41BB-CSR: a CSR comprising anti-MUC1 scFv EC, CD28 TM, and 4-1BB IC
Construct: anti-MUC-CD28T-DAP10-CSR: a CSR comprising anti-MUC1 scFv EC, CD28 TM, and DAP10 IC
Construct: anti-MUC1-CD27-CSR a CSR comprising anti-MUC1 scFv EC, CD27 TM, and CD27 IC
Construct: anti-MUC-OX40-CSR: a CSR comprising anti-MUC scFv EC, OX40 TM, and OX40 IC
Construct: anti-MUC1-41BB-CSR a CSR comprising anti-MUC1 scFv EC, 4-1BB TM, and 4-1BB IC
Construct: anti-MUC1-DAP10-CSR: a CSR comprising anti-MUC1 scFv EC, DAP10 TM, and DAP10 IC
Constructs: anti-MUC16 CSR comprising CD30, CD28, 4-1BB, or DAP10 costimulatory fragments and various TCRs (e.g., those targeting WT1, NY-ESO-1, p53, DPY19L4, MAGE-A4 or RNF19B as listed in Table 2) co-expressed with such CSRs. The anti-MUC16 CSR can comprise an anti-MUC16 scFv.
Construct: anti-MUC16-CD30-CSR: a CSR comprising anti-MUC16 scFv extracellular (EC), CD30 transmembrane (TM), and CD30 intracellular (IC) domains
Construct: anti-MUC16-CD28T-CD30-CSR: a CSR comprising anti-MUC16 scFv EC, CD28 TM, and CD30 IC
Construct: anti-MUC16-CD8T-CD30-CSR: a CSR comprising anti-MUC16 scFv EC, CD8 TM, and CD30 IC
Construct: anti-MUC16-CD27T-CD30-CSR: a CSR comprising anti-MUC16 scFv EC, CD27 TM, and CD30 IC
Construct: anti-MUC16-OX40T-CD30-CSR: a CSR comprising anti-MUC16 scFv EC, OX40 TM, and CD30 IC
Construct: anti-MUC16-41BBT-CD30-CSR: a CSR comprising anti-MUC16 scFv EC, 4-1BB TM, and CD30 IC
Construct: anti-MUC16-CD28-CSR: a CSR comprising anti-MUC16 scFv EC, CD28 TM, and CD28 IC
Construct: anti-MUC16-CD28T-41BB-CSR: a CSR comprising anti-MUC16 scFv EC, CD28 TM, and 4-1BB IC
Construct: anti-MUC16-CD28T-DAP10-CSR: a CSR comprising anti-MUC16 scFv EC, CD28 TM, and DAP10 IC
Construct: anti-MUC16-CD27-CSR: a CSR comprising anti-MUC16 scFv EC, CD27 TM, and CD27 IC
Construct: anti-MUC16-OX40-CSR a CSR comprising anti-MUC16 scFv EC, OX40 TM, and OX40 IC
Construct: anti-MUC16-41BB-CSR: a CSR comprising anti-MUC16 scFv EC, 4-1BB TM, and 4-1BB IC
Construct: anti-MUC16-DAP10-CSR: a CSR comprising anti-MUC16 scFv EC, DAP10 TM, and DAP10 IC
Constructs: anti-FRα CSR comprising CD30, CD28, 4-1BB, or DAP10 costimulatory fragments and various TCRs (e.g., those targeting WT1, NY-ESO-1, p53, DPY19L4, or RNF19B as listed in Table 2) co-expressed with such CSRs. The anti-FRα CSR can comprise an anti-FRα scFv.
Construct: anti-FRα-CD30-CSR: a CSR comprising anti-FRα scFv extracellular (EC), CD30 transmembrane (TM), and CD30 intracellular (IC) domains
Construct: anti-FRα-CD28T-CD30-CSR: a CSR comprising anti-FRα scFv EC, CD28 TM, and CD30 IC
Construct: anti-FRα-CD8T-CD30-CSR: a CSR comprising anti-FRα scFv EC, CD8 TM, and CD30 IC
Construct: anti-FRα-CD27T-CD30-CSR: a CSR comprising anti-FRα scFv EC, CD27 TM, and CD30 IC
Construct: anti-FRα-OX40T-CD30-CSR: a CSR comprising anti-FRα scFv EC, OX40 TM, and CD30 IC
Construct: anti-FRα-41BBT-CD30-CSR: a CSR comprising anti-FRα scFv EC, 4-1BB TM, and CD30 IC
Construct: anti-FRα-CD28-CSR: a CSR comprising anti-FRα scFv EC, CD28 TM, and CD28 IC
Construct: anti-FRα-CD28T-41BB-CSR a CSR comprising anti-FRα scFv EC, CD28 TM, and 4-1BB IC
Construct: anti-FRα-CD28T-DAP10-CSR: a CSR comprising anti-FRα scFv EC, CD28 TM, and DAP10 IC
Construct: anti-FRα-CD27-CSR: a CSR comprising anti-FRα scFv EC, CD27 TM, and CD27 IC
Construct: anti-FRα-OX40-CSR: a CSR comprising anti-FRα scFv EC, OX40 TM, and OX40 IC
Construct: anti-FRα-41BB-CSR: a CSR comprising anti-FRα scFv EC, 4-1BB TM, and 4-1BB IC
Construct: anti-FRα-DAP10-CSR: a CSR comprising anti-FRα scFv EC, DAP10 TM, and DAP10 IC
Constructs: anti-ROR1 CSR comprising CD30, CD28, 4-1BB, or DAP10 costimulatory fragments (as disclosed above under “For pancreatic cancer”) and various TCRs (e.g., those targeting WT1, NY-ESO-1, p53, DPY19L4, MAGE-A4, or RNF19B as listed in Table 2) co-expressed with such CSRs. The anti-ROR1 CSR can comprise an anti-ROR1 scFv.
Constructs: anti-MSLN CSR comprising CD30, CD28, 4-1BB, or DAP10 costimulatory fragments (as disclosed above under “For pancreatic cancer”) and anti-MAGE-A4 TCR co-expressed with such anti-MSLN CSRs. The anti-MSLN CSR can comprise an anti MSLN scFv.
Constructs: anti-EGFR CSR comprising CD30, CD28, 4-1BB, or DAP10 costimulatory fragments (as disclosed below under “For colorectal cancer”) and an anti-MAGE-A4 TCR co-expressed with such CSRs. The anti-EGFR CSR can comprise an anti-EGFR scFv.

For Colorectal Cancer:
Constructs: anti-EFGR CSR comprising CD30, CD28, 4-1BB, or DAP10 costimulatory fragments and various TCRs (e.g. those targeting p53 or KRAS as listed in Table 2) co-expressed with such CSRs. The anti-EGFR CSR can comprise an anti-EGFR scFv.
Construct: anti-EGFR-CD30-CSR: a CSR comprising anti-EGFR scFv extracellular (EC), CD30 transmembrane (TM), and CD30 intracellular (IC) domains
Construct: anti-EGFR-CD28T-CD30-CSR: a CSR comprising anti-EGFR scFv EC, CD28 TM, and CD30 IC
Construct: anti-EGFR-CD8T-CD30-CSR: a CSR comprising anti-EGFR scFv EC, CD8 TM, and CD30 IC
Construct: anti-EGFR-CD27T-CD30-CSR: a CSR comprising anti-EGFR scFv EC, CD27 TM, and CD30 IC
Construct: anti-EGFR-OX40T-CD30-CSR: a CSR comprising anti-EGFR scFv EC, OX40 TM, and CD30 IC
Construct: anti-EGFR-41BBT-CD30-CSR a CSR comprising anti-EGFR scFv EC, 4-1BB TM, and CD30 IC
Construct: anti-EGFR-CD28-CSR: a CSR comprising anti-EGFR scFv EC, CD28 TM, and CD28 IC
Construct: anti-EGFR-CD28T-41BB-CSR a CSR comprising anti-EGFR scFv EC, CD28 TM, and 4-1BB IC
Construct: anti-EGFR-CD28T-DAP10-CSR: a CSR comprising anti-EGFR scFv EC, CD28 TM, and DAP10 IC
Construct: anti-EGFR-CD27-CSR: a CSR comprising anti-EGFR scFv EC, CD27 TM, and CD27 IC
Construct: anti-EGFR-OX40-CSR: a CSR comprising anti-EGFR scFv EC, OX40 TM, and OX40 IC
Construct: anti-EGFR-41BB-CSR: a CSR comprising anti-EGFR scFv EC, 4-1BB TM, and 4-1 BB IC
Construct: anti-EGFR-DAP10-CSR: a CSR comprising anti-EGFR scFv EC, DAP10 TM, and DAP10 IC
Constructs: anti-MUC1 CSR comprising CD30, CD28, 4-1BB, or DAP10 costimulatory fragments and a TCR targeting WT1 co-expressed with such CSRs. The anti-MUC1 CSR can comprise an anti-MUC1 scFv.
Construct: anti-MUC1-CD30-CSR: a CSR comprising anti-MUC1 scFv extracellular (EC), CD30 transmembrane (TM), and CD30 intracellular (IC) domains
Construct: anti-MUC1-CD28T-CD30-CSR: a CSR comprising anti-MUC1 scFv EC, CD28 TM, and CD30 IC
Construct: anti-MUC1-CD8T-CD30-CSR: a CSR comprising anti-MUC1 scFv EC, CD8 TM, and CD30 IC
Construct: anti-MUC1-CD27T-CD30-CSR: a CSR comprising anti-MUC1 scFv EC, CD27 TM, and CD30 IC
Construct: anti-MUC1-OX40T-CD30-CSR: a CSR comprising anti-MUC1 scFv EC, OX40 TM, and CD30 IC
Construct: anti-MUC1-41BBT-CD30-CSR a CSR comprising anti-MUC1 scFv EC, 4-1BB TM, and CD30 IC
Construct: anti-MUC1-CD28-CSR: a CSR comprising anti-MUC1 scFv EC, CD28 TM, and CD28 IC
Construct: anti-MUC1-CD28T-41BB-CSR: a CSR comprising anti-MUC1 scFv EC, CD28 TM, and 4-1BB IC
Construct: anti-MUC1-CD28T-DAP10-CSR: a CSR comprising anti-MUC1 scFv EC, CD28 TM, and DAP10 IC
Construct: anti-MUC1-CD27-CSR: a CSR comprising anti-MUC1 scFv EC, CD27 TM, and CD27 IC
Construct: anti-MUC1-OX40-CSR: a CSR comprising anti-MUC1 scFv EC, OX40 TM, and OX40 IC
Construct: anti-MUC1-41BB-CSR: a CSR comprising anti-MUC1 scFv EC, 4-1BB TM, and 4-1 BB IC
Construct: anti-MUC1-DAP10-CSR: a CSR comprising anti-MUC1 scFv EC, DAP10 TM, and DAP10 IC
Construct: anti WT1-TCR construct: (a.k.a. anti-WT1-TCR or anti-WT1/MHC-TCR): alpha beta TCR pair/chains comprising anti-WT1/MHC TCR binding domains and alpha beta TCR transmembrane (TM) and intracellular (IC) domains, without CSR

For Glioblastoma Cancer:
Constructs: anti-EGFR CSR comprising CD30, CD28, 4-1BB, or DAP10 costimulatory fragments (as disclosed above under “For colorectal cancer”) and various TCRs (e.g., those targeting ARHGAP35 or Histone H3.3 as listed in Table 2) co-expressed with such CSRs. The anti-EGFR CSR can comprise an anti-EGFR scFv.
Constructs: anti-EGFRvIII CSR comprising CD30, CD28, 4-1BB, or DAP10 costimulatory fragments and various TCRs (e.g., those targeting ARHGAP35 or Histone H3.3 as listed in Table 2) co-expressed with such CSRs. The anti-EGFRvIII CSR can comprise an anti-EGFRvIII scFv.
Construct: anti-EGFRvIII-CD30-CSR a CSR comprising anti-EGFRvIII scFv extracellular (EC), CD30 transmembrane (TM), and CD30 intracellular (IC) domains
Construct: anti-EGFRvIII-CD28T-CD30-CSR: a CSR comprising anti-EGFRvIII scFv EC, CD28 TM, and CD30 IC
Construct: anti-EGFRvIII-CD8T-CD30-CSR: a CSR comprising anti-EGFRvIII scFv EC, CD8 TM, and CD30 IC
Construct: anti-EGFRvIII-CD27T-CD30-CSR: a CSR comprising anti-EGFRvIII scFv EC, CD27 TM, and CD30 IC
Construct: anti-EGFRvIII-OX40T-CD30-CSR: a CSR comprising anti-EGFRvIII scFv EC, OX40 TM, and CD30 IC
Construct: anti-EGFRvIII-41BBT-CD30-CSR: a CSR comprising anti-EGFRvIII scFv EC, 4-1BB TM, and CD30 IC
Construct: anti-EGFRvIII-CD28-CSR: a CSR comprising anti-EGFRvIII scFv EC, CD28 TM, and CD28 IC
Construct: anti-EGFRvIII-CD28T-41BB-CSR: a CSR comprising anti-EGFRvIII scFv EC. CD28 TM, and 4-1BB IC
Construct: anti-EGFRvIII-CD28T-DAP10-CSR: a CSR comprising anti-EGFRvIII scFv EC, CD28 TM, and DAP10 IC
Construct: anti-EGFRvIII-CD27-CSR: a CSR comprising anti-EGFRvIII scFv EC, CD27 TM, and CD27 IC
Construct: anti-EGFRvIII-OX40-CSR: a CSR comprising anti-EGFRvIII scFv EC, OX40 TM, and OX40 IC
Construct: anti-EGFRvIII-41BB-CSR: a CSR comprising anti-EGFRvIII scFv EC, 4-1BB TM, and 4-1BB IC
Construct: anti-EGFRvIII-DAP10-CSR: a CSR comprising anti-EGFRvIII scFv EC, DAP10 TM, and DAP10 IC

For Lung Cancer:
Constructs: anti-HER3 CSR comprising CD30, CD28, 4-1BB, or DAP10 costimulatory fragments and various TCRs (e.g., those targeting KRAS, HER2, NY-ESO-1, MAGE-A4, or p53 as listed in Table 2) co-expressed with such CSRs. The ani-HER3 CSR can comprise an anti-HER3 scFv.
Construct: anti-HER3-CD30-CSR: a CSR comprising anti-HER3 scFv extracellular (EC), CD30 transmembrane (TM), and CD30 intracellular (IC) domains
Construct: anti-HER3-CD28T-CD30-CSR: a CSR comprising anti-HER3 scFv EC, CD28 TM, and CD30 IC
Construct: anti-HER3-CD8T-CD30-CSR: a CSR comprising anti-HER3 scFv EC, CD8 TM, and CD30 IC
Construct: anti-HER3-CD27T-CD30-CSR a CSR comprising anti-HER3 scFv EC, CD27 TM, and CD30 IC
Construct: anti-HER3-OX40T-CD30-CSR: a CSR comprising anti-HER3 scFv EC, OX40 TM, and CD30 IC
Construct: anti-HER3-41BBT-CD30-CSR: a CSR comprising anti-HER3 scFv EC, 4-1BB TM, and CD30 IC
Construct: anti-HER3-CD28-CSR: a CSR comprising anti-HER3 scFv EC, CD28 TM, and CD28 IC
Construct: anti-HER3-CD28T-41BB-CSR: a CSR comprising anti-HER3 scFv EC, CD28 TM, and 4-1BB IC
Construct: anti-HER3-CD28T-DAP10-CSR: a CSR comprising anti-HER3 scFv EC, CD28 TM, and DAP10 IC
Construct: anti-HER3-CD27-CSR: a CSR comprising anti-HER3 scFv EC, CD27 TM, and CD27 IC
Construct: anti-HER3-OX40-CSR: a CSR comprising anti-HER3 scFv EC, OX40 TM, and OX40 IC
Construct: anti-HER3-41BB-CSR: a CSR comprising anti-HER3 scFv EC, 4-1BB TM, and 4-1BB IC
Construct: anti-HER3-DA P10-CSR: a CSR comprising anti-HER3 scFv EC, DAP10 TM, and DAP10 IC
Constructs: anti-DLL3 CSR comprising CD30, CD28, 4-1BB, or DAP10 costimulatory fragments and various TCRs (e.g., those targeting KRAS, HER2, NY-ESO-1, or p53 as listed in Table 2) co-expressed with such CSRs. The anti-DLL3 CSR can comprise an anti-DLL3 scFv.
Construct: anti-DLL3-CD30-CSR: a CSR comprising anti-DLL3 scFv extracellular (EC), CD30 transmembrane (TM), and CD30 intracellular (IC) domains
Construct: anti-DLL3-CD28T-CD30-CSR: a CSR comprising anti-DLL3 scFv EC, CD28 TM, and CD30 IC
Construct: anti-DLL3-CD8T-CD30-CSR: a CSR comprising anti-DLL3 scFv EC, CD8 TM, and CD30 IC
Construct: anti-DLL3-CD27T-CD30-CSR: a CSR comprising anti-DLL3 scFv EC, CD27 TM, and CD30 IC
Construct: anti-DLL3-OX40T-CD30-CSR: a CSR comprising anti-DLL3 scFv EC, OX40 TM, and CD30 IC
Construct: anti-DLL3-41BBT-CD30-CSR: a CSR comprising anti-DLL3 scFv EC, 4-1BB TM, and CD30 IC
Construct: anti-DLL3-CD28-CSR a CSR comprising anti-DLL3 scFv EC, CD28 TM, and CD28 IC
Construct: anti-DLL3-CD28T-41BB-CSR: a CSR comprising anti-DLL3 scFv EC, CD28 TM, and 4-1BB IC
Construct: anti-DLL3-CD28T-DAP10-CSR a CSR comprising anti-DLL3 scFv EC, CD28 TM, and DAP10 IC
Construct: anti-DLL3-CD27-CSR: a CSR comprising anti-DLL3 scFv EC, CD27 TM, and CD27 IC
Construct: anti-DLL3-OX40-CSR: a CSR comprising anti-DLL3 scFv EC, OX40 TM, and OX40 IC
Construct: anti-DLL3-41BB-CSR: a CSR comprising anti-DLL3 scFv EC, 4-1BB TM, and 4-1BB IC
Construct: anti-DLL3-DAP10-CSR: a CSR comprising anti-DLL3 scFv EC, DAP10 TM, and DAP10 IC
Constructs: anti-C-MET CSR comprising CD30, CD28, 4-1BB, or DAP10 costimulatory fragments and various TCRs (e.g., those targeting KRAS, HER2, NY-ESO-1, or p53 as listed in Table 2) co-expressed with such CSRs. The anti-C-MET CSR can comprise an anti-C-MET scFv.
Construct: anti-C-MET-CD30-CSR: a CSR comprising anti-C-MET scFv extracellular (EC), CD30 transmembrane (TM), and CD30 intracellular (IC) domains
Construct: anti-C-MET-CD28T-CD30-CSR: a CSR comprising anti-C-MET scFv EC, CD28 TM, and CD30 IC
Construct: anti-C-MET-CD8T-CD30-CSR: a CSR comprising anti-C-MET scFv EC, CD8 TM, and CD30 IC
Construct: anti-C-MET-CD27T-CD30-CSR: a CSR comprising anti-C-MET scFv EC, CD27 TM, and CD30 IC
Construct: anti-C-MET-OX40T-CD30-CSR: a CSR comprising anti-C-MET scFv EC, OX40 TM, and CD30 IC
Construct: anti-C-MET-41BBT-CD30-CSR a CSR comprising anti-C-MET scFv EC, 4-1BB TM, and CD30 IC
Construct: anti-C-MET-CD28-CSR: a CSR comprising anti-C-MET scFv EC, CD28 TM, and CD28 IC
Construct: anti-C-MET-CD28T-41BB-CSR: a CSR comprising anti-C-MET scFv EC, CD28 TM, and 4-1BB IC
Construct: anti-C-MET-CD28T-DAP10-CSR: a CSR comprising anti-C-MET scFv EC, CD28 TM, and DAP10 IC
Construct: anti-C-MET-CD27-CSR: a CSR comprising anti-C-MET scFv EC, CD27 TM, and CD27 IC
Construct: anti-C-MET-OX40-CSR: a CSR comprising anti-C-MET scFv EC, OX40 TM, and OX40 IC
Construct: anti-C-MET-41BB-CSR: a CSR comprising anti-C-MET scFv EC, 4-1BB TM, and 4-1BB IC
Construct: anti-C-MET-DAP10-CSR: a CSR comprising anti-C-MET scFv EC, DAP10 TM, and DAP10 IC
Constructs: anti-ROR1 CSR comprising CD30, CD28, 4-1BB, or DAP10 costimulatory fragments (as disclosed above under “For pancreatic cancer”) and various TCRs (e.g., those targeting KRAS, HER2, NY-ESO-1, or p53 as listed in Table 2) co-expressed with such CSRs. The anti-ROR1 CSR can comprise an anti-ROR1 scFv.
Constructs: anti-EGFR CSR comprising CD30, CD28, 4-1BB, or DAP10 costimulatory fragments (as disclosed above under “For colorectal cancer”) and a TCR targeting MAGE-A4 co-expressed with such CSRs. The anti-EGFR CSR can comprise an anti-EGFR scFv.

For Renal Cell Carcinoma:
Constructs: anti-ROR2 CSR comprising CD30, CD28, 4-1BB, or DAP10 costimulatory fragments (as disclosed above under “For melanoma or gastrointestinal cancer”) and various TCRs (e.g., those targeting 5T4 or PRAME as listed in Table 2) co-expressed with such CSRs. The anti-ROR2 CSR can comprise an anti-ROR2 scFv.
Constructs: anti-CD70 CSR comprising CD30, CD28, 4-1BB, or DAP10 costimulatory fragments and various TCRs (e.g., those targeting 5T4 or PRAME as listed in Table 2) co-expressed with such CSRs. The anti-CD70 CSR can comprise an anti-CD70 scFv.
Construct: anti-CD70-CD30-CSR: a CSR comprising anti-CD70 scFv extracellular (EC), CD30 transmembrane (TM), and CD30 intracellular (IC) domains
Construct: anti-CD70-CD28T-CD30-CSR: a CSR comprising anti-CD70 scFv EC, CD28 TM, and CD30 IC
Construct: anti-CD70-CD8T-CD30-CSR: a CSR comprising anti-CD70 scFv EC, CD8 TM, and CD30 IC
Construct: anti-CD70-CD27T-CD30-CSR: a CSR comprising anti-CD70 scFv EC, CD27 TM, and CD30 IC
Construct: anti-CD70-OX40T-CD30-CSR: a CSR comprising anti-CD70 scFv EC, OX40 TM, and CD30 IC
Construct: anti-CD70-41BBT-CD30-CSR: a CSR comprising anti-CD70 scFv EC, 4-1BB TM, and CD30 IC
Construct: anti-CD70-CD28-CSR: a CSR comprising anti-CD70 scFv EC, CD28 TM, and CD28 IC
Construct: anti-CD70-CD28T-41BB-CSR: a CSR comprising anti-CD70 scFv EC, CD28 TM, and 4-1BB IC
Construct: anti-CD70-CD28T-DAP10-CSR: a CSR comprising anti-CD70 scFv EC, CD28 TM, and DAP10 IC
Construct: anti-CD70-CD27-CSR: a CSR comprising anti-CD70 scFv EC, CD27 TM, and CD27 IC
Construct: anti-CD70-OX40-CSR: a CSR comprising anti-CD70 scFv EC, OX40 TM, and OX40 IC
Construct: anti-CD70-41BB-CSR: a CSR comprising anti-CD70 scFv EC, 4-1BB TM, and 4-1BB IC
Construct: anti-CD70-DAP10-CSR a CSR comprising anti-CD70 scFv EC, DAP10 TM, and DAP10 IC
Constructs: anti-MCT4 CSR comprising CD30, CD28, 4-1BB, or DAP10 costimulatory fragments and various TCRs (e.g. those targeting 5T4 or PRAME as listed in Table 2) co-expressed with such CSRs. The anti-MCT4 CSR can comprise an anti-MCT4 scFv.
Construct: anti-MCT4-CD30-CSR: a CSR comprising anti-MCT4 scFv extracellular (EC), CD30 transmembrane (TM), and CD30 intracellular (IC) domains
Construct: anti-MCT4-CD28T-CD30-CSR: a CSR comprising anti-MCT4 scFv EC, CD28 TM, and CD30 IC
Construct: anti-MCT4-CD8T-CD30-CSR: a CSR comprising anti-MCT4 scFv EC, CD8 TM, and CD30 IC
Construct: anti-MCT4-CD27T-CD30-CSR: a CSR comprising anti-MCT4 scFv EC, CD27 TM, and CD30 IC
Construct: anti-MCT4-OX40T-CD30-CSR: a CSR comprising anti-MCT4 scFv EC, OX40 TM, and CD30 IC
Construct: anti-MCT4-41BBT-CD30-CSR: a CSR comprising anti-MCT4 scFv EC, 4-1BB TM, and CD30 IC
Construct: anti-MCT4-CD28-CSR: a CSR comprising anti-MCT4 scFv EC, CD28 TM, and CD28 IC
Construct: anti-MCT4-CD28T-41BB-CSR: a CSR comprising anti-MCT4 scFv EC, CD28 TM, and 4-1BB IC
Construct: anti-MCT4-CD28T-DAP10-CSR: a CSR comprising anti-MCT4 scFv EC, CD28 TM, and DAP10 IC
Construct: anti-MCT4-CD27-CSR: a CSR comprising anti-MCT4 scFv EC, CD27 TM, and CD27 IC
Construct: anti-MCT4-OX40-CSR: a CSR comprising anti-MCT4 scFv EC, OX40 TM, and OX40 IC
Construct: anti-MCT4-41BB-CSR: a CSR comprising anti-MCT4 scFv EC, 4-1BB TM, and 4-1BB IC
Construct: anti-MCT4-DAP10-CSR: a CSR comprising anti-MCT4 scFv EC, DAP10 TM, and DAP10 IC

### For Myeloma:

Constructs: anti-EGFR CSR comprising CD30, CD28, 4-1BB, or DAP10 costimulatory fragments and a TCR targeting MAGE-A4. The anti-EGFR CSR can comprise an anti-EGFR scFv.
Construct: anti-EGFR-CD30-CSR: a CSR comprising anti-EGFR scFv extracellular (EC), CD30 transmembrane (TM), and CD30 intracellular (IC) domains
Construct: anti-EGFR-CD28T-CD30-CSR: a CSR comprising anti-EGFR scFv EC, CD28 TM, and CD30 IC
Construct: anti-EGFR-CD8T-CD30-CSR: a CSR comprising anti-EGFR scFv EC, CD8 TM, and CD30 IC
Construct: anti-EGFR-CD27T-CD30-CSR: a CSR comprising anti-EGFR scFv EC, CD27 TM, and CD30 IC
Construct: anti-EGFR-OX40T-CD30-CSR: a CSR comprising anti-EGFR scFv EC, OX40 TM, and CD30 IC
Construct: anti-EGFR-41BBT-CD30-CSR: a CSR comprising anti-EGFR scFv EC, 4-1BB TM, and CD30 IC
Construct: anti-EGFR-CD28-CSR: a CSR comprising anti-EGFR scFv EC, CD28 TM, and CD28 IC
Construct: anti-EGFR-CD28T-41BB-CSR: a CSR comprising anti-EGFR scFv EC, CD28 TM, and 4-1BB IC
Construct: anti-EGFR-CD28T-DAP10-CSR: a CSR comprising anti-EGFR scFv EC, CD28 TM, and DAP10 IC
Construct: anti-EGFR-CD27-CSR: a CSR comprising anti-EGFR scFv EC, CD27 TM, and CD27 IC
Construct: anti-EGFR-OX40-CSR: a CSR comprising anti-EGFR scFv EC, OX40 TM, and OX40 IC
Construct: anti-EGFR-41BB-CSR: a CSR comprising anti-EGFR scFv EC, 4-1BB TM, and 4-1BB IC
Construct: anti-EGFR-DAP10-CSR: a CSR comprising anti-EGFR scFv EC, DAP10 TM, and DAP10 IC

### Example 9—Short-Term Killing of Target Cells by Anti-AFP/MHC TCR+Anti-GPC3-CSR T Cells

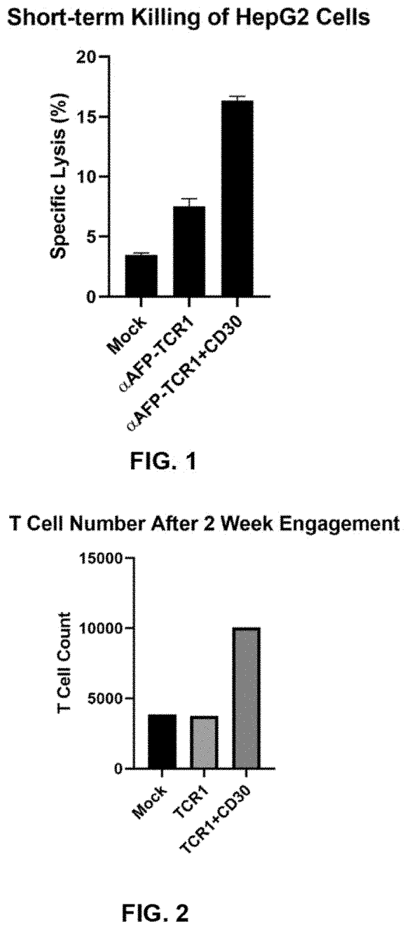

This example shows that TCR+CD30-CSR expressing T cells have higher specific tumor cell killing efficacies than TCR T cells without CSR. Primary T cells were mock-transduced (no DNA added) or transduced for 7 days with lentiviral vectors encoding: (1) anti-AFP-TCR1 (comprising SEQ ID NO:1 and SEQ ID NO:2) on one vector; or (2) anti-AFP-TCR1 (comprising SEQ ID NO:1 and SEQ ID NO:2), and anti-GPC3-CD30-CSR (comprising SEQ ID NO:181), on two vectors. The TCR T cells were tested for their abilities to kill cancer cells using the Cytox 96 Non-radioactive Cytotoxicity Assay (Promega). Briefly, the total transduced T cells and target cells HepG2 (AFP⁺, HLA-A2⁺, GPC3⁺) were co-cultured at an effector-to-target ratio of 2:1. Specific lysis was determined by measuring LDH activity in culture supernatants after 24 hr incubation. As shown in FIG. 1, T cells transduced with vectors encoding both TCR and CD30-CSR had higher in vitro tumor cell killing efficacies than corresponding TCR T cells without CSR.

### Example 10—Long-Term Killing of Target Cells by Anti-AFP/MHC TCR+Anti-GPC3-CSR T Cells and T Cell Survival

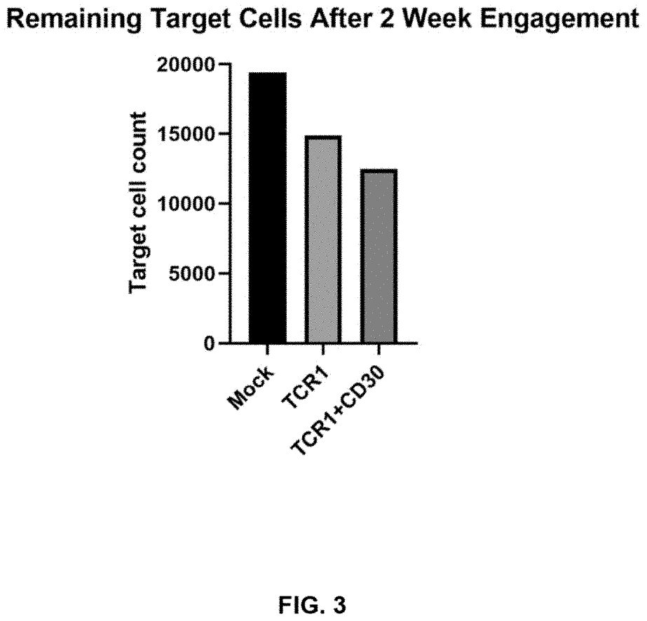

A FACS based assay for counting target cells was used to compare the long-term killing potential of TCR T cells. The effector cells used were primary T cells from donor subjects transduced with vectors encoding various TCR constructs. The effector cells were transduced for 7 days with vectors encoding: (1) anti-AFP-TCR1 (SEQ ID NO:1); or (2) anti-AFP-TCR1 and anti-GPC3-CD30-CSR (SEQ ID NO:1 and SEQ ID NO:181, respectively) on two vectors. The target cells used were HepG2 (A2⁺/AFP⁺/GPC3⁺) cells. The effector to target ratio (E:T ratio) in this experiment was 2:1. Specifically, 50,000 total transduced T cells and 25,000 HepG2 cells were incubated together in each well in RPMI+10% FBS with no cytokine. The cells were rechallenged with 50,000 HepG2 cells per well after 7 days (the 2^{nd} target cell engagement). The numbers of remaining target cells and total T cells were quantified 7 days after the 2^{nd} target cell engagement. The results of T cell survival (total T cell numbers) and the long-term killing (represented by remaining target cells) are shown in FIG. 2 and FIG. 3, respectively. FIG. 2 shows that T cells expressing anti-AFP-TCR+anti-GPC3-CD30-CSR survived much better than mock-transduced T cells and T cells expressing only anti-AFP-TCR. FIG. 3 shows that T cells expressing anti-AFP-TCR together with anti-GPC3-CD30-CSR killed more target cells than T cells expressing anti-AFP-TCR alone.

### Example 11—Further In Vitro Assays with Anti-AFP/MHC TCR+Anti-GPC3-CSR T Cells

### A. Short-Term In Vitro Tumor Cell Killing Assay

An LDH-based assay comparing the short-term killing ability of the various AFP-TCR T cells with and without a GPC3-CSR was performed. Effector cells used in this example and the following examples included α/βTCR-knock-out T cells transduced with lentiviral vectors encoding the following constructs:
(1) anti-AFP-TCR1 (or “AFP-TCR”), as disclosed in Example 9;
(2) anti-AFP-TCR1+anti-GPC3-CD30-CSR (or “AFP-TCR+GPC3-CD30-CSR”), as disclosed in Example 9;
(3) anti-AFP-TCR1+anti-GPC3-CD30T-CD28-CSR (or “AFP-TCR+GPC3-CD30T-CD28-CSR”); TCR sequences identical to (1), CSR comprising the same GPC3 binding moiety as in (2), myc tag (SEQ ID NO:261), and CD30T-CD28IC (SEQ ID NOs:232 and 237);
(4) anti-AFP-TCR1+anti-GPC3-CD28T-CD30-CSR (or “AFP-TCR+GPC3-CD28T-CD30-CSR”); TCR sequences identical to (1), CSR comprising the same GPC3 binding moiety as in (2), myc tag (SEQ ID NO:261); and CD28T-CD30 IC (SEQ ID NO:223);
(5) anti-AFP-TCR1+anti-GPC3-CD28T-41BB-CSR (or “AFP-TCR+GPC3-CD28T-41BB-CSR”); TCR sequences identical to (1), CSR comprising the same GPC3 binding moiety as in (2), myc tag (SEQ ID NO:261); and CD28T-41BB (SEQ ID NOS:232 and 236); or
(6) anti-AFP-TCR1+anti-GPC3-CD28T-DAP10-CSR (or “AFP-TCR+GPC3-CD28T-DAP10-CSR”); TCR sequences identical to (1), CSR comprising the same GPC3 binding moiety as in (2), myc tag (SEQ ID NO:261); and CD28T-DAP10IC (SEQ ID NOS:232 and 240).

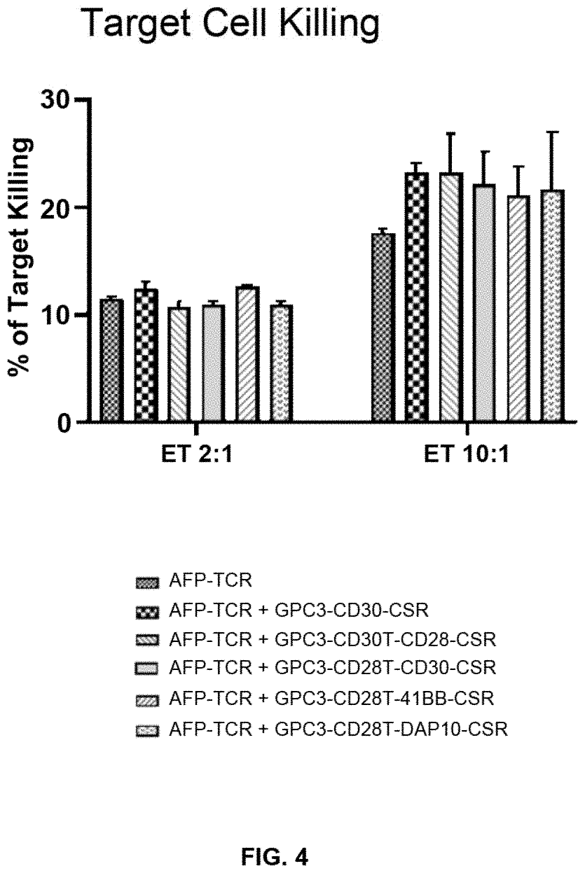

TCR-KO T cells expressing the above-described TCR or TCR+CSR constructs were activated and expanded with CD3/CD28 Dynabeads (Invitrogen) according to the manufacturer's protocol. Activated T cells were cultured and maintained in RPMI 1640 medium with 10% FBS plus 100 U/ml IL-2 and used at day 12. The T cells were >99% CD3⁺ by FACS analysis. Activated T cells (effector cells) and the target cells (HepG2 cells) were co-cultured at a 2:1 or 10:1 ratio for 16 hours. Cytotoxicities were then determined by measuring LDH activity in culture supernatants using an LDH Cytotoxicity Assay (Promega). The result is shown in FIG. 4. T cells expressing AFP-TCR+GPC3-CD30-CSR showed higher short-term cytotoxicity than T cells expressing only AFP-TCR at both E:T ratios (significantly higher at the higher E:T ratio), while at the lower E:T ratio, showed higher short-term cytotoxicity than T cells co-expressing AFP-TCR and GPC3-CD30T-CD28-CSR which has the same CD30 transmembrane domains (TM) but a different intracellular domain (IC). T cells expressing AFP-TCR+GPC3-CD28T-30-CSR showed higher short-term cytotoxicity than T cells expressing only AFP-TCR or expressing a GPC3-CSR with the same TM domain but an IC domain from other costimulatory molecules (4-1BB or DAP10) at the higher E:T ratio.

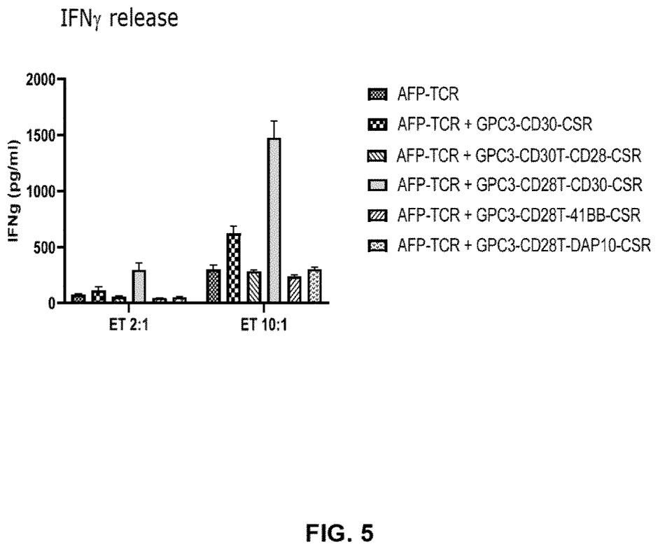

The short-term killing ability of the T cells expressing the various TCR and TCR+CSR constructs disclosed in this example was also determined by measuring the amounts/levels of cytokines released from T cells upon engagement with target cells. T cells with high cytotoxic potency secrete high levels of cytokines which serves as a measure of T cell activity. The level of cytokine IFNγ released into the culture supernatant after a 16 hour co-culture was quantified in a Luminex MAGPIX® multiplex system with the Bio-plex Pro™ Human Cytokine 8-plex Assay (BioRad). Shown in FIG. 5 is the IFNγ release from T cells expressing AFP-TCR alone or various AFP-TCR+GPC3-CSR combinations at two effector:target ratios (2:1 and 10:1). At both E:T ratios, T cells expressing AFP-TCR and GPC3-CSRs comprising the CD30 intracellular signaling/costimulatory domain (regardless of the TM domain) showed a greater degree of killing capability with significantly higher levels of IFNγ release compared to T cells expressing AFP-TCR alone or co-expressing AFP-TCR and a GPC3-CSR comprising a different intracellular domain (from CD28, 4-1BB, or DAP10).

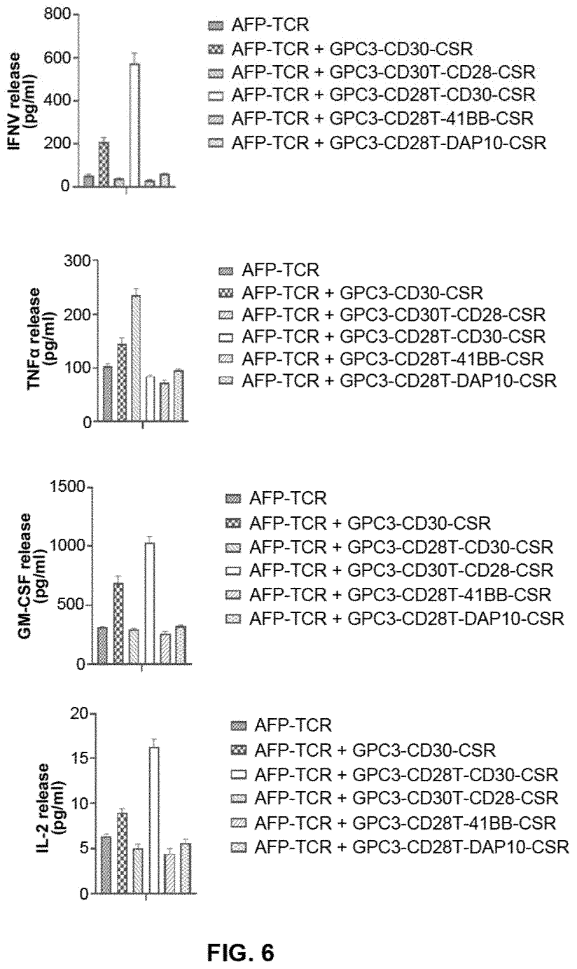

Short-term killing induced cytokine release was further investigated by concurrently measuring the levels of cytokines TNFα, GM-CSF, IFNγ, and IL-2 in culture supernatants. T cells expressing the various TCR-CSR combinations were incubated with target HepG2 cells and an E:T ratio of 10:1 for 16 hours. Culture supernatants were analysed for cytokine levels using the ELISA-based Bio-Plex Pro™ assay as described above. As shown in FIG. 6, AFP-TCR T cells with a GPC3-CSR comprising the CD30 IC signaling/costimulatory domain, regardless of the TM domain, showed a higher killing efficacy than corresponding AFP-TCR T cells without a GPC3-CSR or corresponding AFP-TCR T cells with GPC3-CSRs that had the same antigen binding region and respective TM domain but with an IC signaling/costimulatory domain derived from CD28, 4-1BB, or DAP10.

### B. Long-Term In Vitro Target Cell Killing

To assess the long-term tumor cell killing capability of T cells expressing AFP-TCR and GPC3-CSR combinations, a series of viability, killing, differentiation and proliferation assays were performed following a multi-week exposure of HepG2 target cells to the various populations of effector T cells.

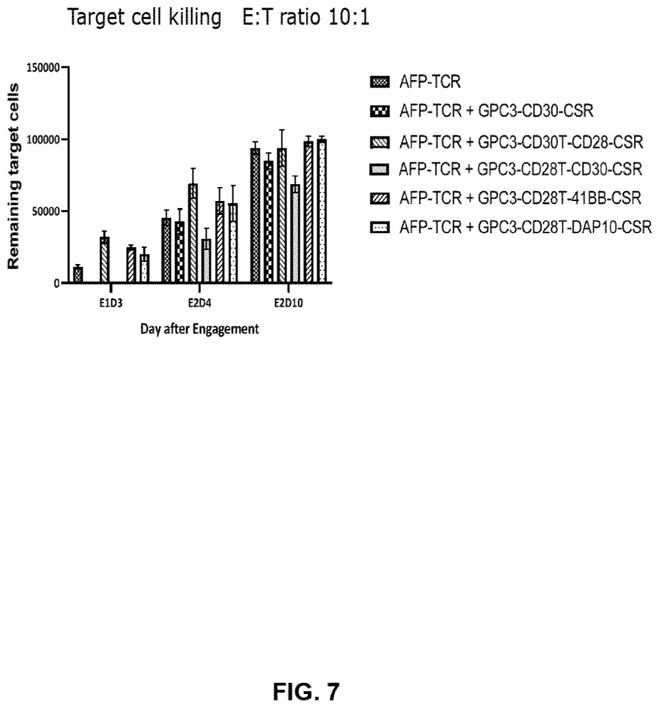

To assess target cell killing, a crystal-violet cell viability assay was used to count target cells and to compare the long-term target-cell killing potential of AFP-TCR+GPC3-CD30-CSR expressing T cells, TCR T cells without CSRs or in TCR T cells with CSRs comprising other costimulatory fragments. In this experiment, 500,000 T cells were incubated with HepG2 target cells at an effector cell to target cell ratio (E:T ratio) of 10:1 (the first engagement or “E1”). The cells were rechallenged with target cells at 3 days following the first engagement (the second engagement or “E2”). The numbers of remaining target cells were quantified using crystal-violet staining on various days after each engagement. Briefly, adherent cells were gently washed with PBS and replaced with a 0.5% solution of Crystal Violet (in ethanol) for 15 min. at room temperature. The cells were washed 3 times in ddH₂O. Elution buffer (10% acetic acid) was added to each well and the plate was gently shaken for 15 min. The plates were then centrifuged for 5 min. at 1600 rpm. A fraction of the elution buffer was transferred to a flat bottom well, diluted with an equal volume of ddH2O, mixed and the absorbance measured at 590 nm. The result is shown in FIG. 7. At every timepoint, T cells expressing AFP-TCR together with GPC3-CSRs comprising the CD30 intracellular domain, regardless of the TM domain, killed more target cells (as shown by the depletion of HepG2 cells) than T cells expressing the AFP-TCR alone or the AFP-TCR together with a CSR bearing the same GPC3 binding domain and respective TM domain but the IC costimulatory/signaling region of CD28, 4-1BB or DAP10.

### C. In Vitro T Cell Survival and Proliferation

The survival, proliferation and persistence of genetically modified T cells are crucial for the success of adoptive T-cell transfer therapies when treating cancers. To assay the effect of the various CSRs on T-cell survival and proliferation, we counted the number of T cells (CD3+^{cells}) on various days after engagement with target cells.

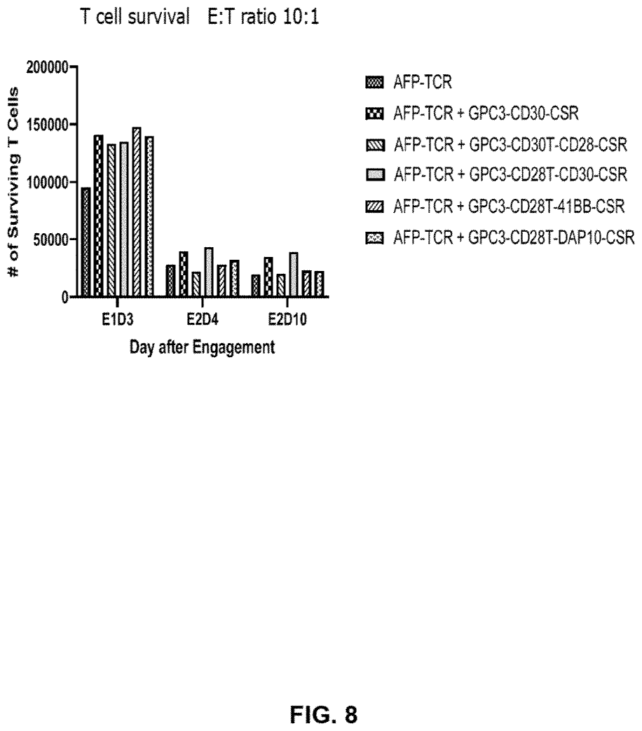

To this end, T cells expressing the AFP-TCR+GPC3-CD30-CSR was compared to the AFP-TCR alone, or the AFP-TCR+GPC3-CSR with another signaling moiety (CD28, 4-1BB or DAP10). The effector cell (T cell) population was counted using an antibody to T cell marker CD3 using flow cytometry. The results in FIG. 8 showed that at any time point after T cell engagement with target cells, the T cell number from the AFP-TCR+GPC3-CD30-CSR or the AFP-TCR+GPC3-CD28T-CD30-CSR group is significantly higher than that from T cells expressing the TCR only. Both of the TCR+CD30-CSR groups also had significantly higher T cell numbers than the T cell groups expressing the TCR and a CSR bearing the same GPC3 binding domain and respective TM domain, but with the IC region of CD28, 4-1BB or DAP10 at the two later timepoints. The result indicates that coexpressing a CSR containing the CD30 IC domain significantly increased the survival and proliferation capability of T cells expressing TCR, while coexpressing a CSR containing a different costimulatory molecule's IC domain did not have any significant increase in survival and proliferation (and no increase at all at later timepoints).

### D. Memory T Cell Quantification and T Cell Persistence

This example shows that AFP-TCR+GPC3-CD30-CSR T cells develop into and maintain a high central memory T cell population after target stimulation. To determine the effect of expressing TCR+CD30-CSR on T cells' ability to develop into and maintain memory T cells as compared to T cells expressing the AFP-TCR only or the AFP-TCR co-expressed with an GPC3-CSR comprising a different costimulatory fragment, i.e., CD28, 4-1BB, or DAP10's IC domain, we measured the cell surface expression of memory T cell markers CCR7 and CD45RA. As known in the field, T cells with high CCR7 expression levels and low CD45RA expression levels are considered as central memory T cells, T cells with low CCR7 and low CD45RA expression levels are effector memory T cells, T cells with low CCR7 and high CD45RA expression levels are effector T cells, while T cells with high CCR7 and high CD45RA are naïve T cells which are the initial type of T cells before target/antigen challenge/recognition (Mahnke et al., Eur. J Immunol. 43(11):2797-809, 2013). When in response to antigen encounter, naïve T cells proliferate and differentiate into effector cells, most of which carry out the role of destroying targets and then die, while a small pool of T cells ultimately develops into long-lived memory T cells which can store the T cell immunity against the specific target. Among the memory T cells, the central memory T cells are found to have longer lives than effector memory T cells and be capable of generating effector memory T cells, but not vice versa. Therefore, the ability to develop into and maintain memory T cells, especially central memory T cells, indicating the persistence of target-specific T cells, is an important and desired feature for potentially successful T cell therapies.

The effector cells expressing AFP-TCR constructs alone or AFP-TCR+GPC3-CD30-CSR expressing T cells were incubated with target cells at an E:T ratio of 10:1 (e.g., 500,000 receptor⁺ T cells and 50,000 target cells in each well on a 96-well plate) for 3 days.

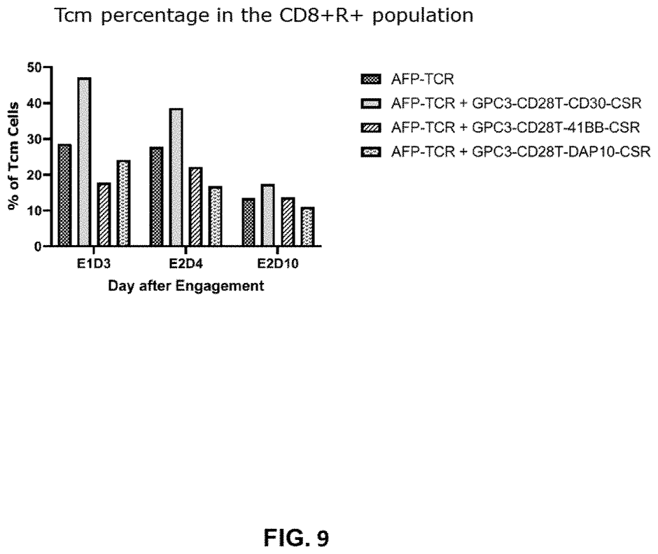

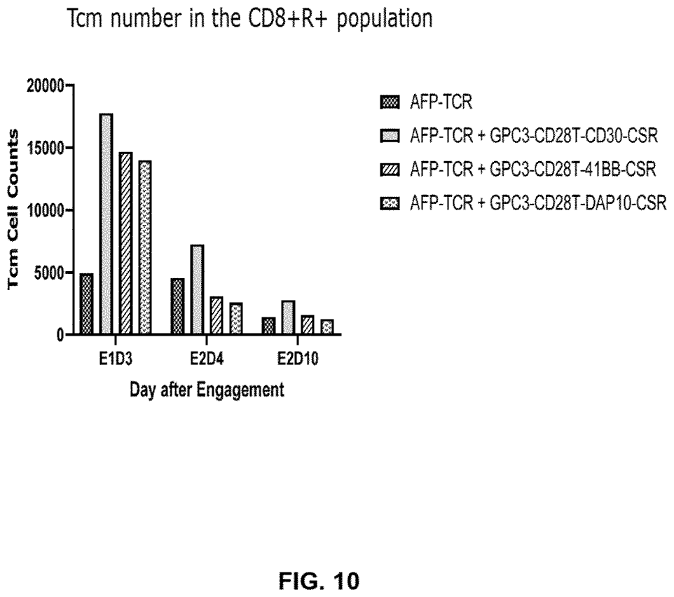

On selected days after each target cell engagement, each sample was stained with antibodies against CCR7 and CD45RA and analyzed by flow cytometry. Receptor⁺ CD8+ T cell numbers were counted and grouped into various T cell types based on their CCR7 and CD45RA expression levels: central memory T cells (CD45RA⁻ CCR7⁺), effector memory T cells (CD45RA⁻ CCR7⁻), effector T cells (CD45RA⁺ CCR7⁻), and naïve T cells (CD45RA⁺ CCR7⁺). Percentages of various types of T cells among the total number of receptor⁺ CD8+ T cells were calculated. In these experiments, the cells were stained with antibody to CD8 as a gate for cytotoxic T cells. The percentages or numbers of memory T cells from early in the assay (E1D3), an intermediate point (E2D4) and final day of the assay (E2D10) of the various TCR or TCR+CSR T cell groups are compared in FIGS. 9 and 10, respectively.

To better compare the effects of expression of the various AFP-TCR and GPC3-CSRs on the central memory T cell population during long-term assays, the values (cell number and percent of total) of receptor positive and CD8 positive T cells were compared and the results were used to prepare Tables 3, 4, 5, and 6.

TABLE 3
CD8⁺R⁺ Central Memory T Cell (Tcm) Counts of T Cells
Expressing anti-AFP-TCR and anti-GPC3-CSR with CD28TM.

| | | | | | | Ratio of Tcm |
|---|---|---|---|---|---|---|
| | | | | | | Counts TCR + |
| | | AFP-TCR + | AFP-TCR + | AFP-TCR + | Ratio of Tcm | CD28T-CD30- |
| | AFP-TCR | GPC3-CD28T- | GPC3-CD28T- | GPC3-CD28T- | Counts TCR + | CSR/TCR + |
| | Tcm | CD30-CSR | 41BB-CSR | DAP10-CSR | CD28T-CD30- | CD28T-41BB- |
| Date | Count | Tcm Count | Tcm Count | Tcm Count | CSR/TCR | CSR |
| E1D3 | 4,929 | 17,777 | 14,688 | 14,002 | 3.61 | 1.21 |
| E2D4 | 4,555 | 7,270 | 3,092 | 2,569 | 1.60 | 2.35 |
| E2D10 | 1,404 | 2,761 | 1,552 | 1,247 | 1.97 | 1.78 |

TABLE 4
Central Memory T Cell (Tcm) Percentages among CD8⁺R⁺ T Cells
Expressing anti-AFP-TCR and anti-GPC3-CSR with CD28TM.

| | | | | | | Ratio of Tcm |
|---|---|---|---|---|---|---|
| | | AFP-TCR + | AFP-TCR + | AFP-TCR + | Ratio of Tcm | Percentages |
| | | GPC3-CD28T- | GPC3-CD28T- | GPC3-CD28T- | Percentages | TCR + |
| | AFP-TCR | CD30-CSR | 41BB-CSR | DAP10-CSR | TCR + | CD28T-CD30- |
| | Tcm | Tcm | Tcm | Tcm | CD28T-CD30- | CSR/TCR + |
| Date | Percentage | Percentage | Percentage | Percentage | CSR/TCR | CD28T-41BB-CSR |
| E1D3 | 28.60 | 47.10 | 17.80 | 24.10 | 1.65 | 2.65 |
| E2D4 | 27.70 | 38.50 | 22.20 | 16.80 | 1.39 | 1.73 |
| E2D10 | 13.50 | 17.40 | 13.70 | 10.90 | 1.29 | 1.27 |

TABLE 5
Central Memory T Cell (Tcm) Percentages among CD8⁺R⁺ T Cells
Expressing anti-AFP-TCR and anti-GPC3-CSR with CD30TM.

| | | | | | Ratio of Tcm |
|---|---|---|---|---|---|
| | | AFP-TCR + | AFP-TCR + | Ratio of Tcm | Percentages |
| | AFP-TCR | GPC3-CD30- | GPC3-CD30T- | Percentages | TCR + CD30-CSR/ |
| | Tcm | CSR Tcm | CD28-CSR Tcm | TCR + CD30- | TCR + CD30T- |
| Date | Percentage | Percentage | Percentage | CSR/TCR | CD28-CSR |
| E1D3 | 28.60 | 30.70 | 20.50 | 1.07 | 1.50 |
| E2D10 | 13.50 | 14.20 | 2.95 | 1.05 | 4.81 |

TABLE 6
CD8⁺R⁺ Central Memory T Cell (Tcm) Counts of T Cells
Expressing anti-AFP-TCR and anti-GPC3-CSR with CD30TM.

| | | | | | Ratio of |
|---|---|---|---|---|---|
| | | | | | Tcm Counts |
| | | AFP-TCR + | AFP-TCR + | Ratio of | TCR + CD30- |
| | | GPC3-CD30- | GPC3-CD30T- | Tcm Counts | CSR/TCR + |
| | AFP-TCR | CSR Tcm | CD28-CSR | TCR + CD30- | CD30T-CD28- |
| Date | Tcm Count | Count | Tcm Count | CSR/TCR | CSR |
| E1D3 | 4,929 | 17,973 | 12,041 | 3.65 | 1.49 |
| E2D10 | 1,404 | 2,948 | 366 | 2.10 | 8.05 |

Table 3: T cells expressing AFP-TCR and GPC3-CD28T-CD30-CSR showed higher cell counts of central memory T cells in the receptor+ CD8+ population at all the time points tested than T cells expressing AFP-TCR only or T cells expressing AFP-TCR and GPC3-CD28T41BB or CD28T-DAP10 CSR (each CSR has the same CD28 TM domain), suggesting better T cell persistence contributed by the CD30IC domain.

Table 4: T cells expressing AFP-TCR and GPC3-CD28T-CD30-CSR showed higher percentage of central memory T cells in the receptor+ CD8+ population at all the time points tested than T cells expressing AFP-TCR only or T cells expressing AFP-TCR and GPC3-CD28T41BB or CD28T-DAP10 CSR (each CSR has the same CD28-TM domain), suggesting better T cell persistence contributed by the CD30IC domain.

Table 5: T cells expressing AFP-TCR and GPC3-CD30-CSR showed higher cell counts of central memory T cells in the receptor+ CD8+ population at all the time points tested than T cells expressing AFP-TCR only or T cells expressing AFP-TCR and GPC3-CD30T-CD28-CSR which has the same CD30-TM domain, suggesting better T cell persistence contributed by the CD30IC domain.

Table 6: T cells expressing AFP-TCR and GPC3-CD30-CSR showed higher percentage of central memory T cells in the receptor+ CD8+ population at all the time points tested than T cells expressing AFP-TCR only or T cells expressing AFP-TCR and GPC3-CD30T-CD28-CSR which has the same CD30-TM domain, suggesting potentially better persistence contributed by the CD30IC domain.

E. Expression of T Cell Exhaustion Markers in T Cells after Co-Culture with Target Cells

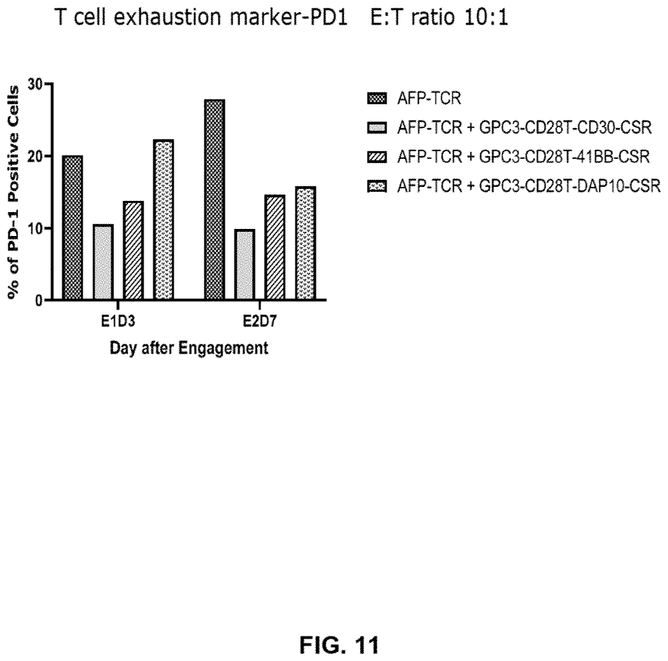

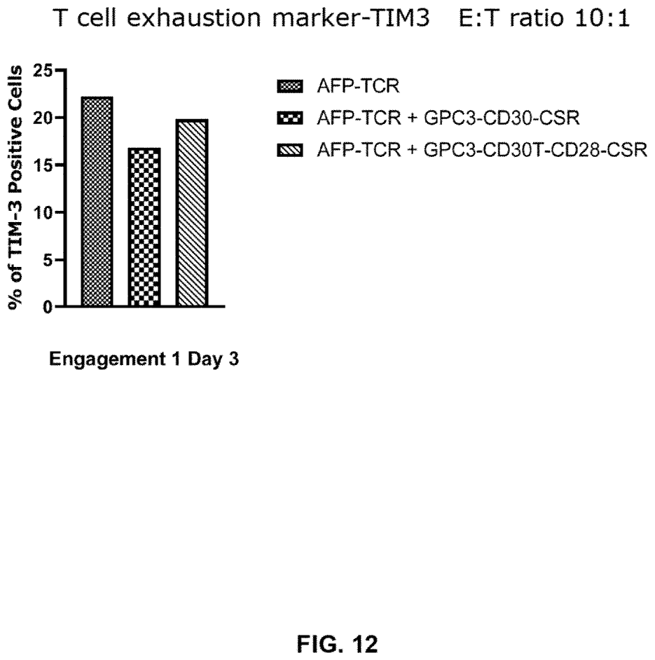

Molecules such as PD-land TIM-3 are inhibitory receptors that accumulate on T cells as T cells lose function. Because of this phenomenon, the expression of these molecules is seen as a marker of exhausted T cell function (also called T cell anergy). To examine the level of exhaustion markers expressed on AFP-TCR+GPC3-CSR-transduced cells upon antigen stimulation, the various T cell groups described earlier in this example were co-cultured with target cells for 3 days at an effector-to-target ratio of 10:1 (the first engagement or “E1”). On E1D3, the T cells were analyzed for exhaustion marker expression by FACS using antibodies to exhaustion marker PD-1 or TIM-3. Then, on the same day. T cells were rechallenged with more target cells (the second engagement or “E2”) and analyzed for PD-1 expression on day 7 post E2. The PD-1 positive cell percentages are shown in FIG. 11 (see, also Tables 7 and 8), while TIM-3 positive cell percentages in FIG. 12 (see also, Table 9).

TABLE 7
PD-1-Positive T Cell Percentages among CD8⁺R⁺ T Cells
Expressing anti-AFP-TCR and anti-GPC3-CSR with CD30TM.

| | | | | | Ratio PD-1 |
|---|---|---|---|---|---|
| | | | | | Percentages |
| | | AFP-TCR + | AFP-TCR + | Ratio PD-1 | TCR + CD30- |
| | AFP-TCR | GPC3-CD30- | GPC3-CD30T- | Percentages | CSR/TCR + |
| | PD-1 | CSR PD-1 | CD28-CSR PD-1 | TCR + CD30- | CD30T + CD28- |
| Date | Percentage* | Percentage | Percentage | CSR/TCR | CSR |
| E1D3 | 20.1 | 13.4 | 16.9 | 0.67 | 0.79 |
| E2D7 | 27.9 | 13.4 | 19.1 | 0.48 | 0.70 |

*PD-1 Percentage: PD-1-Positive T Cell Percentage among CD8⁺R⁺ T Cells

TABLE 8
PD-1-Positive T Cell Percentages among CD8⁺R⁺ T Cells
Expressing anti-AFP-TCR and anti-GPC3-CSR with CD28TM.

| | | | | | | Ratio PD-1 | Ratio PD-1 |
|---|---|---|---|---|---|---|---|
| | | | | | | Percentages | Percentages |
| | | AFP-TCR + | AFP-TCR + | AFP-TCR + | Ratio PD-1 | TCR + | TCR + |
| | | GPC3-CD28T- | GPC3-CD28T- | GPC3-CD28T- | Percentages | CD28T-CD30- | CD28T-CD30- |
| | AFP-TCR | CD30-CSR | 41BB-CSR | DAP10IC- | TCR + | CSR/TCR + | CSR/TCR + |
| | PD-1 | PD-1 | PD-1 | CSR PD-1 | CD28T-CD30- | CD28T-41BB- | CD28T + |
| Date | Percentage* | Percentage | Percentage | Percentage | CSR/TCR | CSR | DAP10-CSR |
| E1D3 | 20.1 | 10.6 | 13.8 | 22.3 | 0.53 | 0.77 | 0.48 |
| E2D7 | 27.9 | 9.87 | 14.6 | 15.8 | 0.35 | 0.68 | 0.62 |

*PD-1 Percentage: PD-1-Positive T Cell Percentage among CD8⁺R⁺ T Cells

TABLE 9
TIM-3-Positive T Cell Percentages among CD8+R+ T Cells
Expressing anti-AFP-TCR and anti-GPC3-CSR with CD30TM.

| | | | | | Ratio TIM-3 |
|---|---|---|---|---|---|
| | | | | | Percentages |
| | | AFP-TCR + | AFP-TCR + | Ratio TIM-3 | TCR + CD30- |
| | AFP-TCR | GPC3-CD30- | GPC3-CD30T- | Percentages | CSR/TCR + |
| | TIM-3 | CSR TIM-3 | CD28-CSR TIM-3 | TCR + CD30- | CD30T + CD28- |
| Date | Percentage* | Percentage | Percentage | CSR/TCR | CSR |
| E1D3 | 22.2 | 16.8 | 19.8 | 0.76 | 0.85 |

*TIM-3 Percentage: PD-1-Positive T Cell Percentage among CD8+R+ T Cells

The result shows that AFP-TCR Tcells coexpressing GPC3-CD28T-CD30-CSR had significantly lower percentages of PD-1 positive cells than did T cells expressing the TCR alone or expressing the TCR and a CSR comprising the same antigen recognition moiety and TM region but an IC signaling domain from a different costimulatory molecule i.e., 4-1BB or DAP10.

The result also shows that AFP-TCR T cells coexpressing GPC3-CD30-CSR had a significantly lower percentage of TIM-3 positive cells than did T cells expressing only the TCR, as well as a lower percentage of TIM-3 positive cells than did AFP-TCR T cells coexpressing GPC3-CD30T-CD28-CSR which comprises the same antigen recognition moiety and TM region but the IC signaling domain from CD28.

These results indicate that coexpressing a CSR containing the CD30 IC domain significantly decreased the exhaustion levels of T cells expressing TCR, while coexpressing a CSR containing a different costimulatory molecule's IC domain did not have such a significant exhaustion-decreasing effect.

### Example 12—Enrichment of T Cells Expressing AFP-TCR+GPC3-CD30-CSR in a Xenograft Mouse Model

This example shows that T cells co-expressing anti-AFP/MHC TCR and anti-GPC3-CD30-CSR persisted or proliferated better than T cells co-expressing the same TCR but CD28-CSR in vivo.

In this example, primary T cells were engineered to express the following constructs with CSRs comprising CD30 or CD28 costimulatory domains:
anti-AFP-TCR1+anti-GPC3-CD28-CSR (or “AFP-TCR+GPC3-CD28-CSR), or
anti-AFP-TCR1+anti-GPC3-CD30-CSR (or “AFP-TCR+GPC3-CD30-CSR).

Engineered T cells expressing these constructs were injected to animals bearing a human liver cancer xenograft. The engineered T cells were produced as described in Materials and Methods. A liver cancer xenograft was transplanted into animals which were then dosed with engineered T cells, and was performed as follows:

About 10⁷ tumor cells comprising liver cancer HepG2 cells (AFP⁺ GPC3⁺) were implanted subcutaneously into NSG mice and allowed to form a solid tumor with a mass of about 200 mm³. Then, 10×10⁶ engineered T cells produced from healthy human primary T cells expressing the TCR+CSR combinations were injected (i.v.) into each tumor-bearing mouse. 17 days after T cell dosing, the T cells (CD3⁺ cells) in the peripheral blood of the engrafted mice were isolated and analyzed, and the result is shown in Table 10.

TABLE 10
Percentages of TCR⁺ CSR⁺ T cells among all T cells
before and after T cell dosing of tumor-bearing mice.

| Construct expressed | Prior to T | 17 days post T |
|---|---|---|
| by T cells | cell dosing | cell dosing |
| AFP-TCR + GPC3- | 31.8% | 23.1% |
| CD28-CSR | | |
| AFP-TCR + GPC3- | 29.1% | 37.9% |
| CD30-CSR | | |

The percentage of T cells co-expressing AFP-TCR and GPC3-CD30-CSR among total T cells increased compared to the percentage present prior to injection into mice, implying that the TCR+CD30-CSR T cells proliferated following injection. At the same time, the percentage of T cells co-expressing the same TCR and CD28-CSR with the same GPC3 binding sequence among total T cells had decreased (Table 10). The number of T cells co-expressing AFP-TCR and GPC3-CD30-CSR circulating in the whole blood of each engrafted mouse (average 8.3 cells per μl blood) was also higher than that of T cells co-expressing the same TCR and CD28-CSR with the same GPC3 binding sequence (average 6.3 cells per μl blood). This example shows that T cells expressing TCR and CD30-CSR persisted or proliferated better than T cells expressing the same TCR but the CD28-CSR in vivo.

### Example 13—Tumor Cell Killing by Anti-AFP/MHC TCR+Anti-MSLN-CSR T Cells

This example shows that T cells co-expressing anti-AFP-TCR and a CD30-CSR targeting another antigen, MSLN, have higher specific tumor cell killing efficacies than T cells expressing the TCR alone or the TCR and a CD28-CSR, especially over long-term. In this example, T cells expressing the following constructs were generated and compared for target cell killing capability.
(1) anti-AFP-TCR1 (or “anti-AFP-TCR”),
(2) anti-AFP-TCR1+anti-MSLN-CD28-CSR, or
(3) anti-AFP-TCR1+anti-MSLN-CD30-CSR.

A cell line HepG2-MSLN was generated to be used as target cells for this example by engineering the HepG2 cell line to artificially express human mesothelin protein.

Lentiviruses encoding TCRs or TCR+CSR were produced as described in Materials and Methods. T cells from two donors, donor code L110042668 (abbreviated as “R68”) and donor code L110048074 (“R74”), were purchased from Allcells, and their endogenous TCRs were knocked out (“TCR-KO”) for this example. These TCR-KO T cells were transduced with these lentiviruses as described in Materials and Methods. Transduction efficiencies were assessed by flow cytometry. For anti-AFP TCRs, an antibody that binds to alpha/beta TCRs was used. For anti-MSLN CSR, an anti-myc antibody was used.

### A—Short-Term In Vitro Tumor Cell Killing Assay (LDH-Based)

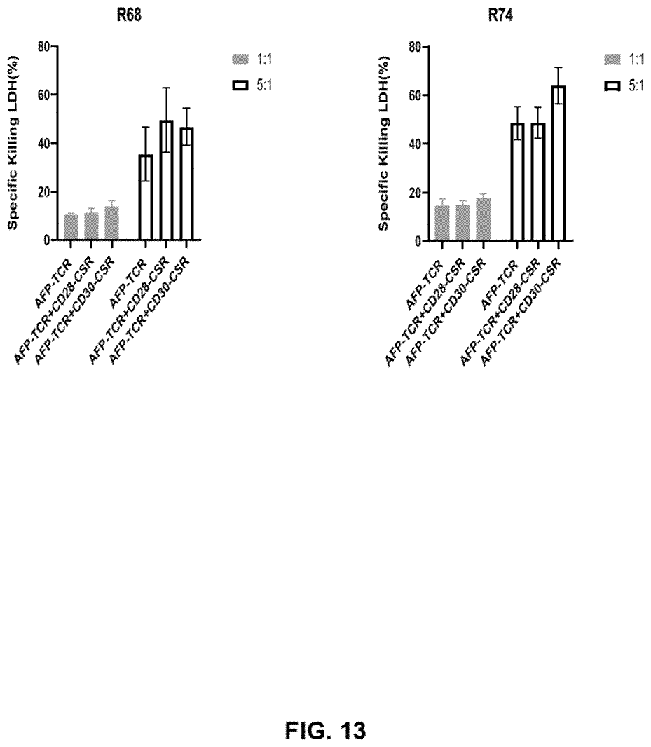

TCR-KO T cells expressing the TCR or TCR+CSR constructs were activated and expanded with CD3/CD28 Dynabeads (Invitrogen) according to manufacturer's protocol. Activated T cells (ATC) are cultured and maintained in RPMI 1640 medium with 10% FBS plus 100 U/ml IL-2 and used on day 12. The T cells were >99% CD3⁺ by FACS analysis. Activated effector cells and the target cells (HepG2-MSLN) were co-cultured at an E:T ratio between 1:1 to 5:1 for 16-24 hours. Specific killing was determined by measuring LDH activity in culture supernatants. Tumor cytotoxicity was assayed using an LDH Cytotoxicity Assay (Promega). The results are shown in FIG. 13. T cells expressing anti-AFP-TCR1+anti-MSLN-CD30-CSR (“AFP-TCR+CD30-CSR”) showed higher short-term target-specific cytotoxicity than T cells expressing only anti-AFP-TCR1 (“AFP-TCR”) or T cells expressing anti-AFP-TCR1+anti-MSLN-CD28-CSR (“AFP-TCR+CD28-CSR”) at an E:T ratio of 1:1 using T cells from both donors. At an E:T ratio of 5:1, T cells expressing AFP-TCR+CD30-CSR showed higher cytotoxicity than T cells expressing only AFP-TCR using T cells from both donors, while showing a higher cytotoxicity than T cells expressing AFP-TCR+CD28-CSR with T cells from a single donor.

### B—Short-Term In Vitro Tumor-Specific T Cell Cytokine Release Assay (IFN-γ)

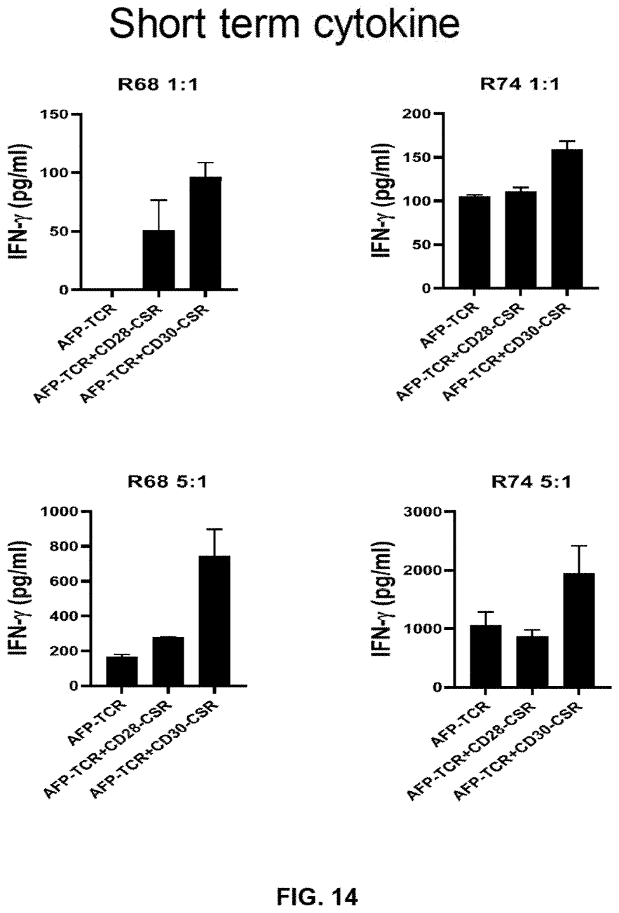

The short-term killing ability of the three TCR or TCR+CSR T cell groups (the effector cells) described in this example was also determined by measuring the amounts/levels of the cytokine IFNγ released from T cells upon engagement with target cells as described in Example 1. The levels of IFNγ release in the supernatant after 16 hour co-culture were quantified by ELISA. T cells with high cytotoxic potency secrete high levels of IFNγ. The results are shown in FIG. 14. T cells expressing anti-AFP-TCR1+anti-MSLN-CD30-CSR (“AFP-TCR+CD30-CSR”) showed higher levels of IFNγ release, and thus higher short-term tumor-cell-killing capability, than T cells expressing only anti-AFP-TCR1 (“AFP-TCR”) or T cells expressing anti-AFP-TCR1+anti-MSLN-CD28-CSR (“AFP-TCR+CD28-CSR”) at the E:T ratios of both 1:1 and 5:1 with T cells from both donors.

### C—Long-Term In Vitro Tumor Cell Killing Assay

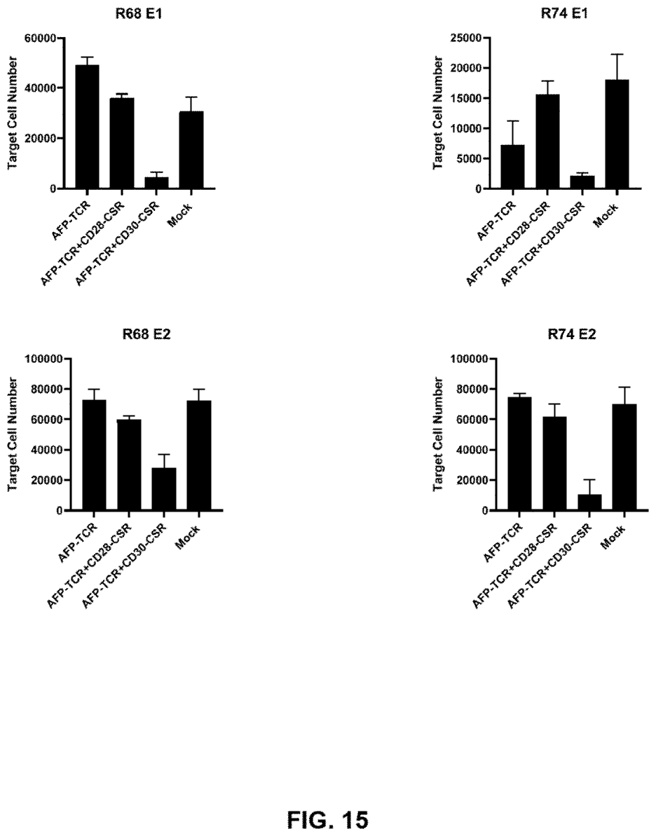

A crystal-violet staining-based assay for counting target cells was used to compare the long-term target-cell killing capability of the three TCR or TCR+CSR T cell groups (the effector cells) described in this example, in addition to “Mock” T cells, which are the same T cells not transduced with lentivirus. In this experiment, 250,000 total T cells of each T cell group were incubated with target cells at an effector cell-to-target cell ratio (E:T ratio) of 5:1 (the first target cell engagement). The numbers of remaining target cells were quantified with crystal-violet staining three days after the first target cell engagement. Then, on the same day, the mixtures of T cells and target cells were rechallenged with 50,000 fresh target cells (the second target cell engagement). The numbers of remaining target cells were quantified with crystal-violet staining four days after the second target cell engagement. The results are shown in FIG. 15. E1 represents data obtained three days after the first engagement, while E2 represents data obtained four days after the second engagement. T cells expressing anti-AFP-TCR1+anti-MSLN-CD30-CSR (“AFP-TCR+CD30-CSR”) killed more target cells than T cells expressing only anti-AFP-TCR1 (“AFP-TCR”) or T cells expressing anti-AFP-TCR1+anti-MSLN-CD28-CSR (“AFP-TCR+CD28-CSR”) after both the first and second T cell and target cell engagements. This higher long-term target cell killing capability of the AFP-TCR+MSIN-CD30-CSR was observed with T cells derived from both donors.

### Example 14—Long-Term In Vitro Assays with Anti-MSLN/MHC-TCR+Anti-MSLN-CSR T Cells

### A. Long-Term In Vitro Tumor Cell Killing Assay

In this example, the degree of killing of HepG2-MSLN target cells by T cells expressing a TCR targeting another antigen, a complex of an MSLN peptide and MHC, with or without a CSR targeting a full-length MSLN, was measured and compared. The following constructs were employed:
(1) anti-MSLN/MHC-TCR (a.k.a. anti-MSLN-TCR or MSLN-TCR);
(2) anti-MSLN-TCR+anti-MSLN-CD30-CSR (or “MSLN-TCR+MSLN-CD30-CSR”), or
(3) anti-MSLN-TCR+anti-MSLN-CD28-CSR (or “MSLN-TCR+MSLN-CD28-CSR”).

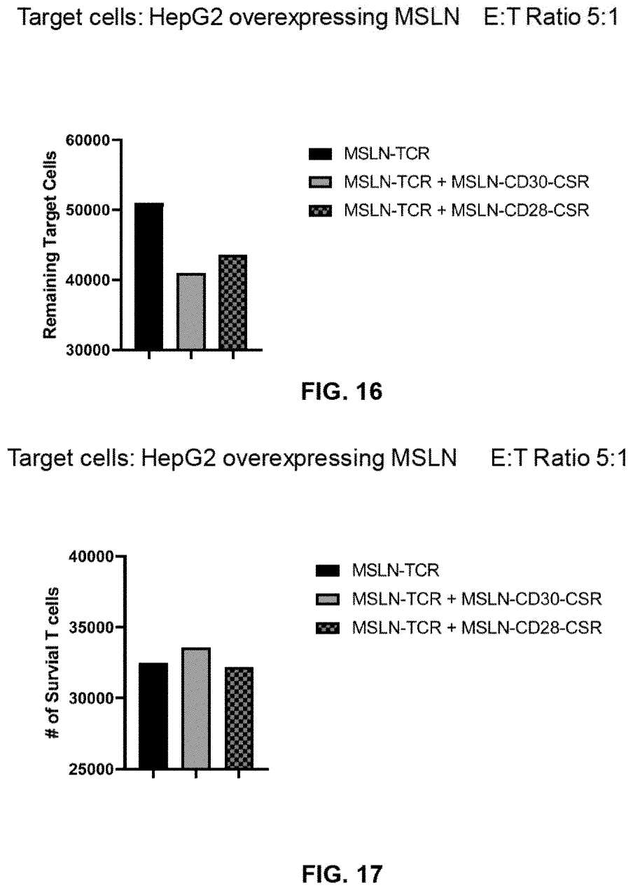

In this experiment, 250,000 total T cells of each T cell group were incubated/engaged with target cells at an effector cell to target cell ratio (E:T ratio) of 5:1. Using a FACS assay, the sample cells were stained for the T cell marker CD3 which was used to gate and count the CD3-negative HepG2-MSLN target cells. The numbers of remaining target cells were quantified at one week after the engagement with T cells, and the result is shown in FIG. 16. The result shows that T cells expressing anti-MSLN-TCR together with anti-MSLN-CD30-CSR killed more target cells (as shown by the depletion of HepG2-MSLN cells) than did T cells expressing the anti-MSLN-TCR alone or T cells expressing anti-MSLN-TCR with anti-MSLN-CD28-CSR.

### B. Long-Term In Vitro T Cell Survival Assay

To examine the ability of the effector T cells to survive (and/or to multiply) during the one-week challenge period (described in section A of this example), the numbers of T cells expressing anti-MSLN TCR alone or expressing anti-MSLN TCR with an anti-MSLN-CSR were quantified by FACS using an antibody to the T cell marker CD3; the result is shown in FIG. 17. The result demonstrates that T cells expressing anti-MSLN-TCR together with anti-MSLN-CD30-CSR shows a higher level of survival and proliferation compared to the corresponding T cells expressing the TCR alone or T cells expressing the TCR with anti-MSLN-CD28-CSR

### C. Long-Term In Vitro T Cell Exhaustion Marker Assay

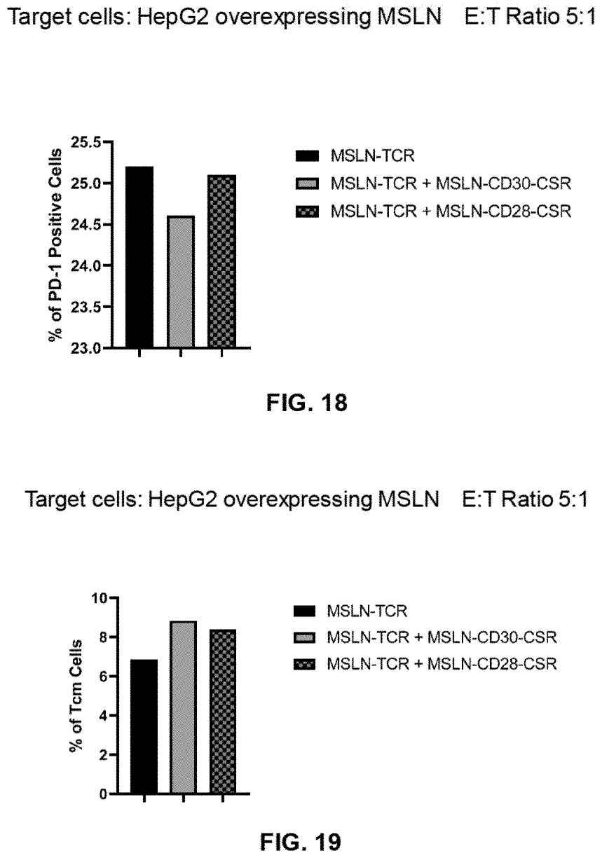

To determine the exhaustion levels of T cells expressing anti-MSLN-TCR alone and T cells also expressing anti-MSLN-CD30-CSR or anti-MSLN-CD28-CSR, a marker of T cell anergy was used. In this example, the inhibitory receptor, T cell exhaustion marker, PD-1, was measured in the T cells using an anti-PD-1 antibody-based FACS assay. T cells expressing anti-MSLN-TCR together with anti-MSLN-CD30-CSR had a lower percentage of PD-1 positive cells than did cells expressing the TCR alone or cells expressing the TCR with anti-MSLN-CD28-CSR, as shown in FIG. 18. This result indicates that T cells expressing anti-MSLN-TCR and anti-MSLN-CD30-CSR are less likely to exhibit T cell exhaustion.

### D. Long-Term In Vitro Memory T Cell Quantification

To assess the ability of the T cells expressing the TCR+CSR combinations to persist during the long-term target cell killing assay described earlier in this example, and therefore to predict their ability to persist in patients, a measure of the memory subset of T cells was conducted using markers CCR7 and CD45RA. As explained earlier in this application, T cells with high CCR7 expression levels and low CD45RA expression levels represent central memory T cells, which have longer lives than effector memory T cells, and are capable of generating effector memory T cells, but not vice versa. Therefore, the ability to develop into and maintain central memory T cells is an important and desired feature for potentially successful T cell therapies. As demonstrated by the results shown in FIG. 19, T cells expressing anti-MSLN-TCR together with anti-MSLN-CD30-CSR comprise a higher percentage of central memory T cells among CD8⁺ T cells (the cytotoxic T cell population) than did cells expressing the TCR alone or expressing the TCR with anti-MSLN-CD28-CSR. This result suggests that T cells expressing anti-MSLN-TCR and anti-MSLN-CD30-CSR persist and function for a longer period.

### Example 15—Long-Term In Vitro Assays with Anti-NY-ESO-1/MHC-TCR+Anti-MUC16-CSR T Cells

In this example, T cells expressing a TCR targeting another antigen, a complex of an NY-ESO-1 peptide and MHC, with or without a CSR targeting yet another antigen, MUC16, were generated, and their long-term persistence and target-cell-killing capabilities were measure and compared. The following constructs were employed:
(1) anti-NY-ESO-1/MHC-TCR (a.k.a. anti-NY-ESO-1-TCR or NY-ESO-1-TCR), comprising SEQ ID NO:362 (TCR alpha chain variable region sequence) fused to SEQ ID NO:709 (TCR alpha chain constant region sequence), and SEQ ID NO:366 (TCR beta chain variable region sequence) fused to SEQ ID NO:710 (TCR beta chain constant region sequence);
(2) anti-NY-ESO-1-TCR+anti-MUC16-CD30-CSR (or “NY-ESO-1-TCR+MUC16-CD30-CSR”), the CSR comprising SEQ ID NO:130 (V_{H}), SEQ ID NO:138 (V_{L}), and SEQ ID NO:217 (CD30-CSR); or
(3) anti-NY-ESO-1-TCR+anti-MUC16-41BB-CSR (or “NY-ESO-1-TCR+MUC16-41BB-CSR”), the CSR comprising SEQ ID NO:130 (V_{H}), SEQ ID NO:138 (V_{L}), and SEQ ID NO:219 (41BB TM and IC).

### A. Long-Term In Vitro Tumor Cell Killing Assay

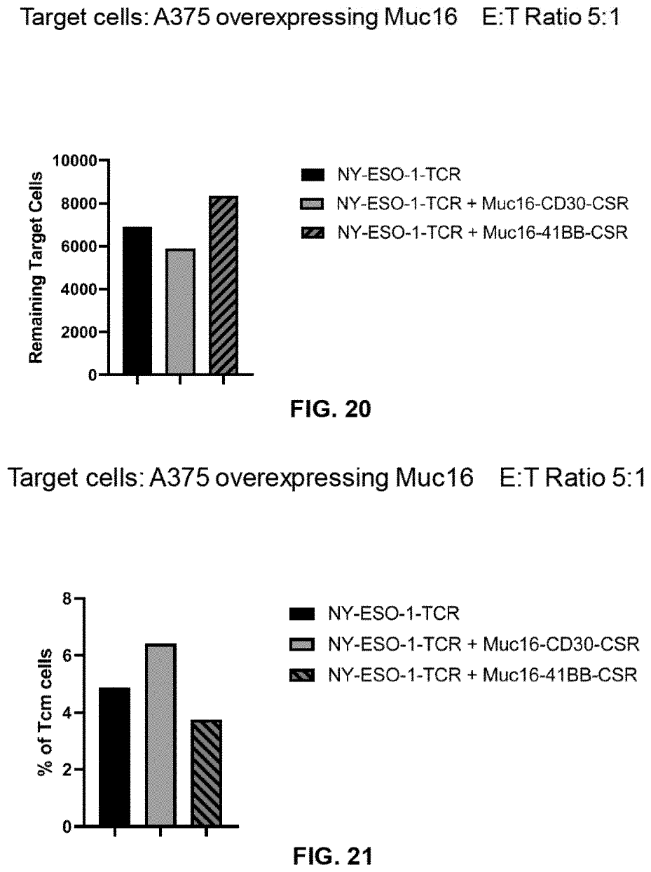

In this experiment, 250,000 total T cells of each T cell group were incubated/engaged with A375-Muc16 target cells at an effector cell-to-target cell ratio (E:T ratio) of 5:1 (the first engagement or “E1”), and then rechallenged with 50.000 target cells four days after E1 (the second engagement or “E2”). Using a FACS assay, the sample cells were stained for the T cell marker CD3 which was used to gate and count the CD3 negative A375-Muc16 target cells. The numbers of remaining target cells were quantified four days after the second engagement with T cells, and the result is shown in FIG. 20.

The result showed that T cells expressing anti-NY-ESO-1-TCR together with anti-MUC16-CD30-CSR killed more target cells (as shown by the depletion of A375-Muc16 cells) than T cells expressing the anti-NY-ESO-1-TCR alone or the anti-NY-ESO-1-TCR together with an anti-MUC16-41BB-CSR, indicating that the CSR with CD30 transmembrane and intracellular signaling domains aids in target cell killing.

In addition, to ask if the effector cell populations were surviving/proliferating during the course of the assay period, the numbers of T cells were measured around one week after the first engagement. The result shows that T cells expressing an anti-NY-ESO-1-TCR together with an anti-MUC16-CD30-CSR proliferated more than corresponding T cells expressing TCR only (data not shown). This indicates that the CSR with CD30 transmembrane and intracellular signaling domains increases T cell survival and proliferation.

### B. Long-Term In Vitro Memory T Cell Quantification

This example shows that a different CSR with a CD30 signaling domain allowed more central memory T cells (Tcm) to develop and persist. A measure of the memory subset of T cells expressing anti-NY-ESO-1-TCR with or without anti-MUC16-CSR was conducted. The receptor⁺ (TCR⁺ and CSR⁺), CD8⁺ T cell population of each sample group was assayed for the expression of the CCR7/CD45RA markers after six days in culture with the A375-Muc16 target cells and the percentages of central memory T cells (Tcm, T cells with high CCR7 levels and low CDR45RA levels) were calculated and shown in FIG. 21. See also Table 11. The result shows that T cells expressing anti-NY-ESO-1-TCR together with anti-MUC16-CD30-CSR comprise a higher percentage of central memory T cells among CD8˜ T cells (cytotoxic T cells) than did cells expressing the TCR alone or expressing a combination of the TCR and a corresponding CSR with a 4-1BB transmembrane domain and intracellular signaling domain (anti-NY-ESO-1-TCR+anti-MUC16-41BB CSR). This result suggests that T cells expressing anti-NY-ESO-1-TCR together with anti-MUC16-CD30-CSR were able to persist and function longer.

TABLE 11
Central memory T Cell (Tcm) percentages among CD8⁺R⁺ T cells
expressing anti-NY-ESO-1-TCR and anti-Muc16-CSRs.

| | | | | | Ratio of Tcm |
|---|---|---|---|---|---|
| | | NY-ESO-1- | NY-ESO-1- | Ratio of Tcm | Percentages |
| | NY-ESO-1 | TCR + Muc16- | TCR + Muc16- | Percentages | TCR + CD30- |
| | TCR Tcm | CD30-CSR Tcm | 41BB-CSR Tcm | TCR + CD30- | CSR/TCR + |
| Date | Percentage | Percentage | Percentage | CSR/TCR | 41BB-CSR |
| E1D6 | 4.88 | 6.4 | 3.75 | 1.31 | 1.71 |

### Materials and Methods for Examples 14 and 15

### Cell Samples, Cell Lines, Antibodies, TCRs, and CSRs

A cell line HepG2-MSLN was generated to be used as target cells for testing T cells expressing anti-MSLN/MHC-TCR with or without CSR by engineering the HepG2 cell line to artificially express full length human mesothelin protein. Another cell line, A375-Muc16, was generated to be used as target cells for testing T cells expressing anti-NY-ESO-1/MHC-TCR (with or without a CSR) by engineering the A375 cell line to artificially express the full-length human Muc16 protein. The cell line A375 (ATCC CRL-1619™; HLA-A2+, NY-ESO-1+) was obtained from the American Type Culture Collection. Cells were cultured in DMEM supplemented with 10% FBS and 2 mM glutamine at 37° C./5% CO2.

Antibodies against human or mouse TCR, CD3, CD4, CD8, CCR7, CD45RA, and PD-1 used in these assays were purchased from BioLegend or Cell Signaling Technology.

T cells from healthy donors were first activated as previously described. The endogenous TCRs in these activated T cells were knocked out (“TCR-KO”) using a CRISPR-Cas-9 system as described in the MATERIALS AND METHODS section at the beginning of the EXAMPLES. The TCR-KO T cells were transduced with lentiviruses expressing the TCR or TCR+CSR. T cells were cultured and maintained in RPMI 1640 medium with 10% FBS plus 100 U/ml IL-2 and used at day 9. The T cells were >99% CD3+ by FACS analysis. For the long-term killing assay, 250.000 total T cells of each TCR or TCR+CSR cell group were incubated with target cells at an effector cell-to-target cell ratio (E:T ratio) of 5:1 for 1-2 weeks. Following the E:T challenge, the numbers of remaining target cells and surviving T cells were stained with anti-CD3 and quantified by FACS. The samples were also stained for the presence of the TCR and T cell markers CD3, CD4, CD8, CCR7, CD45RA, and PD-1 to determine T cell subsets, T cell persistence and T cell exhaustion levels.

### Informal Sequence Listing

(Note: For SEQ ID NOS:1-5 and 178-180, signal sequence: plain text; variable region: bold; CDRs: bold and underlined; constant region: italicized; transmembrane and cytoplasmic region: italicized and underlined). Illustrative references regarding SEQ ID NOS:485-708 include Xu, Y. et al. Cancer Immunol Immunother 2019; 68(12):1979-1993; Keskin, D. et al. Nature 2019; 565(7738):234-239; Stronen, E. et al. Science 2016; 352(6291):1337-41; Zacharakis, N. et al. Nat Med 2018; 24(6):724-730; Tran, E. et al. Science 2015; 350(6266):1387-90; Parkhurst, M. Clin Cancer Res 2017; 23(10):2491-2505; Kato. T. et al Oncotarget 2018; 9(13):11009-11019; Veatch, J. et al. Cancer Immunol Res 2019; 7(6):910-922; Tran, E., et al. N Engl J Med 2016; 375(23):2255-2262; Gros, A. et al. Nat Med 2016; 22(4):433-8; Lo, W. et al. Cancer Immunol Res 2019; 7(4):534-543; Malekzadeh, P. et al. J Clin Invest 2019; 129(3):1109-1114; Parkhurst, M. et al. Cancer Discov 2019; 9(8):1022-1035; and Jaigirdar, A. et al. J Immunother 2016; 39(3):105-16.

| SEQ | | |
|---|---|---|
| ID | | |
| NO. | Sequence | Notes |
| 1 | MMKSLRVLLVILWLQLSWVWSQQKEVEQNSGPLSVPEGAIASLNCTYSDRGSQSF | Anti-AFP-TCR1 alpha |
| | FWYRQYSGKSPELIMSIYSNGDKEDGRFTAQLNKASQYVSLLIRDSQPSDSATYL | chain |
| | CAVNSDSGYALNFGKGTSLLVTPHIQNPDPAVYQLRDSKSSDKSVCLFTDFDSQT | |
| | NVSQSKDSDVYITDKTVLDMRSMDFKSNSAVAWSNKSDFACANAFNNSIIPEDTF | |
| | FPSPESSCDVKLVEKSFETDTNLNFQNLSVIGFRILLLKVAGFNLLMTLRLWSS | |
| 2 | MGFRLLCCVAFCLLGAGPVDSGVTQTPKHLITATGQRVTLRCSPRSGDLSVYWYQ | Anti-AFP-TCR1 and |
| | QSLDQGLQFLIQYYNGEERAKGNILERFSAQQFPDLHSELNLSSLELGDSALYFC | Anti-AFP-TCR2 beta |
| | ASSLGGESEQYFGPGTRLTVTEDLKNVFPPEVAVFEPSEAEISHTQKATLVCLAT | chain |
| | GFYPDHVELSWWVNGKEVHSGVSTDPQPLKEQPALNDSRYCLSSRLRVSATFWQN | |
| | PRNHFRCQVQFYGLSENDEWTODRAKPVTQIVSAEAWGRADCGFTSESYQQGVLS | |
| | ATILYEILLGKATLYAVLVSALVLMAMVKRKDSRG | |
| 3 | MMKSLRVLLVILWLQLSWVWSQQKEVEQNSGPLSVPEGAIASLNCTYSDRGSQAF | Anti-AFP-TCR2 alpha |
| | FWYRQYSGKSPELIMSIYSNGDKEDGRFTAQLNKASQYVSLLIRDSQPSDSATYL | chain, affinity- |
| | CAVNSQSGYALNFGKGTSLLVTPHIQNPDPAVYQLRDSKSSDKSVCLFTDFDSQT | enhanced version of |
| | NVSQSKDSDVYITDKTVLDMRSMDFKSNSAVAWSNKSDFACANAFNNSIIPEDTF | Anti-AFP-TCR1 alpha |
| | FPSPESSCDVKLVEKSFETDTNLNFQNLSVIGFRILLLKVAGFNLLMTLRLWSS | chain |
| 4 | METLLGLLILWLQLQWVSSKQEVTQIPAALSVPEGENLVLNCSFTDSAIYNLQWF | Anti-NY-ESO-1 TCR |
| | RQDPGKGLTSLLLIQSSQREQTSGRLNASLDKSSGRSTLYIAASQPGDSATYLCA | alpha chain |
| | VRPTSGGSYIPTFGRGTSLIVHPYIQNPDPAVYQLRDSKSSDKSVCLFTDFDSQT | |
| | NVSQSKDSDVYITDKTVLDMRSMDFKSNSAVAWSNKSDFACANAFNNSIIPEDTF | |
| | FPSPESSCDVKLVEKSFETDTNLNFQNLSVIGFRILLLKVAGFNLLMTLRLWSS | |
| 5 | MSIGLLCCAALSLLWAGPVNAGVTQTPKFQVLKTGQSMTLQCAQDMNHEYMSWYR | Anti-NY-ESO-1 TCR |
| | QDPGMGLRLIHYSVGAGITDQGEVPNGYNVSRSTTEDFPLRLLSAAPSQTSVYFC | beta chain |
| | ASSYVGNTGELFFGEGSRLTVLEDLKNVFPPEVAVFEPSEAEISHTQKATLVCLA | |
| | TGFYPDHVELSWWVNGKEVHSGVSTDPQPLKEQPALNDSRYCLSSRLRVSATFWQ | |
| | NPRNHFRCQVQFYGLSENDEWTQDRAKPVTQIVSAEAWGRADCGFTSESYQQGVL | |
| | SATILYEILLGKATLYAVLVSALVLMAMVKRKDSR | |
| 6 | QKEVEQNSGPLSVPEGAIASLNCTYSDRGSQAFFWYRQYSGKSPELIMSIYSNGDKED | anti-AFP-TCR alpha |
| | GRETAQLNKASQYVSLLIRDSQPSDSATYLCAVNSDSGYALNFGKGTSLLVTPHIQNP | chain variant |
| | DPAVYQLRDSKSSDKSVCLFTDFDSQTNVSQSKDSDVYITDKTVLDMRSMDFKSNSAV | |
| | AWSNKSDFACANAFNNSIIPEDTFFPSPESS | |
| 7 | QKEVEQNSGPLSVPEGAIASLNCTYSDRGSQSFFWYRQYSGKSPELIMSIYSNGDKED | anti-AFP-TCR alpha |
| | GRFTAQLNKASQYVSLLIRDSQPSDSATYLCAVNSDSSYALNFGKGTSLLVTPHIQNP | chain variant |
| | DPAVYQLRDSKSSDKSVCLFTDFDSQTNVSQSKDSDVYITDKTVLDMRSMDFKSNSAV | |
| | AWSNKSDFACANAFNNSHIPEDTFFPSPESS | |
| 8 | QKEVEQNSGPLSVPEGAIASLNCTYSDRGSQSFFWYRQYSGKSPELIMSIYSNGDKED | anti-AFP-TCR alpha |
| | GRETAQLNKASQYVSLLIRDSQPSDSATYLCAVNSDSGVALNFGKGTSLLVTPHIQNP | chain variant |
| | DPAVYQLEDSKSSDKSVCLFTDFDSQTNVSQSKDSDVYITDKTVLDMRSMDFKSNSAV | |
| | AWSNKSDFACANAFNNSHIPEDTFFPSPESS | |
| 9 | QKEVEQNSGPLSVPEGAIASLNCTYSDRGSQAFFWYRQYSGKSPELIMSTYSNGDKED | anti-AFP-TCR alpha |
| | GRETAQLNKASQYVSLLIRDSQPSDSATYLCAVNSDSGVALNFGKGTSLLVTPHIQNP | chain variant |
| | DPAVYQLRDSKSSDKSVCLFTDFDSQTNVSQSKDSDVYITDKTVLDMRSMDFKSNSAV | |
| | AWSNKSDFACANAFNNSHIPEDTFFPSPESS | |
| 10 | QKEVEQNSGPLSVPEGAIASLNCTYSDRGSQSFFWYRQYSGKSPELIMSIYSNGDKED | anti-AFP-TCR alpha |
| | GRFTAQLNKASQYVSLLIRDSQPSDSATYLCAVNSQSGYALNFGKGTSLLVTPHIQNP | chain variant |
| | DPAVYQLRDSKSSDKSVCLFTDFDSQTNVSQSKDSDVYITDKTVLDMRSMDFKSNSAV | |
| | AWSNKSDFACANAFNNSIIPEDTFFPSPESS | |
| 11 | QKEVEQNSGPLSVPEGAIASLNCTYSDRGSQSFFWYRQYSGKSPELIMSIYSNGDKED | anti-AFP-TCR alpha |
| | GRETAQLNKASQYVSLLIRDSQPSDSATYLCAVNSQSGYSLNFGKGTSLLVIPHIQNP | chain variant |
| | DPAVYQLEDSKSSDKSVCLFTDFDSQTNVSQSKDSDVYITDKTVLDMRSMDFKSNSAV | |
| | AWSNKSDFACANAFNNSHIPEDTFFPSPESS | |
| 12 | QKEVEQNSGPLSVPEGAIASLNCTYSDRGSQSFFWYRQYSGKSPELIMSIYSNGDKED | anti-AFP-TCR alpha |
| | GRETAQLNKASQYVSLLIRDSQPSDSATYLCAVNSQSSYALNFGKGTSLLVTPHIQNP | chain variant |
| | DPAVYQLRDSKSSDKSVCLFTDFDSQTNVSQSKDSDVYITDKTVLDMRSMDFKSNSAV | |
| | AWSNKSDFACANAFNNSIIPEDTFFPSPESS | |
| 13 | QKEVEQNSGPLSVPEGAIASLNCTYSDRGSQSFFWYRQYSGKSPELIMSIYSNGDKED | anti-AFP-TCR alpha |
| | GRFTAQLNKASQYVSLLIRDSQPSDSATYLCAVNSQSGVALNFGKGTSLLVTPHIQNP | chain variant |
| | DPAVYQLRDSKSSDKSVCLFTDFDSQTNVSQSKDSDVYITDKTVLDMRSMDFKSNSAV | |
| | AWSNKSDFACANAFNNSHIPEDTFFPSPESS | |
| 14 | QKEVEQNSGPLSVPEGAIASLNCTYSDRGSQSFFWYRQYSGKSPELIMSIYSNGDKED | anti-AFP-TCR alpha |
| | GRETAQLNKASQYVSLLIRDSQPSDSATYLCAVNSQNGYALNFGKGTSLLVTPHIQNP | chain variant |
| | DPAVYQLRDSKSSDKSVCLFIDFDSQTNVSQSKDSDVYITDKTVLDMRSMDFKSNSAV | |
| | AWSNKSDFACANAFNNSIIPEDTFFPSPESS | |
| 15 | QKEVEQNSGPLSVPEGAIASLNCTYSDRGSFSFFWYRQYSGKSPELIMSIYSNGDKED | anti-AFP-TCR alpha |
| | GRFTAQLNKASQYVSLLIRDSQPSDSATYLCAVNSDSGYALNFGKGTSLLVTPHIQNP | chain variant |
| | DPAVYQLRDSKSSDKSVCLFTDFDSQTNVSQSKDSDVYITDKTVLDMRSMDFKSNSAV | |
| | AWSNKSDFACANAFNNSIIPEDTFFPSPESS | |
| 16 | QKEVEQNSGPLSVPEGAIASLNCTYSDRGSYSFFWYRQYSGKSPELIMSTYSNGDKED | anti-AFP-TCR alpha |
| | GRFTAQLNKASQYVSLLIRDSQPSDSATYLCAVNSDSGYALNFGKGTSLLVTPHIQNP | chain variant |
| | DPAVYQLRDSKSSDKSVCLFTDFDSQTNVSQSKDSDVYITDKTVLDMRSMDFKSNSAV | |
| | AWSNKSDFACANAFNNSHIPEDTFFPSPESS | |
| 17 | QKEVEQNSGPLSVPEGAIASLNCTYSDRGSYSFFWYRQYSGKSPELIMSIYSNGDKED | anti-AFP-TCR alpha |
| | GRETAQLNKASQYVSLLIRDSQPSDSATYLCAVNSDSSYALNFGKGTSLLVTPHIQNP | chain variant |
| | DPAVYQLEDSKSSDKSVCLFTDFDSQTNVSQSKDSDVYITDKTVLDMRSMDFKSNSAV | |
| | AWSNKSDFACANAFNNSHIPEDTFFPSPESS | |
| 18 | KEVEQNSGPLSVPEGAIASLNCTYSDRGSYSFFWYRQYSGKSPELIMSIYSNGDKEDG | anti-AFP-TCR alpha |
| | RFTAQLNKASQYVSLLIRDSQPSDSATYLCAVNSDSGVALNFGKGTSLLVTPHIQNPD | chain variant |
| | PAVYQLRDSKSSDKSVCLFTDFDSQTNVSQSKDSDVYITDKTVLDMRSMDFKSNSAVA | |
| | WSNKSDFACANAFNNSIIPEDTFFPSPESS | |
| 19 | QKEVEQNSGPLSVPEGAIASLNCTYSDRGSYSFFWYRQYSGKSPELIMSIYSNGDKED | anti-AFP-TCR alpha |
| | GRETAQLNKASQYVSLLIRDSQPSDSATYLCAVNSQSGYALNFGKGTSLLVTPHIQNP | chain variant |
| | DPAVYQLRDSKSSDKSVCLFTDFDSQTNVSQSKDSDVYITDKTVLDMRSMDFKSNSAV | |
| | AWSNKSDFACANAFNNSIIPEDTFFPSPESS | |
| 20 | QKEVEQNSGPLSVPEGAIASLNCTYSDRGSQSFFWYRQYSGKSPELIMSIYSNGDKED | Anti-PSA TCR1 alpha |
| | GRFTAQLNKASQYVSLLIRDSQPSDSATYLCAVWGKSGGYNKLIFGAGTRLAVHP | chain |
| 21 | NAGVTQTPKFGILKTGQSMTLQCAQDMNHEYMSWYRQDPGMGLRLIHYSVGAGITDKG | Anti-PSA TCR1 beta |
| | EVPNGYNVSRSTTEDFPLRLELAAPSQTSVYFCASSYSSPLRQDNSPLHFGNGTRLTV | chain |
| | T | |
| 22 | QKEVEQNSGPLSVPEGAIASLNCTYSDRGSQSFFWYRQYSGKSPELIMSIYSNGDKED | Anti-PSA TCR2 alpha |
| | GRFTAQLNKASQYVSLLIRDSQPSDSATYLCAVNSPGNARLMFGDGTQLVVKP | chain |
| 23 | NAGVTQTPKFQVLKTGQSMTLQCAQDMNHEYMSWYRQDPGMGLRLIHYSVGAGITDQG | Anti-PSA TCR2 beta |
| | EVPNGYNVSRSTTEDFPLRLLSAAPSQTSVYFCASSYWRGGLEQYFGPGTRLTVT | chain |
| 24 | AQSVTQLDSHVSVSEGTPVLLRCNYSSSVPPYLFWYVQYPNQGLQLLLKYTTGATLVK | Anti-PSA TCR3 alpha |
| | GINGFEAEFKKSETSFHLTKPSAHMSDAAEYFCAVSEDGNKLVFGAGTILRVKS | chain |
| 25 | AAGVIQSPRHLIKEKRETATLKCYPIPRHDTVYWYQQGPGQDPQFLISFYEKMQSDKG | Anti-PSA TCR3 beta |
| | SIPDRFSAQQFSDYHSELNMSSLELGDSALYFCASRLTGVSGEGDYGYTFGSGTRLTV | chain |
| | V | |
| 26 | PLFQVPEPV | AFP peptide can be |
| | | targeted by TCR; |
| | | hAFP137-145 |
| 27 | FMNKFIYEI | AFP peptide can be |
| | | targeted by TCR; |
| | | hAFP158-166 |
| 28 | GLSPNLNRFL | AFP peptide can be |
| | | targeted by TCR; |
| | | hAFP325-334 |
| 29 | GVALQTMKQ | AFP peptide can be |
| | | targeted by TCR; |
| | | hAFP542-550 |
| 30 | AMNKFIYEI | AFP peptide can be |
| | | targeted by TCR; |
| | | hAFP158 A1 |
| 31 | FMAKFIYEI | AFP peptide can be |
| | | targeted by TCR; |
| | | hAFP158 A3 |
| 32 | FMNAFIYEI | AFP peptide can be |
| | | targeted by TCR; |
| | | hAFP158 A4 |
| 33 | FMNKAIYEI | AFP peptide can be |
| | | targeted by TCR; |
| | | hAFP158 A5 |
| 34 | FMNKFAYEI | AFP peptide can be |
| | | targeted by TCR; |
| | | hAFP158 A6 |
| 35 | FMNKFIAEI | hAFP158 A7 |
| 36 | FMNKFIYAI | AFP peptide can be |
| | | targeted by TCR; |
| | | hAFP158 A8 |
| 37 | SLLMWITQC | NY-ESO-1 peptide can |
| | | be targeted by TCR |
| 38 | FLTPKKLQCV | PSA peptide can be |
| | | targeted by TCR |
| 39 | VISNDVCAQV | PSA peptide can be |
| | | targeted by TCR |
| 40 | KVMDLPTQEPAL | PSA peptide can be |
| | | targeted by TCR |
| 41 | EVQLLESGGGLVQPGGSLRLSCAASGFTFSSYAMTWVRQAPGKGLEWVSAISGSGYS | Anti-HER2_V_{H} |
| | TYYADSEKGRFTISRDNSKNTLYLQMNSLRAEDTAVYYCAKGFQYGSGSYYTHFDY | |
| | WGQGTLVTVSS | |
| 42 | DIQMTQSPSSLSASVGDRVTITCRASQGISSWLAWYQQKPEKAPKSLIYAASSLQSGVP | Anti-HER2_V_{L} |
| | SRFSGSGSGTDFTLTISSLQPEDFATYYCQQYNSYPYTFGQGTKLEIK | |
| 43 | MKYLLPTAAAGLLLLAAQPAMAQVQLVQSGGGLVQPGRSLRLSCAASGFTDDYAM | Anti-HER3_scFv |
| | HWVRQAPGKGLEWVSGISWNSGSIGYADSVKGRFTISRDNAKNSLYLQMNSLRPEDT | |
| | AVYYCARDLGAKQWLEGFDYWGQGTLVTVSSGGGGSGGGGSGGGGSNFMLTQDPA | |
| | VSVALGQTVRITCQGDSLRSYYASWYQQKPGQAPVLVIYGKNNRPSGIPDRFSGSTSG | |
| | NSASLTITGAQAEDEADYYCNSRDSSGNHWVFGGGTKVTVLGAAAEQKLISEEDLNG | |
| | AAHHHHHH | |
| 44 | MKLWLNWIFLVTLLNGIQCEVKIVESGGGLVQPGGSLSLSCAASGFTFTDYYMNWV | Anti-DLL3_Heavy |
| | RQPPGKALEWLALIRNKANGYTTEYNASVKGRFTISRDNSQNILYLQMNALRAEDSA | Chain |
| | TYYCARDSDGYYEYYFDYWGQGTILTVSSASTKGPSVFPLAPSSKSTSGGTAALGCL | |
| | VKDYFPEPVTVSWNSGALTSGVHTFPAVLQSSGLYSLSSVVTVPSSSLGTQTYICNVN | |
| | HKPSNTKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQDWLNGK | |
| | EYKCKVSNKALPAPIEKTISKAKGQPREPQVYTLPPSRDELTKNQVSLTCLVKGFYPSD | |
| | IAVEWESNGQPENNYKTTPPVLDSDGSFFLYSKLTVDKSRWQQGNVFSCSVMHEALH | |
| | NHYTQKSLSLSPGK | |
| 45 | DYYMN | Anti-DLL3_HCDR1 |
| 46 | LIRNKANGYTTEYNASVKG | Anti-DLL3_HCDR2 |
| 47 | DSDGYYEYYFDY | Anti-DLL3_HCDR3 |
| 48 | MDMRVPAHVFGFLLLWFPGTRCDIQMTQSPSSLSASLGERVSLTCRASQEISDYLSWL | Anti-DLL3_Light |
| | QQKPDGTIKRLIFAASTLDSGVPKRFSGSRSGSDFSLSISSLESEDFADYYCLQYASYPY | Chain |
| | TFGSGTKLEIKRTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGLSSPVTKSFNRG | |
| | EC | |
| 49 | RASQEISDYLS | Anti-DLL3_LCDR1 |
| 50 | AASTLDS | Anti-DLL3_LCDR2 |
| 51 | LQYASYPYT | Anti-DLL3_LCDR3 |
| 52 | MKHLWFFLLLVAAPRWVLSQVQLQESGPGLVKPSQTLSLTCTVSGGSISSGGYYWSW | Anti-C-MET_Heavy |
| | IRQHPGKGLEWIGYIYYSGSTYYNPSLKSRVTISVDTSKNQFSLKLSSVTAADTAVYYC | Chain |
| | ARDGPLGYCSSTSCPVTGEYYYYGMDVWGQGTTVTVSSASTKGPSVFPLAPCSRSTS | |
| | ESTAALGCLVKDYFPEPVTVSWNSGALTSGVHTFPAVLQSSGLYSLSSVVTVPSSNFG | |
| | TQTYTCNVDHKPSNTKVDKTVERKCCVECPPCPAPPVAGPSVFLFPPKPKDTLMISRT | |
| | PEVTCVVVDVSHEDPEVQFNWYVDGVEVHNAKTKPREEQFNSTFRVVSVLTVVHQD | |
| | WLNGKEYKCKVSNKGLPAPIEKTISKTKGQPREPQVYTLPPSREEMTKNQVSLTCLVK | |
| | GFYPSDIAVEWESNGQPENNYKTTPPMLDSDGSFFLYSKLTVDKSRWQQGNVFSCSV | |
| | MHEALHNHYTQKSLSLSPGK | |
| 53 | SGGYYWS | Anti-C-MET_HCDR1 |
| 54 | YIYYSGSTYYNPSLKS | Anti-C-MET_HCDR2 |
| 55 | LGPLGYCSSTSCPVTGEYYYYGMDV | Anti-C-MET_HCDR3 |
| 56 | METPAQLLFLLLLWLPDTTGEIVLTQSPGTLSLSPGERATLSCRASQSVSNNYLAWYQ | Anti-C-MET_Light |
| | QKPGQAPRLLIFGASSRATGIPDRFSGSGSGTDFTLTISRLEPEDFAVYYCQQYDISPMY | Chain |
| | SFGQGTKLEMKRTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQGLSSPVTKSFNR | |
| | GEC | |
| 57 | RASQSVSNNYLA | Anti-C-MET_LCDR1 |
| 58 | GASSRAT | Anti-C-MET_LCDR2 |
| 59 | QQYDISPMYS | Anti-C-MET_LCDR3 |
| 60 | EVQLLEQSGAELVRPGTSVKISCKASGYAFTNYWLGWVKQRPGHGLEWIGDIFPGSG | Anti-EPCAM_V_{H} |
| | NIHYNEKFKGKATLTADKSSSTAYMQLSSLTFEDSAVYFCARLRNWDEPMDYWGQG | |
| | TTVTVSS | |
| 61 | ELVMTQSPSSLTVTAGEKVTMSCKSSQSLLNSGNQKNYLTWYQQKPGQPPKLLIYWA | Anti-EPCAM_V_{L} |
| | STRESGVPDRFTGSGSGTDFTLTISSVQAEDLAVYYCQNDYSYPLTFGAGTKLEIK | |
| 62 | MAWVWTLLFLMAAAQSAGAQIQLVQSGPEVKKPGETVKISCKASGYTFTNYGMNW | Anti-CD70_V_{H} |
| | VKQAPGKGLKWMGWINTYTGEPTYADAFKGRFAFSLETSASTAYLQINNLKNEDTA | |
| | TYFCARDYGDYGMDYWGQGTSVTVSS | |
| 63 | GYTFTNYGMN | Anti-CD70_HCDR1 |
| 64 | WINTYTGEPTYADAFK | Anti-CD70_HCDR2 |
| 65 | DYGDYGMDY | Anti-CD70_HCDR3 |
| 66 | METDTLLLWVLLLWVPGSTGDIVLTQSPASLAVSLGQRATISCRASKSVSTSGYSFMH | Anti-CD70_V_{L} |
| | WYQQKPGQPPKLLIYLASNLESGVPARFSGSGSGTDFTLNIHPVEEEDAATYYCQHSR | |
| | EVPWTFGGGTKLEIKR | |
| 67 | RASKSVSTSGYSFMH | Anti-CD70_LCDR1 |
| 68 | LASNLES | Anti-CD70_LCDR2 |
| 69 | QHSREVPWT | Anti-CD70_LCDR3 |
| 70 | QVQLQESGPGLVKPSQTLSLTCTVSGGSINNNNYYWTWIRQHPGKGLEWIGYIYYSGS | Anti-MSLN_V_{H} |
| | TFYNPSLKSRVTISVDTSKTQFSLKLSSVTAADTAVYYCAREDTMTGLDVWGQGTTV | |
| | TVSS | |
| 71 | NNNYYWT | Anti-MSLN_HCDR1 |
| 72 | YIYYSGSTFYNPSLKS | Anti-MSLN_HCDR2 |
| 73 | EDTMTGLDV | Anti-MSLN_HCDR3 |
| 74 | DIQMTQSPSSLSASVGDRVTTTCRASQSINNYLNWYQQKPGKAPTLLIYAASSLQSGVP | Anti-MSLN_V_{L} |
| | SRFSGSRSGTDFTLTISSLQPEDFAAYFCQQTYSNPTFGQGTKVEVK | |
| 75 | RASQSINNYLN | Anti-MSLN_LCDR1 |
| 76 | AASSLQS | Anti-MSLN_LCDR2 |
| 77 | QQTYSNPT | Anti-MSLN_LCDR3 |
| 78 | EVQLVESGGGLVQPGGSLRLSCAVSGFSLTNYGVHWVRQATGKGLEWLGVIWSGGN | Anti-EGFR_V_{H} |
| | TDYNTPFTSRLTISKENAKNSVYLQMNSLRAGDTAVYYCARALTYYDYEFAYWGQG | |
| | TMVTVSS | |
| 79 | NYGVH | Anti-EGFR_HCDR1 |
| 80 | GVIWSGGNTDYNTPFT | Anti-EGFR_HCDR2 |
| 81 | RALTYYDYEFAYW | Anti-EGFR_HCDR3 |
| 82 | EIVLTQSPATLSLSPGERATLSCRASQSIGTNIHWYQQRPGQAPRLLIYYASESISGIPAR | Anti-EGFR_V_{L} |
| | FSGSGSGTDFTLTISSLEPEDFAVYYCQQNNNWPTTFGGGTKVEIK | |
| 83 | RASQSIGTNIH | Anti-EGFR_LCDR1 |
| 84 | YASESIS | Anti-EGFR_LCDR2 |
| 85 | QQNNNWP | Anti-EGFR_LCDR3 |
| 86 | QVKLQQSGGGLVKPGASLKLSCVTSGFTFRKFGMSWVRQTSDKRLEWVASISTGGY | Anti-EGFR VIII_scFv |
| | NTYYSDNVKGRFTISRENAKNTLYLQMSSLKSEDTALYYCTRGYSSTSYAMDYWGQ | |
| | GTTVTVSSSGGGSGGGGSGGGGSDIELTQSPASLSVATGEKVTIRCMTSTDIDDDMNW | |
| | YQQKPGEPPKFLISEGNTLRPGVPSRFSSSGTGTDFVFTIENTLSEDVGDYYCLQSFNVP | |
| | LTFGDGTKLEIK | |
| 87 | GYSSTSYAM | Anti- |
| | | EGFR VIII_HCDR3 |
| 88 | LQSFNVPLT | Anti- |
| | | EGFR VIII_LCDR3 |
| 89 | KTITATGVLFVRLGP | ROR2 epitope |
| 90 | EVQLVQSGAEVKKPGESLKISCQGSGYRFSKYWIGWVRQMPGKGLEWMGIIYPGDSD | Anti-ROR2_V_{H} |
| | TRYSPSFQGQVTISADKSISTAYLQWSSLKASDTAMYYCARSFSSFIYDYWGQGTLVT | |
| | VSS | |
| 91 | GYRFSKYW | Anti-ROR2_HCDR1 |
| 92 | IYPGDSDT | Anti-ROR2_HCDR2 |
| 93 | ARSFSSFIYDY | Anti-ROR2_HCDR3 |
| 94 | QVQLVESGAEVKKPGESLKISCKASGYSFSNYWIGWVRQMPGKGLEWMGIIYPDDSD | Anti-ROR2_V_{H} |
| | TRYSPSFQGQVTISADKSISTAYLQWYSLKVADTAKYYCVRPRGAFDIWGQGTTVTV | |
| | SS | |
| 95 | GYSFSNYW | Anti-ROR2_HCDR1 |
| 96 | IYPDDSDT | Anti-ROR2_HCDR2 |
| 97 | VRPRGAFDI | Anti-ROR2_HCDR3 |
| 98 | EVQLVESGPGLVKPSQTLSLTCTVSGGSISSGGYYWSWIRQHPGKGLEWIGYIYYSGS | Anti-ROR2_V_{H} |
| | TYYNPSLKSRVTISVDTSKNQFSLKLSSVTAADTAMYYCARGGLYWTYSQDVWGQG | |
| | TLVTVSS | |
| 99 | GGSISSGGYY | Anti-ROR2_HCDR1 |
| 100 | IYYSGST | Anti-ROR2_HCDR2 |
| 101 | ARGGLYWTYSQDV | Anti-ROR2_HCDR3 |
| 102 | QITLKESGPELVKPTQTLTLTCTFSGFSLSTSGMSVSWIRQPPGKALEWLARIDWDDD | Anti-ROR2_V_{H} |
| | KYYSTSLKTRLTISKDTSKNQVVLTMTNTDPVDTATYYCARGFYLAYGSYDSWGQG | |
| | TLVTVSS | |
| 103 | GFSLSTSGMS | Anti-ROR2_HCDR1 |
| 104 | IDWDDDK | Anti-ROR2_HCDR2 |
| 105 | ARGFYLAYGSYDS | Anti-ROR2_HCDR3 |
| 106 | EVQLVQSGAEVKKPGASVKVSCKASGYTFTNYYIHWVRQAPGQGLEWMGIINPTSG | Anti-ROR2_V_{H} |
| | RTRYAQRFQGRVTMTRDTSTNTVYMDLSSLRSEDTAMYYCARSGYYWGVNGDQW | |
| | GQGTLVTVSS | |
| 107 | GYTFTNYY | Anti-ROR2_HCDR1 |
| 108 | INPTSGRT | Anti-ROR2_HCDR2 |
| 109 | ARSGYYWGVNGDQ | Anti-ROR2_HCDR3 |
| 110 | ETTLTQSPGTLSVSPGERATLSCRASQSVSSNLAWYQQKRGQAPRLLIYGASTRATGIP | Anti-ROR2_V_{L} |
| | VRFSGSGSGTEFTLTISRLEPEDFAVYYCQQYGRSPLTFGGGTKVDIKR | |
| 111 | QSVSSN | Anti-ROR2_LCDR1 |
| 112 | GAS | Anti-ROR2_LCDR2 |
| 113 | QQYGRSPLT | Anti-ROR2_LCDR3 |
| 114 | QSVLTQPPSVSGAPGQRVTISCTGSSSNIGAGHAVHWYQQLPGTAPKLLIYDNANRPS | Anti-ROR2_V_{L} |
| | GVPDRFSGSQSGTSASLAITGLQTGDEADYYCGTWDDSPSAYVFGTGTKVTVLG | |
| 115 | SSNIGAGHA | Anti-ROR2_LCDR1 |
| 116 | DNA | Anti-ROR2_LCDR2 |
| 117 | GTWDDSPSAYV | Anti-ROR2_LCDR3 |
| 118 | QPVLTQPPSASGTPGQRVTISCSGSSSNIGSDYVSWYQQLPGTAPKLLIYRNDQRPSGV | Anti-ROR2_V_{L} |
| | PDRFSGSKSGTSASLAISGLRSEDEADYYCVAWDDSLSGYVFGSGTKVTVLG | |
| 119 | SSNIGSDY | Anti-ROR2_LCDR1 |
| 120 | RND | Anti-ROR2_LCDR2 |
| 121 | VAWDDSLSGYV | Anti-ROR2_LCDR3 |
| 122 | QSALTQPASVSGSPGQSITISCTGTSGDVGGYNYVSWYQHHPGKAPKLIIYDVNKRPS | Anti-ROR2_V_{L} |
| | GFSDRFSGSKSGNTASLTISGLQAEDEADYYCSSYTSTSTVFGGGTKLTVLG | |
| 123 | SGDVGGYNY | Anti-ROR2_LCDR1 |
| 124 | DVN | Anti-ROR2_LCDR2 |
| 125 | SSYTSTSTV | Anti-ROR2_LCDR3 |
| 126 | SYELTQPLSVSVALGQTARITCGGNNIGSKNVHWYQQKPGQAPVLVIYRDSNRPSGIP | Anti-ROR2_V_{L} |
| | ERFSGSNSGNTATLTISRAQAGDEADYYCQVWDSSIVVFGGGTKLTVLG | |
| 127 | NIGSKN | Anti-ROR2_LCDR1 |
| 128 | RDS | Anti-ROR2_LCDR2 |
| 129 | QVWDSSIVV | Anti-ROR2_LCDR3 |
| 130 | QVQLQQWGAGLLKPSETLSLTCAVYGGSFSGYYWSWIRQPPGKGLEWIGEINHSGST | Anti-MUC16_V_{H} |
| | NYNPSLKSRVTISVDTSKNQFSLKLSSVTAADTAVYYCARQSYITDSWGQGTLVTVSS | |
| 131 | GGSFSGYY | Anti-MUC16_HCDR1 |
| 132 | INHSGST | Anti-MUC16_HCDR2 |
| 133 | ARQSYITDS | Anti-MUC16_HCDR3 |
| 134 | QVQLQQWGAGLLKPSETLSLTCAVYGGSFSGYYWSWIRQPPGKGLEWIGEINHSGST | Anti-MUC16_V_{H} |
| | NYNPSLKSRIIMSVDTSKRQFSLKLRSATAADTAVYYCARWSPFSYKQMYDYWGQG | |
| | TLVTVSS | |
| 135 | GGSFSGYY | Anti-MUC16_HCDR1 |
| 136 | INHSGST | Anti-MUC16_HCDR2 |
| 137 | ARWSPFSYKQMYDY | Anti-MUC16_HCDR3 |
| 138 | DIQLTQSPSAVSASVGDRVTITCRASQDVSKWLAWYQQKPGKAPRLLISAASGLQSW | Anti-MUC16_V_{L} |
| | VPSRFSGSGSGTEFTLSISSLQPEDFATYYCQQANSFPWTFGQGTKVEIKR | |
| 139 | QDVSKW | Anti-MUC16_LCDR1 |
| 140 | AAS | Anti-MUC16_LCDR2 |
| 141 | QQANSFPWT | Anti-MUC16_LCDR3 |
| 142 | NFMLTQPHSVSESPGKTVTISCTRSRGSIASAYVQWYQQRPGSAPITVIYEDYERPSEIP | Anti-MUC16_V_{L} |
| | DRFSGSIDSSSNSASLTISGLKTEDEADYYCQSYDDNDHVIFGGGTKVTVLG | |
| 143 | RGSIASAY | Anti-MUC16_LCDR1 |
| 144 | EDY | Anti-MUC16_LCDR2 |
| 145 | QSYDDNDHVI | Anti-MUC16_LCDR3 |
| 146 | EVKLQESGGGFVKPGGSLRVSCAASGFTFSSYAMSWVRLAPEMRLEWVATISSAGGY | Anti-MUC16_V_{H} |
| | IFYSDSVQGRFTISRDNAKNSLHLQMGSLRSGDTAMYYCARQGFGNYGDYYAMDY | |
| | WGQGTTVTVSS | |
| 147 | EVQLVESGGGLVKPGGSLRVSCAASGFTFSSYAMSWVRLAPGKGLEWVATISSAGGY | Anti-MUC16_V_{H} |
| | IFYSDSVQGRFTISRDNAKNSLYLQMNSLRAEDTAMYYCARQGFGNYGDYYAMDY | |
| | WGQGTLVTVSS | |
| 148 | QVTLKESGPGILQPTQTLTLTCTFSGFSLSTVGMGVGWSRQPSGKGLEWLAHIWWDD | Anti-MUC16_V_{H} |
| | EDKYYNPALKSRLTITKDTSKNQVFLKITNVDTADTATYYCTRIGTAQATDALDYWG | |
| | QGTLVTVSS | |

| | | |
|---|---|---|
| 149 | QVTLKESGPTLVKPTQTLTLTCTFSGFSLSTVGMGVGWSRQPSGKGLEWLAHIWWDD | Anti-MUC16_V_{H} |
| | EDKYYNPALKSRLTITKDTSKNQVVLTITNVDPVDTATYYCTRIGTAQATDALDYWG | |
| | QGTLVTVSS | |
| 150 | DIELTQSPSSLAVSAGERVTMNCKSSQSLLNSRTRKNQLAWYQQKPGQSPELLIYWAS | Anti-MUC16_V_{L} |
| | TRQSGVPDRFSGSGSGTDFTLTISSVQAEDVAVYYCQQSYNLLTFGPGTKLEIKR | |
| 151 | DIVLTQSPDSLAVSLGERVTMNCKSSQSLLNSRTRKNQLAWYQQKPGQSPELLIYWA | Anti-MUC16_V_{L} |
| | STRQSGVPDRFSGSGSGTDFTLTISSVQAEDVAVYYCQQSYNLLTFGQGTKLEIKR | |
| 152 | DIVMTQSAPSVPVTPGESVSISCRSSKSLLHSNGNTYLYWFLQKPGQSPQRLIYYMSNL | Anti-MUC16_V_{L} |
| | ASGVPDRFSGRGSGTDFTLKISRVEAEDVGVYYCMQSLEYPLTFGGGTKLEIKR | |
| 153 | DIVMTQSALSLPVTPGEPVSISCRSSKSLLHSNGNTYLYWFLQKPGQSPQRLIYYMSNL | Anti-MUC16_V_{L} |
| | ASGVPDRFSGSGSGTDFTLKISRVEAEDVGVYYCMQSLEYPLTFGGGTKLEIKR | |
| 154 | EVQLVQSGVEVKKPGESLKISCKGSGYDFSDYWITWVRQMPGRGLEWMARIDPSDS | Anti-MCT4_V_{H} |
| | NIDYSPSFQGHVTISADKSISTAYLQWSSLKASDTAMYYCARSHGGGDYWGQGTLVT | |
| | VSS | |
| 155 | GYDFSDYW | Anti-MCT4_HCDR1 |
| 156 | IDPSDSNI | Anti-MCT4_HCDR2 |
| 157 | ARSHGGGDY | Anti-MCT4_HCDR3 |
| 158 | EVQLVESGGGVVQPGRSLRLSCVASGFSFSTYAMHWVRQAPGKGLEWVASILDDGS | Anti-MCT4_V_{H} |
| | KKDYADSVEGRFVISRDNSKNTLFLEVNSLSPEDTAVYYCARYSSWSSFGYPDYWGQ | |
| | GTLVTVSS | |
| 159 | GFSFSTYA | Anti-MCT4_HCDR1 |
| 160 | ILDDGSKK | Anti-MCT4_HCDR2 |
| 161 | ARYSSWSSFGYPDY | Anti-MCT4_HCDR3 |
| 162 | EVQLVQSGAEVKKPGSSVKVSCKASGGTFSSYAISWVRQAPGQGLEWMGWMNPNS | Anti-MCT4_V_{H} |
| | GNTGYAQKFQGRVTMTRNTSISTAYMELSSLRSEDTAVYYCARRSRTTGYDHWGQG | |
| | TLVTVSS | |
| 163 | GGTFSSYA | Anti-MCT4_HCDR1 |
| 164 | MNPNSGNT | Anti-MCT4_HCDR2 |
| 165 | ARRSRTTGYDH | Anti-MCT4_HCDR3 |
| 166 | SYVLTQPPSVSVSPGQTASITCSGDNLGDKYVSWYQQKPGQSPVLVIYQDTKRPSRIPE | Anti-MCT4_V_{L} |
| | RFSGSNSGNTATLTISGTQAMDDADYYCLTWDSSTGVFGAGTKVTVLG | |
| 167 | NLGDKY | Anti-MCT4_LCDR1 |
| 168 | QDT | Anti-MCT4_LCDR2 |
| 169 | LTWDSSTGV | Anti-MCT4_LCDR3 |
| 170 | NFMLTQPHSVSESLGKTVTISCTRSSGSIGTGYVQWYQLRPGSAPIMVIYEDNRRPSGV | Anti-MCT4_V_{L} |
| | PGRFSGSIDSSSNSASLTISGLMSEDEADYYCQSFDYFNQGVFGGGTKVTVLG | |
| 171 | SGSIGTGY | Anti-MCT4_LCDR1 |
| 172 | EDN | Anti-MCT4_LCDR2 |
| 173 | QSFDYFNQGV | Anti-MCT4_LCDR3 |
| 174 | DIQMTQSPSTLSASVGDRVTITCQANQDISNDLNWYQQKPGKAPKLLIYDTSTLEIGVP | Anti-MCT4_V_{L} |
| | SRFSGSGSGTNFTFTISSLQPEDIATYYCQQFYNLPITFGQGTRLEIKR | |
| 175 | QDISND | Anti-MCT4_LCDR1 |
| 176 | DTS | Anti-MCT4_LCDR2 |
| 177 | QQFYNLPIT | Anti-MCT4_LCDR3 |
| 178 | MMKSLRVLLVILWLQLSWVWSQQKEVEQNSGPLSVPEGAIASLNCTYSDRGSQSF | Anti-AFP-TCR1 alpha |
| | FWYRQYSGKSPELIMSIYSNGDKEDGRFTAQLNKASQYVSLLIRDSQPSDSATYL | chain |
| | CAVNSDSGYALNFGKGTSLLVTPHIQNPDPAVYQLRDSKSSDKSVCLFTDFDSQTNVSQ | |
| | SKDSDVYITDKTVLDMRSMDFKSNSAVAWSNKSDFACANAFNNSIIPEDTFFPSPESSCDVK | |
| | LVEKSFETDTNLNFQNLSVIGFRILLLKVAGFNLLMTLRLWSS | |
| 179 | MGFRLLCCVAFCLLGAGPVDSGVTQTPKHLITATGQRVTLRCSPRSGDLSVYWYQ | Anti-AFP-TCR1 and |
| | QSLDQGLQFLIQYYNGEERAKGNILERFSAQQFPDLHSELNLSSLELGDSALYFC | Anti-AFP-TCR2 beta |
| | ASSLGGESEQYFGPGTRLTVTEDLKNVFPPEVAVFEPSEAEISHTQKATLVCLATGFYP | chain |
| | DHVELSWWVNGKEVHSGVSTDPQPLKEQPALNDSRYCLSSRLRVSATFWQNPRNHFRCQV | |
| | QFYGLSENDEWTQDRAKPVTQIVSAEAWGRADCGFTSESYQQGVLSATILYEILLGKATLYA | |
| | VLVSALVLMAMVKRKDSRG | |
| 180 | MMKSLRVLLVILWLQLSWVWSQQKEVEQNSGPLSVPEGAIASLNCTYSDRGSQAF | Anti-AFP-TCR2 alpha |
| | FWYRQYSGKSPELIMSIYSNGDKEDGRFTAQLNKASQYVSLLIRDSQPSDSATYL | chain, affinity- |
| | CAVNSQSGYALNFGKGTSLLVTPHIQNPDPAVYQLRDSKSSDKSVCLFTDFDSQTNVS | enhanced version of |
| | QSKDSDVYITDKTVLDMRSMDFKSNSAVAWSNKSDFACANAFNNSIIPEDTFFPSPESSCDV | Anti-AFP-TCR1 alpha |
| | KLVEKSFETDTNLNFQNLSVIGFRILLLKVAGFNLLMTLRLWSS | chain |
| 181 | METDTLLLWVLLLWVPGSTGQSVLTQPPSVSAAPGQRVTISCSGTRSNIGSDYVSWY | αGPC3-CD30-CSR |
| | QHLPGTAPKLLVYGDNLRPSGIPDRFSASKSGTSATLGITGLQTGDEADYYCGTWDYT | Signal peptide-GPC3- |
| | LNGVVFGGGTKLTVLGSRGGGGSGGGGSGGGGSLEMAQVQLVESGGGLVQPGGSLR | 37 scFv-myc tag- |
| | LSCAASGFTFSSYAMSWVRQAPGKGLEWVSVIYSGGSSTYYADSVKGRFTISRDNSK | linker-truncated CD30 |
| | NTLYLQMNSLRAEDTAVYYCARTSYLNHGDYWGQGTLVTVSSEQKLISEEDLAAAT | (clone 37 anti-GPC3- |
| | GAPPLGTQPDCNPTPENGEAPASTSPTQSLLVDSQASKTLPIPTSAPVALSSTGKPVLD | CD30-CSR with SP |
| | AGPVLFWVILVLVVVVGSSAFLLCHRRACRKRIRQKLHLCYPVQTSQPKLELVDSRPR | and myc tag) |
| | RSSTQLRSGASVTEPVAEERGLMSQPLMETCHSVGAAYLESLPLQDASPAGGPSSPRD | |
| | LPEPRVSTEHTNNKIEKIYIMKADTVIVGTVKAELPEGRGLAGPAEPELEEELEADHTP | |
| | HYPEQETEPPLGSCSDVMLSVEEEGKEDPLPTAASGK | |
| 182 | METDTLLLWVLLLWVPGSTGQSVLTQPPSVSAAPGQRVTISCSGTRSNIGSDYVSWY | αGPC3-CD28-CSR |
| | QHLPGTAPKLLVYGDNLRPSGIPDRFSASKSGTSATLGITGLQTGDEADYYCGTWDYT | Signal peptide-GPC3- |
| | LNGVVFGGGTKLTVLGSRGGGGSGGGGSGGGGSLEMAQVQLVESGGGLVQPGGSLR | 37 scFv-myc tag- |
| | LSCAASGFTFSSYAMSWVRQAPGKGLEWVSVIYSGGSSTYYADSVKGRFTISRDNSK | linker-truncated CD28 |
| | NTLYLQMNSLRAEDTAVYYCARTSYLNHGDYWGQGTLVTVSSEQKLISEEDLAAAIE | |
| | VMYPPPYLDNEKSNGTIIHVKGKHILCPSPLFPGPSKPFWVLVVVGGVLACYSLLVTVA | |
| | FIIFWVRSKRSRLLHSDYMNMTPRRPGPTRKHYQPYAPPRDFAAYRS | |
| 183 | METDTLLLWVLLLWVPGSTGQSVLTQPPSVSAAPGQRVTISCSGTRSNIGSDYVSWY | αGPC3-41BB-CSR |
| | QHLPGTAPKLLVYGDNLRPSGIPDRFSASKSGTSATLGITGLQTGDEADYYCGTWDYT | Signal peptide-GPC3- |
| | LNGVVFGGGTKLTVLGSRGGGGSGGGGSGGGGSLEMAQVQLVESGGGLVQPGGSLR | 37 scFv-myc tag- |
| | LSCAASGFTFSSYAMSWVRQAPGKGLEWVSVIYSGGSSTYYADSVKGRFTISRDNSK | linker-truncated 41BB |
| | NTLYLQMNSLRAEDTAVYYCARTSYLNHGDYWGQGTLVTVSSEQKLISEEDLAAAT | |
| | GPADLSPGASSVTPPAPAREPGHSPQIISFFLALTSTALLFLLFFLTLRFSVVKRGRKKLL | |
| | YIFKQPFMRPVQTTQEEDGCSCRFPEEEEGGCEL | |
| 184 | METDTLLLWVLLLWVPGSTGQSVLTQPPSVSAAPGQRVTISCSGTRSNIGSDYVSWY | αGPC3-OX40-CSR |
| | QHLPGTAPKLLVYGDNLRPSGIPDRFSASKSGTSATLGITGLQTGDEADYYCGTWDYT | Signal peptide-GPC3- |
| | LNGVVFGGGTKLTVLGSRGGGGSGGGGSGGGGSLEMAQVQLVESGGGLVQPGGSLR | 37 scFv-myc tag- |
| | LSCAASGFTFSSYAMSWVRQAPGKGLEWVSVIYSGGSSTYYADSVKGRFTISRDNSK | linker-truncated OX40 |
| | NTLYLQMNSLRAEDTAVYYCARTSYLNHGDYWGQGTLVTVSSEQKLISEEDLAAAT | |
| | GDRDPPATQPQETQGPPARPITVQPTEAWPRTSQGPSTRPVEVPGGRAVAAILGLGLV | |
| | LGLLGPLAILLALYLLRRDQRLPPDAHKPPGGGSFRTPIQEEQADAHSTLAKI | |
| 185 | METDTLLLWVLLLWVPGSTGQSVLTQPPSVSAAPGQRVTISCSGTRSNIGSDYVSWY | αGPC3-CD27-CSR |
| | QHLPGTAPKLLVYGDNLRPSGIPDRFSASKSGTSATLGITGLQTGDEADYYCGTWDYT | Signal peptide-GPC3- |
| | LNGVVFGGGTKLTVLGSRGGGGSGGGGSGGGGSLEMAQVQLVESGGGLVQPGGSLR | 37 scFv-myc tag- |
| | LSCAASGFTFSSYAMSWVRQAPGKGLEWVSVIYSGGSSTYYADSVKGRFTISRDNSK | linker-trucated CD27 |
| | NTLYLQMNSLRAEDTAVYYCARTSYLNHGDYWGQGTLVTVSSEQKLISEEDLAAAT | |
| | GPTHLPYVSEMLEARTAGHMQTLADFRQLPARTLSTHWPPQRSLCSSDFIRILVIFSGM | |
| | FLVFTLAGALFLHQRRKYRSNKGESPVEPAEPCRYSCPREEEGSTIPIQEDYRKPEPACSP | |
| 186 | METDTLLLWVLLLWVPGSTGQSVLTQPPSVSAAPGQRVTISCSGTRSNIGSDYVSWY | αGPC3-CD30T-CD28- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD30T-CD28 |
| | GAPPLGTQPDCNPTPENGEAPASTSPTQSLLVDSQASKTLPIPTSAPVALSSTGKPVLD | IC |
| | AGPVLFWVILVLVVVVGSSAFLLCRSKRSRLLHSDYMNMTPRRPGPTRKHYQPYAPP | |
| | RDFAAYRS | |
| 187 | METDTLLLWVLLLWVPGSTGQSVLTQPPSVSAAPGQRVTISCSGTRSNIGSDYVSWY | αGPC3-CD30T-41BB- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD30T-41BB |
| | GAPPLGTQPDCNPTPENGEAPASTSPTQSLLVDSQASKTLPIPTSAPVALSSTGKPVLD | IC |
| | AGPVLFWVILVLVVVVGSSAFLLCKRGRKKLLYIFKQPFMRPVQTTQEEDGCSCRFPE | |
| | EEEGGCEL | |
| 188 | METDTLLLWVLLLWVPGSTGQSVLTQPPSVSAAPGQRVTISCSGTRSNIGSDYVSWY | αGPC3-CD30T-OX40- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD30T-OX40 |
| | GAPPLGTQPDCNPTPENGEAPASTSPTQSLLVDSQASKTLPIPTSAPVALSSTGKPVLD | IC |
| | AGPVLFWVILVLVVVVGSSAFLLCALYLLRRDQRLPPDAHKPPGGGSFRTPIQEEQAD | |
| | AHSTLAKI | |
| 189 | METDTLLLWVLLLWVPGSTGQSVLTQPPSVSAAPGQRVTISCSGTRSNIGSDYVSWY | αGPC3-CD30T-CD27- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD30T-CD27 |
| | GAPPLGTQPDCNPTPENGEAPASTSPTQSLLVDSQASKTLPIPTSAPVALSSTGKPVLD | IC |
| | AGPVLFWVILVLVVVVGSSAFLLCQRRKYRSNKGESPVEPAEPCRYSCPREEEGSTIPI | |
| | QEDYRKPEPACSP | |
| 190 | METDTLLLWVLLLWVPGSTGQSVLTQPPSVSAAPGQRVTISCSGTRSNIGSDYVSWY | αGPC3-CD28T-CD30- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | Linker-CD28T-CD30 |
| | GIEVMYPPPYLDNEKSNGTIIHVKGKHLCPSPLFPGPSKPFWVLVVVGGVLACYSLLV | IC |
| | TVAFIIFWVHRRACRKRIRQKLHLCYPVQTSQPKLELVDSRPRRSSTQLRSGASVTEPV | |
| | AEERGLMSQPLMETCHSVGAAYLESLPLQDASPAGGPSSPRDLPEPRVSTEHTNNKIE | |
| | KIYIMKADTVIVGTVKAELPEGRGLAGPAEPELEEELEADHTPHYPEQETEPPLGSCSD | |
| | VMLSVEEEGKEDPLPTAASGK | |
| 191 | METDTLLLWVLLLWVPGSTGQSVLTQPPSVSAAPGQRVTISCSGTRSNIGSDYVSWY | αGPC3-CD28T-41BB- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD28T-41BB |
| | GIEVMYPPPYLDNEKSNGTIIHVKGKHLCPSPLFPGPSKPFWVLVVVGGVLACYSLLV | IC |
| | TVAFIIFWVKRGRKKLLYIFKQPFMRPVQTTQEEDGCSCRFPEEEEGGCEL | |
| 192 | METDTLLLWVLLLWVPGSTGQSVLTQPPSVSAAPGQRVTISCSGTRSNIGSDYVSWY | αGPC3-CD28T-OX40- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD28T-OX40 |
| | GIEVMYPPPYLDNEKSNGTIIHVKGKHLCPSPLFPGPSKPFWVLVVVGGVLACYSLLV | IC |
| | TVAFIIFWVALYLLRRDQRLPPDAHKPPGGGSFRTPIQEEQADAHISTLAKI | |
| 193 | METDTLLLWVLLLWVPGSTGQSVLTQPPSVSAAPGQRVTISCSGTRSNIGSDYVSWY | αGPC3-CD28T-CD27- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD28T-CD27 |
| | GIEVMYPPPYLDNEKSNGTIIHVKGKHLCPSPLFPGPSKPFWVLVVVGGVLACYSLLV | IC |
| | TVAFIIFWVQRRKYRSNKGESPVEPAEPCRYSCPREFEGSTIPIQEDYRKPEPACSP | |
| 194 | METDTLLLWVLLLWVPGSTGQSVLTQPPSVSAAPGQRVTISCSGTRSNIGSDYVSWYQHL | αGPC3-CD28T- |
| | PGTAPKLLVYGDNLRPSGIPDRFSASKSGTSATLGITGLQTGDEADYYCGTWDYTLNGVVF | DAP10-CSR |
| | GGGTKLTVLGSRGGGGSGGGGSGGGGSLEMAQVQLVESGGGLVQPGGSLRLSCAASGFT | Signal peptide-GPC3- |
| | FSSYAMSWVRQAPGKGLEWVSVIYSGGSSTYYADSVKGRFTISRDNSKNTLYLQMNSLRAED | 37 scFv-myc tag- |
| | TAVYYCARTSYLNHGDYWGQGTLVTVSSEQKLISEEDLAAATGIEVMYPPPYLDNEKSNG | linker-CD28T-DAP10 |
| | TIIHVKGKHLCPSPLFPGPSKPFWVLVVVGGVLACYSLLVTVAFIIFWVCARPRRSPAQ | IC |
| | EDGKVYINMPGRG | |
| 195 | METDTLLLWVLLLWVPGSTGQSVLTQPPSVSAAPGQRVTISCSGTRSNIGSDYVSWY | αQGPC3-41BBT-CD30- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-41BBT-CD30 |
| | GPADLSPGASSVTPPAPAREPGHSPQIISFFLALTSTALLFLLFFLTLRFSVVHRRACRKR | IC |
| | IRQKLHLCYPVQTSQPKLELVDSRPRRSSTQLRSGASVTEPVAEERGLMSQPLMETCH | |
| | SVGAAYLESLPLQDASPAGGPSSPRDLPEPRVSTEHTNNKIEKIYIMKADTVIVGTVKA | |
| | ELPEGRGLAGPAEPELEEELEADHTPHYPEQETEPPLGSCSDVMLSVEEEGKEDPLPTA | |
| | ASGK | |
| 196 | METDTLLLWVLLLWVPGSTGQSVLTQPPSVSAAPGQRVTISCSGTRSNIGSDYVSWY | αGPC3-41BBT-CD28- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-41BBT-CD28 |
| | GPADLSPGASSVTPPAPAREPGHSPQIISFFLALTSTALLFLLFFLTLRFSVVRSKRSRLL | IC |
| | HSDYMNMTPRRPGPTRKHYQPYAPPRDFAAYRS | |
| 197 | METDTLLLWVLLLWVPGSTQQSVLTQPPSVSAAPGQRVTISCSGTRSNIGSDYVSWY | αGPC3-41BBT-OX40- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-41BBT-OX40 |
| | GPADLSPGASSVTPPAPAREPGHSPQIISFFLALTSTALLFLLFFLTLRFSVVALYLLRRD | IC |
| | QRLPPDAHKPPGGGSFRTPIQEEQADAHSTLAKI | |
| 198 | METDTLLLWVLLLWVPGSTGQSVLTQPPSVSAAPGQRVTISCSGTRSNIGSDYVSWY | αGPC3-41BBT-CD27- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-4IBBT-CD27 |
| | GPADLSPGASSVTPPAPAREPGHSPQIISFFLALTSTALLFLLFFLTLRFSVVQRRKYRSN | IC |
| | KGESPVEPAEPCRYSCPREEEGSTIPIQEDYRKPEPACSP | |
| 199 | METDTLLLWVLLLWVPGSTGQSVLTQPPSVSAAPGQRVTISCSGTRSNIGSDYVSWY | αGPC3-OX40T-CD30- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNIGDYWGQGTLVTVSSEQKLISEEDLAAAT | linker-OX40T-CD30 |
| | GDRDPPATQPQETQGPPARPITVQPTEAWPRTSQGPSTRPVEVPGGRAVAAILGLGLV | IC |
| | LGLLGPLAILLHRRACRKRIRQKLHLCYPVQTSQPKLELVDSRPRRSSTQLRSGASVTE | |
| | PVAEERGLMSQPLMETCHSVGAAYLESLPLQDASPAGGPSSPRDLPEPRVSTEHTNNK | |
| | IEKIYIMKADTVIVGTVKAELPEGRGLAGPAEPELEEELEADHTPHYPEQETEPPLGSCS | |
| | DVMLSVEEEGKEDPLPTAASGK | |
| 200 | METDTLLLWVLLLWVPGSTGQSVLTQPPSVSAAPGQRVTISCSGTRSNIGSDYVSWY | αGPC3-OX40T-CD28- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-OX40T-CD28 |
| | GDRDPPATQPQETQGPPARPITVQPTEAWPRTSQGPSTRPVEVPGGRAVAAILGLGLV | IC |
| | LGLLGPLAILLRSKRSRLLHSDYMNMTPRRPGPTRKHYQPYAPPRDFAAYRS | |
| 201 | METDTLLLWVLLLWVPGSTGQSVLTQPPSVSAAPGQRVTISCSGTRSNIGSDYVSWY | αGPC3-OX40T-41BB- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-OX40T-41BB |
| | GDRDPPATQPQETQGPPARPITVQPTEAWPRTSQGPSTRPVEVPGGRAVAAILGLGLV | IC |
| | LGLLGPLAILLKRGRKKLLYIFKQPFMRPVQTTQEEDGCSCRFPEEEEGGCEL | |
| 202 | METDTLLLWVLLLWVPGSTGQSVLTQPPSVSAAPGQRVTISCSGTRSNIGSDYVSWY | αGPC3-OX40T- |
| | QHLPGTAPKLLVYGDNLRPSGIPDRFSASKSGTSATLGITGLQTGDEADYYCGTWDYT | CD27-CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-OX40T-CD27 |
| | GDRDPPATQPQETQGPPARPITVQPTEAWPRTSQGPSTRPVEVPGGRAVAAILGLGLV | IC |
| | LGLLGPLAILLQRRKYRSNKGESPVEPAEPCRYSCPREEEGSTIPIQEDYRKPEPACSP | |
| 203 | METDTLLLWVLLLWVPGSTGQSVLTQPPSVSAAPGQRVTISCSGTRSNIGSDYVSWY | αGPC3-CD27T-CD30- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD27T-CD30 |
| | GPTHLPYVSEMLEARTAGHMQTLADFRQLPARTLSTHWPPQRSLCSSDFIRILVIFSGM | IC |
| | FLVFTLAGALFLHHRRACRKRIRQKLHLCYPVQTSQPKLELVDSRPRRSSTQLRSGAS | |
| | VTEPVAEERGLMSQPLMETCHSVGAAYLESLPLQDASPAGGPSSPRDLPEPRVSTEHT | |
| | NNKIEKIYIMKADTVIVGTVKAELPEGRGLAGPAEPELEEELEADHTPHYPEQETEPPL | |
| | GSCSDVMLSVEEEGKEDPLPTAASGK | |
| 204 | METDTLLLWVLLLWVPGSTGQSVLTQPPSVSAAPGQRVTISCSGTRSNIGSDYVSWY | αGPC3-CD27T- |
| | QHLPGTAPKLLVYGDNLRPSGIPDRFSASKSGTSATLGITGLQTGDEADYYCGTWDYT | CD28-CSR |
| | LNGVVFGGGTKLTVLGSRGGGGSGGGGSGGGGSLEMAQVQLVESGGGLVQPGGSLR | Signal peptide-GPC3- |
| | LSCAASGFIFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD27T-CD28 |
| | GPTHLPYVSEMLEARTAGHMQTLADFRQLPARTLSTHWPPQRSLCSSDFIRILVIFSGM | IC |
| | FLVFTLAGALFLHRSKRSRLLHSDYMNMTPRRPGPTRKHYQPYAPPRDFAAYRS | |
| 205 | METDTLLLWVLLLWVPGSTGQSVLTQPPSVSAAPGQRVTISCSGTRSNIGSDYVSWY | αGPC3-CD27T- |
| | QHLPGTAPKLIVYGDNLRPSGIPDRFSASKSGTSATLGITGLQTGDEADYYCGTWDYT | 41BB-CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD27T-41BB |
| | GPTHLPYVSEMLEARTAGHMQTLADFRQLPARTLSTHWPPQRSLCSSDFIRILVIFSGM | IC |
| | FLVFTLAGALFLHKRGRKKLLYIFKQPFMRPVQTTQEEDGCSCRFPEEEEGGCEL | |
| 206 | METDTLLLWVLLLWVPGSTGQSVITQPPSVSAAPGQRVTISCSGTRSNIGSDYVSWY | αGPC3-CD27T- |
| | QHLPGTAPKLLVYGDNLRPSGIPDRFSASKSGTSATLGITGLQTGDEADYYCGTWDYT | OX40-CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD27T-OX40 |
| | GPTHLPYVSEMLEARTAGHMQTLADFRQLPARTLSTHWPPQRSLCSSDFIRILVIFSGM | IC |
| | FLVFTLAGALFLHALYLLRRDQRLPPDAHKPPGGGSFRTPIQEEQADAHSTLAKI | |
| 207 | METDTLLLWVLLLWVPGSTGQSVLTQPPSVSAAPGQRVTISCSGTRSNIGSDYVSWY | αGPC3-CD8T-CD30- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD8T-CD30 |
| | GTTTPAPRPPTPAPTIASQPLSLRPEACRPAAGGAVHTRGLDFACDIYIWAPLAGTCGV | IC |
| | LLLSLVITLYCHRRACRKRIRQKLHLCYPVQTSQPKLELVDSRPRESSTQLRSGASVTE | |
| | PVAEERGLMSQPLMETCHSVGAAYLESLPLQDASPAGGPSSPRDLPEPRVSTEHTNNK | |
| | IEKIYIMKADTVIVGTVKAELPEGRGLAGPAEPELEEELEADHTPHYPEQETEPPLGSCS | |
| | DVMLSVEEEGKEDPLPTAASGK | |
| 208 | METDTLLLWVLLLWVPGSTGQSVLTQPPSVSAAPGQRVTISCSGTRSNIGSDYVSWY | αGPC3-CD8T-CD28- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD8T-CD28 |
| | GTTTPAPRPPTPAPTIASQPLSLRPEACRPAAGGAVHTRGLDFACDIYIWAPLAGTCGV | IC |
| | LLLSLVITLYCRSKRSRLLHSDYMNMTPRRPGPTRKHYQPYAPPRDFAAYRS | |
| 209 | METDTLLLWVLLLWVPGSTGQSVLTQPPSVSAAPGQRVTISCSGTRSNIGSDYVSWY | αGPC3-CD8T-41BB- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD8T-41BB |
| | GTTTPAPRPPTPAPTIASQPLSLRPEACRPAAGGAVHTRGLDFACDIYIWAPLAGTCGV | IC |
| | LLLSLVITLYCKRGRKKLLYIFKQPFMRPVQTTQEEDGCSCRFPEEEEGGCEL | |
| 210 | METDTLLLWVLLLWVPGSTGQSVLTQPPSVSAAPGQRVTISCSGTRSNIGSDYVSWY | αGPC3-CD8T-OX40- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD8T-OX40 |
| | GTTTPAPRPPTPAPTIASQPLSLRPEACRPAAGGAVHTRGLDFACDIYIWAPLAGTCGV | IC |
| | LLLSLVITLYCALYLLRRDQRLPPDAHKPPGGGSFRTPIQEEQADAHSTLAKI | |
| 211 | METDTLLLWVLLLWVPGSTGQSVLTQPPSVSAAPGQRVTISCSGTRSNIGSDYVSWY | αGPC3-CD8T-CD27- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD8T-CD27 |
| | GTTTPAPRPPTPAPTIASQPLSLRPEACRPAAGGAVHTRGLDFACDIYIWAPLAGTCGV | IC |
| | LLLSLVITLYCQRRKYRSNKGESPVEPAEPCRYSCPREEEGSTIPIQEDYRKPEPACSP | |
| 212 | QPVLTQPPSVSVAPGKTARITCGGNNIGSKSVHWYQQKPGQAPVLVIYYDSDRPSGIP | αGPC3-55 scFv |
| | ERFSGSNSGNTATLTISRVEAGDEADYYCQVWDSSSDHYVFGTGTKVTVLGSRGGGG | (αGPC3 scFv_clone |
| | SGGGGSGGGGSLEMAQVQLVQSGAEVKKPGASVKVSCKASGYTFTSYYMHWVRQA | 55) |
| | PGQGLEWMGIINPSGGSTSYAQKFQGRVTMTRDTSTSTVYMELSSLRSEDTAVYYCA | |
| | RWHGGPYDYWGQGTLVTVSS | |
| 213 | QSVLTQPPSVSVAPGKTARITCGGNNIGSKSVHWYQQKPGQAPVLVVYDDSDRPSGIP | αGPC3-58 scFv |
| | ERFSGSNSGNTATLTISRVEAGDEADYYCQVWDSSSDHVFGTGTKVTVLGSRGGGGS | (αGPC3 scFv_clone |
| | GGGGSGGGGSLEMAQVQLVQSGADVRKPGASVKVSCKASGYTFASHGISWVRQAPG | 58) |
| | QGLEWLGWISPYTGNTNYAQKFQGRVTMATDTSTSTAYMELRSLRSDDTAIYYCAR | |
| | GKRTLASCFDYWGQGTLVTVSS | |
| 214 | QSVITQPPSVSGAPGQRVTISCTGSSSNIGAGYDVHWYQQLPGTAPKLLIYGNSNRPS | Anti-PSMA-A scFv |
| | GVPDRFSGSKSGTSASLAITGLQAEDEADYYCQSYDSSLSGYVFGTGTKVTVLGSRGG | |
| | GGSGGGGSGGGGSLEMAEVQLVQSGAEVKKPGESLKISCKGSGYSFTSYWIGWVRQ | |
| | MPGKGLEWMGIIYPGDSDTRYSPSFQGQVTISADKSISTAYLQWSSLKASDTAMYYC | |
| | ARSMGSSLYASSDVWGQGTLVTVSS | |
| 215 | QAVLTQPPSASGTPGQRVTISCSGSSSNIGSNTVNWYQQLPGTAPKLLMYSNNQRPSG | Anti-PSMA-B scFv |
| | VPDRFSGSKSGTSASLAISGLQSEDEADYYCAAWDDSLNGYVFGTGTKVTVLGSRGG | |
| | GGSGGGGSGGGGSLEMAEVQLVQSGAEMKKPGESLKISCKGSGYNFASYWVGWVR | |
| | QMPGKGLEWMGTIYPDDSDTRYGPAFQGQVTISADKSISTAYLQWSSLKASDTAMY | |
| | YCARDSYYGIDVWGQGTLVTVSS | |
| 216 | DILLTQSPVILSVSPGERVSFSCRASQSIGTNIHWYQQRTNGSPRLLIKYASESISGIPSRF | Anti-EGFR scFv |
| | SGSGSGTDFTLSINSVESEDIADYYCQQNNNWPTTFGAGTKLELKRSRGGGGSGGGGS | |
| | GGGGSLEMAQVQLKQSGPGLVQPSQSLSITCTVSGFSLTNYGVHWVRQSPGKGLEWL | |
| | GVIWSGGNTDYNTPFTSRLSINKDNSKSQVFFKMNSLQSNDTAIYYCARALTYYDYEF | |
| | AYWGQGTLVTVSS | |
| 217 | APPLGTQPDCNPTPENGEAPASTSPTQSLLVDSQASKTLPIPTSAPVALSSTGKPVLDA | CD30 TM + IC |
| | GPVLFWVILVLVVVVGSSAFLLCHRRACRKRIRQKLHLCYPVQTSQPKLELVDSRPRR | |
| | SSTQLRSGASVTEPVAEERGLMSQPLMETCHSVGAAYLESLPLQDASPAGGPSSPRDL | |
| | PEPRVSTEHTNNKIEKIYIMKADTVIVGTVKAELPEGRGLAGPAEPELEEELEADHTPH | |
| | YPEQETEPPLGSCSDVMLSVEEEGKEDPLPTAASGK | |
| 218 | IEVMYPPPYLDNEKSNGTIIHVKGKHLCPSPLFPGPSKPFWVLVVVGGVLACYSLLVT | CD28 TM + IC |
| | VAFIIFWVRSKRSRLLHSDYMNMTPRRPGPTRKHYQPYAPPRDFAAYRS | |
| 219 | PADLSPGASSVTPPAPAREPGHSPQIISFFLALTSTALLFLLFFLTLRFSVVKRGRKKLLY | 41BB TM + IC |
| | IFKQPFMRPVQTTQEEDGCSCRFPEEEEGGCEL | |
| 220 | DRDPPATQPQETQGPPARPITVQPTEAWPRTSQGPSTRPVEVPGGRAVAAILGLGLVL | OX40 TM + IC |
| | GLLGPLAILLALYLLRRDQRLPPDAHKPPGGGSFRTPIQEEQADAHSTLAKI | |
| 221 | PTHLPYVSEMLEARTAGHMQTLADFRQLPARTLSTHWPPQRSLCSSDFIRILVIFSGMF | CD27 TM + HC |
| | LVFTLAGALFLHQRRKYRSNKGESPVEPAEPCRYSCPREEEGSTIPIQEDYRKPEPACSP | |
| 222 | LLAGLVAADAVASLLIVGAVFLCARPRRSPAQEDGKVYINMPGRG | DAP10 TM + IC |
| 223 | IEVMYPPPYLDNEKSNGTIIHVKGKHLCPSPLFPGPSKPFWVLVVVGGVLACYSLLVT | CD28T-CD30 IC |
| | VAFIIFWVHRRACRKRIRQKLHLCYPVQTSQPKLELVDSRPRRSSTQLRSGASVTEPVA | |
| | EERGLMSQPLMETCHSVGAAYLESLPLQDASPAGGPSSPRDLPEPRVSTEHTNNKIEKI | |
| | YIMKADTVIVGTVKAELPEGRGLAGPAEPELEEELEADHTPHYPEQETEPPLGSCSDV | |
| | MLSVEEEGKEDPLPTAASGK | |
| 224 | PADLSPGASSVTPPAPAREPGHSPQIISFFLALTSTALLFLLFFLTLRFSVVHRRACRKRI | 41BBT-CD30 IC |
| | RQKLHLCYPVQTSQPKLELVDSRPRRSSTQLRSGASVTEPVAEERGLMSQPLMETCHS | |
| | VGAAYLESLPLQDASPAGGPSSPRDLPEPRVSTEHTNNKIEKIYIMKADTVIVGTVKAE | |
| | LPEGRGLAGPAEPELEEELEADHTPHYPEQETEPPLGSCSDVMLSVEEEGKEDPLPTAA | |
| | SGK | |
| 225 | DRDPPATQPQETQGPPARPITVQPTEAWPRTSQGPSTRPVEVPGGRAVAAILGLGLVL | OX40T-CD30 IC |
| | GLLGPLAILLHRRACRKRIRQKLHLCYPVQTSQPKLELVDSRPRRSSTQLRSGASVTEP | |
| | VAEERGLMSQPLMETCHSVGAAYLESLPLQDASPAGGPSSPRDLPEPRVSTEHTNNKI | |
| | EKIYIMKADTVIVGTVKAELPEGRGLAGPAEPELEEELEADHTPHYPEQETEPPLGSCS | |
| | DVMLSVEEEGKEDPLPTAASGK | |
| 226 | PTHLPYVSEMLEARTAGHMQTLADFRQLPARTLSTHWPPQRSLCSSDFIRILVIFSGMF | CD27T-CD30 IC |
| | LVFTLAGALFLHHRRACRKRIRQKLHLCYPVQTSQPKLELVDSRPRRSSTQLRSGASV | |
| | TEPVAEERGLMSQPLMETCHSVGAAYLESLPLQDASPAGGPSSPRDLPEPRVSTEHTN | |
| | NKIEKIYIMKADTVIVGTVKAELPEGRGLAGPAEPELEEELEADHTPHYPEQETEPPLG | |
| | SCSDVMLSVEEEGKEDPLPTAASGK | |
| 227 | TTTPAPRPPTPAPTIASQPLSLRPEACRPAAGGAVHTRGLDFACDIYIWAPLAGTCGVL | CD8T-CD30 IC |
| | LLSLVITLYCHRRACRKRIRQKLHLCYPVQTSQPKLELVDSRPRRSSTQLRSGASVTEP | |
| | VAEERGLMSQPLMETCHSVGAAYLESLPLQDASPAGGPSSPRDLPEPRVSTEHTNNKI | |
| | EKIYIMKADTVIVGTVKAELPEGRGLAGPAEPELEEELEADHTPHYPEQETEPPLGSCS | |
| | DVMLSVEEEGKEDPLPTAASGK | |
| 228 | MRVLLAALGLLFLGALRAFPQDRPFEDTCHGNPSHYYDKAVRRCCYRCPMGLFPTQ | Full length CD30 |
| | QCPQRPTDCRKQCEPDYYLDEADRCTACVTCSRDDLVEKTPCAWNSSRVCECRPGM | (NP_001234.3) |
| | FCSTSAVNSCARCFFHSVCPAGMIVKFPGTAQKNTVCEPASPGVSPACASPENCKEPSS | |
| | GTIPQAKPTPVSPATSSASTMPVRGGTRLAQEAASKLTRAPDSPSSVGRPSSDPGLSPT | |
| | QPCPEGSGDCRKQCEPDYYLDEAGRCTACVSCSRDDLVEKTPCAWNSSRTCECRPGM | |
| | ICATSATNSCARCVPYPICAAETVTKPQDMAEKDTTFEAPPLGTQPDCNPTPENGEAP | |
| | ASTSPTQSLLVDSQASKTLPIPTSAPVALSSTGKPVLDAGPVLFWVILVLVVVVGSSAF | |
| | LLCHRRACRKRIRQKLHLCYPVQTSQPKLELVDSRPRRSSTQLRSGASVTEPVAEERG | |
| | LMSQPLMETCHSVGAAYLESLPLQDASPAGGPSSPRDLPEPRVSTEHTNNKIEKIYIMK | |
| | ADTVIVGTVKAELPEGRGLAGPAEPELEEELEADHTPHYPEQETEPPLGSCSDVMLSV | |
| | EEEGKEDPLPTAASGK | |
| 229 | IYIWAPLAGTCGVLLLSLVIT | CD8 transmembrane |
| | | (TM) sequence |
| 230 | IISFFLALTSTALLFLLFFLTLRFSVV | 4-1BB TM sequence |
| 231 | ILVIFSGMFLVFTLAGALFLH | CD27 TM sequence |
| 232 | FWVLVVVGGVLACYSLLVTVAFIIFWV | CD28 TM sequence |
| 233 | PVLDAGPVLFWVILVLVVVVGSSAFLLC | CD30 TM sequence |
| 234 | VAAILGLGLVLGLLGPLAILL | OX40 TM sequence |
| 235 | KRGRKKLLYIFKQPFMRPVQTTQEEDGCSCRFPEEEEGGCEL | 4-1BB IC signaling |
| | | sequence |
| 236 | QRRKYRSNKGESPVEPAEPCRYSCPREEEGSTIPIQEDYRKPEPACSP | CD27 IC signaling |
| | | sequence |
| 237 | RSKRSRLLHSDYMNMTPRRPGPTRKHYQPYAPPRDFAAYRS | CD28 IC signaling |
| | | sequence |
| 238 | HRRACRKRIRQKLHLCYPVQTSQPKLELVDSRPRRSSTQLRSGASVTEPVAEERGLMS | CD30 IC signaling |
| | QPLMETCHSVGAAYLESLPLQDASPAGGPSSPRDLPEPRVSTEHTNNKIEKIYIMKADT | sequence |
| | VIVGTVKAELPEGRGLAGPAEPELEEELEADHTPHYPEQETEPPLGSCSDVMLSVEEE | |
| | GKEDPLPTAASGK | |
| 239 | ALYLLRRDQRLPPDAHKPPGGGSFRTPIQEEQADAHSTLAKI | OX40 IC signaling |
| | | sequence |
| 240 | CARPRRSPAQEDGKVYINMPGRG | DAP10 IC signaling |
| | | sequence |
| 241 | RVKFSRSADAPAYQQGQNQLYNELNLGRREEYDVLDKRRGRDPEMGGKPRRKNPQE | CD3ζ IC signaling |
| | GLYNELQKDKMAEAYSEIGMKGERRRGKGHDGLYQGLSTATKDTYDALHMQALPP | sequence |
| | R | |
| 242 | SRGGGGSGGGGSGGGGSLEMA | Peptide linker |
| 243 | GGGGS | Peptide linker |
| 244 | GGSG | Peptide linker |
| 245 | SGGG | Peptide linker |
| 246 | GSGS | Peptide linker |
| 247 | GSGSGS | Peptide linker |
| 248 | GSGSGSGS | Peptide linker |
| 249 | GSGSGSGSGS | Peptide linker |
| 250 | GGSGGS | Peptide linker |
| 251 | GGSGGSGGS | Peptide linker |
| 252 | GGSGGSGGSGGS | Peptide linker |
| 253 | GGSG | Peptide linker |
| 254 | GGSGGGSG | Peptide linker |
| 255 | GGSGGGSGGGSG | Peptide linker |
| 256 | SRGGGGSGGGGSGGGGSLEMA | Peptide linker |
| 257 | HHHHHH | 6xHis Tag |
| 258 | YPYDVPDYA | HA peptide |
| 259 | YPYDVPDYAS | HA peptide |
| 260 | DYKDDDDK | FLAG peptide |
| 261 | EQKLISEEDL | Myc peptide |
| 262 | QVQLVQSGAEVKKPGASVKVSCKASGYTFTSYYMHWVRQAPGQGLEWMGIINPSGG | Anti-GPC3 V_{H} region |
| | STSYAQKFQGRVTMTRDTSTSTVYMELSSLRSEDTAVYYCARWHGGPYDYWGQGTL | |
| | VTVSS | |
| 263 | QPVLTQPPSVSVAPGKTARITCGGNNIGSKSVHWYQQKPGQAPVLVIYYDSDRPSGIP | Anti-GPC3 V_{L} region |
| | ERFSGSNSGNTATLTISRVEAGDEADYYCQVWDSSSDHYVFGTGTKVTVLG | |
| 264 | QVQLVQSGADVRKPGASVKVSCKASGYTFASHGISWVRQAPGQGLEWLGWISPYTG | Anti-GPC3 V_{H} region |
| | NTNYAQKFQGRVTMATDTSTSTAYMELRSLRSDDTAIYYCARGKRTLASCFDYWGQ | |
| | GTLVTVSS | |
| 265 | QSVLTQPPSVSVAPGKTARITCGGNNIGSKSVHWYQQKPGQAPVLVVYDDSDRPSGIP | Anti-GPC3 V_{L} region |
| | ERFSGSNSGNTATLTISRVEAGDEADYYCQVWDSSSDHVFGTGTKVTVLG | |
| 266 | GGAGGAAAAACTGTTTCATACAGAAGGCGTGGAGGAAAAACTGTTTCATACAGA | 6NFAT response |
| | AGGCGTGGAGGAAAAACTGTTTCATACAGAAGGCGTGGAGGAAAAACTGTTTCAT | element |
| | ACAGAAGGCGTGGAGGAAAAACTGTTTCATACAGAAGGCGTGGAGGAAAAACTG | |
| | TTTCATACAGAAGGCGT | |
| 267 | GCCGCCCCGACTGCATCTGCGTGTTCCAATTCGCCAATGACAAGACGCTGGGCGG | TA promoter |
| | GGTTTGTGTCATCATAGAACTAAAGACATGCAAATATATTTCITCCGGGGACACC | |
| | GCCAGCAAACGCGAGCAACGGGCCACGGGGATGAAGCAG | |
| 268 | GGAGGAAAAACTGTTTCATACAGAAGGCGTGGAGGAAAAACTGTTTCATACAGA | NFAT-derived |
| | AGGCGTGGAGGAAAAACTGTTTCATACAGAAGGCGTGGAGGAAAAACTGTTTCAT | promoter |
| | ACAGAAGGCGTGGAGGAAAAACTGTTTCATACAGAAGGCGTGGAGGAAAAACTG | |
| | TTTCATACAGAAGGCGTCTCGAGGCCGCCCCGACTGCATCTGCGTGTTCCAATTCG | |
| | CCAATGACAAGACGCTGGGCGGGGTTTGTGTCATCATAGAACTAAAGACATGCAA | |
| | ATATATTTCTTCCGGGGACACCGCCAGCAAACGCGAGCAACGGGCCACGGGGATG | |
| | AAGCAG | |
| 269 | QPVLTQPPSASGTPGQRVTISCSGSSSNIGSNNVIWYQQLPGAAPKLLIYSNHRRPSGVP | anti-GPC3 scFv_clone |
| | DRFSGSRSGTSASLAISGLQSEDEADYYCAAWDDSLDGYLFGTGTKVTVLGSRGGGG | 34 |
| | SGGGGSGGGGSLEMAQVQLQQWGAGLLKPSETLSLTCAVYGGSFSGYYWSWIRQPP | |
| | GKGLEWIGEINHSGSTNYNPSLKSRVTISVDTSKNQFSLELSSVTAADTAVYYCARGY | |
| | GGRFDYWGQGTLVTVSS | |
| 270 | QSVLTQPPSVSAAPGQRVTISCSGTRSNIGSDYVSWYQHLPGTAPKLLVYGDNLRPSGI | anti-GPC3 scFv_clone |
| | PDRFSASKSGTSATLGITGLQTGDEADYYCGTWDYTLNGVVFGGGTKLTVLGSRGGG | 37 |
| | GSGGGGSGGGGSLEMAQVQLVESGGGLVQPGGSLRLSCAASGFTFSSYAMSWVRQA | |
| | PGKGLEWVSVIYSGGSSTYYADSVKGRFTISRDNSKNTLYLQMNSLRAEDTAVYYCA | |
| | RTSYLNHGDYWGQGTLVTVSS | |
| 271 | QSVLTQPPSVSGTPGQRVIISCPGSTSNIGTNTVNWYQQFPGTAPKLLIYSNNQRPSGVP | anti-GPC3 scFv_clone |
| | DRFSGSKSGTSASLAISGLQSEDEADYYCAAWDDSLNGVVFGGGTKLTVLGSRGGGG | 45 |
| | SGGGGSGGGGSLEMAQMQLVQSGGGLVKPGGSLRLSCAASGFTFSDYYMSWIRQAP | |
| | GKGLEWVSYISSSGSTIYYADSVKGRFTISRDNAKNSLYLQMNSLRAEDTAVYYCAR | |
| | ASDLYGDWGQGTLVTVSS | |
| 272 | QAVLTQPPSVSTPGQRVTISCSGSSSNFGSNTVHWYQQVPGTAPKLLIFSNTQRPSEIPD | anti-GPC3 scFv_clone |
| | RFSGSKSGTSASLAISGLQSEDEADYYCAAWDDSLTGVVFGGGTKLTVLGSRGGGGS | 46 |
| | GGGGSGGGGSLEMAQVQLVQSGAEVKKPGASVTVSCKASGYRFSNYGVSWVRQAP | |
| | GQGLEWMGWISGSNGNTNYAQKFLGRVTMTTDTSTTTAYMELSSLRSDDTAVYYCA | |
| | RGNRRYYSPIIDPWGQGTLVTVSS | |
| 273 | DVVMTQSPLSLPVTPGEPASVSCRSSQSLLHSNGYNYLDWYLQKPGQSPQLLIYLGSN | anti-GPC3 scFv_clone |
| | RASGVPDRFSGSGSGTDFTLKISRVEAEDVGVYYCMQALQTPWTFGQGTKVEIKRSR | 87 |
| | GGGGSGGGGSGGGGSLEMAEVQLVQSGAEVRKPGSSVKVSCQASGGTFGSYAISWV | |
| | RQAPGQGLEWMGRIIPVLGRTKYAQKFQGRVTVTADTSTSTVYMELTSLTSEDTAVY | |
| | YCARTNDSWGQGTLVTVSS | |
| 274 | QVQLQQWGAGLLKPSETLSLTCAVYGGSFSGYYWSWIRQPPGKGLEWIGEINHSGST | anti-GPC3 V_{H}_clone |
| | NYNPSLKSRVTISVDTSKNQFSLELSSVTAADTAVYYCARGYGGRFDYWGQGTLVTV | 34 |
| | SS | |
| 275 | QPVLTQPPSASGTPGQRVTISCSGSSSNIGSNNVIWYQQLPGAAPKLLIYSNHRRPSGVP | anti-GPC3 V_{L}_clone |
| | DRFSGSRSGTSASLAISGLQSEDEADYYCAAWDDSLDGYLFGTGTKVTVLG | 34 |
| 276 | QVQLVESGGGLVQPGGSLRLSCAASGFTFSSYAMSWVRQAPGKGLEWVSVIYSGGSS | anti-GPC3 V_{H}_clone |
| | TYYADSVKGRFTISRDNSKNTLYLQMNSLRAEDTAVYYCARTSYLNHGDYWGQGTL | 37 |
| | VTVSS | |
| 277 | QSVLTQPPSVSAAPGQRVTISCSGTRSNIGSDYVSWYQHLPGTAPKLLVYGDNLRPSGI | anti-GPC3 V_{L}_clone |
| | PDRFSASKSGTSATLGITGLQTGDEADYYCGTWDYTLNGVVFGGGTKLTVLG | 37 |
| 278 | QMQLVQSGGGLVKPGGSLRLSCAASGFTFSDYYMSWIRQAPGKGLEWVSYISSSGSTI | anti-GPC3 V_{H}_clone |
| | YYADSVKGRFTISRDNAKNSLYLQMNSLRAEDTAVYYCARASDLYGDWGQGTLVT | 45 |
| | VSS | |
| 279 | QSVLTQPPSVSGTPGQRVIISCPGSTSNIGTNTVNWYQQFPGTAPKLLIYSNNQRPSGVP | anti-GPC3 V_{L}_clone |
| | DRFSGSKSGTSASLAISGLQSEDEADYYCAAWDDSLNGVVFGGGTKLTVLG | 45 |
| 280 | QVQLVQSGAEVKKPGASVTVSCKASGYRFSNYGVSWVRQAPGQGLEWMGWISGSN | anti-GPC3 V_{H}_clone |
| | GNTNYAQKFLGRVTMTTDTSTTTAYMELSSLRSDDTAVYYCARGNRRYYSPIIDPWG | 46 |
| | QGTLVTVSS | |
| 281 | QAVLTQPPSVSGTPGQRVTISCSGSSSNFGSNTVHWYQQVPGTAPKLLIFSNTQRPSEIP | anti-GPC3 V_{L}_clone |
| | DRFSGSKSGTSASLAISGLQSEDEADYYCAAWDDSLTGVVFGGGTKLTVLG | 46 |
| 282 | QVQLVQSGAEVKKPGASVKVSCKASGYTFTSYYMHWVRQAPGQGLEWMGIINPSGG | anti-GPC3 V_{H}_clone |
| | STSYAQKFQGRVTMTRDTSTSTVYMELSSLRSEDTAVYYCARWHGGPYDYWGQGTL | 55 |
| | VTVSS | |
| 283 | QPVLTQPPSVSVAPGKTARITCGGNNIGSKSVHWYQQKPGQAPVLVIYYDSDRPSGIP | anti-GPC3 V_{L}_clone |
| | ERFSGSNSGNTATLTISRVEAGDEADYYCQVWDSSSDHYVFGTGTKVTVLG | 55 |
| 284 | QVQLVQSGADVRKPGASVKVSCKASGYTFASHGISWVRQAPGQGLEWLGWISPYTG | anti-GPC3 V_{H}_clone |
| | NTNYAQKFQGRVTMATDTSTSTAYMELRSLRSDDTAIYYCARGKRTLASCFDYWGQ | 58 |
| | GTLVTVSS | |
| 285 | QSVLTQPPSVSVAPGKTARITCGGNNIGSKSVHWYQQKPGQAPVLVVYDDSDRPSGIP | anti-GPC3 V_{L}_clone |
| | ERFSGSNSGNTATLTISRVEAGDEADYYCQVWDSSSDHVFGTGTKVTVLG | 58 |
| 286 | EVQLVQSGAEVRKPGSSVKVSCQASGGTFGSYAISWVRQAPGQGLEWMGRIIPVLGR | anti-GPC3 V_{H}_clone |
| | TKYAQKFQGRVTVTADTSTSTVYMELTSLTSEDTAVYYCARTNDSWGQGTLVTVSS | 87 |
| 287 | DVVMTQSPLSLPVTPGEPASVSCRSSQSLLHSNGYNYLDWYLQKPGQSPQLLIYLGSN | anti-GPC3 V_{L}_clone |
| | RASGVPDRFSGSGSGTDFTLKISRVEAEDVGVYYCMQALQTPWTFGQGTKVEIKR | 87 |
| 288 | METDTLLLWVLLLWVPGSTGQPVLTQPPSASGTPGQRVTISCSGSSSNIGSNNVIWYQ | anti-GPC3-CD30-CSR |
| | QLPGAAPKLLIYSNHRRPSGVPDRFSGSRSGTSASLAISGLQSEDEADYYCAAWDDSL | with SP and myc |
| | DGYLFGTGTKVTVLGSRGGGGSGGGGSGGGGSLEMAQVQLQQWGAGLLKPSETLSL | tag_clone 34 |
| | TCAVYGGSFSGYYWSWIRQPPGKGLEWIGEINHISGSTNYNPSLKSRVTISVDTSKNQF | |
| | SLELSSVTAADTAVYYCARGYGGRFDYWGQGTLVTVSSEQKLISEEDLAAATGAPPL | |
| | GTQPDCNPTPENGEAPASTSPTQSLLVDSQASKTLPIPTSAPVALSSTGKPVLDAGPVL | |
| | FWVILVLVVVVGSSAFLLCHRRACRKRIRQKLHLCYPVQTSQPKLELVDSRPRRSSTQ | |
| | LRSGASVTEPVAEERGLMSQPLMETCHSVGAAYLESLPLQDASPAGGPSSPRDLPEPR | |
| | VSTEHTNNKIEKIYIMKADTVIVGTVKAELPEGRGLAGPAEPELEEELEADHTPHYPEQ | |
| | ETEPPLGSCSDVMLSVEEEGKEDPLPTAASGK | |
| 289 | METDTLLLWVLLLWVPGSTGQSVLTQPPSVSGTPGQRVIISCPGSTSNIGTNTVNWYQ | anti-GPC3-CD30-CSR |
| | QFPGTAPKLLIYSNNQRPSGVPDRFSGSKSGTSASLAISGLQSEDEADYYCAAWDDSL | with SP and myc |
| | NGVVFGGGTKLTVLGSRGGGGSGGGGSGGGGSLEMAQMQLVQSGGGLVKPGGSLR | tag_clone 45 |
| | LSCAASGFTFSDYYMSWIRQAPGKGLEWVSYISSSGSTIYYADSVKGRFTISRDNAKN | |
| | SLYLQMNSLRAEDTAVYYCARASDLYGDWGQGTLVTVSSEQKLISEEDLAAATGAPP | |
| | LGTQPDCNPTPENGEAPASTSPTQSLLVDSQASKTLPIPTSAPVALSSTGKPVLDAGPV | |
| | LFWVILVLVVVVGSSAFLLCHRRACRKRIRQKLHLCYPVQTSQPKLELVDSRPRRSST | |
| | QLRSGASVTEPVAEERGLMSQPLMETCHSVGAAYLESLPLQDASPAGGPSSPRDLPEP | |
| | RVSTEHTNNKIEKIYIMKADTVIVGTVKAELPEGRGLAGPAEPELEEELEADHTPHYPE | |
| | QETEPPLGSCSDVMLSVEEEGKEDPLPTAASGK | |
| 290 | METDTLLLWVLLLWVPGSTGQAVLTQPPSVSTPGQRVTISCSGSSSNFGSNTVHWYQ | anti-GPC3-CD30-CSR |
| | QVPGTAPKLLIFSNTQRPSEIPDRFSGSKSGTSASLAISGLQSEDEADYYCAAWDDSLT | with SP and myc |
| | GVVFGGGTKLTVLGSRGGGGSGGGGSGGGGSLEMAQVQLVQSGAEVKKPGASVTV | tag_clone 46 |
| | TTTAYMELSSLRSDDTAVYYCARGNRRYYSPIIDPWGQGTLVTVSSEQKLISEEDLAA | |
| | ATGAPPLGTQPDCNPTPENGEAPASTSPTQSLLVDSQASKTLPIPTSAPVALSSTGKPVL | |
| | DAGPVLFWVILVLVVVVGSSAFLLCHRRACRKRIRQKLHLCYPVQTSQPKLELVDSRP | |
| | RRSSTQLRSGASVTEPVAEERGLMSQPLMETCHSVGAAYLESLPLQDASPAGGPSSPR | |
| | DLPEPRVSTEHTNNKIEKIYIMKADTVIVGTVKAELPEGRGLAGPAEPELEEELEADHT | |
| | PHYPEQETEPPLGSCSDVMLSVEEEGKEDPLPTAASGK | |
| 291 | METDTLLLWVLLLWVPGSTGQPVLTQPPSVSVAPGKTARITCGGNNIGSKSVHWYQQ | anti-GPC3-CD30-CSR |
| | KPGQAPVLVIYYDSDRPSGIPERFSGSNSGNTATLTISRVEAGDEADYYCQVWDSSSD | with SP and myc |
| | HYVFGTGTKVTVLGSRGGGGSGGGGSGGGGSLEMAQVQLVQSGAEVKKPGASVKV | tag_clone 55 |
| | SCKASGYTFTSYYMHWVRQAPGQGLEWMGIINPSGGSTSYAQKFQGRVTMTRDTST | |
| | STVYMELSSLRSEDTAVYYCARWHGGPYDYWGQGTLVTVSSEQKLISEEDLAAATG | |
| | GPVLFWVILVLVVVVGSSAFLLCHRRACRKRIRQKLHLCYPVQTSQPKLELVDSRPRR | |
| | SSTQLRSGASVTEPVAEERGLMSQPLMETCHSVGAAYLESLPLQDASPAGGPSSPRDL | |
| | PEPRVSTEHTNNKIEKIYIMKADTVIVGTVKAELPEGRGLAGPAEPELEEELEADHTPH | |
| | YPEQETEPPLGSCSDVMLSVEEEGKEDPLPTAASGK | |
| 292 | METDTLLLWVLLLWVPGSTGQSVLTQPPSVSVAPGKTARITCGGNNIGSKSVHWYQQ | anti-GPC3-CD30-CSR |
| | KPGQAPVLVVYDDSDRPSGIPERFSGSNSGNTATLTISRVEAGDEADYYCQVWDSSSD | with SP and myc |
| | HVFGTGTKVTVLGSRGGGGSGGGGSGGGGSLEMAQVQLVQSGADVRKPGASVKVS | tag_clone 58 |
| | CKASGYTFASHGISWVRQAPGQGLEWLGWISPYTGNTNYAQKFQGRVTMATDTSTS | |
| | TAYMELRSLRSDDTAIYYCARGKRTLASCFDYWGQGTLVTVSSEQKLISEEDLAAAT | |
| | GAPPIGTQPDCNPTPENGEAPASTSPTQSLLVDSQASKTLPIPTSAPVALSSTGKPVLD | |
| | AGPVLFWVILVLVVVVGSSAFLLCHRRACRKRIRQKLHLCYPVQTSQPKLELVDSRPR | |
| | RSSTQLRSGASVTEPVAEERGLMSQPLMETCHSVGAAYLESLPLQDASPAGGPSSPRD | |
| | LPEPRVSTEHTNNKIEKIYIMKADTVIVGTVKAELPEGRGLAGPAEPELEEELEADHTP | |
| | HYPEQETEPPLGSCSDVMLSVEEEGKEDPLPTAASGK | |
| 293 | METDTLLLWVLLLWVPGSTGDVVMTQSPLSLPVTPGEPASVSCRSSQSLLHSNGYNY | anti-GPC3-CD30-CSR |
| | LDWYLQKPGQSPQLLIYLGSNRASGVPDRFSGSGSGTDFTLKISRVEAEDVGVYYCM | with SP and myc |
| | QALQTPWTFGQGTKVEIKRSRGGGGSGGGGSGGGGSLEMAEVQLVQSGAEVRKPGS | tag_clone 87 |
| | SVKVSCQASGGTFGSYAISWVRQAPGQGLEWMGRIIPVLGRTKYAQKFQGRVTVTA | |
| | DTSTSTVYMELTSLTSEDTAVYYCARTNDSWGQGTLVTVSSEQKLISEEDLAAATGA | |
| | PPLGTQPDCNPTPENGEAPASTSPTQSLLVDSQASKTLPIPTSAPVALSSTGKPVLDAGP | |
| | VLFWVILVLVVVVGSSAFLLCHRRACRKRIRQKLHLCYPVQTSQPKLELVDSRPRRSS | |
| | TQLRSGASVTEPVAEERGLMSQPLMETCHSVGAAYLESLPLQDASPAGGPSSPRDLPE | |
| | PRVSTEHTNNKIEKIYIMKADTVIVGTVKAELPEGRGLAGPAEPELEEELEADHTPHYP | |
| | EQETEPPLGSCSDVMLSVEEEGKEDPLPTAASGK | |
| 294 | METDTLLLWVLLLWVPGSTG | Signal peptide |
| 295 | EVQLVQSGAEVKKPGASVKVSCKASGYTFTSYGISWVRQAPGQGLEWMGWISAYNG | Anti-AFP V_{H} region |
| | NTNYAQKLQGRVTMTTDTSTSTAYMELRSLRSDDTAVYYCARDSYYYYYGMDVWG | |
| | QGTTVTVSS | |
| 296 | QAVVTQEPSLTVSPGGTVTLTCGSSTGAVTSGHYPYWFQQKPGQAPRTLIYDASDKH | Anti-AFP V_{L} region |
| | SWTPARFSGSLLGGKAALTLSGAQPEDEAEYYCLLSYSDALVFGGGTKLTVLG | |
| 297 | EVQLVQSGAEVKKPGESLTISCKASGYSFPNYWITWVRQMSGGGLEWMGRIDPGDSY | Anti-AFP V_{H} region |
| | TTYNPSFQGHVTISIDKSTNTAYLHWNSLKASDTAMYYCARYYVSLVDIWGQGTLVT | |
| | VSS | |
| 298 | QSVLTQPASVSGSPGQSITISCTGTSSDVGGYNYVSWYQQHPGKAPKLMIYDVNNRPS | Anti-AFP V_{L} region |
| | EVSNRFSGSKSGNTASLTISGLQAEDEADYYCSSYTTGSRAVFGGGTKLTVLG | |
| 299 | EVQLVESGGGLVKPGGSLRLSCAASGFTFSNAWMSWVRQAPGKGLEWVGFIRSKAY | Anti-AFP V_{H} region |
| | GGTTEYAASVKGRFTISRDDSKSIAYLQMNNLKTEDTAVYYCARDGLYSSSWYDSDY | |
| | WGQGTLVTVSS | |
| 300 | QSVVTQPPSVSAAPGQKVTISCSGSSSNIGNNYVSWYQQLPGTAPKLLIYDNNKRPSGI | Anti-AFP V_{L} region |
| | PDRFSGSKSGTSATLGITGLQTGDEADYYCGTWDGSLYTMLFGGGTKLTVLG | |
| 301 | QMQLVQSGGGLVQPGRSLRLSCAASGFTFDDYAMHWVRQAPGKGLEWVSGISWNS | Anti-AFP V_{H} region |
| | GSIGYADSVKGRFTISRDNAKNSLYLQMNSLRAEDTALYYCAKDIHSGSYYGLLYYA | |
| | MDVWGQGTTVTVSS | |
| 302 | QSVLTQPPSVSGAPGQRVTISCTGSSSNIGAGYDVHWYQQLPGTAPKLLIFGNSNRPSG | Anti-AFP V_{L} region |
| | VPDRFSGFKSGTSASLAITGLQAEDEADYFCQSYDSSLSGSGVFGTGTKVTVLG | |
| 303 | EVQLVQSGAEVKKPGASVKVSCKASGYTFTSYGISWVRQAPGQGLEWMGWISAYNG | Anti-AFP V_{H} region |
| | NTNYAQKLQGRVTMTTDTSTSTAYMELRSLRSDDTAVYYCARFQDWWYLGQFDQW | |
| | GQGILVTVSS | |
| 304 | QSALTQPASVSGSPGQSITISCTATGSDVGVYYYVSWYQQHPGKAPKVMIYDVDNRP | Anti-AFP V_{L} region |
| | PGVSNRFSGSKSGNTASLTISGLQAEDEADYYCASYTNRNSLGYVFGTGTKVTVLG | |
| 305 | DRGSQS | Anti-AFP-TCR1 |
| | | alpha_CDR1 |
| 306 | IYSNGD | Anti-AFP-TCR1 |
| | | alpha_CDR2 |
| 307 | AVNSDSGYALNF | Anti-AFP-TCR1 |
| | | alpha_CDR3 |
| 308 | SGDLS | Anti-AFP-TCR1 and |
| | | Anti-AFP-TCR2 |
| | | beta_CDR1 |
| 309 | YYNGEE | Anti-AFP-TCR1 and |
| | | Anti-AFP-TCR2 |
| | | beta_CDR2 |
| 310 | CASSLGGESEQYF | Anti-AFP-TCR1 and |
| | | Anti-AFP-TCR2 |
| | | beta_CDR3 |
| 311 | DRGSQ | Anti-AFP-TCR2 |
| | | alpha_CDR1 |
| 312 | IYSNGD | Anti-AFP-TCR2 |
| | | alpha_CDR2 |
| 313 | AVNSQSGYALNF | Anti-AFP-TCR2 |
| | | alpha_CDR3 |
| 314 | QKEVEQNSGPLSVPEGAIASLNCTYSDRGSQSFFWYRQYSGKSPELIMSIYSNGDKED | Anti-AFP-TCR1 alpha |
| | GRETAQLNKASQYVSLLIRDSQPSDSATYLCAVNSDSGYALNFGKGTSLLVT | variable region |
| 315 | VTQTPKHLITATGQRVTLRCSPRSGDLSVYWYQQSLDQGLQFLIQYYNGEERAKGNIL | Anti-AFP-TCR1 and |
| | ERFSAQQFPDLHSELNLSSLELGDSALYFCASSLGGESEQYFGPGTRLTVT | Anti-AFP-TCR2 beta |
| | | variable region |
| 316 | QKEVEQNSGPLSVPEGAIASLNCTYSDRGSQAFFWYRQYSGKSPELIMSIYSNGDKED | Anti-AFP-TCR2 alpha |
| | GRFTAQLNKASQYVSLLIRDSQPSDSATYLCAVNSQSGYALNFGKGTSLLVT | variable region |
| 317 | GGSFSGYY | Anti-GPC3_HCDR1 |
| 318 | INHSGST | Anti-GPC3_HCDR2 |
| 319 | ARGYGGRFDY | Anti-GPC3_HCDR3 |
| 320 | SSNIGSNN | Anti-GPC3_LCDR1 |
| 321 | SNH | Anti-GPC3_LCDR2 |
| 322 | AAWDDSLDGYL | Anti-GPC3_LCDR3 |
| 323 | GFTFSSYA | Anti-GPC3_HCDR1 |
| 324 | IYSGGSST | Anti-GPC3_HCDR2 |
| 325 | ARTSYLNHGDY | Anti-GPC3_HCDR3 |
| 326 | RSNIGSDY | Anti-GPC3_LCDR1 |
| 327 | GDN | Anti-GPC3_LCDR2 |
| 328 | GTWDYTLNGVV | Anti-GPC3_LCDR3 |
| 329 | GFTFSDYY | Anti-GPC3_HCDR1 |
| 330 | ISSSGSTI | Anti-GPC3_HCDR2 |
| 331 | ARASDLYGD | Anti-GPC3_HCDR3 |
| 332 | TSNIGTNT | Anti-GPC3_LCDR1 |
| 333 | SNN | Anti-GPC3_LCDR2 |
| 334 | AAWDDSLNGVV | Anti-GPC3_LCDR3 |
| 335 | GYRFSNYG | Anti-GPC3_HCDR1 |
| 336 | ISGSNGNT | Anti-GPC3_HCDR2 |
| 337 | ARGNRRYYSPIIDP | Anti-GPC3_HCDR3 |
| 338 | SSNFGSNT | Anti-GPC3_LCDR1 |
| 339 | SNT | Anti-GPC3_LCDR2 |
| 340 | AAWDDSLTGVV | Anti-GPC3_LCDR3 |
| 341 | GYTFTSYY | Anti-GPC3_HCDR1 |
| 342 | INPSGGST | Anti-GPC3_HCDR2 |
| 343 | ARWHGGPYDY | Anti-GPC3_HCDR3 |
| 344 | NIGSKS | Anti-GPC3_LCDR1 |
| 345 | YDS | Anti-GPC3_LCDR2 |
| 346 | QVWDSSSDHYV | Anti-GPC3_LCDR3 |
| 347 | GYTFASHG | Anti-GPC3_HCDR1 |
| 348 | ISPYTGNT | Anti-GPC3_HCDR2 |
| 349 | ARGKRTLASCFDY | Anti-GPC3_HCDR3 |
| 350 | NIGSKS | Anti-GPC3_LCDR1 |
| 351 | DDS | Anti-GPC3_LCDR2 |
| 352 | QVWDSSSDHV | Anti-GPC3_LCDR3 |
| 353 | GGTFGSYA | Anti-GPC3_HCDR1 |
| 354 | IIPVLGRT | Anti-GPC3_HCDR2 |
| 355 | ARTNDS | Anti-GPC3_HCDR3 |
| 356 | QSLLHSNGYNY | Anti-GPC3_LCDR1 |
| 357 | LGS | Anti-GPC3_LCDR2 |
| 358 | MQALQTPWT | Anti-GPC3_LCDR3 |
| 359 | DSAIYN | Anti-NY-ESO-1 TCR |
| | | alpha_CDR1 |
| 360 | IQSSQRE | Anti-NY-ESO-1 TCR |
| | | alpha_CDR2 |
| 361 | AVRPTSGGSYIPT | Anti-NY-ESO-1 TCR |
| | | alpha_CDR3 |
| 362 | QEVTQIPAALSVPEGENLVLNCSFTLQWFRQDPGKGLTSLLLQTSGRLNASLDKSSGR | Anti-NY-ESO-1 TCR |
| | STLYIAASQPGDSATYLCAVRPTSGGSYIPTFGRGTSLIVHP | alpha_variable region |
| 363 | MNHEYMS | Anti-NY-ESO-1 TCR |
| | | beta_CDR1 |
| 364 | SVGAGI | Anti-NY-ESO-1 TCR |
| | | beta_CDR2 |
| 365 | ASSYVGNTGELF | Anti-NY-ESO-1 TCR |
| | | beta_CDR3 |
| 366 | VTQTPKFQVLKTGQSMTLQCAQDMNHEYMSWYRQDPGMGLRLIHYSVGAGITDQG | Anti-NY-ESO-1 TCR |
| | EVPNGYNVSRSTTEDFPLRLLSAAPSQTSVYFCASSYVGNTGELFFGEGSRLTV | beta_variable region |
| 367 | QVKLQQSGTEVVKPGASVKLSCKASGYIFTSYDIDWVRQTPEQGLEWIGWIFPGEGST | Anti-MUC1_V_{H} |
| | EYNEKFKGRATLSVDKSSSTAYMELTRLTSEDSAVYFCARGDYYRRYFDLWGQGTT | |
| | VTVSS | |
| 368 | DIELTQSPAIMSASPGERVTMTCSASSSIRYTYWYQQKPGSSPRLLIYDTSNVAPGVPFR | Anti-MUC1_V_{L} |
| | FSGSGSGTSYSLTINRMEAEDAATYYCQEWSGYPYTFGGGTKLELKRAAA | |
| 369 | EVQLVESGGGVVQPGRSLRLSCSASGFTFSGYGLSWVRQAPGKGLEWVAMISSGGSY | Anti-FRα_V_{H} |
| | TYYADSVKGRFAISRDNAKNTLFLQMDSLRPEDTGVYFCARHGDDPAWFAYWGQGT | |
| | PVTVSS | |
| 370 | EVQLVESGGGVVQPGRSLRLSCSASGFTFSGYGLSWVRQAPGKGLEWVAMISSGGSY | Anti-FRα_heavy chain |
| | TYYADSVKGRFAISRDNAKNTLFLQMDSLRPEDTGVYFCARHGDDPAWFAYWGQGT | |
| | PVTVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHTCPP | |
| | CPAPELLGGPSVFLFPPKPKDTLMISRTPEVTCVVVDVSHEDPEVKFNWYVDGVEVHI | |
| | NAKTKPREEQYNSTYRVVSVLTVLHQDWLNGKEYKCKVSNKALPAPIEKTISKAKGQ | |
| | PREPQVYTLPPSRDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSPGK | |
| 371 | DIQLTQSPSSLSASVGDRVTITCSVSSSISSNNLHWYQQKPGKAPKPWIYGTSNLASGV | Anti-FRα_V_{L} |
| | PSRFSGSGSGTDYTFTISSLQPEDIATYYCQQWSSYPYMYTFGQGTKVEIK | |
| 372 | DIQLTQSPSSLSASVGDRVTITCSVSSSISSNNLHWYQQKPGKAPKPWIYGTSNLASGV | Anti-FRα_light chain |
| | PSRFSGSGSGTDYTFTISSLQPEDIATYYCQQWSSYPYMYTFGQGTKVEIKRTVAAPSV | |
| | FIFPPSDEQLKSGTASVVCLLNNFYPREAKVQWKVDNALQSGNSQESVTEQDSKDST | |
| | YSLSSTLTLSKADYEKHKVYACEVTHQGLSSPVTKSFNRGEC | |
| 373 | NIGAGYD | Anti-PSMA- |
| | | A_HCDR1 |
| 374 | GNS | Anti-PSMA- |
| | | A_HCDR2 |
| 375 | QSYDSSLSGYV | Anti-PSMA- |
| | | A_HCDR3 |
| 376 | QSVLTQPPSVSGAPGQRVTISCTGSSSNIGAGYDVHWYQQLPGTAPKLLIYGNSNRPS | Anti-PSMA- |
| | GVPDRFSGSKSGTSASLAITGLQAEDEADYYCQSYDSSLSGYVFGTGTKVTVLGSR | A_V_{H} |
| 377 | GYSFTSYW | Anti-PSMA- |
| | | A_LCDR1 |
| 378 | IYPGDSDT | Anti-PSMA- |
| | | A_LCDR2 |
| 379 | ARSMGSSLYASSDV | Anti-PSMA- |
| | | A_LCDR3 |
| 380 | LEMAEVQLVQSGAEVKKPGESLKISCKGSGYSFTSYWIGWVRQMPGKGLEWMGIIYP | Anti-PSMA- |
| | GDSDTRYSPSFQGQVTISADKSISTAYLQWSSLKASDTAMYYCARSMGSSLYASSDV | A_V_{L} |
| | WGQGTLVTVSS | |
| 381 | SSNIGSNT | Anti-PSMA- |
| | | B_HCDR1 |
| 382 | SNN | Anti-PSMA- |
| | | B_HCDR2 |
| 383 | AAWDDSLNGYV | Anti-PSMA- |
| | | B_HCDR3 |
| 384 | QAVLTQPPSASGTPGQRVTISCSGSSSNIGSNTVNWYQQLPGTAPKLLMYSNNQRPSG | Anti-PSMA- |
| | VPDRFSGSKSGTSASLAISGLQSEDEADYYCAAWDDSLNGYVFGTGTKVTVLGSR | B_V_{H} |
| 385 | GYNFASYW | Anti-PSMA- |
| | | B_LCDR1 |
| 386 | IYPDDSDT | Anti-PSMA- |
| | | B_LCDR2 |
| 387 | ARDSYYGIDV | Anti-PSMA- |
| | | B_LCDR3 |
| 388 | LEMAEVQLVQSGAEMKKPGESLKISCKGSGYNFASYWVGWVRQMPGKGLEWMGTI | Anti-PSMA- |
| | YPDDSDTRYGPAFQGQVTISADKSISTAYLQWSSLKASDTAMYYCARDSYYGIDVWG | B_V_{L} |
| | QGTLVTVSS | |
| 389 | GFTFSSYA | Anti-HER2_HCDR1 |
| 390 | ISGSGYST | Anti-HER2_HCDR2 |
| 391 | AKGFQYGSGSYYTHFDY | Anti-HER2_HCDR3 |
| 392 | QGISSW | Anti-HER2_LCDR1 |
| 393 | AAS | Anti-HER2_LCDR2 |
| 394 | QQYNSYPYT | Anti-HER2_LCDR3 |
| 395 | GFTFDDYA | Ant-HER3_HCDR1 |
| 396 | ISWNSGSI | Ant-HER3_HCDR2 |
| 397 | ARDLGAKQWLEGFDY | Ant-HER3_HCDR3 |
| 398 | MKYLLPTAAAGLLLLAAQPAMAQVQLVQSGGGLVQPGRSLRLSCAASGFTFDDYAM | Ant-HER3_V_{H} |
| | HWVRQAPGKGLEWVSGISWNSGSIGYADSVKGRFTISRDNAKNSLYLQMNSLRPEDT | |
| | AVYYCARDLGAKQWLEGFDYWGQGTLVTV | |
| 399 | SLRSYY | Ant-HER3_LCDR1 |
| 400 | GKN | Ant-HER3_LCDR2 |
| 401 | NSRDSSGNHWV | Ant-HER3_LCDR3 |
| 402 | NFMLTQDPAVSVALGQTVRITCQGDSLRSYYASWYQQKPGQAPVLVIYGKNNRPSGI | Ant-HER3_V_{L} |
| | PDRFSGSTSGNSASLTITGAQAEDEADYYCNSRDSSGNHWVFGGGTKVTVLGAAAEQ | |
| | KLISEEDLNGAA | |
| 403 | GYAFTNYW | Anti-EpCAM_HCDR1 |
| 404 | IFPGSGNI | Anti-EpCAM_HCDR2 |
| 405 | ARLRNWDEPMDY | Anti-EpCAM_HCDR3 |
| 406 | QSLLNSGNQKNY | Anti-EpCAM_LCDR1 |
| 407 | WAS | Anti-EpCAM_LCDR2 |
| 408 | QNDYSYPLT | Anti-EpCAM_LCDR3 |
| 409 | GFTFRKFG | Anti- |
| | | EGFR VIII_HCDR1 |
| 410 | ISTGGYNT | Anti- |
| | | EGFR VIII_HCDR2 |
| 411 | TRGYSSTSYAMDY | Anti- |
| | | EGFR VIII_HCDR3 |
| 412 | QVKLQQSGGGLVKPGASLKLSCVTSGFTFRKFGMSWVRQTSDKRLEWVASISTGGY | Anti-EGFR VIII_V_{H} |
| | NTYYSDNVKGRFTISRENAKNTLYLQMSSLKSEDTALYYCTRGYSSTSYAMDYWGQ | |
| | GTTVTV | |
| 413 | TDIDDD | Anti- |
| | | EGFR VIII_LCDR1 |
| 414 | EGN | Anti- |
| | | EGFR VIII_LCDR2 |
| 415 | LQSFNVPLT | Anti- |
| | | EGFRVIII_LCDR3 |
| 416 | DIELTQSPASLSVATGEKVTIRCMTSTDIDDDMNWYQQKPGEPPKFLISEGNTLRPGVP | Anti-EGFR VIII_V_{L} |
| | SRFSSSGTGTDFVFTIENTLSEDVGDYYCLQSFNVPLTFGDGTKLEIK | |
| 417 | GYIFTSYD | Anti-MUC1_HCDR1 |
| 418 | IFPGEGST | Anti-MUC1_HCDR2 |
| 419 | ARGDYYRRYFDL | Anti-MUC1_HCDR3 |
| 420 | ASSSIRY | Anti-MUC1_LCDR1 |
| 421 | DTS | Anti-MUC1_LCDR2 |
| 422 | QEWSGYPYT | Anti-MUC1_LCDR3 |
| 423 | GYGLS | Anti-FRα_HCDR1 |
| 424 | MISSGGSYTYYAD | Anti-FRα_HCDR2 |
| 425 | HGDDPAWFAY | Anti-FRα_HCDR3 |
| 426 | SVSSSISSNNLH | Anti-FRα_LCDR1 |
| 427 | GTSNLAS | Anti-FRα_LCDR2 |
| 428 | QQWSSYPYMYT | Anti-FRα_LCDR3 |
| 429 | GFTFSSYA | Anti-MUC16_HCDR1 |
| 430 | ISSAGGYI | Anti-MUC16_HCDR2 |
| 431 | ARQGFGNYGDYYAMDY | Anti-MUC16_HCDR3 |
| 432 | QSLLNSRTRKNQ | Anti-MUC16_LCDR1 |
| 433 | WAS | Anti-MUC16_LCDR2 |
| 434 | QQSYNLLT | Anti-MUC16_LCDR3 |
| 435 | GFSLSTVGMG | Anti-MUC16_HCDR1 |
| 436 | IWWDDEDK | Anti-MUC16_HCDR2 |
| 437 | TRIGTAQATDALDY | Anti-MUC16_HCDR3 |
| 438 | KSLLHSNGNTY | Anti-MUC16_LCDR1 |
| 439 | YMS | Anti-MUC16_LCDR2 |
| 440 | MQSLEYPLT | Anti-MUC16_LCDR3 |
| 441 | EIVLTQSPGTLSLSPGERATLSCRASQSVSSSYLAWYQQKPGQAPRLLIYGASSRATGIP | ROR1-18 scFv |
| | DRFSGSGSGTDFTLTISRLEPEDFAVYYCQQYGSSFGPGTKVDIKRSRGGGGSGGGGS | |
| | GGGGSLEMAQVQLVQSGTEVKKPGSSVKVSCQASGGSLSSHGVSWLRQAPGQGLEW | |
| | VGRIIPMFGVTDYAQKFQDRVTITADKSTSTVYMELISLGSDDTAVYFCARESRGATF | |
| | EYWGQGTLVTVSS | |
| 442 | QSVLTQPASVSGSPGQSITISCTGTSSDFGDYDYVSWYQQHPGKAPKLMIYDVSDRPS | ROR1-56 scFv |
| | GVSNRFSGSKSGNTASLTISGLQAEDEADYFCSSLTTSSTLVFGGGTKLTVLGSRGGG | |
| | GSGGGGSGGGGSLEMAQLQLQESGPGLVKPSETLSLTCTVSGGSISSSSYYWGWIRQP | |
| | PGKGLEWIGSIYYSGSTYYNPSLKSRVTISVDTSKNQFSLKLGSVTAADTAVYYCARH | |
| | DGTDAFDIWGQGTTVTVSS | |
| 443 | KNDAPVVQEPRRLSFRSTIYGSR | ROR1 peptide can be |
| | | targeted by CAR |
| 444 | AANCIRIGIPMADPI | ROR1 peptide can be |
| | | targeted by CAR |
| 445 | SSTGVLFVKFGPPPTASPG | ROR1 peptide can be |
| | | targeted by CAR |
| 446 | SNPMILMRLKLPNCE | ROR1 peptide can be |
| | | targeted by CAR |
| 447 | GGSLSSHGVS | Anti-ROR1_HCDR1 |
| 448 | RIIPMFGVTDYAQKFQD | Anti-ROR1_HCDR2 |
| 449 | ESRGATFEY | Anti-ROR1_HCDR3 |
| 450 | QVQLVQSGTEVKKPGSSVKVSCQASGGSLSSHGVSWLRQAPGQGLEWVGRIIPMFGV | Anti-ROR1_V_{H} |
| | TDYAQKFQDRVTITADKSTSTVYMELISLGSDDTAVYFCARESRGATFEYWGQGTLV | |
| | TVSS | |
| 451 | RASQSVSSSYLA | Anti-ROR1_LCDR1 |
| 452 | GASSRAT | Anti-ROR1_LCDR2 |
| 453 | QQYGSS | Anti-ROR1_LCDR3 |
| 454 | EIVLTQSPGTLSLSPGERATLSCRASQSVSSSYLAWYQQKPGQAPRLLIYGASSRATGIP | Anti-ROR1_V_{L} |
| | DRFSGSGSGTDFTLTISRLEPEDFAVYYCQQYGSSFGPGTKVDIKR | |
| 455 | GGSISSSSYYWG | Anti-ROR1_HCDR1 |
| 456 | SIYYSGSTYYNPSLKS | Anti-ROR1_HCDR2 |
| 457 | HDGTDAFDI | Anti-ROR1_HCDR3 |
| 458 | QLQLQESGPGLVKPSETLSLTCTVSGGSISSSSYYWGWIRQPPGKGLEWIGSIYYSGST | Anti-ROR1_V_{H} |
| | YYNPSLKSRVTISVDTSKNQFSLKLGSVTAADTAVYYCARHDGTDAFDIWGQGTTVT | |
| | VSS | |
| 459 | TGTSSDFGDYDYVS | Anti-ROR1_LCDR1 |
| 460 | DVSDRPS | Anti-ROR1_LCDR2 |
| 461 | SSLTTSSTLV | Anti-ROR1_LCDR3 |
| 462 | QSVLTQPASVSGSPGQSITISCTGTSSDFGDYDYVSWYQQHPGKAPKLMIYDVSDRPS | Anti-ROR1_V_{L} |
| | GVSNRFSGSKSGNTASLTISGLQAEDEADYFCSSLTTSSTLVFGGGTKLTVLG | |
| 463 | LVVVGAGGV | KRAS TCR target |
| | | peptide, RAS9-WT |
| 464 | KLVVVGAGGV | KRAS target peptide, |
| | | RAS10-WT |
| 465 | LVVVGAVGV | KRAS TCR target |
| | | peptide, RAS9-G12V |
| 466 | LVVVGACGV | KRAS TCR target |
| | | peptide, RAS9-G12C |
| 467 | LVVVGADGV | KRAS TCR target |
| | | peptide, RAS9-G12D |
| 468 | KLVVVGAVGV | KRAS TCR target |
| | | peptide, RAS10-G12V |
| 469 | KLVVVGACGV | KRAS TCR target |
| | | peptide, RAS10-G12C |
| 470 | KLVVVGADGV | KRAS TCR target |
| | | peptide, RAS10-G12D |
| 471 | KLVVVGASGV | KRAS TCR target |
| | | peptide, RAS10-G12S |
| 472 | VLPLTVAEV | MSLN TCR target |
| | | peptide, |
| | | MSLN530-538 HLA- |
| | | A2 |
| 473 | SLLFLLFSL | MSLN TCR target |
| | | peptide, |
| | | MSLN20-28 HLA-A2 |
| 474 | ALYVDSLFFL | PRAME TCR target |
| | | peptide, |
| | | PRA300-309 |
| 475 | NLTHVLYPV | PRAME TCR target |
| | | peptide, |
| | | PRA435-443 |
| 476 | VLDGLDVLL | PRAME TCR target |
| | | peptide, |
| | | PRA100-108 |
| 477 | SLYSFPEPEA | PRAME TCR target |
| | | peptide, |
| | | PRA142-151 |
| 478 | SLLQHLIGL | PRAME TCR target |
| | | peptide, |
| | | PRA425-433 |
| 479 | RMFPNAPYL | WT1 TCR target |
| | | peptide |
| 480 | RKSAPSTGGV | Histone H3.3 TCR |
| | | target peptide, |
| | | WT H3.3 26-25 |
| 481 | RMSAPSTGGV | Histone H3.3 TCR |
| | | target peptide, |
| | | Mutant K27M-H3.3 |
| | | 26-35 |
| 482 | GVYDGREHTV | MAGE-A4 TCR target |
| | | peptide, HLA-A02:01 |
| 483 | KVLEHVVRV | MAGE-A4 TCR target |
| | | peptide, HLA-A02:01 |
| 484 | NYKRCFPVI | MAGE-A4 TCR target |
| | | peptide, HLA-A24:02 |
| | | MAGE-A4 143-151) |
| 485 | RLARLALVL | 5T4 antigenic peptide |
| | | targeted by TCR |
| 486 | CAVPDDAGNMLTF | CDR3 of alpha chain |
| | | of a TCR that targets |
| | | SEQ ID NO: 485 |
| 487 | CASSELPAGGTNEQFF | CDR3 of beta chain of |
| | | a TCR that targets SEQ |
| | | ID NO: 485 |
| 488 | CASMYSGGGADGLTF | CDR3 of alpha chain |
| | | of a TCR that targets |
| | | SEQ ID NO: 485 |
| 489 | CASSFFSNTGELFF | CDR3 of beta chain of |
| | | a TCR that targets SEQ |
| | | ID NO: 485 |
| 490 | CASGGGADGLTF | CDR3 of alpha chain |
| | | of a TCR that targets |
| | | SEQ ID NO: 485 |
| 491 | CASSFLTDTQYF | CDR3 of beta chain of |
| | | a TCR that targets SEQ |
| | | ID NO: 485 |
| 492 | CAGGGGADGLTF | CDR3 of alpha chain |
| | | of a TCR that targets |
| | | SEQ ID NO: 485 |
| 493 | CASSYMGPEAFF | CDR3 of beta chain of |
| | | a TCR that targets SEQ |
| | | ID NO: 485 |
| 494 | CSSGGGADGLTF | CDR3 of alpha chain |
| | | of a TCR that targets |
| | | SEQ ID NO: 485 |
| 495 | CASMDLAFKQYF | CDR3 of beta chain of |
| | | a TCR that targets SEQ |
| | | ID NO: 485 |
| 496 | CAYRSGSDGGSQGNLIF | CDR3 of alpha chain |
| | | of a TCR that targets |
| | | SEQ ID NO: 485 |
| 497 | CASSQVSGYEQYF | CDR3 of beta chain of |
| | | a TCR that targets SEQ |
| | | ID NO: 485 |
| 498 | CAVRDDYGQNFVF | CDR3 of alpha chain |
| | | of a TCR that targets |
| | | SEQ ID NO: 485 |
| 499 | CASSPQGDNEQFF | CDR3 of beta chain of |
| | | a TCR that targets SEQ |
| | | ID NO: 485 |
| 500 | MVNTVAGAMK | ARHGAP35 antigenic |
| | | peptide targeted by |
| | | TCR |
| 501 | CALSSQTGANNLFF | CDR3 of alpha chain |
| | | of a TCR that targets |
| | | SEQ ID NO: 500 |
| 502 | CASSLVSPSSGSYTGELFF | CDR3 of beta chain of |
| | | a TCR that targets SEQ |
| | | ID NO: 500 |
| 503 | CAASSNNNDMRF | CDR3 of alpha chain |
| | | of a TCR that targets |
| | | SEQ ID NO: 500 |
| 504 | CASSLVRGSTEAFF | CDR3 of beta chain of |
| | | a TCR that targets SEQ |
| | | ID NO: 500 |
| 505 | KLYGLDWAEL | ASTN1 antigenic |
| | | peptide targeted by |
| | | TCR |
| 506 | CAYFGAQKLVF | CDR3 of alpha chain |
| | | of a TCR that targets |
| | | SEQ ID NO: 505 |
| 507 | CASRPSRGTNYGYTF | CDR3 of beta chain of |
| | | a TCR that targets SEQ |
| | | ID NO: 505 |
| 508 | CARYYGQNFVF | CDR3 of alpha chain |
| | | of a TCR that targets |
| | | SEQ ID NO: 505 |
| 509 | CASSERGMVEAFF | CDR3 of beta chain of |
| | | a TCR that targets SEQ |
| | | ID NO: 505 |
| 510 | CALDDRGSTLGRLYF | CDR3 of alpha chain |
| | | of a TCR that targets |
| | | SEQ ID NO: 505 |
| 511 | CASSPRNLGPSGSYEQYF | CDR3 of beta chain of |
| | | a TCR that targets SEQ |
| | | ID NO: 505 |
| 512 | TYDTVHRHL | CADPS2 antigenic |
| | | peptide targeted by |
| | | TCR |
| 513 | CILRDVGNYQLIF | CDR3 of alpha chain |
| | | of a TCR that targets |
| | | SEQ ID NO: 512 |
| 514 | CASSLDREDEQYF | CDR3 of beta chain of |
| | | a TCR that targets SEQ |
| | | ID NO: 512 |
| 515 | ELLVRINRL | CASP8 antigenic |
| | | peptide targeted by |
| | | TCR |
| 516 | CAVRDRGTGGFKTIF | CDR3 of alpha chain |
| | | of a TCR that targets |
| | | SEQ ID NO: 515 |
| 517 | CASITKDRAYEQYF | CDR3 of beta chain of |
| | | a TCR that targets SEQ |
| | | ID NO: 515 |
| 518 | ALDPHSGHFV | CDK4 antigenic |
| | | peptide targeted by |
| | | TCR |
| 519 | CVVSDLYNTDKLIF | CDR3 of alpha chain |
| | | of a TCR that targets |
| | | SEQ ID NO: 518 |
| 520 | CASSQNYEQYF | CDR3 of beta chain of |
| | | a TCR that targets SEQ |
| | | ID NO: 518 |
| 521 | CAMSSLSGNTGKLIF | CDR3 of alpha chain |
| | | of a TCR that targets |
| | | SEQ ID NO: 518 |
| 523 | CASSYSWGAGYEQYF | CDR3 of beta chain of |
| | | a TCR that targets SEQ |
| | | ID NO: 518 |
| 524 | CALPYGNKLVF | CDR3 of alpha chain |
| | | of a TCR that targets |
| | | SEQ ID NO: 518 |
| 525 | CASTPTGAYEQYF | CDR3 of beta chain of |
| | | a TCR that targets SEQ |
| | | ID NO: 518 |
| 526 | CAVILRSNDYKLSF | CDR3 of alpha chain |
| | | of a TCR that targets |
| | | SEQ ID NO: 518 |
| 527 | CSAGTGELFF | CDR3 of beta chain of |
| | | a TCR that targets SEQ |
| | | ID NO: 518 |
| 528 | CALPYGNKLVF | CDR3 of alpha chain |
| | | of a TCR that targets |
| | | SEQ ID NO: 518 |
| 529 | CASTPTGAYEQYF | CDR3 of beta chain of |
| | | a TCR that targets SEQ |
| | | ID NO: 518 |
| 530 | VLLGVKLFGV | COL18A1 antigenic |
| | | peptide targeted by |
| | | TCR |
| 531 | CAVEGDTGFQKLVF | CDR3 of alpha chain |
| | | of a TCR that targets |
| | | SEQ ID NO: 530 |
| 532 | CASSSPRSSRDTDTQYF | CDR3 of beta chain of |
| | | a TCR that targets SEQ |
| | | ID NO: 530 |
| 533 | CADVPYGQNFVF | CDR3 of alpha chain |
| | | of a TCR that targets |
| | | CORO7 antigen |
| 534 | CASSYSKAGGPGEDTQYF | CDR3 of beta chain of |
| | | a TCR that targets |
| | | CORO7 antigen |
| 535 | GYNSYSVSNSEKHIM | CTSB antigenic |
| | | peptide targeted by |
| | | TCR |
| 536 | CVVFTGGGNKLTF | CDR3 of alpha chain |
| | | of a TCR that targets |
| | | SEQ ID NO: 535 |
| 537 | CASTPQVNYGYTF | CDR3 of beta chain of |
| | | a TCR that targets SEQ |
| | | ID NO: 535 |
| 538 | CVVFGGNNARLMF | CDR3 of alpha chain |
| | | of a TCR that targets |
| | | SEQ ID NO: 535 |
| 539 | CASTLQANYGYTF | CDR3 of beta chain of |
| | | a TCR that targets SEQ |
| | | ID NO: 535 |
| 540 | LYPEFIASI | DPY19L4 antigenic |
| | | peptide targeted by |
| | | TCR |
| 541 | CAVRGTSGYALNF | CDR3 of alpha chain |
| | | of a TCR that targets |
| | | SEQ ID NO: 540 |
| 542 | CATSFAPGQGEHGYTF | CDR3 of beta chain of |
| | | a TCR that targets SEQ |
| | | ID NO: 540 |
| 543 | CAASGISGSRLTF | CDR3 of alpha chain |
| | | of a TCR that targets |
| | | FBXO21 antigen |
| 544 | CASRDHRVIYGYTF | CDR3 of beta chain of |
| | | a TCR that targets |
| | | FBXO21 antigen |
| 545 | DMKTALALYEFPSMGLLSALDHGII | GNB5 antigenic |
| | | peptide targeted by |
| | | TCR |
| 546 | CAVRDNYGQNFVF | CDR3 of alpha chain |
| | | of a TCR that targets |
| | | SEQ ID NO: 545 |
| 547 | CASSLSWGSSYNEQFF | CDR3 of beta chain of |
| | | a TCR that targets SEQ |
| | | ID NO: 545 |
| 548 | NHVVDISKSGLITIA | GPD2 antigenic |
| | | peptide targeted by |
| | | TCR |
| 549 | CAVTPNSGGYQKVTF | CDR3 of alpha chain |
| | | of a TCR that targets |
| | | SEQ ID NO: 548 |
| 550 | CASTAGLLGSSYNEQFF | CDR3 of beta chain of |
| | | a TCR that targets SEQ |
| | | ID NO: 548 |
| 551 | YRPGTVTL | H3F3B antigenic |
| | | peptide targeted by |
| | | TCR |
| 552 | CAMREDNNARLMF | CDR3 of alpha chain |
| | | of a TCR that targets |
| | | SEQ ID NO: 551 |
| 553 | CASSQEFVGAVLDTQYF | CDR3 of beta chain of |
| | | a TCR that targets SEQ |
| | | ID NO: 551 |
| 554 | YVMAYVMAGVGS | HER2 antigenic |
| | | peptide targeted by |
| | | TCR |
| 555 | CAVSVNTDKLIF | CDR3 of alpha chain |
| | | of a TCR that targets |
| | | SEQ ID NO: 554 |
| 556 | CSAPPLAGDETQYF | CDR3 of beta chain of |
| | | a TCR that targets SEQ |
| | | ID NO: 554 |
| 557 | MPYGYVLNEF | KIAA0368 antigenic |
| | | peptide targeted by |
| | | TCR |
| 558 | CAVGTSYDKVIF | CDR3 of alpha chain |
| | | of a TCR that targets |
| | | SEQ ID NO: 557 |
| 559 | CASRPWTGANEKLFF | CDR3 of beta chain of |
| | | a TCR that targets SEQ |
| | | ID NO: 557 |
| 560 | CAVHRDYKLSF | CDR3 of alpha chain |
| | | of a TCR that targets |
| | | KIAA1279 antigen |
| 561 | CASSPGPNQPQHF | CDR3 of beta chain of |
| | | a TCR that targets |
| | | KIAA1279 antigen |
| 562 | CAVSADYKLSF | CDR3 of alpha chain |
| | | of a TCR that targets |
| | | KIAA1279 antigen |
| 563 | CASSPGPNQPQHF | CDR3 of beta chain of |
| | | a TCR that targets |
| | | KIAA1279 antigen |
| 564 | CAVSADYKLSF | CDR3 of alpha chain |
| | | of a TCR that targets |
| | | KIAA1279 antigen |
| 565 | CASTPTGNTEAFF | CDR3 of beta chain of |
| | | a TCR that targets |
| | | KIAA1279 antigen |
| 566 | KKPRHDLPPYRVYLTPYTVDSPICD | KIAA1967 antigenic |
| | | peptide targeted by |
| | | TCR |
| 567 | CAVADGQKLLF | CDR3 of alpha chain |
| | | of a TCR that targets |
| | | SEQ ID NO: 566 |
| 568 | CASSSTGADSHEQFF | CDR3 of beta chain of |
| | | a TCR that targets SEQ |
| | | ID NO: 566 |
| 569 | REKQQREALERAPARLERRHSALQR | KIF16B antigenic |
| | | peptide targeted by |
| | | TCR |
| 570 | CGADFLMNRDDKIIF | CDR3 of alpha chain |
| | | of a TCR that targets |
| | | SEQ ID NO: 569 |
| 571 | CASRAVINTDTQYF | CDR3 of beta chain of |
| | | a TCR that targets SEQ |
| | | ID NO: 569 |
| 572 | CAMRERGTSYDKVIF | CDR3 of alpha chain |
| | | of a TCR that targets |
| | | SEQ ID NO: 569 |
| 573 | CASRDGRVHQPQHF | CDR3 of beta chain of |
| | | a TCR that targets SEQ |
| | | ID NO: 569 |
| 574 | CATDPSYSSASKIIF | CDR3 of alpha chain |
| | | of a TCR that targets |
| | | SEQ ID NO: 569 |
| 575 | CASSLLKAANYGYTF | CDR3 of beta chain of |
| | | a TCR that targets SEQ |
| | | ID NO: 569 |
| 576 | MTEYKLVVVGAVGVGKSALTIQLIQ | KRAS antigenic |
| | | peptide targeted by |
| | | TCR |
| 577 | CLVGDMDQAGTALIF | CDR3 of alpha chain |
| | | of a TCR that targets |
| | | SEQ ID NO: 576 |
| 578 | CASSLGQTNYGYTF | CDR3 of beta chain of |
| | | a TCR that targets SEQ |
| | | ID NO: 576 |
| 579 | CAVGRSNSGGYQKVTF | CDR3 of alpha chain |
| | | of a TCR that targets |
| | | SEQ ID NO: 576 |
| 580 | CAWSALAGARDTQYF | CDR3 of beta chain of |
| | | a TCR that targets SEQ |
| | | ID NO: 576 |
| 581 | CAVTVVNAGNNRKLIW | CDR3 of alpha chain |
| | | of a TCR that targets |
| | | SEQ ID NO: 576 |
| 582 | CASSLGLPGTDTQYF | CDR3 of beta chain of |
| | | a TCR that targets SEQ |
| | | ID NO: 576 |
| 583 | CLVGDMDQAGTALIF | CDR3 of alpha chain |
| | | of a TCR that targets |
| | | SEQ ID NO: 576 |
| 584 | CASSLGEGRVDGYTF | CDR3 of beta chain of |
| | | a TCR that targets SEQ |
| | | ID NO: 576 |
| 585 | CAAAMDSSYKLIF | CDR3 of alpha chain |
| | | of a TCR that targets |
| | | SEQ ID NO: 576 |
| 586 | CASSDPGTEAFF | CDR3 of beta chain of |
| | | a TCR that targets SEQ |
| | | ID NO: 576 |
| 587 | CLVGDRDQAGTALIF | CDR3 of alpha chain |
| | | of a TCR that targets |
| | | SEQ ID NO: 576 |
| 588 | CASSFGQSSTYGYTF | CDR3 of beta chain of |
| | | a TCR that targets SEQ |
| | | ID NO: 576 |
| 589 | CLVGDMDQAGTALIF | CDR3 of alpha chain |
| | | of a TCR that targets |
| | | SEQ ID NO: 576 |
| 590 | CASSLGRASNQPQHF | CDR3 of beta chain of |
| | | a TCR that targets SEQ |
| | | ID NO: 576 |
| 591 | GVYDGREHTV | MAGE-A4 antigenic |
| | | peptide targeted by |
| | | TCR |
| 592 | MKNQVEQSPQSLIILEGKNVTLQCNYTVSPFSNLRWYKQDTGRGPVSLTIMTFSENTK | Anti-MAGE-A4 alpha |
| | SNGRYTATLDADTKQSSLHITASQLSDSASYICVVNHSGGSYIPTFGRGTSLIVHPYIQK | chain of TCR that |
| | PDPAVYQLRDSKSSDKSVCLFTDFDSQTNVSQSKDSDVYITDKCVLDMRSMDFKSNS | targets SEQ ID |
| | AVAWSNKSDFACANAFNNSIIPEDTFFPSPESS | NO: 591 |
| 593 | MDVKVTQSSRYLVKRTGEKVFLECVQDMDHENMFWYRQDPGLGLRLIYFSYDVKM | Anti-MAGE-A4 beta |
| | KEKGDIPEGYSVSREKKERFSLILESASTNQTSMYLCASSFLMTSGDPYEQYFGPGTRL | chain of TCR that |
| | TVTEDLKNVFPPEVAVFEPSEAEISHTQKATLVCLATGFYPDHVELSWWVNGKEVHS | targets SEQ ID |
| | GVCTDPQPLKEQPALNDSRYALSSRLRVSATFWQDPRNHFRCQVQFYGLSENDEWTQ | NO: 591 |
| | DRAKPVTQIVSAEAWGRAD | |
| 594 | LMKVDPIGHVY | MAGE-A6 antigenic |
| | | peptide targeted by |
| | | TCR |
| 595 | CAVDNFNKFYF | CDR3 of alpha chain |
| | | of a TCR that targets |
| | | SEQ ID NO: 594 |
| 596 | CASSSQGGYGYTF | CDR3 of beta chain of |
| | | a TCR that targets SEQ |
| | | ID NO: 594 |
| 597 | CAGSGSRLTF | CDR3 of alpha chain |
| | | of a TCR that targets |
| | | SEQ ID NO: 594 |
| 598 | CASSFDRGYGYTF | CDR3 of beta chain of |
| | | a TCR that targets SEQ |
| | | ID NO: 594 |
| 599 | VQIISCQY | MED13 antigenic |
| | | peptide recognized by |
| | | TCR |
| 600 | CVVNTNAGKSTF | CDR3 of alpha chain |
| | | of a TCR that targets |
| | | SEQ ID NO: 599 |
| 601 | CASSGRVTGGFYNEQFF | CDR3 of beta chain of |
| | | a TCR that targets SEQ |
| | | ID NO: 599 |
| 602 | CASSGGNTPLVF | CDR3 of alpha chain |
| | | of a TCR that targets |
| | | SEQ ID NO: 599 |
| 603 | CASSFGGAYEQYF | CDR3 of beta chain of |
| | | a TCR that targets SEQ |
| | | ID NO: 599 |
| 604 | ALSPVIPHI | MLL2 antigenic |
| | | peptide recognized by |
| | | TCR |
| 605 | CALNPFNAGNMLTF | CDR3 of alpha chain |
| | | of a TCR that targets |
| | | SEQ ID NO: 604 |
| 606 | CSARVGDTGELFF | CDR3 of beta chain of |
| | | a TCR that targets SEQ |
| | | ID NO: 604 |
| 607 | CAVGEAGGGNKLTF | CDR3 of alpha chain |
| | | of a TCR that targets |
| | | SEQ ID NO: 604 |
| 608 | CASSYDSTTGELFF | CDR3 of beta chain of |
| | | a TCR that targets SEQ |
| | | ID NO: 604 |
| 609 | TGLFGQTNTGFGDVGSTLFGNNKLT | NUP98 antigenic |
| | | peptide recognized by |
| | | TCR |
| 610 | CASTAGPNFGNEKLTF | CDR3 of alpha chain |
| | | of a TCR that targets |
| | | SEQ ID NO: 609 |
| 611 | CASSANRGPDTEAFF | CDR3 of beta chain of |
| | | a TCR that targets SEQ |
| | | ID NO: 609 |
| 612 | HMTEVVRHC | p53 antigenic peptide |
| | | recognized by TCR |
| 613 | CVVQPGGYQKVTF | CDR3 of alpha chain |
| | | of a TCR that targets |
| | | SEQ ID NO: 612 |
| 614 | CASSEGLWQVGDEQYF | CDR3 of beta chain of |
| | | a TCR that targets SEQ |
| | | ID NO: 612 |
| 615 | CALDIYPHDMRF | CDR3 of alpha chain |
| | | of a TCR that targets |
| | | SEQ ID NO: 612 |
| 616 | CASSLDPGDTGELFF | CDR3 of beta chain of |
| | | a TCR that targets SEQ |
| | | ID NO: 612 |
| 617 | CAFMGYSGAGSYQLTF | CDR3 of alpha chain |
| | | of a TCR that targets |
| | | SEQ ID NO: 612 |
| 618 | CAISELVTGDSPLHF | CDR3 of beta chain of |
| | | a TCR that targets SEQ |
| | | ID NO: 612 |
| 619 | CAASKSAIMVVLQTSSSL | CDR3 of alpha chain |
| | | of a TCR that targets |
| | | SEQ ID NO: 6012 |
| 620 | CASSIQQGADTQYF | CDR3 of beta chain of |
| | | a TCR that targets SEQ |
| | | ID NO: 612 |
| 621 | CALITGGGNKLTF | CDR3 of alpha chain |
| | | of a TCR that targets |
| | | SEQ ID NO: 612 |
| 622 | CASRLQGWNSPLHF | CDR3 of beta chain of |
| | | a TCR that targets SEQ |
| | | ID NO: 612 |
| 623 | CAWNSGGSNYKLTF | CDR3 of alpha chain |
| | | of a TCR that targets |
| | | SEQ ID NO: 612 |
| 624 | CASSYSQAWGQPQHF | CDR3 of beta chain of |
| | | a TCR that targets SEQ |
| | | ID NO: 612 |
| 625 | CAVRVWDYKLSF | CDR3 of alpha chain |
| | | of a TCR that targets |
| | | SEQ ID NO: 612 |
| 626 | CASSISAGGDGYTF | CDR3 of beta chain of |
| | | a TCR that targets SEQ |
| | | ID NO: 612 |
| 627 | CAVKGDYKLSF | CDR3 of alpha chain |
| | | of a TCR that targets |
| | | SEQ ID NO: 612 |
| 628 | CASSLVNTEAFF | CDR3 of beta chain of |
| | | a TCR that targets SEQ |
| | | ID NO: 612 |
| 629 | CAVYTGGFKTIF | CDR3 of alpha chain |
| | | of a TCR that targets |
| | | SEQ ID NO: 612 |
| 630 | CASNLGGGSTDTQYF | CDR3 of beta chain of |
| | | a TCR that targets SEQ |
| | | ID NO: 612 |
| 631 | CAFYYGGSQGNLIF | CDR3 of alpha chain |
| | | of a TCR that targets |
| | | SEQ ID NO: 612 |
| 632 | CASSFGSGSTDTQYF | CDR3 of beta chain of |
| | | a TCR that targets SEQ |
| | | ID NO: 612 |
| 633 | CAFYYGGSQGNLIF | CDR3 of alpha chain |
| | | of a TCR that targets |
| | | SEQ ID NO: 612 |
| 634 | CASSLGTGSTDTQYF | CDR3 of beta chain of |
| | | a TCR that targets SEQ |
| | | ID NO: 612 |
| 635 | FVVPYMIYLL | PDS5A antigenic |
| | | peptide recognized by |
| | | TCR |
| 636 | CAFTELNSGGSNYKLTF | CDR3 of alpha chain |
| | | of a TCR that targets |
| | | SEQ ID NO: 635 |
| 637 | CASSLSGGLLRTGELFF | CDR3 of beta chain of |
| | | a TCR that targets SEQ |
| | | ID NO: 635 |
| 638 | ILCHQNPVTGLLLASYDQKDAWVRD | PHKA1 antigenic |
| | | peptide recognized by |
| | | TCR |
| 639 | CVGDFNNAGNMLTF | CDR3 of alpha chain |
| | | of a TCR that targets |
| | | SEQ ID NO: 638 |
| 640 | CASSPGREQYF | CDR3 of beta chain of |
| | | a TCR that targets SEQ |
| | | ID NO: 638 |
| 641 | GNISILQENDFGVFGMDDREIMREG | RAD21 antigenic |
| | | peptide recognized by |
| | | TCR |
| 642 | CAARMEYGNKLVF | CDR3 of alpha chain |
| | | of a TCR that targets |
| | | SEQ ID NO: 641 |
| 643 | CASSFGTGRGDTEAFF | CDR3 of beta chain of |
| | | a TCR that targets SEQ |
| | | ID NO: 641 |
| 644 | CAVRSSPTGNQFYF | CDR3 of alpha chain |
| | | of a TCR that targets |
| | | SEQ ID NO: 641 |
| 645 | CASSFGTGRGDTEAFF | CDR3 of beta chain of |
| | | a TCR that targets SEQ |
| | | ID NO: 641 |
| 646 | CAESRMDSSYKLIF | CDR3 of alpha chain |
| | | of a TCR that targets |
| | | SEQ ID NO: 641 |
| 647 | CASSLVAGGNTEAFF | CDR3 of beta chain of |
| | | a TCR that targets SEQ |
| | | ID NO: 641 |
| 648 | CAVRSAAAGNKLTF | CDR3 of alpha chain |
| | | of a TCR that targets |
| | | RECQL5 antigen |
| 649 | CASSRSTGGSGNTIYF | CDR3 of beta chain of |
| | | a TCR that targets |
| | | RECQL5 antigen |
| 650 | CAETRGGATNKLIF | CDR3 of alpha chain |
| | | of a TCR that targets |
| | | RECQL5 antigen |
| 651 | CASSQAGGTGELFF | CDR3 of beta chain of |
| | | a TCR that targets |
| | | RECQL5 antigen |
| 652 | CAMSPLLETGANNLFF | CDR3 of alpha chain |
| | | of a TCR that targets |
| | | RNF19B antigen |
| 653 | CASSTSTGQGWHYGYTF | CDR3 of alpha chain |
| | | of a TCR that targets |
| | | RNF19B antigen |
| 654 | MLFSHGLVK | SKIV2L antigenic |
| | | peptide recognized by |
| | | TCR |
| 655 | CAPSYSSASKIIF | CDR3 of alpha chain |
| | | of a TCR that targets |
| | | SEQ ID NO: 654 |
| 656 | CASSPGTVKETQYF | CDR3 of beta chain of |
| | | a TCR that targets SEQ |
| | | ID NO: 654 |
| 657 | CAMSGYTNAGKSTF | CDR3 of alpha chain |
| | | of a TCR that targets |
| | | SEQ ID NO: 654 |
| 658 | CAWSAASGGAQDTQYF | CDR3 of beta chain of |
| | | a TCR that targets SEQ |
| | | ID NO: 654 |
| 659 | DPPALASTNAEVT | SLC3A2 antigenic |
| | | peptide recognized by |
| | | TCR |
| 660 | CVVSAAQAGTALIF | CDR3 of alpha chain |
| | | of a TCR that targets |
| | | SEQ ID NO: 659 |
| 661 | CASSASTGRNQPQHF | CDR3 of beta chain of |
| | | a TCR that targets SEQ |
| | | ID NO: 659 |
| 662 | CAMSAGANTGNQFYF | CDR3 of alpha chain |
| | | of a TCR that targets |
| | | SEQ ID NO: 659 |
| 663 | CASSLARRQYGYTF | CDR3 of beta chain of |
| | | a TCR that targets SEQ |
| | | ID NO: 659 |
| 664 | CILRAPSGNTPLVF | CDR3 of alpha chain |
| | | of a TCR that targets |
| | | SEQ ID NO: 659 |
| 665 | CASSSQGFYNIQYF | CDR3 of beta chain of |
| | | a TCR that targets SEQ |
| | | ID NO: 659 |
| 666 | CVVSPSGGSYIPTF | CDR3 of alpha chain |
| | | of a TCR that targets |
| | | SEQ ID NO: 659 |
| 667 | CASSPGSPSSYEQYF | CDR3 of beta chain of |
| | | a TCR that targets SEQ |
| | | ID NO: 659 |
| 668 | CALSDPQIKAAGNKLTF | CDR3 of alpha chain |
| | | of a TCR that targets |
| | | SEQ ID NO: 659 |
| 669 | CASSIRNYSNQPQHF | CDR3 of beta chain of |
| | | a TCR that targets SEQ |
| | | ID NO: 659 |
| 670 | CAVAPSQAGTALIF | CDR3 of alpha chain |
| | | of a TCR that targets |
| | | SEQ ID NO: 659 |
| 671 | CASSLRTGQNTEAFF | CDR3 of beta chain of |
| | | a TCR that targets SEQ |
| | | ID NO: 659 |
| 672 | CAFMKRTNRDDKIIF | CDR3 of alpha chain |
| | | of a TCR that targets |
| | | SEQ ID NO: 659 |
| 673 | CASSGGTPYNSPLHF | CDR3 of beta chain of |
| | | a TCR that targets SEQ |
| | | ID NO: 659 |
| 674 | FLVYGVRPGM | SMARCD3 antigenic |
| | | peptide recognized by |
| | | TCR |
| 675 | CAFMQFDMRF | CDR3 of alpha chain |
| | | of a TCR that targets |
| | | SEQ ID NO: 674 |
| 676 | CASTPGGYEQYF | CDR3 of beta chain of |
| | | a TCR that targets SEQ |
| | | ID NO: 674 |
| 677 | CAVDTNTDKLIF | CDR3 of alpha chain |
| | | of a TCR that targets |
| | | SEQ ID NO: 674 |
| 678 | CASSQGYEQYF | CDR3 of beta chain of |
| | | a TCR that targets SEQ |
| | | ID NO: 674 |
| 679 | RKTVRARSRTPSCRSRSHTPSRRRR | SON antigenic peptide |
| | | recognized by TCR |
| 680 | CALQISSGSARQLTF | CDR3 of alpha chain |
| | | of a TCR that targets |
| | | SEQ ID NO: 679 |
| 681 | CASSLIRSEAFF | CDR3 of beta chain of |
| | | a TCR that targets SEQ |
| | | ID NO: 679 |
| 682 | CALRESGYSTLTF | CDR3 of alpha chain |
| | | of a TCR that targets |
| | | SEQ ID NO: 679 |
| 683 | CASSRDRSNEQFF | CDR3 of beta chain of |
| | | a TCR that targets SEQ |
| | | ID NO: 679 |
| 684 | CAGQDNPASGNTGKLIF | CDR3 of alpha chain |
| | | of a TCR that targets |
| | | SEQ ID NO: 679 |
| 685 | CASSFIRTGQYF | CDR3 of beta chain of |
| | | a TCR that targets SEQ |
| | | ID NO: 679 |
| 686 | CAAPARGGSYIPTF | CDR3 of alpha chain |
| | | of a TCR that targets |
| | | SEQ ID NO: 679 |
| 687 | CASSLVDRRGEKLFF | CDR3 of beta chain of |
| | | a TCR that targets SEQ |
| | | ID NO: 679 |
| 688 | TLWCSPIKV | SRPX antigenic |
| | | peptide recognized by |
| | | TCR |
| 689 | CAASLSGGGADGLTF | CDR3 of alpha chain |
| | | of a TCR that targets |
| | | SEQ ID NO: 688 |
| 690 | CASSLDRKAFF | CDR3 of beta chain of |
| | | a TCR that targets SEQ |
| | | ID NO: 688 |
| 691 | CALSDSGGSNYKLTF | CDR3 of alpha chain |
| | | of a TCR that targets |
| | | TFDP2 |
| 692 | CASRFVAPSDGYNEQFF | CDR3 of beta chain of |
| | | a TCR that targets |
| | | TFDP2 |
| 693 | CALSDPQDSGYSTLTF | CDR3 of alpha chain |
| | | of a TCR that targets |
| | | TFDP2 |
| 694 | CASSLGQGRVEQYF | CDR3 of beta chain of |
| | | a TCR that targets |
| | | TFDP2 |
| 695 | CAVSPGAGGSYIPTF | CDR3 of alpha chain |
| | | of a TCR that targets |
| | | TFDP2 |
| 696 | CAISEVGTGSSGNTIYF | CDR3 of beta chain of |
| | | a TCR that targets |
| | | TFDP2 |
| 697 | CAVRPSGYSTLTF | CDR3 of alpha chain |
| | | of a TCR that targets |
| | | TFDP2 |
| 698 | CAITSGQGNNEQYF | CDR3 of beta chain of |
| | | a TCR that targets |
| | | TFDP2 |
| 699 | CAVQAWGFGNVLHC | CDR3 of alpha chain |
| | | of a TCR that targets |
| | | UGGT2 |
| 700 | CASSDRNTNYGYTF | CDR3 of beta chain of |
| | | a TCR that targets |
| | | UGGT2 |
| 701 | CAVKSWSGPGWGNQAGTALIF | CDR3 of alpha chain |
| | | of a TCR that targets |
| | | UGGT2 |
| 702 | CASSMRVQSNTGELFF | CDR3 of beta chain of |
| | | a TCR that targets |
| | | UGGT2 |
| 703 | CVVNTPPNTDKLIF | CDR3 of alpha chain |
| | | of a TCR that targets |
| | | SEQ ID NO: 479 |
| 704 | CASTPFTSGSGWDEQFF | CDR3 of beta chain of |
| | | a TCR that targets SEQ |
| | | ID NO: 479 |
| 705 | CAVPSGSARQLTF | CDR3 of alpha chain |
| | | of a TCR that targets |
| | | XPNPEP1 |
| 706 | CASSLDFSRQETQYF | CDR3 of beta chain of |
| | | a TCR that targets |
| | | XPNPEP1 |
| 707 | CAVIKGYSTLTF | CDR3 of alpha chain |
| | | of a TCR that targets |
| | | XPNPEP1 |
| 708 | CASSLYLASNTGELFF | CDR3 of beta chain of |
| | | a TCR that targets |
| | | XPNPEP1 |
| 709 | YIQNPDPAVYQLRDSKSSDKSVCLFTDFDSQTNVSQSKDSDVYITDKTVLDMRSMDF | TCR alpha chain |
| | KSNSAVAWSNKSDFACANAFNNSIIPEDTFFPSPESSCDVKLVEKSFETDTNLNFQNLS | constant region |
| | VIGFRILLLKVAGFNLLMTLRLWSS | |
| 710 | LEDLKNVFPPEVAVFEPSEAEISHTQKATLVCLATGFYPDHVELSWWVNGKEVHSGV | TCR beta chain |
| | STDPQPLKEQPALNDSRYCLSSRLRVSATFWQNPRNHFRCQVQFYGLSENDEWTQDR | constant region |
| | AKPVTQIVSAEAWGRADCGFTSESYQQGVLSATILYEILLGKATLYAVLVSALVLMA | |
| | MVKRKDSR | |

One or more features from any embodiments described herein or in the figures may be combined with one or more features of any other embodiment described herein in the figures without departing from the scope of the disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed:

1. An immune cell comprising:
(a) an αβ T-cell receptor (TCR), and
(b) a chimeric stimulating receptor (CSR) comprising:
(i) a ligand-binding module that is capable of binding or interacting with a target ligand;
(ii) a transmembrane domain (a CSR transmembrane domain); and
(iii) a CD30 costimulatory domain,
wherein the CSR lacks a functional primary signaling domain derived from the intracellular signaling sequence of CD3ζ, optionally wherein:
the CD30 costimulatory domain comprises a sequence that can bind to an intracellular TRAF signaling protein, e.g., corresponding to residues 561-573 or 578-586 of a full-length CD30 having the sequence of SEQ ID NO:228; and/or
the CD30 costimulatory domain comprises a sequence that is at least 80%, 85, 90%, 95%, or 100% identical to residues 561-573 or 578-586 of SEQ ID NO:228 or a sequence that is at least 70%, 75%, 80%, 85%, 90%, 95%, or 100% identical to the sequence of SEQ ID NO:238.

2. The immune cell of claim 1, wherein the CSR comprises more than one CD30 costimulatory domain; or the CSR further comprises at least one costimulatory domain which comprises the intracellular sequence of a costimulatory molecule that is different from CD30, optionally wherein the costimulatory molecule that is different from CD30 is selected from the group consisting of CD27, CD28, 4-1BB (CD137), OX40, CD40, PD-1, ICOS, lymphocyte function-associated antigen-1 (LFA-1), CD2, CD7, LIGHT, NKG2C, B7-H3, and a ligand that specifically binds with CD83.

3. The immune cell of claim 1 or 2, wherein the ligand-binding module of the CSR is derived from the extracellular domain of a receptor; or the ligand-binding module of the CSR comprises an antibody moiety (a CSR antibody moiety), optionally wherein the CSR antibody moiety is a single chain antibody fragment, such as a single chain Fv (scFv), a single chain Fab, a single chain Fab′, a single domain antibody fragment, a single domain multispecific antibody, an intrabody, a nanobody, or a single chain immunokine.

4. The immune cell of claim 3, wherein the CSR antibody moiety is a single domain multispecific antibody, optionally wherein the single domain multispecific antibody is a single domain bispecific antibody; and/or the CSR antibody moiety is a single chain Fv (scFv), optionally a tandem scFv.

5. The immune cell of any one of claims 1 to 4, wherein the TCR and/or the CSR antibody moiety specifically binds to a disease-related MHC-restricted antigen, optionally wherein the disease-related antigen is a cancer-related antigen.

6. The immune cell of any one of claims 3 to 5, wherein both the TCR and the CSR antibody moiety specifically bind to a MHC-restricted antigen, optionally wherein the TCR and the CSR antibody moiety specifically bind to the same antigen, the TCR and the CSR antibody moiety specifically bind to different peptides from the same antigen; or the TCR and the CSR antibody moiety specifically bind to different antigens.

7. The immune cell of any one of claims 1 to 6, wherein the TCR and/or the CSR antibody moiety specifically binds to a complex comprising a peptide and an MHC protein, and wherein the peptide is derived from a protein selected from the group consisting of WT-1, AFP, HPV16-E7, NY-ESO-1, PRAME, EBV-LMP2A, KRAS, FoxP3, Histone H3.3, PSA, COL18A1, SRPX, KIF16B, TFDP2, KIAA1279, XPNPEP1, UGGT2, PHKA1, KIF16B, SON, GNB5, FBXO21, CORO7, RECQL5, TFDP2, KIAA1967, KIF16B, NUP98, GPD2, CASP8, SKIV2L, H3F3B, MAGE-A4, MAGEA6, PDS5A, MED13, SLC3A2, KIAA0368, CADPS2, CTSB, DPY19L4, RNF19B, ASTN1, CDK4, MLL2, SMARCD3, p53, RAD21, RUSC2, VPS16, MGA, ARHGAP35, HER2, 5T4, and a variant or mutant thereof.

8. The immune cell of claim 7, wherein the TCR specifically binds to the complex; and/or the CSR antibody moiety specifically binds to a cell surface antigen, optionally wherein the cell surface antigen is selected from the group consisting of protein, carbohydrate, and lipid.

9. The immune cell of claim 7 or 8, wherein the TCR specifically binds to a complex comprising an MHC protein and a peptide derived from a cell surface antigen, and wherein the CSR antibody moiety specifically bind to the same cell surface antigen, optionally wherein the cell surface antigen is Glypican 3 (GPC3), HER2/ERBB2, EpCAM, MUC16, folate receptor alpha (FRα), MUC1, EGFR, EGFRvIII, HER3, DLL3, c-Met, ROR2, CD70, MCT4, MSLN, PSMA, or a variant or mutant thereof; and/or the TCR specifically binds to a complex comprising an alpha-fetoprotein (AFP) peptide and an MHC class I protein, optionally wherein the AFP peptide comprises an amino acid sequence of any one of SEQ ID NOS:26-36.

10. The immune cell of any one of claims 1 to 9, wherein the TCR comprises: (1) an anti-AFP-TCRα chain comprising sequences of CDR1, CDR2, and CDR3 of SEQ ID NOS:305-307, respectively; or (2) an anti-AFP-TCRβ chain comprising sequences of CDR1, CDR2, and CDR3 of SEQ ID NOS:308-310, respectively; or (3) an anti-AFP-TCRα chain comprising sequences of CDR1, CDR2, and CDR3 of SEQ ID NOS:311-313, respectively; optionally wherein the TCR comprises: (1) an anti-AFP-TCRα chain variable region comprising a sequence of SEQ ID NO:314; or (2) an anti-AFP-TCRβ chain variable region comprising a sequence of SEQ ID NO:315; or (3) an anti-AFP-TCRα chain variable region comprising a sequence of SEQ ID NO:316; and/or the TCR comprises a sequence of any one of SEQ ID NOS:1-3.

11. The immune cell of any one of claims 1 to 9, wherein the TCR comprises a sequence of any one of SEQ ID NOS:6-19 and 178-180.

12. The immune cell of any one of claims 1 to 11, wherein the CSR specifically binds to glypican 3 (GPC3), optionally wherein the CSR comprises: (1) sequences of HCDR1, HCDR2, HCDR3, LCDR1, LCDR2, and LCDR3 of SEQ ID NOS:317-322, respectively; or (2) sequences of HCDR1, HCDR2, HCDR3, LCDR1, LCDR2, and LCDR3 of SEQ ID NOS:323-328, respectively; or (3) sequences of HCDR1, HCDR2, HCDR3, LCDR1, LCDR2, and LCDR3 of SEQ ID NOS:329-334, respectively; or (4) sequences of HCDR1, HCDR2, HCDR3, LCDR1, LCDR2, and LCDR3 of SEQ ID NOS:335-340, respectively; or (5) sequences of HCDR1, HCDR2, HCDR3, LCDR1, LCDR2, and LCDR3 of SEQ ID NOS:341-346, respectively; or (6) sequences of HCDR1, HCDR2, HCDR3, LCDR1, LCDR2, and LCDR3 of SEQ ID NOS:347-352, respectively; or (7) sequences of HCDR1, HCDR2, HCDR3, LCDR1, LCDR2, and LCDR3 of SEQ ID NOS:353-358, respectively; and/or
the CSR comprises a heavy chain variable region having the sequence of any one of SEQ ID NOS:274, 276, 278, 280, 282, 284, and 286, and a light chain variable region having the sequence of any one of SEQ ID NOS:275, 277, 279, 281, 283, 285, and 287; and/or
the CSR comprises an scFv having the sequence of any one of SEQ ID NOS:212-213 and 269-273; and/or
the CSR comprises an amino acid sequence of any one of SEQ ID NOS:181-211 and 288-293.

13. The immune cell of any one of claims 1 to 11, wherein the CSR specifically binds to MSLN;

14. The immune cell of any one of claims 1 to 7, wherein the TCR specifically binds to a complex comprising a KRAS, p53, or MSLN peptide and an MHC class I protein, optionally, wherein the CSR specifically binds to MSLN and further optionally, wherein the CSR comprises:
sequences of HCDR1, HCDR2, and HCDR3 of SEQ ID NOS:71-73, respectively and/or a heavy chain variable region having the sequence of SEQ ID NO:70; and/or
sequences of LCDR1, LCDR2, and LCDR3 of SEQ ID NOS:75-77, respectively, and optionally a light chain variable region having the sequence of SEQ ID NO:74.

15. The immune cell of any one of claims 1 to 7 or an immune cell of 14 wherein the TCR specifically binds to a complex comprising a KRAS, p53, or MSLN peptide and an MHC class I protein, wherein the CSR specifically binds to ROR1; and optionally wherein: the CSR specifically binds to a ROR1 peptide having a sequence of any one of SEQ ID NOS:443-446; and further optionally wherein the CSR comprises:
sequences of HCDR1, HCDR2, and HCDR3 of SEQ ID NOS:447-449, respectively, and/or a heavy chain variable region having the sequence of SEQ ID NO:450; and/or sequences of LCDR1, LCDR2, and LCDR3 of SEQ ID NOS:451-453, respectively, and/or a light chain variable region having the sequence of SEQ ID NO:454; and/or optionally an scFv having the sequence of SEQ ID NO:441; or the CSR comprises sequences of HCDR1, HCDR2, and HCDR3 of SEQ ID NOS:455-457, respectively, and optionally a heavy chain variable region having the sequence of SEQ ID NO:458; and/or sequences of LCDR1, LCDR2, and LCDR3 of SEQ ID NOS:459-461, respectively, and optionally a light chain variable region having the sequence of SEQ ID NO:462; and/or optionally an scFv having the sequence of SEQ ID NO:442.

16. The immune cell of any one of claims 1 to 7, wherein the TCR specifically binds to a complex comprising a PSA peptide and an MHC class I protein, optionally wherein:
the CSR specifically binds to PSMA; or
the CSR specifically binds to ROR1, optionally wherein
the CSR specifically binds to a ROR1 peptide having a sequence of any one of SEQ ID NOS:443-446; and/or the CSR comprises sequences of HCDR1, HCDR2, and HCDR3 of SEQ ID NOS:447-449, respectively, and optionally a heavy chain variable region having the sequence of SEQ ID NO:450; and/or sequences of LCDR1, LCDR2, and LCDR3 of SEQ ID NOS:451-453, respectively, and optionally a light chain variable region having the sequence of SEQ ID NO:454; and/or optionally an scFv having the sequence of SEQ ID NO:441 or the CSR comprises sequences of HCDR1, HCDR2, and HCDR3 of SEQ ID NOS:455-457, respectively, and optionally a heavy chain variable region having the sequence of SEQ ID NO:458; and/or sequences of LCDR1, LCDR2, and LCDR3 of SEQ ID NOS:459-461, respectively, and optionally a light chain variable region having the sequence of SEQ ID NO:462; and/or optionally an scFv having the sequence of SEQ ID NO:442; and/or
the TCR comprises a sequence of any one of SEQ ID NOS:20-25; and optionally
wherein the CSR comprises sequences of HCDR1, HCDR2, and HCDR3 of SEQ ID NOS:373-375, respectively, and optionally a heavy chain variable region having the sequence of SEQ ID NO:376; and/or the CSR comprises sequences of LCDR1, LCDR2, and LCDR3 of SEQ ID NOS:377-379, respectively, and optionally a light chain variable region having the sequence of SEQ ID NO:380; or wherein the CSR comprises a sequence of SEQ ID NO:214; or
the CSR comprises sequences of HCDR1, HCDR2, and HCDR3 of SEQ ID NOS:381-383, respectively, and optionally a heavy chain variable region having the sequence of SEQ ID NO:384; and/or the CSR comprises sequences of LCDR1, LCDR2, and LCDR3 of SEQ ID NOS:385-387, respectively, and optionally a light chain variable region having the sequence of SEQ ID NO:388 or wherein the CSR comprises a sequence of SEQ ID NO:215.

17. The immune cell of any one of claims 1 to 7, wherein the TCR specifically binds to a complex comprising a COL18A1, SRPX, KIF16B, TFDP2, KIAA1279, XPNPEP1, UGGT2, PHKA1, KIF16B, SON, GNB5, FBXO21, CORO7, RECQL5, TFDP2, KIAA1967, KIF16B, MAGEA6, PDS5A, MED13, ASTN1, CDK4, MLL2, SMARCD3, NY-ESO-1, or PRAME peptide and an MHC class I protein.

18. The immune cell of any one of claims 1 to 7 and 17, wherein the CSR specifically binds to ROR2.

19. The immune cell of claim 17 or 18, wherein the TCR specifically binds to a complex comprising NY-ESO-1 and the MHC claims I protein and comprises: (1) sequences of CDR1, CDR2, and CDR3 of SEQ ID NOS:359-361, respectively, and optionally a variable region having the sequence of SEQ ID NO:362, and further optionally the sequence of SEQ ID NO:4; or (2) sequences of CDR1, CDR2, and CDR3 of SEQ ID NOS:363-365, respectively, and optionally a variable region having the sequence of SEQ ID NO:366, and further optionally the sequence of SEQ ID NO:5; optionally wherein the CSR comprises: (1) sequences of HCDR1, HCDR2, and HCDR3 of SEQ ID NOS:91-93, respectively, and optionally a heavy chain variable region having the sequence of SEQ ID NO:90; or (2) sequences of HCDR1, HCDR2, and HCDR3 of SEQ ID NOS:95-97, respectively, and optionally a heavy chain variable region having the sequence of SEQ ID NO:94; or (3) sequences of HCDR1, HCDR2, and HCDR3 of SEQ ID NOS:99-101, respectively, and optionally a heavy chain variable region having the sequence of SEQ ID NO:98; or (4) sequences of HCDR1, HCDR2, and HCDR3 of SEQ ID NOS:103-105, respectively, and optionally a heavy chain variable region having the sequence of SEQ ID NO:102; or (5) sequences of HCDR1, HCDR2, and HCDR3 of SEQ ID NOS:107-109, respectively, and optionally a heavy chain variable region having the sequence of SEQ ID NO:106; and/or the CSR comprises: (1) sequences of LCDR1, LCDR2, and LCDR3 of SEQ ID NOS: 111-113, respectively, and optionally a light chain variable region having the sequence of SEQ ID NO:110; or (2) sequences of LCDR1, LCDR2, and LCDR3 of SEQ ID NOS:115-117, respectively, and optionally a light chain variable region having the sequence of SEQ ID NO:114; or (3) sequences of LCDR1, LCDR2, and LCDR3 of SEQ ID NOS:119-121, respectively, and optionally a light chain variable region having the sequence of SEQ ID NO:118; or (4) sequences of LCDR1, LCDR2, and LCDR3 of SEQ ID NOS:123-125, respectively, and optionally a light chain variable region having the sequence of SEQ ID NO:122; or (5) sequences of LCDR1, LCDR2, and LCDR3 of SEQ ID NOS:127-129, respectively, and optionally a light chain variable region having the sequence of SEQ ID NO:126.

20. The immune cell of any one of claims 1 to 7, wherein the TCR specifically binds to a complex comprising a NUP98, GPD2, CASP8, KRAS, SKIV2L, H3F3B, RAD21, or PRAME peptide and an MHC class I protein.

21. The immune cell of any one of claims 1 to 7 and 20, wherein the CSR specifically binds to ROR2, optionally wherein the CSR comprises: (1) sequences of HCDR1, HCDR2, and HCDR3 of SEQ ID NOS:91-93, respectively, and optionally a heavy chain variable region having the sequence of SEQ ID NO:90; or (2) sequences of HCDR1, HCDR2, and HCDR3 of SEQ ID NOS:95-97, respectively, and optionally a heavy chain variable region having the sequence of SEQ ID NO:94; or (3) sequences of HCDR1, HCDR2, and HCDR3 of SEQ ID NOS:99-101, respectively, and optionally a heavy chain variable region having the sequence of SEQ ID NO:98; or (4) sequences of HCDR1, HCDR2, and HCDR3 of SEQ ID NOS:103-105, respectively, and optionally a heavy chain variable region having the sequence of SEQ ID NO:102; or (5) sequences of HCDR1, HCDR2, and HCDR3 of SEQ ID NOS:107-109, respectively, and optionally a heavy chain variable region having the sequence of SEQ ID NO:106; and/or the CSR comprises: (1) sequences of LCDR1, LCDR2, and LCDR3 of SEQ ID NOS:111-113, respectively, and optionally a light chain variable region having the sequence of SEQ ID NO:110; or (2) sequences of LCDR1, LCDR2, and LCDR3 of SEQ ID NOS:115-117, respectively, and optionally a light chain variable region having the sequence of SEQ ID NO:114; or (3) sequences of LCDR1, LCDR2, and LCDR3 of SEQ ID NOS:119-121, respectively, and optionally a light chain variable region having the sequence of SEQ ID NO:118; or (4) sequences of LCDR1, LCDR2, and LCDR3 of SEQ ID NOS:123-125, respectively, and optionally a light chain variable region having the sequence of SEQ ID NO:122; or (5) sequences of LCDR1, LCDR2, and LCDR3 of SEQ ID NOS:127-129, respectively, and optionally a light chain variable region having the sequence of SEQ ID NO:126.

22. The immune cell of any one of claims 1 to 7, wherein the TCR specifically binds to a complex comprising a SLC3A2, KIAA0368, CADPS2, CTSB, PRAME, p53, or PSA peptide and an MHC class I protein.

23. The immune cell of any one of claims 1 to 7 and 22, wherein the CSR specifically binds to HER2, EpCAM, or ROR1.

24. The immune cell of claim 22 or 23, wherein the TCR comprises a sequence of any one of SEQ ID NOS:20-25, optionally wherein:
the CSR binds to HER2 and comprises sequences of HCDR1, HCDR2, and HCDR3 of SEQ ID NOS:389-391, respectively, and optionally a heavy chain variable region having the sequence of SEQ ID NO:41; and/or the CSR binds to HER2 and comprises sequences of LCDR1, LCDR2, and LCDR3 of SEQ ID NOS:392-394, respectively, and optionally a light chain variable region having the sequence of SEQ ID NO:42; or
the CSR specifically binds to EpCAM and comprises sequences of HCDR1, HCDR2, and HCDR3 of SEQ ID NOS:403-405, respectively, and optionally a heavy chain variable region having the sequence of SEQ ID NO:60; and/or the CSR binds to EpCAM and comprises sequences of LCDR1, LCDR2, and LCDR3 of SEQ ID NOS:406-408, respectively, and optionally a light chain variable region having the sequence of SEQ ID NO:61; or
the CSR binds to ROR1 and comprises sequences of HCDR1, HCDR2, and HCDR3 of SEQ ID NOS:447-449, respectively, and optionally a heavy chain variable region having the sequence of SEQ ID NO:450; and/or sequences of LCDR1, LCDR2, and LCDR3 of SEQ ID NOS:451-453, respectively, and optionally a light chain variable region having the sequence of SEQ ID NO:454; and/or optionally an scFv having the sequence of SEQ ID NO:441.

25. The immune cell of claim 22 or 23, optionally wherein the TCR comprises a sequence of any one of SEQ ID NOS:20-25, and further optionally, wherein the CSR comprises sequences of HCDR1, HCDR2, and HCDR3 of SEQ ID NOS:455-457, respectively, and optionally a heavy chain variable region having the sequence of SEQ ID NO:458; and/or sequences of LCDR1, LCDR2, and LCDR3 of SEQ ID NOS:459-461, respectively, and optionally a light chain variable region having the sequence of SEQ ID NO:462; and/or optionally an scFv having the sequence of SEQ ID NO:442.

26. The immune cell of claim 25, wherein the CSR specifically binds to a ROR1 peptide having a sequence of any one of SEQ ID NOS:443-446.

27. The immune cell of any one of claims 1 to 7, wherein the TCR specifically binds to a complex comprising a WT1, NY-ESO-1, p53, DPY19L4, or RNF19B peptide and an MHC class I protein.

28. The immune cell of any one of claims 1 to 7 and 27, wherein the CSR specifically binds to MUC1, MUC16, FRα, or ROR1.

29. The immune cell of claim 27 or 28, wherein the TCR specifically binds to a complex comprising NY-ESO-1 and the MHC claims I protein and comprises: (1) sequences of CDR1, CDR2, and CDR3 of SEQ ID NOS:359-361, respectively, and optionally a variable region having the sequence of SEQ ID NO:362, and further optionally the sequence of SEQ ID NO:4; or (2) sequences of CDR1, CDR2, and CDR3 of SEQ ID NOS:363-365, respectively, and optionally a variable region having the sequence of SEQ ID NO:366, and further optionally the sequence of SEQ ID NO:5.

30. The immune cell of any one of claims 27 to 29, wherein the CSR:
specifically binds to MUC1 and comprises sequences of HCDR1, HCDR2, and HCDR3 of SEQ ID NOS:417-419, respectively, and optionally a heavy chain variable region having the sequence of SEQ ID NO:367; and/or the CSR specifically binds to MUC1 and comprises sequences of LCDR1, LCDR2, and LCDR3 of SEQ ID NOS:420-422, respectively, and optionally a light chain variable region having the sequence of SEQ ID NO:368; or
specifically binds to MUC16 and comprises: (1) sequences of HCDR1, HCDR2, and HCDR3 of SEQ ID NOS:131-133, respectively, and optionally a heavy chain variable region having the sequence of SEQ ID NO:130; or (2) sequences of HCDR1, HCDR2, and HCDR3 of SEQ ID NOS:135-137, respectively, and optionally a heavy chain variable region having the sequence of SEQ ID NO:134; (3) sequences of HCDR1, HCDR2, and HCDR3 of SEQ ID NOS:429-431, respectively, and optionally a heavy chain variable region having the sequence of any one of SEQ ID NOS:146-147; or (4) sequences of HCDR1, HCDR2, and HCDR3 of SEQ ID NOS:435-437, respectively, and optionally a heavy chain variable region having the sequence of any one of SEQ ID NOS:148-149; and/or comprises; (1) sequences of LCDR1, LCDR2, and LCDR3 of SEQ ID NOS:139-141, respectively, and optionally a light chain variable region having the sequence of SEQ ID NO:138; or (2) sequences of LCDR1, LCDR2, and LCDR3 of SEQ ID NOS:143-145, respectively, and optionally a light chain variable region having the sequence of SEQ ID NO:142; (3) sequences of LCDR1, LCDR2, and LCDR3 of SEQ ID NOS:432-434, respectively, and optionally a light chain variable region having the sequence of any one of SEQ ID NOS:150-151; or (4) sequences of LCDR1, LCDR2, and LCDR3 of SEQ ID NOS:438-440, respectively, and optionally a light chain variable region having the sequence of any one of SEQ ID NOS:152-153.

31. The immune cell of any one of claims 27 to 29, wherein the CSR:
specifically binds to FRα and comprises sequences of HCDR1, HCDR2, and HCDR3 of SEQ ID NOS:423-425, respectively, and optionally a heavy chain variable region having the sequence of SEQ ID NO:369 and further optionally a heavy chain having the sequence of SEQ ID NO:370; and/or comprises sequences of LCDR1, LCDR2, and LCDR3 of SEQ ID NOS:426-428, respectively, and optionally a light chain variable region having the sequence of SEQ ID NO:371 and further optionally a light chain having the sequence of SEQ ID NO:372; or
specifically binds to ROR1 and comprises sequences of HCDR1, HCDR2, and HCDR3 of SEQ ID NOS:447-449, respectively, and optionally a heavy chain variable region having the sequence of SEQ ID NO:450; and/or sequences of LCDR1, LCDR2, and LCDR3 of SEQ ID NOS:451-453, respectively, and optionally a light chain variable region having the sequence of SEQ ID NO:454; and/or optionally an scFv having the sequence of SEQ ID NO:441; or
comprises sequences of HCDR1, HCDR2, and HCDR3 of SEQ ID NOS:455-457, respectively, and optionally a heavy chain variable region having the sequence of SEQ ID NO:458; and/or sequences of LCDR1, LCDR2, and LCDR3 of SEQ ID NOS:459-461, respectively, and optionally a light chain variable region having the sequence of SEQ ID NO:462; and/or optionally an scFv having the sequence of SEQ ID NO:442.

32. The immune cell of claim 31, wherein the CSR specifically binds to a ROR1 peptide having a sequence of any one of SEQ ID NOS:443-446.

33. The immune cell of any one of claims 1 to 7, wherein the TCR
specifically binds to a complex comprising a WT1 peptide and an MHC class I protein; optionally wherein the CSR specifically binds to MUC1; or
specifically binds to a complex comprising a p53 or KRAS peptide and an MHC class I protein, optionally wherein the CSR specifically binds to EGFR; and further optionally wherein the CSR comprises sequences of HCDR1, HCDR2, and HCDR3 of SEQ ID NOS:79-81, respectively, and optionally a heavy chain variable region having the sequence of SEQ ID NO:78; and or sequences of LCDR1, LCDR2, and LCDR3 of SEQ ID NOS:83-85, respectively, and optionally a light chain variable region having the sequence of SEQ ID NO:82.

34. The immune cell of any one of claims 1 to 7, wherein the TCR specifically binds to a complex comprising a ARHGAP35 or Histone H3.3 peptide and an MHC class I protein.

35. The immune cell of any one of claims 1 to 7 and 34, wherein the CSR specifically binds to EGFR or EGFRvIII, optionally, wherein the CSR
specifically binds to EGFR and comprises sequences of HCDR1, HCDR2, and HCDR3 of SEQ ID NOS:79-81, respectively, and optionally a heavy chain variable region having the sequence of SEQ ID NO:78; and/or comprises sequences of LCDR1, LCDR2, and LCDR3 of SEQ ID NOS:83-85, respectively, and optionally a light chain variable region having the sequence of SEQ ID NO:82; or
specifically binds to EGFRvIII and comprises sequences of HCDR1, HCDR2, and HCDR3 of SEQ ID NOS:409-411, respectively, and optionally a heavy chain variable region having the sequence of SEQ ID NO:412; and/or comprises sequences of LCDR1, LCDR2, and LCDR3 of SEQ ID NOS:413-415, respectively, and optionally a light chain variable region having the sequence of SEQ ID NO:416 or specifically binds to EGFRvIII and comprises comprises the sequence of SEQ ID NO:86.

36. The immune cell of any one of claims 1 to 7, wherein the TCR specifically binds to a complex comprising a KRAS, HER2, NY-ESO-1, or p53 peptide and an MHC class I protein.

37. The immune cell of any one of claims 1 to 7 and 36, wherein the CSR specifically binds to HER3, DLL3, c-Met, or ROR1.

38. The immune cell of claim 36 or 37, wherein the TCR specifically binds to a complex comprising NY-ESO-1 and the MHC claims I protein and comprises: (1) sequences of CDR1, CDR2, and CDR3 of SEQ ID NOS:359-361, respectively, and optionally a variable region having the sequence of SEQ ID NO:362, and further optionally the sequence of SEQ ID NO:4; or (2) sequences of CDR1, CDR2, and CDR3 of SEQ ID NOS:363-365, respectively, and optionally a variable region having the sequence of SEQ ID NO:366, and further optionally the sequence of SEQ ID NO:5, optionally wherein the CSR
specifically binds to HER3 and comprises sequences of HCDR1, HCDR2, and HCDR3 of SEQ ID NOS:395-397, respectively, and optionally a heavy chain having the sequence of SEQ ID NO:398; and/or comprises sequences of LCDR1, LCDR2, and LCDR3 of SEQ ID NOS:399-401, respectively, and optionally a light chain having the sequence of SEQ ID NO:402; or specifically binds to HER3 and comprises the sequence of SEQ ID NO:43.

39. The immune cell of any one of claims 36 to 38, wherein:
(1) the CSR specifically binds to DLL3 and comprises sequences of HCDR1, HCDR2, and HCDR3 of SEQ ID NOS:45-47, respectively, and optionally a heavy chain having the sequence of SEQ ID NO:44; and/or comprises sequences of LCDR1, LCDR2, and LCDR3 of SEQ ID NOS:49-51, respectively, and optionally a light chain having the sequence of SEQ ID NO:48;
(2) the CSR binds to ROR1 and comprises sequences of HCDR1, HCDR2, and HCDR3 of SEQ ID NOS:447-449, respectively, and optionally a heavy chain variable region having the sequence of SEQ ID NO:450; and/or sequences of LCDR1, LCDR2, and LCDR3 of SEQ ID NOS:451-453, respectively, and optionally a light chain variable region having the sequence of SEQ ID NO:454; and/or optionally an scFv having the sequence of SEQ ID NO:441;
(3) the CSR comprises sequences of HCDR1, HCDR2, and HCDR3 of SEQ ID NOS:455-457, respectively, and optionally a heavy chain variable region having the sequence of SEQ ID NO:458; and/or sequences of LCDR1, LCDR2, and LCDR3 of SEQ ID NOS:459-461, respectively, and optionally a light chain variable region having the sequence of SEQ ID NO:462; and/or optionally an scFv having the sequence of SEQ ID NO:442; or
(4) the CSR specifically binds to a ROR1 peptide having a sequence of any one of SEQ ID NOS:443-446.

40. The immune cell of any one of claims 1 to 7, wherein the TCR specifically binds to a complex comprising a 5T4 or PRAME peptide and an MHC class I protein.

41. The immune cell of any one of claims 1 to 7 and 40, wherein the CSR specifically binds to ROR2, CD70, or MCT4.

42. The immune cell of claim 40 or 41, wherein the CSR:
(a) specifically binds to ROR2 and comprises: (1) sequences of HCDR1, HCDR2, and HCDR3 of SEQ ID NOS:91-93, respectively, and optionally a heavy chain variable region having the sequence of SEQ ID NO:90; or (2) sequences of HCDR1, HCDR2, and HCDR3 of SEQ ID NOS:95-97, respectively, and optionally a heavy chain variable region having the sequence of SEQ ID NO:94; or (3) sequences of HCDR1, HCDR2, and HCDR3 of SEQ ID NOS:99-101, respectively, and optionally a heavy chain variable region having the sequence of SEQ ID NO:98; or (4) sequences of HCDR1, HCDR2, and HCDR3 of SEQ ID NOS:103-105, respectively, and optionally a heavy chain variable region having the sequence of SEQ ID NO:102; or (5) sequences of HCDR1, HCDR2, and HCDR3 of SEQ ID NOS:107-109; respectively, and optionally a heavy chain variable region having the sequence of SEQ ID NO:106; and/or comprises: (1) sequences of LCDR1, LCDR2, and LCDR3 of SEQ ID NOS: 111-113, respectively, and optionally a light chain variable region having the sequence of SEQ ID NO:110; or (2) sequences of LCDR1, LCDR2, and LCDR3 of SEQ ID NOS:115-117, respectively, and optionally a light chain variable region having the sequence of SEQ ID NO:114; or (3) sequences of LCDR1, LCDR2, and LCDR3 of SEQ ID NOS: 119-121, respectively, and optionally a light chain variable region having the sequence of SEQ ID NO:118; or (4) sequences of LCDR1, LCDR2, and LCDR3 of SEQ ID NOS:123-125, respectively, and optionally a light chain variable region having the sequence of SEQ ID NO:122; or (5) sequences of LCDR1, LCDR2, and LCDR3 of SEQ ID NOS:127-129, respectively, and optionally a light chain variable region having the sequence of SEQ ID NO:126; or
(b) specifically binds to CD70 and comprises sequences of HCDR1, HCDR2, and HCDR3 of SEQ ID NOS:63-65, respectively, and optionally a heavy chain having the sequence of SEQ ID NO:62; and/or comprises sequences of LCDR1, LCDR2, and LCDR3 of SEQ ID NOS:67-69, respectively, and optionally a light chain having the sequence of SEQ ID NO:66; or
(c) specifically binds to MCT4 and comprises: (1) sequences of HCDR1, HCDR2, and HCDR3 of SEQ ID NOS:155-157, respectively, and optionally a heavy chain having the sequence of SEQ ID NO:154; or (2) sequences of HCDR1, HCDR2, and HCDR3 of SEQ ID NOS:159-161, respectively, and optionally a heavy chain having the sequence of SEQ ID NO:158; or (3) sequences of HCDR1, HCDR2, and HCDR3 of SEQ ID NOS:163-165, respectively, and optionally a heavy chain having the sequence of SEQ ID NO:162; and/or (1) sequences of LCDR1, LCDR2, and LCDR3 of SEQ ID NOS:167-169, respectively, and optionally a light chain having the sequence of SEQ ID NO:166; or (2) sequences of LCDR1, LCDR2, and LCDR3 of SEQ ID NOS:171-173, respectively, and optionally a light chain having the sequence of SEQ ID NO:170; or (3) sequences of LCDR1, LCDR2, and LCDR3 of SEQ ID NOS:175-177, respectively, and optionally a light chain having the sequence of SEQ ID NO:174.

43. The immune cell of any one of claims 1 to 7, wherein the TCR specifically binds to a complex comprising a MAGE-A4 peptide and an MHC class I protein.

44. The immune cell of any one of claims 1 to 7 and claim 43, wherein the CSR specifically binds to MSLN, MUC16, EGFR, or RORA.

45. The immune cell of any one of claims 1 to 44, wherein the CSR transmembrane domain is derived from the transmembrane domain of a TCR co-receptor or a T cell costimulatory molecule, optionally wherein the TCR co-receptor or T cell costimulatory molecule is selected from the group consisting of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, and CD154.

46. The immune cell of claim 45, wherein the TCR co-receptor or T cell costimulatory molecule is CD30, CD28, or CD8.

47. The immune cell of any one of claims 1 to 46, wherein the CSR transmembrane domain is the transmembrane domain of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, or CD154.

48. The immune cell of claim 47, wherein the CSR transmembrane domain is the transmembrane domain of CD30, CD28, or CD8.

49. The immune cell of any one of claims 1 to 48, wherein the CSR transmembrane domain comprises an amino acid sequence selected from the group consisting of SEQ ID NOS:66-71; and/or the CSR lacks a functional primary signaling domain derived from the intracellular signaling sequence of a molecule selected from the group consisting of FcRγ, FcRβ, CD3γ, CD3δ, CD3ε, CD5, CD22, CD79a, CD79b, and CD66d; and/or further comprises a peptide linker between the ligand-binding module and the transmembrane domain of the CSR; and or a peptide linker between the transmembrane domain and the CD30 costimulatory domain of the CSR.

50. The immune cell of any one of claims 1 to 49, wherein the expression of the CSR is inducible, optionally wherein the expression of the CSR is inducible upon activation of the immune cell.

51. The immune cell of any one of claims 1 to 50, wherein the immune cell is selected from the group consisting of a cytotoxic T cell, a helper T cell, a natural killer T cell, a tumor infiltrating T cell (TIL T cell), and a suppressor T cell.

52. One or more nucleic acids encoding the TCR and CSR comprised by the immune cell of any one of claims 1 to 51.

53. One or more vectors comprising the one or more nucleic acids of claim 52.

54. A pharmaceutical composition comprising: (a) the immune cell of any one of claims 1 to 51, the nucleic acid(s) of claim 52, or the vector(s) of claim 53, and (b) a pharmaceutically acceptable carrier or diluent.

55. A method of killing target cells, comprising:
contacting one or more target cells with the immune cell of any one of claims 1 to 51 under conditions and for a time sufficient so that the immune cells mediate killing of the target cells,
wherein the target cells express an antigen specific to the immune cell, and
wherein the immune cell does not express a cell exhaustion marker upon contacting the target cells; and optionally,

56. The method of claim 55, wherein the immune cell is capable of developing into a population of immune cells that have a low percentage of cells expressing the cell exhaustion marker upon contacting the target cells, optionally wherein the immune cell is capable of developing into a population of immune cells that have a lower percentage of cells expressing the cell exhaustion marker compared to a population of immune cells that develops from a corresponding immune cell expressing a CSR comprising a CD28, 4-1BB, or DAP10 costimulatory domain, optionally wherein the ratio of the exhaustion marker expression level of the immune cell to the corresponding CD28 CSR immune cell is 0.9, 0.8, 0.7, 0.6, 0.5, 0.4, 0.3, 0.2, 0.1 or lower; and further optionally, wherein the exhaustion marker is selected from the group consisting of PD-1, TIM-3, TIGIT, and LAG-3; and/or the immune cell is a T cell.

57. A method of killing target cells, comprising:
contacting one or more target cells with the immune cell of any one of claims 1 to 51 under conditions and for a time sufficient so that the immune cells mediate killing of the target cells,
wherein the target cells express an antigen specific to the immune cell, and
wherein the immune cell expresses a low cell exhaustion level upon contacting the target cells, optionally wherein the immune cell is a T cell.

58. The method of claim 57, wherein:
(1) the immune cell expresses a lower level of PD-1, TIM-3, TIGIT, or LAG-3 than corresponding immune cell expressing a CSR comprising a CD28 costimulatory domain, optionally wherein the ratio of PD-1 expression level of the immune cell to the corresponding CD28 CSR immune cell is 0.9, 0.8, 0.7, 0.6, 0.5, 0.4, 0.3, 0.2, 0.1 or lower; the ratio of TIM-3 expression level of the immune cell to the corresponding CD28 CSR immune cell is 0.9, 0.8, 0.7, 0.6, 0.5, 0.4, 0.3, 0.2, 0.1 or lower; the ratio of LAG-3 expression level of the immune cell to the corresponding CD28 CSR immune cell is 0.9, 0.8, 0.7, 0.6, 0.5, 0.4, 0.3, 0.2, 0.1 or lower; and/or the ratio of TIGIT expression level of the immune cell to the corresponding CD28 CSR immune cell is 0.9, 0.8, 0.7, 0.6, 0.5, 0.4, 0.3, 0.2, 0.1 or lower;
(2) the immune cell expresses a lower level of PD-1, TIM-3, TIGIT, or LAG-3 than corresponding immune cell expressing a CSR comprising a 4-1BB costimulatory domain, optionally wherein the ratio of PD-1 expression level of the immune cell to the corresponding 4-1BB CSR immune cell is 0.9, 0.8, 0.7, 0.6, 0.5, 0.4, 0.3, 0.2, 0.1 or lower; the ratio of TIM-3 expression level of the immune cell to the corresponding 4-1BB CSR immune cell is 0.9, 0.8, 0.7, 0.6, 0.5, 0.4, 0.3, 0.2, 0.1 or lower; the ratio of LAG-3 expression level of the immune cell to the corresponding 4-1BB CSR immune cell is 0.9, 0.8, 0.7, 0.6, 0.5, 0.4, 0.3, 0.2, 0.1 or lower; and/or the ratio of TIGIT expression level of the immune cell to the corresponding 4-1BB CSR immune cell is 0.9, 0.8, 0.7, 0.6, 0.5, 0.4, 0.3, 0.2, 0.1 or lower;
(3) the immune cell expresses a lower level of PD-1, TIM-3, TIGIT, or LAG-3 than corresponding immune cell expressing a CSR comprising a DAP10 costimulatory domain, optionally wherein the ratio of PD-1 expression level of the immune cell to the corresponding DAP10 CSR immune cell is 0.9, 0.8, 0.7, 0.6, 0.5, 0.4, 0.3, 0.2, 0.1 or lower; the ratio of TIM-3 expression level of the immune cell to the corresponding DAP10 CSR immune cell is 0.9, 0.8, 0.7, 0.6, 0.5, 0.4, 0.3, 0.2, 0.1 or lower; the ratio of LAG-3 expression level of the immune cell to the corresponding DAP10 CSR immune cell is 0.9, 0.8, 0.7, 0.6, 0.5, 0.4, 0.3, 0.2, 0.1 or lower; and/or the ratio of TIGIT expression level of the immune cell to the corresponding DAP10 CSR immune cell is 0.9, 0.8, 0.7, 0.6, 0.5, 0.4, 0.3, 0.2, 0.1 or lower.

59. The method of any one of claims 57 and 58, wherein the target cells are cancer cells, optionally wherein the cancer cells are from a cancer selected from the group consisting of liver cancer, gastrointestinal cancer, bile duct cancer, renal cell carcinoma, adrenocortical carcinoma, bladder cancer, breast cancer, cervical cancer, cholangiocarcinoma, colorectal cancer, esophageal cancer, glioblastoma, glioma, hepatocellular carcinoma, head and neck cancer, kidney cancer, lung cancer, melanoma, mesothelioma, myeloma, pancreatic cancer, pheochromocytoma, plasmacytoma, neuroblastoma, ovarian cancer, prostate cancer, sarcoma, stomach cancer, uterine cancer, and thyroid cancer, for example, wherein the cancer cells are solid tumor cells.

60. A method of treating a disease, the method comprising a step of administering to a subject the immune cell of any one of claims 1 to 51, the nucleic acid(s) of claim 52, or the vector(s) of claim 53, or the pharmaceutical composition of claim 54 to the subject, optionally wherein the disease is cancer, and further optionally, wherein the cancer is a solid tumor cancer; and/or the subject has a higher density of the immune cell of any one of claims 1 to 51 in the solid tumor cancer than in the rest of the subject's body.

61. The method of claim 60, wherein administration of the immune cell results in a population of immune cells in the subject that arise from the immune cell, optionally wherein the population of immune cells arising from the immune cell in the subject is larger than a population of immune cells that can arise from administration of a corresponding immune cell expressing a CSR comprising a CD28 costimulatory domain, if the corresponding immune cell is administered to the same subject.

62. A method of treating a solid tumor cancer in a subject, the method comprising the steps of:
(a) transducing tumor infiltrating T cells (TIL T cells) obtained from the subject, or progenies of the TIL T cells, with a nucleic acid encoding, or a vector comprising a nucleic acid encoding, a chimeric stimulating receptor (CSR) comprising:
(i) a ligand-binding module that is capable of binding or interacting with a target ligand;
(ii) a transmembrane domain (a CSR transmembrane domain); and
(iii) a CD30 costimulatory domain,
wherein the CSR lacks a functional primary signaling domain; and
(b) administering to the subject transduced TIL T cells or progenies thereof.

63. The method of claim 62, wherein the ligand-binding module of the CSR comprises an antibody moiety (a CSR antibody moiety); optionally wherein the CD30 costimulatory domain comprises a sequence that is at least 80%, 85%, 90%, 95%, or 100% identical to residues 561-573 or 578-586 of SEQ ID NO:228; or the CD30 costimulatory domain comprises a sequence that is at least 70%, 75%, 80%, 85%, 90%, 95%, or 100% identical to the sequence of SEQ ID NO:238.

64. The method of any one of claims 62 and 63, wherein the target ligand is a cell surface antigen on a solid tumor, optionally wherein the cell surface antigen is Glypican 3 (GPC3), HER2/ERBB2, EpCAM, MUC16, folate receptor alpha (FRα), MUC1, EGFR, EGFRvIII, HER3, DLL3, c-Met, ROR2, CD70, MCT4, MSLN, PSMA, or a variant or mutant thereof.

65. The method of any one of claims 62 to 64, wherein the TIL T cells comprise an αβ TCR, optionally wherein the TCR specifically binds to a disease-related MHC-restricted antigen, and further optionally wherein the disease-related MHC-restricted antigen is expressed on cell surface of the solid tumor cancer; or the TCR does not specifically bind to a disease-related MHC-restricted antigen on cell surface of the solid tumor cancer.

66. The method of any one of claims 62 to 65, further comprising a step of obtaining TIL T cells from the subject prior to the transducing step, optionally wherein the subject has a higher density of the transduced TIL T cells in the solid tumor cancer than in the rest of the subject's body; and further optionally, wherein the cancer is selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer.

67. A method for preventing and/or reversing T cell exhaustion in a subject, comprising administering to the subject the nucleic acid(s) of claim 52, the vector(s) of claim 53, or the pharmaceutical composition of claim 54 comprising the nucleic acid(s) or the vector(s) to the subject, optionally wherein the method decreases the expression of an exhaustion marker in a T cell, optionally wherein the exhaustion marker is selected from the group consisting of PD-1, TIM-3, TIGIT, and LAG-3.

68. A method of treating a solid tumor cancer in a subject with increased tumor infiltration or immune cell expansion as compared to treating the same type of solid tumor cancer with immune cells expressing a TCR and a CSR comprising a control costimulatory domain, wherein the method comprises administering to the subject corresponding immune cells expressing the same TCR and a corresponding CSR comprising a CD30 costimulatory domain, and wherein the corresponding immune cells comprise the immune cell of any one of claims 1 to 51, optionally wherein the control costimulatory domain is a CD28, 4-1BB, or DAP10 costimulatory domain.

69. A method of treating a solid tumor cancer in a subject with increased tumor regression as compared to treating the same type of solid tumor cancer with immune cells expressing a TCR and a CSR comprising a CD28, 4-1BB, or DAP10 costimulatory domain, wherein the method comprises administering to the subject corresponding immune cells expressing the same TCR and a corresponding CSR comprising a CD30 costimulatory domain, and wherein the corresponding immune cells comprise the immune cell of any one of claims 1 to 51.

70. A method for generating central memory T cells in a subject, comprising administering to the subject the nucleic acid(s) of claim 52, the vector(s) of claim 53, or the pharmaceutical composition of claim 54 comprising the nucleic acid(s) or the vector(s) to the subject, optionally wherein the method increases the number of central memory T cells and/or the percentage of central memory T cells among all T cells in the subject.

71. A method for generating central memory T cells in vitro comprising: contacting one or more target cells with the immune cell of any one of claims 1 to 51 under conditions and for a time sufficient so that the immune cell develops into central memory T cells, wherein the target cells express an antigen specific to the immune cell, optionally wherein the method increases the number of central memory T cells and/or the percentage of central memory T cells among all T cells descended from the immune cell; and further optionally wherein the method generates higher number of central memory T cells and/or higher percentage of central memory T cells than corresponding immune cell expressing a CSR comprising a CD28, 4-1BB, or DAP10 costimulatory domain, optionally wherein the method generates at least 10%, 20%, 30%, 40%, 50%, 60%, 70%, 80%, 90%, 100%, 200%, 300%, 400%, or 500% higher number of central memory T cells and/or percentage of central memory T cells than corresponding immune cell expressing a CSR comprising a CD28 or DAP10 costimulatory domain; and further optionally, the central memory T cells express high levels of CCR7 and low levels of CD45RA; and/or the central memory T cells are CD8⁺ T cells.
